(19) United States
(12) Patent Application Publication
(10) Pub. No.: US 2025/0281611 A1
(43) Pub. Date: Sep. 11, 2025

(54) NOVEL CELL THERAPY SYSTEM

(71) Applicant: BIOSCEPTRE ( AUST ) PTY LTD, North Ryde (AU)

(72) Inventors: Patrick Schlegel, Sydney (AU); Julian Alexander Barden, Sydney (AU); Ziduo Li, Sydney (AU); Sile Fiona Yang, Sydney (AU); Alexander Joechner, Sydney (AU)

(21) Appl. No.: 18/281,045

(22) PCT Filed: Mar. 11, 2022
(86) PCT No.: PCT/AU22/50206

§ 371 Date: Sep. 8, 2023

(30) Foreign Application Priority Data
Mar. 11, 2021 (AU) 2021900708
Aug. 17, 2021 (AU) 2021902565
Sep. 1, 2021 (AU) 2021902830

(51) Int. Cl. A61K 40/31; A61K 40/11; A61K 40/42; A61P 35/00; C07K 14/705; C07K 16/28

(52) U.S. Cl. CPC A61K 40/31; A61K 40/11; A61K 40/4202; A61P 35/00; C07K 14/705; C07K 16/28; A61K 2239/13; A61K 2239/22; C07K 2317/55

(57) ABSTRACT

The present invention relates to therapeutics, compositions, kits and methods of treatment comprising:
(a) an immune cell or progenitor thereof, expressing a receptor comprising an antigen-recognition domain and a signalling domain, wherein the antigen-recognition domain binds to a tumour-specific antigen, preferably a dysfunctional P2X₇ receptor, expressed on a cell surface; and
(b) a bridging molecule comprising:
   (i) a targeting moiety that binds to a cell surface molecule on a target cell; and
   (ii) a tumour-specific epitope moiety, preferably a dysfunctional P2X₇ receptor epitope moiety, that is bound by the antigen recognition domain.

### FIELD OF THE INVENTION

The present invention relates to chimeric antigen receptors, effector cells expressing an antigen receptor and methods of using antigen receptors for the prevention and/or treatment of various conditions, including cancer.

### RELATED APPLICATIONS

This application is a U.S. national-stage application based on International Application No. PCT/AU2022/050206, filed Mar. 11, 2022, which claims priority from Australian provisional applications AU 2021900708, filed Mar. 11, 2021, AU 2021902565, filed Aug. 17, 2021, and AU 2021902830, filed Sep. 1, 2021, the contents of each of which are hereby incorporated by reference in their entirety.

### SEQUENCE LISTING

The instant application contains a Sequence Listing which has been submitted electronically in ASCII format and is hereby incorporated by reference in its entirety. Said ASCII copy, created on Feb. 15, 2024, is named FPB-00601_SL.txt and is 657,916 bytes in size.

### BACKGROUND OF THE INVENTION

Cancer immunotherapy is a rapidly growing field. The development of T cells expressing chimeric antigen receptors (CARs) has revolutionised adoptive cell therapies.

The potential of this approach has been demonstrated in clinical trials, wherein CAR T cells were infused into adult and paediatric patients with B-cell malignancies, neuroblastoma, and sarcoma. To date, over 500 clinical trials have emerged worldwide, designed at testing the efficacy of CAR T cells targeted to bind 64 different tumour associated antigens. Among these, three CD19-specific CAR T cell products have been approved for the treatment of acute lymphoblastic leukaemia (ALL), large B cell lymphoma and mantle cell lymphoma. To date, most of the success with CAR T therapies has been observed in the context of so-called “liquid” tumours, or where the CARs are directed to CD19, CD22 or the B cell maturation antigen (BCMA).

Several challenges remain in the clinical application of CAR T cell therapies. These include difficulties with achieving sufficient therapeutic responses in the context of solid tumours, which may be due to insufficient activation, expansion and persistence of CAR T cells and/or the immunosuppressive tumour microenvironment. In addition, the long-term clinical application of CAR T therapies can be negatively impacted by the selective pressure of mono-specific CAR T cells, antigen-negative escape variants, and antigen depletion. Moreover, low levels of target antigen expression in healthy tissues can result in severe “on-target, off-tumour” toxicities. Finally, cytokine release syndrome (CRS) and CAR T cell-related encephalopathy syndrome (CRES) are frequently observed side effects of CAR T cell therapy.

There is consequently a need for new and improved approaches to CAR, preferably CAR-T, therapies.

Reference to any prior art in the specification is not an acknowledgment or suggestion that this prior art forms part of the common general knowledge in any jurisdiction or that this prior art could reasonably be expected to be understood, regarded as relevant, and/or combined with other pieces of prior art by a skilled person in the art.

### SUMMARY OF THE INVENTION

In one aspect, the present invention provides a two component therapeutic comprising:
(a) an immune cell or progenitor thereof, expressing a receptor comprising an antigen-recognition domain and a signalling domain, wherein the antigen-recognition domain binds to a tumour-specific antigen expressed on a cell surface; and
(b) a bridging molecule comprising:
   (i) a targeting moiety that binds to a cell surface molecule on a target cell; and
   (ii) a tumour-specific antigen epitope moiety that is bound by the antigen recognition domain.

In any aspect, the tumour-specific antigen is an antigen expressed on a solid tumour or liquid tumour. In one embodiment, the tumour-specific antigen is any one of nfP2X₇, EGFRvIII or CLDN6.

In one aspect, the present invention provides a two component therapeutic comprising:
(a) an immune cell or progenitor thereof, expressing a receptor comprising an antigen-recognition domain and a signalling domain, wherein the antigen-recognition domain binds to a dysfunctional P2X₇ receptor expressed on a cell surface; and
(b) a bridging molecule comprising:
   (i) a targeting moiety that binds to a cell surface molecule on a target cell; and
   (ii) a dysfunctional P2X₇ receptor epitope moiety that is bound by the antigen recognition domain.

In another aspect, the present invention provides a composition comprising:
(a) an immune cell or progenitor thereof, expressing a receptor comprising an antigen-recognition domain and a signalling domain, wherein the antigen-recognition domain binds to a dysfunctional P2X₇ receptor expressed on a cell surface; and
(b) a bridging molecule comprising:
   (i) a targeting moiety that binds to a cell surface molecule on a target cell; and
   (ii) a dysfunctional P2X₇ receptor epitope moiety that is bound by the antigen recognition domain.

In another aspect, the present invention provides a kit comprising:
(a) an immune cell or progenitor thereof, expressing a receptor comprising an antigen-recognition domain and a signalling domain, wherein the antigen-recognition domain binds to a dysfunctional P2X₇ receptor expressed on a cell surface; and
(b) a bridging molecule comprising:
   (i) a targeting moiety that binds to a cell surface molecule on a target cell; and
   (ii) a dysfunctional P2X₇ receptor epitope moiety that is bound by the antigen recognition domain.

In any embodiment, the bridging molecule may be a polypeptide, or a polypeptide conjugated to a molecule with the function of a bridging molecule, e.g. a DNA aptamer. The polypeptide may be expressed by the immune cell or progenitor thereof. Alternatively, the therapeutic, composition or kit may comprise the polypeptide, or a nucleic acid encoding said polypeptide.

In a further aspect, the present invention provides an immune cell, or progenitor thereof comprising:
(i) a receptor comprising an antigen-recognition domain and a signalling domain, wherein the antigen-recognition domain binds a first tumour antigen, wherein the first tumour antigen is a tumour-specific antigen (preferably dysfunctional P2X₇ receptor), and
(ii) an inducible expression construct encoding a bridging molecule in the form of a fusion protein comprising (a) an antibody, or antigen binding fragment thereof, that binds a second tumour antigen, and (b) a peptide or a fragment of a tumour-specific antigen (preferably a dysfunctional P2X₇ receptor).

In another aspect, the present invention provides a bridging molecule comprising:
(i) a targeting moiety that binds to a cell surface molecule on a target cell; and
(ii) a tumour-specific antigen epitope moiety (preferably a dysfunctional P2X₇ receptor epitope moiety) that is recognised or capable of being bound by an antigen recognition domain of a receptor, optionally wherein the receptor is a chimeric antigen receptor.

The bridging molecule may be a polypeptide, for example a fusion or chimeric protein. In alternative embodiments, the bridging molecule may comprise polypeptides or peptides that are linked via linking molecules.

In another aspect, the present invention provides a nucleic acid comprising a nucleotide sequence encoding a bridging molecule as described herein. Preferably, the nucleic acid comprises a first nucleotide sequence encoding a targeting moiety and a second nucleotide sequence encoding a tumour-specific antigen epitope moiety. Preferably, the tumour-specific antigen epitope moiety is a dysfunctional P2X₇ receptor epitope moiety.

In another aspect, the present invention provides a vector or expression construct comprising a nucleic acid of the invention. In one embodiment, the vector or expression construct further comprises a nucleotide sequence encoding the receptor comprising an antigen-recognition domain and signalling domain, wherein the antigen-recognition domain recognises a dysfunctional P2X₇ receptor expressed on a cell surface, as described herein.

In another aspect, the present invention provides a method of treating a disorder in a subject, the method comprising administering to the subject:
(a) a cell expressing a receptor comprising an antigen-recognition domain and a signalling domain, wherein the antigen-recognition domain binds to a tumour-specific antigen (preferably, the tumour specific antigen is a dysfunctional P2X₇ receptor) expressed on a cell surface; and
(b) a bridging molecule comprising:
   (i) a targeting moiety that binds to a cell surface molecule on a target cell; and
   (ii) a tumour-specific antigen epitope moiety, preferably a dysfunctional P2X₇ receptor epitope moiety, that is bound by the antigen recognition domain,
   thereby treating the disorder in the subject.

In another aspect, the present invention provides a method of treating cancer in a subject, the method comprising administering:
(a) an immune cell expressing a receptor comprising an antigen-recognition domain and a signalling domain, wherein the antigen-recognition domain binds to a tumour-specific antigen, preferably a dysfunctional P2X₇ receptor, expressed on a cell surface; and
(b) a bridging molecule comprising:
   (i) a targeting moiety that binds to a cell surface molecule on a target cell; and
   (ii) a tumour-specific epitope moiety, preferably a dysfunctional P2X₇ receptor epitope moiety, that is bound by the antigen recognition domain.

In another aspect, the present invention provides a method of killing a target cell, the method including exposing the target cell to:
(a) an immune cell expressing a receptor comprising an antigen-recognition domain and a signalling domain, wherein the antigen-recognition domain binds to a tumour-specific antigen, preferably a dysfunctional P2X₇ receptor, expressed on a cell surface; and
(b) a bridging molecule comprising:
   (i) a targeting moiety that binds to a cell surface molecule on the target cell; and
   (ii) a tumour-specific antigen epitope moiety, preferably a dysfunctional P2X₇ receptor epitope moiety, that is bound by the antigen recognition domain,
   thereby killing the target cell.

The target cell may be a cancer cell, or a cell capable of presenting a peptide from an infectious agent on an MHC class receptor. The target cell may or may not express a tumour-specific antigen, for example a dysfunctional P2X₇ receptor.

In any aspect, the target cell may be any cell expressing a dysfunctional P2X₇ receptor, for example a cancer cell.

In any embodiment, 2 or more bridging molecules may be administered to a subject, each bridging molecule comprising a targeting moiety that binds to a different cell surface molecule on a target cell. For example, in the context of a method of treating cancer, each bridging molecule administered may comprise different targeting moieties and may therefore bind to a different tumour associated antigen present on the cancer cells. Such embodiments facilitate redirection of a single class of CAR T cell to multiple antigens present on tumour antigens (including at the same time) and therefore provide a multi-pronged approach for killing of cancer cells.

Accordingly, in any embodiment, the method of treating cancer comprises administering 2 or more bridging molecules, wherein each bridging molecule comprises targeting moieties for binding to different cell surface antigens on a target cell.

In further embodiments, the bridging molecules may bind to different epitopes on the same cell surface antigen expressed by the cancer cell. Accordingly, in further embodiments, the methods of the invention comprise treating cancer comprises administering 2 or more bridging molecules, wherein each bridging molecule comprises targeting moieties for binding to different epitopes on the same cell surface antigen on a target cell.

Further still, the invention provides for methods wherein bridging molecules for redirecting a CAR T cell to different cancer antigens, can be administered synchronously to a subject in need thereof. This allows for fine-tuning of the therapeutic approach, such that a CAR T cell may be directed to binding cancer cells via different antigens, at different times during the course of the patient's therapeutic regimen.

In further embodiments, a single bridging molecule may comprise more than one targeting moiety, such that a single molecule comprises targeting moieties for more than one cell surface molecule on a target cell.

Further still, a single bridging molecule may comprise more than one targeting moiety, such that a single molecule comprises targeting moieties for the same cell surface molecule on a target cell, but wherein the targeting moieties bind to different epitopes on the cell surface molecule.

In any embodiment of aspects directed to methods of treatment herein, the bridging molecule may be delivered via infusion to the subject or may be expressed by the immune cell expressing the chimeric antigen receptor. The bridging molecule may be a polypeptide, which is encoded in an inducible or a constitutive expression construct contained in the immune cell.

In any embodiment, the antigen-recognition domain binds to an epitope associated with an adenosine triphosphate (ATP)-binding site of the dysfunctional P2X₇ receptor. In some embodiments, the dysfunctional P2X₇ receptor has a reduced capacity to bind ATP at the ATP-binding site compared to an ATP-binding capacity of a functional P2X₇ receptor (e.g., a receptor having wild-type sequence and having a conformation or fold of an ATP-binding receptor). In some embodiments the dysfunctional P2X₇ receptor cannot bind ATP at the ATP-binding site.

In any embodiment, the dysfunctional P2X₇ receptor has a conformational change that renders the receptor dysfunctional. In some embodiments, the conformational change is a change of an amino acid from the trans-conformation to the cis-conformation. In some embodiments, the amino acid that has changed from a trans-conformation to a cis-conformation is proline at amino acid position 210 of the dysfunctional P2X₇ receptor.

In any embodiment, the antigen-recognition domain binds to an epitope that includes the proline at amino acid position 210 of the dysfunctional P2X₇ receptor. In some embodiments, the antigen-recognition domain binds to an epitope that includes one or more amino acid residues spanning from glycine at amino acid position 200 to cysteine at amino acid position 216, inclusive, of the dysfunctional P2X₇ receptor.

The antigen-recognition domain of the receptor can be any suitable molecule that can interact with and specifically binds to a dysfunctional P2X₇ receptor. However, in some embodiments, the antigen-recognition domain includes amino acid sequence homology to the amino acid sequence of an antibody, or a fragment thereof, which binds to the dysfunctional P2X₇ receptor. In some embodiments, the antigen-recognition domain includes amino acid sequence homology to the amino acid sequence of a fragment-antigen binding (Fab) portion of an antibody that binds to a dysfunctional P2X₇ receptor. In some embodiments, the antibody is a humanised antibody.

In any embodiment, the antigen-recognition domain includes amino acid sequence homology to the amino acid sequence of a single-chain variable fragment (scFv) or a multivalent scFv that binds to a dysfunctional P2X₇ receptor. In some embodiments, the multivalent scFv is a divalent or trivalent scFv.

In any embodiment, the antigen-recognition domain includes amino acid sequence homology to a single-antibody domain (sdAb) that binds to a dysfunctional P2X₇ receptor.

In any embodiment, the antigen-recognition domain includes a binding polypeptide that includes amino acid sequence homology to one or more complementarity determining regions (CDRs) of an antibody that binds to a dysfunctional P2X₇ receptor. In any embodiment, the binding polypeptide includes amino acid sequence homology to the CDR1, 2 and 3 domains of the V_{H} and/or V_{L} chain of an antibody that binds to a dysfunctional P2X₇ receptor. In preferred embodiments, the binding polypeptide comprises the amino acid sequence of the CDRs of the V_{H} and/or V_{L} chain of an antibody, or the amino acid sequence of the V_{H} and/or V_{L} chains of an antibody, or the amino acid sequence of an antibody or fragment thereof, wherein the antibody or fragment thereof comprises the amino acid sequences of any antibody described in PCT/AU2002/000061 or PCT/AU2002/001204 (or in any one of the corresponding U.S. Pat. No. 7,326,415, 7,888,473, 7,531,171, 8,080,635, 8,399,617, 8,709,425, 9,663,584, or 10,450,380), PCT/AU2007/001540 (or in corresponding U.S. Pat. No. 8,067,550), PCT/AU2007/001541 (or in corresponding US publication US 2010-0036101), PCT/AU2008/001364 (or in any one of the corresponding U.S. Pat. Nos. 8,440,186, 9,181,320, 9,944,701 or U.S. Pat. No. 10,597,451), PCT/AU2008/001365 (or in any one of the corresponding U.S. Pat. No. 8,293,491 or U.S. Pat. No. 8,658,385), PCT/AU2009/000869 (or in any one of the corresponding U.S. Pat. No. 8,597,643, 9,328,155 or 10,238,716), PCT/AU2010/001070 (or in any one of the corresponding publications WO/2011/020155, U.S. Pat. Nos. 9,127,059, 9,688,771, or U.S. Pat. No. 10,053,508), and PCT/AU2010/001741 (or in any one of the corresponding publications WO 2011/075789 or U.S. Pat. No. 8,835,609) the entire contents of which are hereby incorporated by reference. Preferably the antibody comprises the CDR amino acid sequences of 2-2-1 described in PCT/AU2010/001070 (or in any one of the corresponding U.S. Pat. Nos. 9,127,059, 9,688,771, or U.S. Pat. No. 10,053,508) or BPM09 described in PCT/AU2007/001541 (or in corresponding US publication US 2010-0036101) and produced by the hybridoma AB253 deposited with the European Collection of Cultures (ECACC) under Accession no. 06080101.

In some embodiments, the signalling domain includes a portion derived from an activation receptor. In some embodiments, the activation receptor is a member of the CD3 co-receptor complex or is an Fc receptor. In some embodiments, the portion derived from the CD3 co-receptor complex is CD3-ζ. In some embodiments, the portion derived from the Fc receptor is FcεRI or FcγRI.

In some embodiments, the signalling domain includes a portion derived from a co-stimulatory receptor. In some embodiments, the signalling domain includes a portion derived from an activation receptor and a portion derived from a co-stimulatory receptor.

In some embodiments, the co-stimulatory receptor is selected from the group consisting of CD27, CD28, CD30, CD40, DAP10, OX40, 4-1BB (CD137) and ICOS.

In any aspect, the cell expressing an antigen receptor may be an immune cell, for example an immune cell as described herein, or a cell that is capable of differentiating into an immune cell (e.g., a progenitor of an immune cell). A cell that is capable of differentiating into an immune cell (e.g. T cell that will express the dysfunctional P2X₇ CAR) may be a stem cell, multi-lineage progenitor cell or induced pluripotent stem cell.

In any embodiment of the aforementioned aspects, the receptor comprising an antigen-recognition domain and a signalling domain is a chimeric receptor antigen (CAR).

In any aspect, the targeting moiety that binds to a cell surface molecule on a target cell comprises or consists of a peptide or antibody or antibody fragment. Alternatively, the targeting moiety may comprise a ligand or binding partner for a protein or receptor present on the target cell surface.

The targeting moiety may further comprise a soluble T cell receptor (TcR) or a single chain T cell receptor binding motif or a T cell receptor-like mAb. In such embodiments, the targeting moiety is particularly suitable for the binding of peptides derived from intracellularly processed proteins from infectious agents that are presented on a cell surface via MHC (HLA) I and II molecules. The targeting moiety may also be suitable for binding of peptides presented by MHC molecules, wherein the peptides comprise mutations associated with cancers, such as the cancer testis antigens (WT1, NY-ESO-1, PRAME family (e.g. PRA100, PRA142, PRA300, PRA425 and others), MAGE family (e.g., MAGE-A1, MAGE-A3, MAGE-A4, MAGE-A12 and others), CT83, SSX2, GAGE, BAGE, PAGE) or other cancer specific mutations.

In any embodiment, the cell surface molecule may comprise an antigen, preferably an antigen as described herein.

The cell surface molecule may be selected from a protein, a lipid moiety, a glycoprotein, a glycolipid, a carbohydrate, a polysaccharide, a nucleic acid, an MHC-bound peptide, or a combination thereof.

The cell surface molecule may comprise parts (e.g., coats, capsules, cell walls, flagella, fimbrae, and toxins) of bacteria, viruses, and other microorganisms. The cell surface molecule may be expressed by the target cell.

The cell surface molecule may not be expressed by the target cell. By way of non-limiting example, the cell surface molecule may be a ligand expressed by a cell that is not the target cell and that is bound to the target cell or a cell surface molecule of the target cell. Also, by non-limiting example, the cell surface molecule may be a toxin, exogenous molecule or viral protein that is bound to a cell surface or cell surface receptor of the target cell.

In any aspect or embodiment, the targeting moiety of the bridging molecule does not bind to the same antigen or epitope as the antigen-recognition of the receptor.

For example, the targeting moiety of the bridging molecule does not bind to a dysfunctional P2X₇ receptor, the E200, E300, or E200/E300 composite epitope, or any other epitope present on a dysfunctional P2X₇ receptor as described herein.

The targeting moiety may be a targeting antibody or antibody fragment. The targeting antibody or antibody fragment may be an immunoglobulin (Ig). The immunoglobulin may be selected from an IgG, an IgA, an IgD, an IgE, an IgM, a fragment thereof or a modification thereof. The immunoglobulin may be IgG. The IgG may be IgG1. The IgG may be any IgG subclass.

In any embodiment, a bridging molecule of the invention may comprise more than one targeting moiety. For example, in certain non-limiting embodiments, the bridging molecule may comprise two different antibodies, or fragment thereof. The antibodies may bind different epitopes of the same cell surface molecule on the target cell. Alternatively, the antibodies may bind epitopes of different cell surface molecules on the target cell.

The dysfunctional P2X₇ receptor epitope moiety may be provided in the form of a P2X₇ receptor, or a fragment of a P2X₇ receptor that has at least one of the three ATP binding sites that are formed at the interface between adjacent correctly packed monomers that are unable to bind ATP. Such receptors are unable to extend the opening of the non-selective calcium channels to apoptotic pores.

In any aspect, the dysfunctional P2X₇ receptor epitope moiety comprises or consists of a fragment of a dysfunctional P2X₇ receptor. Exemplary fragments include GHNYTTRNILPGLNITC (SEQ ID NO: 2; also referred to herein as the “E200 epitope”) and variants thereof (exemplary variants are provided in SEQ ID NOs: 3 to 10 and 15 to 30 and 168); KYYKENNVEKRTLIKVF (SEQ ID NO: 12 and 13; also referred to herein as the “E300” epitope); or GHNYTTRNILPGAGAKYYKENNVEK (SEQ ID NO: 14; also referred to herein as the “E200/E300” or “composite” epitope). Further examples are provided in Table 1 herein.

In any aspect, the dysfunctional P2X₇ receptor epitope moiety is bound by an antibody that binds to dysfunctional P2X₇ receptors, but is not bound by antibodies that bind to functional P2X₇ receptors.

In any aspect, a bridging molecule may comprise 2 or more dysfunctional P2X₇ receptor epitope moieties. The 2 or more dysfunctional P2X₇ receptor epitope moieties may comprise or consist of the same sequence, or of different sequences. For example, in any aspect, a bridging molecule may comprise a dysfunctional P2X₇ receptor epitope moiety in the form of the E200 epitope and a further dysfunctional P2X₇ receptor epitope moiety in the form of the E300 epitope. Alternatively, in any aspect, a bridging molecule may comprise a dysfunctional P2X₇ receptor epitope moiety in the form of the E200 epitope and a further dysfunctional P2X₇ receptor epitope moiety in the form of the composite epitope. Still further, in any aspect, a bridging molecule may comprise a first dysfunctional P2X₇ receptor epitope moiety in the form of the E200 epitope and a further dysfunctional P2X₇ receptor epitope moiety in the form of the E200 epitope.

As used herein, except where the context requires otherwise, the term “comprise” and variations of the term, such as “comprising”, “comprises” and “comprised”, are not intended to exclude further additives, components, integers or steps.

Further aspects of the present invention and further embodiments of the aspects described in the preceding paragraphs will become apparent from the following description, given by way of example and with reference to the accompanying drawings.

### BRIEF DESCRIPTION OF THE DRAWINGS

FIG. 1: Diagrammatic representation of exemplary bridging molecules.

FIG. 2: Diagrammatic representation of exemplary embodiments of the present invention utilising nfP2X₇-CAR and Fab-based E200 bridging molecules targeting cancer, infection and immunomodulatory-related target antigens.

FIG. 3: Diagrammatic representation of exemplary embodiments of the present invention utilising nfP2X₇-CAR and single chain TCR-based E200 bridging molecules targeting cancer and infection-related peptides.

FIG. 4: Bridging molecules in Fab format with a single E200 epitope either directly linked to the V_{H} ((a) and (b)) or via a linker ((c) and (d)) binds to CD19 on JeKo-1 (mantle cell lymphoma) cell line and the E200 epitope is available for binding to an antibody (BIL03_2-2-1-AF647). HIS tag is detected by FITC antibody. (a) and (c) show anti-HIS antibody binding, (b) and (d) show binding of antibody to dysfunctional P2X₇ receptor epitope.

FIG. 5: Bridging molecules in scFv format with a single E200 epitope either directly linked to the V_{H} ((a) and (b)) or via a linker ((c) and (d)) binds to CD19 on JeKo-1 (mantle cell lymphoma) cell line and the E200 epitope is available for binding to an antibody. (a) and (c) show anti-HIS antibody binding, (b) and (d) show binding of antibody to dysfunctional P2X₇ receptor epitope.

FIG. 6: Bridging molecules in Fab format with a single E200 epitope either directly linked to the V_{L} ((a) and (b)) or via a linker ((c) and (d)) binds to CD19 on JeKo-1 (mantle cell lymphoma) cell line and the E200 epitope is available for binding to an antibody. (a) and (c) show anti-HIS antibody binding, (b) and (d) show binding of antibody to dysfunctional P2X₇ receptor epitope.

FIG. 7: Bridging molecules in scFv format with a single E200 epitope either directly linked to the V_{L} ((a) and (b)) or via a linker ((c) and (d)) binds to CD19 on JeKo-1 (mantle cell lymphoma) cell line and the E200 epitope is available for binding to an antibody. (a) and (c) show anti-HIS antibody binding, (b) and (d) show binding of antibody to dysfunctional P2X₇ receptor epitope.

FIG. 8: Binding of bridging molecules to various antigens CD37, CD79B, ROR1, CD33, CD38, CD123, CD135, BCMA, EGFR, PDL1, CD22, CD70 and CD20. (a), (c), (e), (g), (i), (k), (m), (o), (q), (s), (u), (w) and (y) show anti-HIS antibody binding, (b), (d), (f), (h), (j), (l), (n), (p), (r), (t), (v), (x) and (z) show binding of antibody to dysfunctional P2X₇ receptor epitope.

FIG. 9: “painting” of JeKo-1 cells with CD19 targeted Fab bridging molecules in the illustrated format as detected by flow cytometry. Cells were incubated at indicated concentrations with Fab bridging molecules. CD33 targeted Fab bridging molecules served as negative control in JeKo-1 at 10 ng/mL and 1000 ng/mL. CD19 targeted Fab bridging molecules were used at 1 ng/mL, 10 ng/mL, 100 ng/mL and 1000 ng/mL.

FIG. 10: “painting” of MOLM-13 cells with CD33 targeted Fab bridging molecules in the illustrated format as detected by flow cytometry. Cells were incubated at indicated concentrations with Fab bridging molecules. CD19 targeted Fab bridging molecules served as negative control in JeKo-1 at 10 ng/mL and 1000 ng/mL. CD33 targeted Fab bridging molecules were used at 1 ng/mL, 10 ng/mL, 100 ng/mL and 1000 ng/mL.

FIG. 11: Illustrates the “painting” of MOLM-13 (AML) cells via CD33 targeted Fab bridging molecules. The flow data shows in black the isotype control, in blue the staining with BILO3 2-2-1 sd-mAb only at 1 ug/mL and in green the increase of staining via the combination of CD33 targeted Fab bridging molecules and BILO3 2-2-1 sd-mAb. The increase of target molecules that can be recognised by the nfP2X₇ BRIDGE CAR expressing effector cells translate into CAR-mediated effector function. The use of the bridging molecules enhances the CAR function by increasing the targeting epitopes on the cancer cells.

FIG. 12: A representative flow cytometric plot with direct comparison of untransduced T cells (left panel), CAR0007_hPGK (middle panel) and CAR0007_EF1a (right panel) expressing T cells. Both CAR T cells containing conditions showed significantly increased expression of the activation markers CD25 and CD69 in incubation with MOLM-13 at 20:1 ET ratio and CD33 targeted Fab bridging molecules at 1000 ng/mL after 48 hours.

FIG. 13: The flow cytometric plots of T cells and MOLM-13 at 20:1 ET ratio and CD33 targeted Fab bridging molecules at 1000 ng/mL after 48 hours corresponding to FIG. 12 showed a complete clearance of leukaemic cells in the conditions containing CAR0007_hPGK and CAR0007_EF1a whereas there was no relevant impact on leukaemic cell number in the untransduced T cell condition.

FIG. 14: Flow cytometric plots that illustrate the dose dependent clearance of leukaemic cells at indicated CD33 targeted Fab bridging molecule concentrations of CAR0007_EF1a containing T cells and MOLM-13 at 20:1 ET ratio after 48 hours. As low as 40 ng/mL showed almost complete elimination of leukaemic cells and complete elimination of leukaemic cells at 200 and 1000 ng/mL.

FIG. 15: (a) Specific lysis of MOLM-13 leukaemic cells by CAR0007_hPGK T cells at an ET ratio of 20:1 after 48 hour incubation with and without EGFR and CD33 targeted bridging molecules at indicated concentrations is illustrated. Significant lysis in a dose dependent manner was found for increasing concentrations (40, 200 and 1000 ng/mL) of CD33 targeted Fab bridging molecules. (b) Specific lysis of MOLM-13 leukaemic cells by CAR0007_hEF1a T cells at an ET ratio of 20:1 after 48 hour incubation with and without EGFR and CD33 targeted bridging molecules at indicated concentrations is illustrated. Significant lysis in a dose dependent manner was found for increasing concentrations (40, 200 and 1000 ng/mL) of CD33 targeted Fab bridging molecules.

FIG. 16: A titration experiment of EGFR and CD33 targeted Fab-bridging molecules was used to test the impact on killing of MOLM-13 by CAR0007_hPGK.

There was a significant impact on the viability of MOLM-13 after 24 hour incubation at 10:1 ET ratio between the condition with EGFR targeted bridging molecules and CD33 targeted bridging molecules at 1000 ng/mL. Statistical analysis was performed by t-test.

FIG. 17: Alternative representation of the data from FIG. 16. There was no significant difference in the titration of the EGFR bridging molecules (data not shown). There were significant differences in the titration of the CD33 bridging molecules. Statistical analyses were performed by One-Way ANOVA and post-hoc test Tukey.

FIG. 18: A titration experiment of EGFR and CD33 targeted Fab-bridging molecules was used to test the impact on killing of MOLM-13 by CAR0007_hPGK. There was a significant impact on the viability of MOLM-13 after 24 hour incubation at 20:1 ET ratio between the condition with EGFR targeted bridging molecules and CD33 targeted bridging molecules at 200 ng/mL and 1000 ng/mL. Statistical analysis was performed by t-test.

FIG. 19: Alternative representation of the data from FIG. 18. There was no significant difference in the titration of the EGFR bridging molecules (data not shown). There were significant differences in the titration of the CD33 bridging molecules. Statistical analyses were performed by One-Way ANOVA and post-hoc test Tukey.

FIG. 20: A titration experiment of EGFR and CD33 targeted Fab-bridging molecules was used to test the impact on killing of MOLM-13 by CAR0007_hPGK. There was a significant impact on the viability of MOLM-13 after 48 hour incubation at 10:1 ET ratio between the condition with EGFR targeted bridging molecules and CD33 targeted bridging molecules at 200 ng/mL and 1000 ng/mL. Statistical analysis was performed by t-test.

FIG. 21: A titration experiment of EGFR and CD33 targeted Fab-bridging molecules was used to test the impact on killing of MOLM-13 by CAR0007_hPGK. There was a significant impact on the viability of MOLM-13 after 48 hour incubation at 20:1 ET ratio between the condition with EGFR targeted bridging molecules and CD33 targeted bridging molecules at 200 ng/mL and 1000 ng/mL. Statistical analysis was performed by t-test.

FIG. 22: A titration experiment of EGFR and CD33 targeted Fab-bridging molecules was used to test the impact on killing of MOLM-13 by CAR0007_EF1a. There was a significant impact on the viability of MOLM-13 after 24 hour incubation at 10:1 ET ratio between the condition with EGFR targeted bridging molecules and CD33 targeted bridging molecules at 200 ng/mL and 1000 ng/mL. Statistical analysis was performed by t-test.

FIG. 23: Alternative representation of the data from FIG. 22. There was no significant difference in the titration of the EGFR bridging molecules (data not shown). There were significant differences in the titration of the CD33 bridging molecules. Statistical analyses were performed by One-Way ANOVA and post-hoc test Tukey.

FIG. 24: A titration experiment of EGFR and CD33 targeted Fab-bridging molecules was used to test the impact on killing of MOLM-13 by CAR0007_EF1a. There was a significant impact on the viability of MOLM-13 after 24 hour incubation at 20:1 ET ratio between the condition with EGFR targeted bridging molecules and CD33 targeted bridging molecules at 40 ng/mL, 200 ng/mL and 1000 ng/mL. Statistical analysis was performed by t-test.

FIG. 25: Alternative representation of the data from FIG. 24. There was no significant difference in the titration of the EGFR bridging molecules (data not shown). There were significant differences in the titration of the CD33 bridging molecules. Statistical analyses were performed by One-Way ANOVA and post-hoc test Tukey.

FIG. 26: A titration experiment of EGFR and CD33 targeted Fab-bridging molecules was used to test the impact on killing of MOLM-13 by CAR0007_EF1a. There was a significant impact on the viability of MOLM-13 after 48 hour incubation at 10:1 ET ratio between the condition with EGFR targeted bridging molecules and CD33 targeted bridging molecules at 40 ng/mL, 200 ng/mL and 1000 ng/mL. Statistical analysis was performed by t-test.

FIG. 27: A titration experiment of EGFR and CD33 targeted Fab-bridging molecules was used to test the impact on killing of MOLM-13 by CAR0007_EF1a. There was a significant impact on the viability of MOLM-13 after 48 hour incubation at 20:1 ET ratio between the condition with EGFR targeted bridging molecules and CD33 targeted bridging molecules at 40 ng/mL, 200 ng/mL and 1000 ng/mL. Statistical analysis was performed by t-test.

FIG. 28: (a) A kill assay (cytotoxicity was measure by quantification of residual leukaemic cells compared with a control) the elimination of leukaemic cells at the EGFR bridging molecule concentrations 40, 200 and 1000 ng/mL showed no significant difference between untransduced T cells compared with CAR T cells. (b) The elimination of leukaemic cells at the CD33 bridging molecule concentrations 40, 200 and 1000 ng/mL showed a significant difference between untransduced T cells compared with both CAR0007 transduced T cells (hPGK and EF1a).

FIG. 29: A titration experiment of CD33 targeted Fab-bridging molecules was used to test the impact on killing of MOLM-13 by untransduced T cells, CAR0007_hPGK and CAR0007_EF1a effector cells at an ET ratio 10:1 after 48 hour incubation.

FIG. 30: Analysis of the results in FIG. 29. There was a consistent significant difference in the titration of the CD33 bridging molecules between the three effector cell populations at 40 ng/mL, 200 ng/mL and 1000 ng/mL. Statistical analyses were performed by One-Way ANOVA and post-hoc test Tukey to compare the named three conditions per concentration of EGFR bridging molecules.

FIG. 31: A titration experiment of CD33 targeted Fab-bridging molecules was used to test the impact on killing of MOLM-13 by untransduced T cells, CAR0007_hPGK and CAR0007_EF1a effector cells at an ET ratio 20:1 after 48 hour incubation

FIG. 32: Analysis of the results in FIG. 31. There was a consistent significant difference in the titration of the CD33 bridging molecules between the three effector cell populations at 8 ng/mL, 40 ng/mL, 200 ng/mL and 1000 ng/mL. Statistical analyses were performed by One-Way ANOVA and post-hoc test Tukey to compare the named three conditions per concentration of EGFR bridging molecules.

FIG. 33: Cell killing by nfP2X₇ CAR-T cells in the presence of CD19-targeting bridging molecules in Fab and IgG1 format and with different dysfunctional P2X₇ receptor epitope moieties. CAR10 versus JeKo-1 Effector cell/Target cell (ET) ratio 10:1. CAR10 to target 2.9:1. N=1 healthy donor. 6 replicates. CAR10 expression (d12)=28.6%. Indicated significance is the group versus the control (****) are all significantly different from the control.

FIG. 34: Cell killing by nfP2X₇ CAR-T cells in the presence of CD19-targeting bridging molecules with different dysfunctional P2X₇ receptor epitope moieties. CAR10 versus JeKo-1 Effector cell/Target cell (ET) ratio 20:1. N=1 healthy donor. 100 ng/mL Fab CD19 bridging molecules. 6 replicates. In all CAR conditions, CAR expressing cells were normalised to ET ratio 1.48:1. Indicated significance is the group versus the control (****) are all significantly different from the control.

FIG. 35: Cell killing by three different classes of nfP2X₇ CAR-T cells (i.e. having different CAR architectures), in the presence of CD19-targeting bridging molecules in Fab and IgG1 format. CAR7, CAR10 and CAR16 versus JeKo-1 Effector cell/Target cell (ET) ratio 10:1. CAR7 to target 3.3:1; CAR10 to target 2.8:1. CAR16 to target 2.2:1. N=1 healthy donor. 6 replicates. CAR7 expression (d12) 33.3%; CAR10 expression (d12)=28.6%; CAR16 expression (d12)=22.6%. Indicated significance is the group versus the control (****) are all significantly different from the control.

FIG. 36: Cell killing by nfP2X₇ CAR-T cells in the presence of A) CD19-targeting bridging molecules in Fab format. (killing of JeKo-1 cells) or b) CD33-targeting bridging molecule in Fab format (killing of MOLM-13 cells).

FIG. 37: Schematic of in vivo evaluation of bridging molecules.

FIG. 38: bioluminescence (total Flux, p/s) as an indicator of tumour burden in mice following inoculation of Jeko-1_LUC_eGFP cells. Mice received treatment with: Group 1=no treatment; Group 2=activated untransduced T cells; Group 3=bridging molecule (E200 epitope+anti-CD19 Fab); Group 4=activated untransduced T cells+bridging molecule; Group 5=3^{rd} generation anti-CD19 CAR T cells; Group 6=T cells expressing anti-nfP2X₇ receptor CAR 1; Group 7=CAR 1-T cells+bridging molecule; Group 8=T cells expressing nfP2X₇ receptor CAR 2; Group 9=CAR 2-T cells+bridging molecule.

TABLE 1
Sequence Information

| | | |
|---|---|---|
| | | SEQ |
| Description | Sequence | ID NO: |

Exemplary dysfunctional P2X₇ receptor epitope moiety sequences

| | | |
|---|---|---|
| Human P2X₇ | MPACCSCSDVFQYETNKVTRIQSMNYGTIKWFFHVIIFSYV | 1 |
| receptor | CFALVSDKLYQRKEPVISSVHTKVKGIAEVKEEIVENGVK | |
| | KLVHSVFDTADYTFPLQGNSFFVMTNFLKTEGQEQRLCPE | |
| | YPTRRTLCSSDRGCKKGWMDPQSKGIQTGRCVVYEGNQKT | |
| | CEVSAWCPIEAVEEAPRPALLNSAENFTVLIKNNIDFPGH | |
| | NYTTRNILPGLNITCTFHKTQNPQCPIFRLGDIFRETGDN | |
| | FSDVAIQGGIMGIEIYWDCNLDRWFHHCRPKYSFRRLDDK | |
| | TTNVSLYPGYNFRYAKYYKENNVEKRTLIKVFGIRFDILV | |
| | FGTGGKFDIIQLVVYIGSTLSYFGLAAVFIDFLIDTYSSN | |
| | CCRSHIYPWCKCCQPCVVNEYYYRKKCESIVEPKPTLKYV | |
| | SFVDESHIRMVNQQLLGRSLQDVKGQEVPRPAMDFTDLSR | |
| | LPLALHDTPPIPGQPEEIQLLRKEATPRSRDSPVWCQCGS | |
| | CLPSQLPESHRCLEELCCRKKPGACITTSELFRKLVLSRH | |
| | VLQFLLLYQEPLLALDVDSTNSRLRHCAYRCYATWRFGSQ | |
| | DMADFAILPSCCRWRIRKEFPKSEGQYSGFKSPY | |
| Exemplary E200 | GHNYTTRNILPGLNITC | 2 |
| epitope | | |
| Variant E200 | GHNYTTRNILPGLNIT | 3 |
| epitope peptide | | |
| (E200′) | | |
| E200 epitope Cys | GHNYTTRNILPGLNITS | 4 |
| to Ser | | |
| modification | | |
| Extended E200 | GHNYTTRNILPGLNITSTFHK | 5 |
| Cys to Ser | | |
| modification | | |
| Extended E200′ | GHNYTTRNILPGLNITSTFHKT | 6 |
| Cys to Ser | | |
| modification | | |
| (22 aa) | | |
| Extended E200″ | GHNYTTRNILPGLNITSTFHKTC | 7 |
| Cys to Ser | | |
| modification | | |
| Pep16 | DFPGHNYTTRNILPGC | 8 |
| Pep17 | GHNYTTRNILPGLNITSTFHKTS | 9 |
| Extended Pep17 | GHNYTTRNILPGLNITSTFHKTSGSGK | 10 |
| (27 aa) | | |
| Minimum | NYTTRNILPGL | 11 |
| sequence E200 | | |
| peptide (target | | |
| epitope) | | |
| Exemplary E300 | KYYKENNVEKRTLIK | 12 |
| epitope | | |
| Variant E300 | KYYKENNVEKRTLIKVF | 13 |
| epitope peptide | | |
| (E30′) | | |
| Exemplary | GHNYTTRNILPGAGAKYYKENNVEK | 14 |
| E200/E300 or | | |
| composite epitope | | |
| E200 + G4S | GHNYTTRNILPGLNITSGGGGS | 15 |
| linker | | |
| E200 + 2xG4S | GHNYTTRNILPGLNITSGGGGGGGGS | 16 |
| linker | | |
| E200 + 3xG4S | GHNYTTRNILPGLNITSGGGGSGGGGSGGGGS | 168 |
| linker | | |
| E200_extended | GHNYTTRNILPGLNITSTFHKTGS | 17 |
| peptide 17v3 (24 | | |
| aa) | | |
| E200_extended | GHNYTTRNILPGLNITSTFHGS | 18 |
| peptide 17v4 (22 | | |
| aa) | | |
| E200_extended | GHNYTTRNILPGLNITSGS | 19 |
| peptide 17v5 (19 | | |
| aa) | | |
| E200_extended | DFPGHNYTTRNILPGLNITSGS | 20 |
| peptide 17v6 (22 | | |
| aa) | | |
| (E200 epitope | | |
| extended into N | | |
| terminal region) | | |
| E200_extended | DFPGHNYTTRNILPGLNITSGGGGS | 21 |
| peptide 17v7 (25 | | |
| aa) + linker | | |
| E200_extended | DFPGHNYTTRNILPGLNITSGGGGSGGGGS | 22 |
| peptide 17v8 (30 | | |
| aa) + linker | | |
| E200_extended | DFPGHNYTTRNILPGLNITSGGGGSGGGGSGGGGS | 23 |
| peptide 17v9 (35 | | |
| aa) + linker | | |
| E200_extended | DFPGHNYTTRNILPGLNITSTFHKTSGSGK | 24 |
| peptide 17v10 (30 | | |
| aa) | | |
| E200_extended | DFPGHNYTTRNILPGLNITSTFHKTSGSGKGS | 25 |
| peptide 17v11 (32 | | |
| aa) + linker | | |
| E200_extended | DFPGHNYTTRNILPGLNITSTFHKTSGSGKGGGGS | 26 |
| peptide 17v12 (35 | | |
| aa) + linker | | |
| E200_extended | DFPGHNYTTRNILPGLNITSTFHGGGGS | 27 |
| peptide 17v13 (25 | | |
| aa) + linker | | |
| E200_extended | GHNYTTRNILPGLNITSTFHGGGGS | 28 |
| peptide 17v14 (22 | | |
| aa) + linker | | |
| E200_extended | DFPGHNYTTRNILPGLNITSTFHKTGGGGS | 29 |
| peptide 17v15 (30 | | |
| aa) + linker | | |
| E200_extended | GHNYTTRNILPGLNITSTFHKTGGGGS | 30 |
| peptide 17v16 (27 | | |
| aa) + linker | | |

Exemplary targeting moiety sequences and exemplary bridging
molecules Constructs based on FMC63 (for binding CD19)

| | | |
|---|---|---|
| CD19 binder | EVKLQESGPGLVAPSQSLSVTCTVSGVSLPDYGVSWIRQPPRKG | 31 |
| heavy chain | LEWLGVIWGSETTYYNSALKSRLTIIKDNSKSQVFLKMNSLQTDDT | |
| CD19, FMC63, | AIYYCAKHYYYGGSYAMDYWGQGTSVTVSSASTKGPSVFPLAPS | |
| B001_Heavy | SKSTSGGTAALGCLVKDYFPEPVTVSWNSGALTSGVHTFPAVLQ | |
| Chain | SSGLYSLSSVVTVPSSSLGTQTYICNVNHKPSNTKVDKKVEPKSC | |
| | HHHHHH | |
| CD19 binder light | DIQMTQTTSSLSASLGDRVTISCRASQDISKYLNWYQQKPDGTVK | 32 |
| chain | LLIYHTSRLHSGVPSRFSGSGSGTDYSLTISNLEQEDIATYFCQQG | |
| CD19, FMC63, | NTLPYTFGGGTKLEITKARTVAAPSVFIFPPSDEQLKSGTASVVCL | |
| B001_Light Chain | LNNFYPREAKVQWKVDNALQSGNSQESVTEQDSKDSTYSLSSTL | |
| | TLSKADYEKHKVYACEVTHQGLSSPVTKSFNRGEC | |
| E200 + CD19 | GHNYTTRNILPGLNITSEVKLQESGPGLVAPSQSLSVTCTVSGVSL | 33 |
| binder (nfP2X₇ | PDYGVSWIRQPPRKGLEWLGVIWGSETTYYNSALKSRLTIIKDNS | |
| epitope | KSQVFLKMNSLQTDDTAIYYCAKHYYYGGSYAMDYWGQGTSVT | |
| underlined) | VSSASTKGPSVFPLAPSSKSTSGGTAALGCLVKDYFPEPVTVSW | |
| CD19, FMC63, | NSGALTSGVHTFPAVLQSSGLYSLSSVVTVPSSSLGTQTYICNVN | |
| B002-1_Heavy | HKPSNTKVDKKVEPKSCHHHHHH | |
| Chain | | |
| E200 + CD19 | GHNYTTRNILPGLNITSGGGGSEVKLQESGPGLVAPSQSLSVTCT | 34 |
| binder (nfP2X₇ | VSGVSLPDYGVSWIRQPPRKGLEWLGVIWGSETTYYNSALKSRL | |
| epitope | TIIKDNSKSQVFLKMNSLQTDDTAIYYCAKHYYYGGSYAMDYWG | |
| underlined) | QGTSVTVSSASTKGPSVFPLAPSSKSTSGGTAALGCLVKDYFPEP | |
| CD19, FMC63, | VTVSWNSGALTSGVHTFPAVLQSSGLYSLSSVVTVPSSSLGTQT | |
| B002-2_Heavy | YICNVNHKPSNTKVDKKVEPKSCHHHHHH | |
| Chain | | |
| E200 + CD19 | GHNYTTRNILPGLNITSDIQMTQTTSSLSASLGDRVTISCRASQDIS | 35 |
| binder (nfP2X₇ | KYLNWYQQKPDGTVKLLIYHTSRLHSGVPSRFSGSGSGTDYSLTI | |
| epitope | SNLEQEDIATYFCQQGNTLPYTFGGGTKLEITKARTVAAPSVFIFP | |
| underlined) | PSDEQLKSGTASVVCLLNNFYPREAKVQWKVDNALQSGNSQES | |
| CD19, FMC63, | VTEQDSKDSTYSLSSTLTLSKADYEKHKVYACEVTHQGLSSPVTK | |
| Light chain B003- | SFNRGEC | |
| 1 | | |
| E200 + CD19 | GHNYTTRNILPGLNITSGGGGSDIQMTQTTSSLSASLGDRVTISCR | 36 |
| binder (nfP2X₇ | ASQDISKYLNWYQQKPDGTVKLLIYHTSRLHSGVPSRFSGSGSG | |
| epitope | TDYSLTISNLEQEDIATYFCQQGNTLPYTFGGGTKLEITKARTVAA | |
| underlined) | PSVFIFPPSDEQLKSGTASVVCLLNNFYPREAKVQWKVDNALQS | |
| CD19, FMC63, | GNSQESVTEQDSKDSTYSLSSTLTLSKADYEKHKVYACEVTHQG | |
| Light chain B003- | LSSPVTKSFNRGEC | |
| 2 | | |
| E200 + CD19 | EVKLQESGPGLVAPSQSLSVTCTVSGVSLPDYGVSWIRQPPRKG | 37 |
| binder (nfP2X₇ | LEWLGVIWGSETTYYNSALKSRLTIIKDNSKSQVFLKMNSLQTDDT | |
| epitope | AIYYCAKHYYYGGSYAMDYWGQGTSVTVSSASTKGPSVFPLAPS | |
| underlined) | | |
| CD19, FMC63, | SKSTSGGTAALGCLVKDYFPEPVTVSWNSGALTSGVHTFPAVLQ | |
| B005_Heavy | SSGLYSLSSVVTVPSSSLGTQTYICNVNHKPSNTKVDKKVEPKSC | |
| Chain | GHNYTTRNILPGLNITSHHHHHH | |
| CD19, FMC63, | DIQMTQTTSSLSASLGDRVTISCRASQDISKYLNWYQQKPDGTVK | 38 |
| B005_Light Chain | LLIYHTSRLHSGVPSRFSGSGSGTDYSLTISNLEQEDIATYFCQQG | |
| (His tagged | NTLPYTFGGGTKLEITKARTVAAPSVFIFPPSDEQLKSGTASVVCL | |
| version of SEQ ID | LNNFYPREAKVQWKVDNALQSGNSQESVTEQDSKDSTYSLSSTL | |
| NO: 32) | TLSKADYEKHKVYACEVTHQGLSSPVTKSFNRGECHHHHHH | |
| E200 + CD19 | DIQMTQTTSSLSASLGDRVTISCRASQDISKYLNWYQQKPDGTVK | 39 |
| binder (nfP2X₇ | LLIYHTSRLHSGVPSRFSGSGSGTDYSLTISNLEQEDIATYFCQQG | |
| epitope | NTLPYTFGGGTKLEITKARTVAAPSVFIFPPSDEQLKSGTASVVCL | |
| underlined) | LNNFYPREAKVQWKVDNALQSGNSQESVTEQDSKDSTYSLSSTL | |
| CD19, FMC63, | TLSKADYEKHKVYACEVTHQGLSSPVTKSFNRGECGHNYTTRNIL | |
| B006_Light Chain | PGLNITS | |
| CD19 binder scFv | DIQMTQTTSSLSASLGDRVTISCRASQDISKYLNWYQQKPDGTVK | 40 |
| format | LLIYHTSRLHSGVPSRFSGSGSGTDYSLTISNLEQEDIATYFCQQG | |
| CD19, FMC63, | NTLPYTFGGGTKLEITGGGGSGGGGSGGGGSEVKLQESGPGLV | |
| B011_scFv_Light/ | APSQSLSVTCTVSGVSLPDYGVSWIRQPPRKGLEWLGVIWGSET | |
| Heavy | TYYNSALKSRLTIIKDNSKSQVFLKMNSLQTDDTAIYYCAKHYYYG | |
| | GSYAMDYWGQGTSVTVSSHHHHHH | |
| CD19 binder scFv | EVKLQESGPGLVAPSQSLSVTCTVSGVSLPDYGVSWIRQPPRKG | 41 |
| format | LEWLGVIWGSETTYYNSALKSRLTIIKDNSKSQVFLKMNSLQTDDT | |
| CD19, FMC63, | AIYYCAKHYYYGGSYAMDYWGQGTSVTVSSGGGGSGGGGSGG | |
| B012_scFv_Heavy/ | GGSDIQMTQTTSSLSASLGDRVTISCRASQDISKYLNWYQQKPD | |
| Light | GTVKLLIYHTSRLHSGVPSRFSGSGSGTDYSLTISNLEQEDIATYF | |
| | CQQGNTLPYTFGGGTKLEITHHHHHH | |
| E200 + CD19 | GHNYTTRNILPGLNITSDIQMTQTTSSLSASLGDRVTISCRASQDI | 42 |
| binder in scFv | SKYLNWYQQKPDGTVKLLIYHTSRLHSGVPSRFSGSGSGTDYSLTI | |
| format (nfP2X₇ | SNLEQEDIATYFCQQGNTLPYTFGGGTKLEITGGGGSGGGGSGG | |
| epitope | GGSEVKLQESGPGLVAPSQSLSVTCTVSGVSLPDYGVSWIRQPP | |
| underlined) | RKGLEWLGVIWGSETTYYNSALKSRLTIIKDNSKSQVFLKMNSLQ | |
| CD19, FMC63, | TDDTAIYYCAKHYYYGGSYAMDYWGQGTSVTVSSHHHHHH | |
| B013-1_scFv_LH | | |
| E200 + CD19 | GHNYTTRNILPGLNITSGGGGSDIQMTQTTSSLSASLGDRVTISC | 43 |
| binder in scFv | RASQDISKYLNWYQQKPDGTVKLLIYHTSRLHSGVPSRFSGSGSG | |
| format (nfP2X₇ | TDYSLTISNLEQEDIATYFCQQGNTLPYTFGGGTKLEITGGGGSG | |
| epitope | GGGSGGGGSEVKLQESGPGLVAPSQSLSVTCTVSGVSLPDYGVS | |
| underlined) | WIRQPPRKGLEWLGVIWGSETTYYNSALKSRLTIIKDNSKSQVFL | |
| CD19, FMC63, | KMNSLQTDDTAIYYCAKHYYYGGSYAMDYWGQGTSVTVSSHHH | |
| B013-2_scFv_LH | HHH | |
| E200 + CD19 | GHNYTTRNILPGLNITSEVKLQESGPGLVAPSQSLSVTCTVSGVS | 44 |
| binder in scFv | LPDYGVSWIRQPPRKGLEWLGVIWGSETTYYNSALKSRLTIIKDN | |
| format (nfP2X₇ | SKSQVFLKMNSLQTDDTAIYYCAKHYYYGGSYAMDYWGQGTSVT | |
| epitope | VSSGGGGSGGGGSGGGGSDIQMTQTTSSLSASLGDRVTISCRA | |
| underlined) | SQDISKYLNWYQQKPDGTVKLLIYHTSRLHSGVPSRFSGSGSGT | |
| CD19, FMC63, | DYSLTISNLEQEDIATYFCQQGNTLPYTFGGGTKLEITHHHHHH | |
| B014-1_scFv_HL | | |
| E200 + CD19 | GHNYTTRNILPGLNITSGGGGSEVKLQESGPGLVAPSQSLSVTCT | 45 |
| binder in scFv | VSGVSLPDYGVSWIRQPPRKGLEWLGVIWGSETTYYNSALKSRL | |
| format (nfP2X₇ | TIIKDNSKSQVFLKMNSLQTDDTAIYYCAKHYYYGGSYAMDYWG | |
| epitope | QGTSVTVSSGGGGSGGGGSGGGGSDIQMTQTTSSLSASLGDRV | |
| underlined) | TISCRASQDISKYLNWYQQKPDGTVKLLIYHTSRLHSGVPSRFSG | |
| CD19, FMC63, | SGSGTDYSLTISNLEQEDIATYFCQQGNTLPYTFGGGTKLEITHHH | |
| B014-2_scFv_HL | HHH | |
| E200 + CD19 | EVKLQESGPGLVAPSQSLSVTCTVSGVSLPDYGVSWIRQPPRKG | 46 |
| binder in scFv | LEWLGVIWGSETTYYNSALKSRLTIIKDNSKSQVFLKMNSLQTDDT | |
| format (nfP2X₇ | AIYYCAKHYYYGGSYAMDYWGQGTSVTVSSGGGGSGGGGSGG | |
| epitope | GGSDIQMTQTTSSLSASLGDRVTISCRASQDISKYLNWYQQKPD | |
| underlined) | GTVKLLIYHTSRLHSGVPSRFSGSGSGTDYSLTISNLEQEDIATYF | |
| CD19, FMC63, | CQQGNTLPYTFGGGTKLEITGHNYTTRNILPGLNITSHHHHHH | |
| B015_scFv_HL | | |
| E200 + CD19 | DIQMTQTTSSLSASLGDRVTISCRASQDISKYLNWYQQKPDGTVK | 309 |
| binder in scFv | LLIYHTSRLHSGVPSRFSGSGSGTDYSLTISNLEQEDIATYFCQQG | |
| format (nfP2X₇ | NTLPYTFGGGTKLEITGGGGSGGGGSGGGGSEVKLQESGPGLV | |
| epitope | APSQSLSVTCTVSGVSLPDYGVSWIRQPPRKGLEWLGVIWGSET | |
| underlined) | TYYNSALKSRLTIIKDNSKSQVFLKMNSLQTDDTAIYYCAKHYYYG | |
| CD19, FMC63, | GSYAMDYWGQGTSVTVSSGHNYTTRNILPGLNITSHHHHHH | |
| B016_scFv_LH | | |
| E200 + CD19 | GHNYTTRNILPGLNITSDIQMTQTTSSLSASLGDRVTISCRASQDIS | 47 |
| binder in scFv | KYLNWYQQKPDGTVKLLIYHTSRLHSGVPSRFSGSGSGTDYSLTI | |
| format (nfP2X₇ | SNLEQEDIATYFCQQGNTLPYTFGGGTKLEITGGGGSGGGGSGG | |
| epitope | GGSEVKLQESGPGLVAPSQSLSVTCTVSGVSLPDYGVSWIRQPP | |
| underlined) | RKGLEWLGVIWGSETTYYNSALKSRLTIIKDNSKSQVFLKMNSLQ | |
| CD19, FMC63, | TDDTAIYYCAKHYYYGGSYAMDYWGQGTSVTVSSGHNYTTRNIL | |
| B017-1_scFv_LH | PGLNITSHHHHHH | |
| E200 + CD19 | GHNYTTRNILPGLNITSGGGGSDIQMTQTTSSLSASLGDRVTISCR | 48 |
| binder in scFv | ASQDISKYLNWYQQKPDGTVKLLIYHTSRLHSGVPSRFSGSGSG | |
| format (nfP2X₇ | TDYSLTISNLEQEDIATYFCQQGNTLPYTFGGGTKLEITGGGGSG | |
| epitope | GGGSGGGGSEVKLQESGPGLVAPSQSLSVTCTVSGVSLPDYGV | |
| underlined) | SWIRQPPRKGLEWLGVIWGSETTYYNSALKSRLTIIKDNSKSQVFL | |
| CD19, FMC63, | KMNSLQTDDTAIYYCAKHYYYGGSYAMDYWGQGTSVTVSSGHN | |
| B017-2_scFv_LH | YTTRNILPGLNITSHHHHHH | |
| E200 + CD19 | GHNYTTRNILPGLNITSEVKLQESGPGLVAPSQSLSVTCTVSGVSL | 49 |
| binder in scFv | PDYGVSWIRQPPRKGLEWLGVIWGSETTYYNSALKSRLTIIKDNS | |
| format (nfP2X₇ | KSQVFLKMNSLQTDDTAIYYCAKHYYYGGSYAMDYWGQGTSVT | |
| epitope | VSSGGGGSGGGGSGGGGSDIQMTQTTSSLSASLGDRVTISCRA | |
| underlined) | SQDISKYLNWYQQKPDGTVKLLIYHTSRLHSGVPSRFSGSGSGT | |
| CD19, FMC63, | DYSLTISNLEQEDIATYFCQQGNTLPYTFGGGTKLEITGHNYTTRN | |
| B018-1_scFv_HL | ILPGLNITSHHHHHH | |
| E200 + CD19 | GHNYTTRNILPGLNITSGGGGSEVKLQESGPGLVAPSQSLSVTCT | 50 |
| binder in scFv | VSGVSLPDYGVSWIRQPPRKGLEWLGVIWGSETTYYNSALKSRL | |
| format (nfP2X₇ | TIIKDNSKSQVFLKMNSLQTDDTAIYYCAKHYYYGGSYAMDYWG | |
| epitope | QGTSVTVSSGGGGSGGGGSGGGGSDIQMTQTTSSLSASLGDRV | |
| underlined) | TISCRASQDISKYLNWYQQKPDGTVKLLIYHTSRLHSGVPSRFSG | |
| CD19, FMC63, | SGSGTDYSLTISNLEQEDIATYFCQQGNTLPYTFGGGTKLEITGHN | |
| B018-2_scFv_HL | YTTRNILPGLNITSHHHHHH | |

Constructs based on Tafasitamab (for binding CD19)

| | | |
|---|---|---|
| E200 + CD19 | GHNYTTRNILPGLNITSDIVMTQSPATLSLSPGERATLSCRSSKSL | 51 |
| binder (nfP2X₇ | QNVNGNTYLYWFQQKPGQSPQLLIYRMSNLNSGVPDRFSGSGS | |
| epitope | GTEFTLTISSLEPEDFAVYYCMQHLEYPITFGAGTKLEIKRTVAAPS | |
| underlined) | VFIFPPSDEQLKSGTASVVCLLNNFYPREAKVQWKVDNALQSGN | |
| CD19, | SQESVTEQDSKDSTYSLSSTLTLSKADYEKHKVYACEVTHQGLSS | |
| Tafasitamab, | PVTKSFNRGECHHHHHH | |
| B020-1_Light | | |
| Chain | | |
| CD19, | EVQLVESGGGLVKPGGSLKLSCAASGYTFTSYVMHWVRQAPGK | 52 |
| Tafasitamab, | GLEWIGYINPYNDGTKYNEKFQGRVTISSDKSISTAYMELSSLRSE | |
| B020-2_Heavy | DTAMYYCARGTYYYGTRVFDYWGQGTLVTVSSASTKGPSVFPLA | |
| Chain | PSSKSTSGGTAALGCLVKDYFPEPVTVSWNSGALTSGVHTFPAV | |
| | LQSSGLYSLSSVVTVPSSSLGTQTYICNVNHKPSNTKVDKKVEPK | |
| | SCHHHHHH | |

Constructs for binding CD20

| | | |
|---|---|---|
| E200 + CD20 | GHNYTTRNILPGLNITSDIQMTQSPSSLSASVGDRVTITCRASSSV | 53 |
| binder (nfP2X₇ | SYMHWYQQKPGKAPKPLIYAPSNLASGVPSRFSGSGSGTDFTLTI | |
| epitope | SSLQPEDFATYYCQQWSFNPPTFGQGTKVEIKRTVAAPSVFIFPP | |
| underlined) | SDEQLKSGTASVVCLLNNFYPREAKVQWKVDNALQSGNSQESV | |
| CD20, | TEQDSKDSTYSLSSTLTLSKADYEKHKVYACEVTHQGLSSPVTKS | |
| Ocrelizumab, | FNRGECHHHHHH | |
| B021-1_Light | | |
| chain | | |
| CD20, | EVQLVESGGGLVQPGGSLRLSCAASGYTFTSYNMHWVRQAPGK | 54 |
| Ocrelizumab, | GLEWVGAIYPGNGDTSYNQKFKGRFTISVDKSKNTLYLQMNSLR | |
| B021-1_Heavy | AEDTAVYYCARVVYYSNSYWYFDVWGQGTLVTVSSASTKGPSV | |
| Chain | FPLAPSSKSTSGGTAALGCLVKDYFPEPVTVSWNSGALTSGVHT | |
| | FPAVLQSSGLYSLSSVVTVPSSSLGTQTYICNVNHKPSNTKVDKK | |
| | VEPKSCHHHHHH | |
| E200 + CD20 | GHNYTTRNILPGLNITSEIVLTQSPATLSLSPGERATLSCRASQSV | 55 |
| binder (nfP2X₇ | SSYLAWYQQKPGQAPRLLIYDASNRATGIPARFSGSGSGTDFTLT | |
| epitope | ISSLEPEDFAVYYCQQRSNWPITFGQGTRLEIKRTVAAPSVFIFPP | |
| underlined) | SDEQLKSGTASVVCLLNNFYPREAKVQWKVDNALQSGNSQESV | |
| CD20, | TEQDSKDSTYSLSSTLTLSKADYEKHKVYACEVTHQGLSSPVTKS | |
| Ofatumumab, | FNRGECHHHHHH | |
| B022-1_Light | | |
| Chain | | |
| CD20, | EVQLVESGGGLVQPGRSLRLSCAASGFTFNDYAMHWVRQAPGK | 56 |
| Ofatumumab, | GLEWVSTISWNSGSIGYADSVKGRFTISRDNAKKSLYLQMNSLRA | |
| B022-2_Heavy | EDTALYYCAKDIQYGNYYYGMDVWGQGTTVTVSSASTKGPSVFP | |
| Chain | LAPSSKSTSGGTAALGCLVKDYFPEPVTVSWNSGALTSGVHTFP | |
| | AVLQSSGLYSLSSVVTVPSSSLGTQTYICNVNHKPSNTKVDKKVE | |
| | PKSCHHHHHH | |

Constructs for binding CD22

| | | |
|---|---|---|
| E200 + CD22 | GHNYTTRNILPGLNITSDIQMIQSPSSLSASVGDRVTITCRASQTIW | 57 |
| binder (nfP2X₇ | SYLNWYRQRPGEAPNLLIYAASSLQSGVPSRFSGRGSGTDFTLTI | |
| epitope | SSLQAEDFATYYCQQSYSIPQTFGQGTKLEIKRTVAAPSVFIFPPS | |
| underlined) | DEQLKSGTASVVCLLNNFYPREAKVQWKVDNALQSGNSQESVT | |
| CD22, m971-L7, | EQDSKDSTYSLSSTLTLSKADYEKHKVYACEVTHQGLSSPVTKSF | |
| B023-1_LC | NRGECHHHHHH | |
| CD22, m971-L7, | QVQLQQSGPGMVKPSQTLSLTCAISGDSVSSNSVAWNWIRQSP | 58 |
| B023-2_Heavy | SRGLEWLGRTYYRSTWYNDYAVSMKSRITINPDTNKNQFSLQLN | |
| Chain | SVTPEDTAVYYCAREVTGDLEDAFDIWGQGTMVTVSSASTKGPS | |
| | VFPLAPSSKSTSGGTAALGCLVKDYFPEPVTVSWNSGALTSGVH | |
| | TFPAVLQSSGLYSLSSVVTVPSSSLGTQTYICNVNHKPSNTKVDK | |
| | KVEPKSCHHHHHH | |
| E200 + CD22 | GHNYTTRNILPGLNITSDVQVTQSPSSLSASVGDRVTITCRSSQSL | 59 |
| binder (nfP2X₇ | ANSYGNTFLSWYLHKPGKAPQLLIYGISNRFSGVPDRFSGSGSGT | |
| epitope | DFTLTISSLQPEDFATYYCLQGTHQPYTFGQGTKVEIKRTVAAPSV | |
| underlined) | FIFPPSDEQLKSGTASVVCLLNNFYPREAKVQWKVDNALQSGNS | |
| CD22, | QESVTEQDSKDSTYSLSSTLTLSKADYEKHKVYACEVTHQGLSSP | |
| Inotuzumab, | VTKSFNRGECHHHHHH | |
| B024-1_LC | | |
| CD22, | EVQLVQSGAEVKKPGASVKVSCKASGYRFTNYWIHWVRQAPGQ | 60 |
| Inotuzumab, | GLEWIGGINPGNNYATYRRKFQGRVTMTADTSTSTVYMELSSLR | |
| B024-2_HC | SEDTAVYYCTREGYGNYGAWFAYWGQGTLVTVSSASTKGPSVF | |
| | PLAPSSKSTSGGTAALGCLVKDYFPEPVTVSWNSGALTSGVHTF | |
| | PAVLQSSGLYSLSSVVTVPSSSLGTQTYICNVNHKPSNTKVDKKV | |
| | EPKSCHHHHHH | |

Constructs for binding CD79B

| | | |
|---|---|---|
| E200 + CD79B | GHNYTTRNILPGLNITSDIQLTQSPSSLSASVGDRVTITCKASQSV | 61 |
| binder (nfP2X₇ | DYEGDSFLNWYQQKPGKAPKLLIYAASNLESGVPSRFSGSGSGT | |
| epitope | DFTLTISSLQPEDFATYYCQQSNEDPLTFGQGTKVEIKRTVAAPSV | |
| underlined) | FIFPPSDEQLKSGTASVVCLLNNFYPREAKVQWKVDNALQSGNS | |
| CD79B, | QESVTEQDSKDSTYSLSSTLTLSKADYEKHKVYACEVTHQGLSSP | |
| Polatuzumab, | VTKSFNRGECHHHHHH | |
| B025-1_LC | | |
| CD79B, | EVQLVESGGGLVQPGGSLRLSCAASGYTFSSYWIEWVRQAPGK | 62 |
| Polatuzumab, | GLEWIGEILPGGGDTNYNEIFKGRATFSADTSKNTAYLQMNSLRA | |
| B025-2_HC | EDTAVYYCTRRVPIRLDYWGQGTLVTVSSASTKGPSVFPLAPSSK | |
| | STSGGTAALGCLVKDYFPEPVTVSWNSGALTSGVHTFPAVLQSS | |
| | GLYSLSSVVTVPSSSLGTQTYICNVNHKPSNTKVDKKVEPKSCHH | |
| | HHHH | |

Constructs for binding CD37

| | | |
|---|---|---|
| E200 + CD37 | GHNYTTRNILPGLNITSEIVLTQSPATLSLSPGERATLSCRASENV | 63 |
| binder (nfP2X₇ | YSYLAWYQQKPGQAPRLLIYFAKTLAEGIPARFSGSGSGTDFTLTI | |
| epitope | SSLEPEDFAVYYCQHHSDNPWTFGQGTKVEIKRTVAAPSVFIFPP | |
| underlined) | SDEQLKSGTASVVCLLNNFYPREAKVQWKVDNALQSGNSQESV | |
| CD37, | TEQDSKDSTYSLSSTLTLSKADYEKHKVYACEVTHQGLSSPVTKS | |
| Otlertuzumab, | FNRGECHHHHHH | |
| B026-1_LC | | |
| CD37, | EVQLVQSGAEVKKPGESLKISCKGSGYSFTGYNMNWVRQMPGK | 64 |
| Otlertuzumab, | GLEWMGNIDPYYGGTTYNRKFKGQVTISADKSISTAYLQWSSLKA | |
| B026-2_HC | SDTAMYYCARSVGPFDSWGQGTLVTVSSASTKGPSVFPLAPSSK | |
| | STSGGTAALGCLVKDYFPEPVTVSWNSGALTSGVHTFPAVLQSS | |
| | GLYSLSSVVTVPSSSLGTQTYICNVNHKPSNTKVDKKVEPKSCHH | |
| | HHHH | |

Constructs for binding CD38

| | | |
|---|---|---|
| E200 + CD38 | GHNYTTRNILPGLNITSEIVLTQSPATLSLSPGERATLSCRASQSV | 65 |
| binder (nfP2X₇ | SSYLAWYQQKPGQAPRLLIYDASNRATGIPARFSGSGSGTDFTLT | |
| epitope | ISSLEPEDFAVYYCQQRSNWPPTFGQGTKVEIKRTVAAPSVFIFP | |
| underlined) | PSDEQLKSGTASVVCLLNNFYPREAKVQWKVDNALQSGNSQES | |
| CD38, | VTEQDSKDSTYSLSSTLTLSKADYEKHKVYACEVTHQGLSSPVTK | |
| Daratumumab, | SFNRGECHHHHHH | |
| B027-1_LC | | |
| CD38, | EVQLLESGGGLVQPGGSLRLSCAVSGFTFNSFAMSWVRQAPGK | 66 |
| Daratumumab, | GLEWVSAISGSGGGTYYADSVKGRFTISRDNSKNTLYLQMNSLR | |
| B027-2_HC | AEDTAVYFCAKDKILWFGEPVFDYWGQGTLVTVSSASTKGPSVF | |
| | PLAPSSKSTSGGTAALGCLVKDYFPEPVTVSWNSGALTSGVHTF | |
| | PAVLQSSGLYSLSSVVTVPSSSLGTQTYICNVNHKPSNTKVDKKV | |
| | EPKSCHHHHHH | |

Constructs for binding CD70

| | | |
|---|---|---|
| E200 + CD70 | GHNYTTRNILPGLNITSQAVVTQEPSLTVSPGGTVTLTCGLKSGS | 67 |
| binder (nfP2X₇ | VTSDNFPTWYQQTPGQAPRLLIYNTNTRHSGVPDRFSGSILGNK | |
| epitope | AALTITGAQADDEAEYFCALFISNPSVEFGGGTQLTVLKRTVAAPS | |
| underlined) | VFIFPPSDEQLKSGTASVVCLLNNFYPREAKVQWKVDNALQSGN | |
| CD70, | SQESVTEQDSKDSTYSLSSTLTLSKADYEKHKVYACEVTHQGLSS | |
| Cusatuzumab, | PVTKSFNRGECHHHHHH | |
| B028-1_LC | | |
| CD70, | EVQLVESGGGLVQPGGSLRLSCAASGFTFSVYYMNWVRQAPGK | 68 |
| Cusatuzumab, | GLEWVSDINNEGGTTYYADSVKGRFTISRDNSKNSLYLQMNSLR | |
| B028-2_HC | AEDTAVYYCARDAGYSNHVPIFDSWGQGTLVTVSSASTKGPSVF | |
| | PLAPSSKSTSGGTAALGCLVKDYFPEPVTVSWNSGALTSGVHTF | |
| | PAVLQSSGLYSLSSVVTVPSSSLGTQTYICNVNHKPSNTKVDKKV | |
| | EPKSCHHHHHH | |

Constructs for binding CD30

| | | |
|---|---|---|
| E200 + CD30 | GHNYTTRNILPGLNITSDIVLTQSPASLAVSLGQRATISCKASQSV | 69 |
| binder (nfP2X₇ | DFDGDSYMNWYQQKPGQPPKVLIYAASNLESGIPARFSGSGSGT | |
| epitope | DFTLNIHPVEEEDAATYYCQQSNEDPWTFGGGTKLEIKRTVAAPS | |
| underlined) | VFIFPPSDEQLKSGTASVVCLLNNFYPREAKVQWKVDNALQSGN | |
| CD30, | SQESVTEQDSKDSTYSLSSTLTLSKADYEKHKVYACEVTHQGLSS | |
| Brentuximab, | PVTKSFNRGECHHHHHH | |
| B029-1_LC | | |
| CD30, | QIQLQQSGPEVVKPGASVKISCKASGYTFTDYYITWVKQKPGQGL | 70 |
| Brentuximab, | EWIGWIYPGSGNTKYNEKFKGKATLTVDTSSSTAFMQLSSLTSED | |
| B029-2_HC | TAVYFCANYGNYWFAYWGQGTQVTVSAASTKGPSVFPLAPSSK | |
| | STSGGTAALGCLVKDYFPEPVTVSWNSGALTSGVHTFPAVLQSS | |
| | GLYSLSSVVTVPSSSLGTQTYICNVNHKPSNTKVDKKVEPKSCHH | |
| | HHHH | |

Constructs for binding CD33

| | | |
|---|---|---|
| E200 + CD33 | GHNYTTRNILPGLNITSDIQLTQSPSTLSASVGDRVTITCRASESLD | 71 |
| binder (nfP2X₇ | NYGIRFLTWFQQKPGKAPKLLMYAASNQGSGVPSRFSGSGSGT | |
| epitope | EFTLTISSLQPDDFATYYCQQTKEVPWSFGQGTKVEVKRTVAAPS | |
| underlined) | VFIFPPSDEQLKSGTASVVCLLNNFYPREAKVQWKVDNALQSGN | |
| CD33, | SQESVTEQDSKDSTYSLSSTLTLSKADYEKHKVYACEVTHQGLSS | |
| Gemtuzumab, | PVTKSFNRGECHHHHHH | |
| B030-1_LC | | |
| CD33, | EVQLVQSGAEVKKPGSSVKVSCKASGYTITDSNIHWVRQAPGQS | 72 |
| Gemtuzumab, | LEWIGYIYPYNGGTDYNQKFKNRATLTVDNPTNTAYMELSSLRSE | |
| B030-2_Heavy | DTAFYYCVNGNPWLAYWGQGTLVTVSSASTKGPSVFPLAPSSKS | |
| Chain | TSGGTAALGCLVKDYFPEPVTVSWNSGALTSGVHTFPAVLQSSG | |
| | LYSLSSVVTVPSSSLGTQTYICNVNHKPSNTKVDKKVEPKSCHHH | |
| | HHH | |
| E200 + CD33 | GHNYTTRNILPGLNITSDIQMTQSPSSLSASVGDRVTITCRASESV | 73 |
| binder (nfP2X₇ | DNYGISFMNWFQQKPGKAPKLLIYAASNQGSGVPSRFSGSGSGT | |
| epitope | DFTLTISSLQPDDFATYYCQQSKEVPWTFGQGTKVEIKRTVAAPS | |
| underlined) | VFIFPPSDEQLKSGTASVVCLLNNFYPREAKVQWKVDNALQSGN | |
| CD33, | SQESVTEQDSKDSTYSLSSTLTLSKADYEKHKVYACEVTHQGLSS | |
| Lintuzumab, | PVTKSFNRGECHHHHHH | |
| B031-1_LC | | |
| CD33, | QVQLVQSGAEVKKPGSSVKVSCKASGYTFTDYNMHWVRQAPG | 74 |
| Lintuzumab, | QGLEWIGYIYPYNGGTGYNQKFKSKATITADESTNTAYMELSSLR | |
| B031-2_HC | SEDTAVYYCARGRPAMDYWGQGTLVTVSSASTKGPSVFPLAPS | |
| | SKSTSGGTAALGCLVKDYFPEPVTVSWNSGALTSGVHTFPAVLQ | |
| | SSGLYSLSSVVTVPSSSLGTQTYICNVNHKPSNTKVDKKVEPKSC | |
| | HHHHHH | |

Constructs for binding Her2

| | | |
|---|---|---|
| E200 + Her2 | GHNYTTRNILPGLNITSDIQMTQSPSSLSASVGDRVTITCKASQDV | 75 |
| binder (nfP2X₇ | SIGVAWYQQKPGKAPKLLIYSASYRYTGVPSRFSGSGSGTDFTLT | |
| epitope | ISSLQPEDFATYYCQQYYIYPYTFGQGTKVEIKRTVAAPSVFIFPPS | |
| underlined) | DEQLKSGTASVVCLLNNFYPREAKVQWKVDNALQSGNSQESVT | |
| Her2, | EQDSKDSTYSLSSTLTLSKADYEKHKVYACEVTHQGLSSPVTKSF | |
| Pertuzumab, | NRGECHHHHHH | |
| B032-1_LC | | |
| Her2, | EVQLVESGGGLVQPGGSLRLSCAASGFTFTDYTMDWVRQAPGK | 76 |
| Pertuzumab, | GLEWVADVNPNSGGSIYNQRFKGRFTLSVDRSKNTLYLQMNSLR | |
| B032-2_HC | AEDTAVYYCARNLGPSFYFDYWGQGTLVTVSSASTKGPSVFPLA | |
| | PSSKSTSGGTAALGCLVKDYFPEPVTVSWNSGALTSGVHTFPAV | |
| | LQSSGLYSLSSVVTVPSSSLGTQTYICNVNHKPSNTKVDKKVEPK | |
| | SCHHHHHH | |
| E200 + Her2 | GHNYTTRNILPGLNITSDIQMTQSPSSLSASVGDRVTITCRASQDV | 77 |
| binder (nfP2X₇ | NTAVAWYQQKPGKAPKLLIYSASFLYSGVPSRFSGSRSGTDFTLT | |
| epitope | ISSLQPEDFATYYCQQHYTTPPTFGQGTKVEIKRTVAAPSVFIFPP | |
| underlined) | SDEQLKSGTASVVCLLNNFYPREAKVQWKVDNALQSGNSQESV | |
| Her2, | TEQDSKDSTYSLSSTLTLSKADYEKHKVYACEVTHQGLSSPVTKS | |
| Trastuzumab, | FNRGECHHHHHH | |
| B033-1_LC | | |
| Her2, | EVQLVESGGGLVQPGGSLRLSCAASGFNIKDTYIHWVRQAPGKG | 78 |
| Trastuzumab, | LEWVARIYPTNGYTRYADSVKGRFTISADTSKNTAYLQMNSLRAE | |
| B033-2_HC | DTAVYYCSRWGGDGFYAMDYWGQGTLVTVSSASTKGPSVFPLA | |
| | PSSKSTSGGTAALGCLVKDYFPEPVTVSWNSGALTSGVHTFPAV | |
| | LQSSGLYSLSSVVTVPSSSLGTQTYICNVNHKPSNTKVDKKVEPK | |
| | SCHHHHHH | |

Constructs for binding EGFR

| | | |
|---|---|---|
| E200 + EGFR | GHNYTTRNILPGLNITSEIVMTQSPATLSLSPGERATLSCRASQSV | 79 |
| binder (nfP2X₇ | SSYLAWYQQKPGQAPRLLIYDASNRATGIPARFSGSGSGTDFTLT | |
| epitope | ISSLEPEDFAVYYCHQYGSTPLTFGGGTKAEIKRTVAAPSVFIFPP | |
| underlined) | SDEQLKSGTASVVCLLNNFYPREAKVQWKVDNALQSGNSQESV | |
| EGFR, | TEQDSKDSTYSLSSTLTLSKADYEKHKVYACEVTHQGLSSPVTKS | |
| Necitumumab, | FNRGECHHHHHH | |
| B034-1_LC | | |
| EGFR, | QVQLQESGPGLVKPSQTLSLTCTVSGGSISSGDYYWSWIRQPPG | 80 |
| Necitumumab, | KGLEWIGYIYYSGSTDYNPSLKSRVTMSVDTSKNQFSLKVNSVTA | |
| B034-2_HC | ADTAVYYCARVSIFGVGTFDYWGQGTLVTVSSASTKGPSVFPLA | |
| | PSSKSTSGGTAALGCLVKDYFPEPVTVSWNSGALTSGVHTFPAV | |
| | LQSSGLYSLSSVVTVPSSSLGTQTYICNVNHKPSNTKVDKKVEPK | |
| | SCHHHHHH | |
| E200 + EGFR | GHNYTTRNILPGLNITSDIQMTQSPSSLSASVGDRVTITCSASSSV | 81 |
| binder (nfP2X₇ | TYMYWYQQKPGKAPKLLIYDTSNLASGVPSRFSGSGSGTDYTFTI | |
| epitope | SSLQPEDIATYYCQQWSSHIFTFGQGTKVEIKRTVAAPSVFIFPPS | |
| underlined) | DEQLKSGTASVVCLLNNFYPREAKVQWKVDNALQSGNSQESVT | |
| EGFR, | EQDSKDSTYSLSSTLTLSKADYEKHKVYACEVTHQGLSSPVTKSF | |
| Matuzumab, | NRGECHHHHHH | |
| B035-1_LC | | |
| EGFR, | QVQLVQSGAEVKKPGASVKVSCKASGYTFTSHWMHWVRQAPG | 82 |
| Matuzumab, | QGLEWIGEFNPSNGRTNYNEKFKSKATMTVDTSTNTAYMELSSL | |
| B035-2_HC | RSEDTAVYYCASRDYDYDGRYFDYWGQGTLVTVSSASTKGPSV | |
| | FPLAPSSKSTSGGTAALGCLVKDYFPEPVTVSWNSGALTSGVHT | |
| | FPAVLQSSGLYSLSSVVTVPSSSLGTQTYICNVNHKPSNTKVDKK | |
| | VEPKSCHHHHHH | |
| E200 + EGFR | GHNYTTRNILPGLNITSDIQMTQSPSSLSASVGDRVTITCQASQDI | 83 |
| binder (nfP2X₇ | SNYLNWYQQKPGKAPKLLIYDASNLETGVPSRFSGSGSGTDFTF | |
| epitope | TISSLQPEDIATYFCQHFDHLPLAFGGGTKVEIKRTVAAPSVFIFPP | |
| underlined) | SDEQLKSGTASVVCLLNNFYPREAKVQWKVDNALQSGNSQESV | |
| EGFR, | TEQDSKDSTYSLSSTLTLSKADYEKHKVYACEVTHQGLSSPVTKS | |
| Panitumumab, | FNRGECHHHHHH | |
| B036-1_LC | | |
| EGFR, | QVQLQESGPGLVKPSETLSLTCTVSGGSVSSGDYYWTWIRQSP | 84 |
| Panitumumab, | GKGLEWIGHIYYSGNTNYNPSLKSRLTISIDTSKTQFSLKLSSVTAA | |
| B036-2_HC | DTAIYYCVRDRVTGAFDIWGQGTMVTVSSASTKGPSVFPLAPSSK | |
| | STSGGTAALGCLVKDYFPEPVTVSWNSGALTSGVHTFPAVLQSS | |
| | GLYSLSSVVTVPSSSLGTQTYICNVNHKPSNTKVDKKVEPKSCHH | |
| | HHHH | |

Constructs for binding CD276

| | | |
|---|---|---|
| E200 + CD276 | GHNYTTRNILPGLNITSEIVMTQSPATLSVSPGERVTLSCRASQSI | 85 |
| binder (nfP2X₇ | SDYLYWYQQKSHESPRLLIKYASQSISGIPARFSGSGSGSEFTLTI | |
| epitope | NSVEPEDVGVYYCQNGHSFPLTFGQGTKLELKRTVAAPSVFIFPP | |
| underlined) | SDEQLKSGTASVVCLLNNFYPREAKVQWKVDNALQSGNSQESV | |
| CD276, hu8H9- | TEQDSKDSTYSLSSTLTLSKADYEKHKVYACEVTHQGLSSPVTKS | |
| 6m, B037-1_LC | FNRGECHHHHHH | |
| CD276, hu8H9- | QVQLVQSGAEVVKPGASVKLSCKTSGYTFTNYDINWVRQRPGQ | 86 |
| 6m, B037-2_HC | GLEWIGWIFPGDDSTQYNEKFKGKATLTTDTSTSTAYMELSSLRS | |
| | EDTAVYFCARQTTGTWFAYWGQGTLVTVSSASTKGPSVFPLAPS | |
| | SKSTSGGTAALGCLVKDYFPEPVTVSWNSGALTSGVHTFPAVLQ | |
| | SSGLYSLSSVVTVPSSSLGTQTYICNVNHKPSNTKVDKKVEPKSC | |
| | HHHHHH | |

Constructs for binding GD2

| | | |
|---|---|---|
| E200 + GD2 binder | GHNYTTRNILPGLNITSKIVMTQTPATLSVSAGERVTITCKASQSV | 87 |
| (nfP2X₇ epitope | SNHVTWYQQKPGQAPRLLIYSASNRYSGVPARFSGSGYGTEFTF | |
| underlined) | TISSVQSEDFAVYFCQQDYSSFGQGTKLEIKRTVAAPSVFIFPPSD | |
| GD2, Naxitamab, | EQLKSGTASVVCLLNNFYPREAKVQWKVDNALQSGNSQESVTE | |
| B038-1_LC | QDSKDSTYSLSSTLTLSKADYEKHKVYACEVTHQGLSSPVTKSFN | |
| | RGECHHHHHH | |
| GD2, Naxitamab, | QVQLVESGPGVVQPGRSLRLSCAVSGFSVTNYGVHWVRQPPGK | 88 |
| B038-2_HC | GLEWLGVIWAGGITNYNSSVKGRLTISKDNSKNTVYLQMNSLRAE | |
| | DTAVYYCASRGGHYGYALDYWGQGTLVTVSSASTKGPSVFPLAP | |
| | SSKSTSGGTAALGCLVKDYFPEPVTVSWNSGALTSGVHTFPAVL | |
| | QSSGLYSLSSVVTVPSSSLGTQTYICNVNHKPSNTKVDKKVEPKS | |
| | CHHHHHH | |

Constructs for binding BCMA

| | | |
|---|---|---|
| E200 + BCMA | GHNYTTRNILPGLNITSDIQMTQSPSSLSASVGDRVTITCSASQDI | 89 |
| binder (nfP2X₇ | SNYLNWYQQKPGKAPKLLIYYTSNLHSGVPSRFSGSGSGTDFTL | |
| epitope | TISSLQPEDFATYYCQQYRKLPWTFGQGTKLEIKRTVAAPSVFIFP | |
| underlined) | PSDEQLKSGTASVVCLLNNFYPREAKVQWKVDNALQSGNSQES | |
| BCMA, clone CA8 | VTEQDSKDSTYSLSSTLTLSKADYEKHKVYACEVTHQGLSSPVTK | |
| J9M0, B039-1_LC | SFNRGECHHHHHH | |
| BCMA, clone CA8 | QVQLVQSGAEVKKPGSSVKVSCKGSGYTFTNYWMHWVRQAPG | 90 |
| J9M0, B039- | QGLEWIGATYRGHSDTYYNQKFKGRATLTADTSTSTAYMELSSL | |
| 2_HC | RSEDTAVYYCTRGAIYDGYDVLDNWGQGTLVTVSSASTKGPSVF | |
| | PLAPSSKSTSGGTAALGCLVKDYFPEPVTVSWNSGALTSGVHTF | |
| | PAVLQSSGLYSLSSVVTVPSSSLGTQTYICNVNHKPSNTKVDKKV | |
| | EPKSCHHHHHH | |

Constructs for binding CD371

| | | |
|---|---|---|
| E200 + CD371 | GHNYTTRNILPGLNITSDIVMTQSPSSVSASVGDRVTITCRASQDI | 91 |
| binder (nfP2X₇ | SSWLAWYQQKPGKAPKLLIYAASSLQSGVPSRFNGSGSGTDFTL | |
| epitope | TISSLQPEDFATYYCQQSYSTPLTFGGGTKVEIKRTVAAPSVFIFP | |
| underlined) | PSDEQLKSGTASVVCLLNNFYPREAKVQWKVDNALQSGNSQES | |
| US10568947_CA | VTEQDSKDSTYSLSSTLTLSKADYEKHKVYACEVTHQGLSSPVTK | |
| R9, B040-1_LC | SFNRGECHHHHHH | |
| CD371, | QVQLVQSGAEVKEPGASVKVSCKAPANTFSDHVMHWVRQAPG | 92 |
| US10568947_CA | QRFEWMGYIHAANGGTHYSQKFQDRVTITRDTSANTVYMDLSSL | |
| R9, B040-2_HC | RSEDTAVYYCARGGYNSDAFDIWGQGTMVTVSSASTKGPSVFPL | |
| | APSSKSTSGGTAALGCLVKDYFPEPVTVSWNSGALTSGVHTFPA | |
| | VLQSSGLYSLSSVVTVPSSSLGTQTYICNVNHKPSNTKVDKKVEP | |
| | KSCHHHHHH | |

Constructs for binding CD135

| | | |
|---|---|---|
| E200 + CD135 | GHNYTTRNILPGLNITSDIVLTQSPATLSVTPGDSVSLSCRASQSIS | 93 |
| binder (nfP2X₇ | NNLHWYQQKSHESPRLLIKYASQSISGIPSRFSGSGSGTDFTLSIN | |
| epitope | SVETEDFGVYFCQQSNTWPYTFGGGTKLEIKRTVAAPSVFIFPPS | |
| underlined) | DEQLKSGTASVVCLLNNFYPREAKVQWKVDNALQSGNSQESVT | |
| CD135, clone | EQDSKDSTYSLSSTLTLSKADYEKHKVYACEVTHQGLSSPVTKSF | |
| 4G8, B041-1_LC | NRGECHHHHHH | |
| CD135, clone | QVQLQQPGAELVKPGASLKLSCKSSGYTFTSYWMHWVRQRPGH | 94 |
| 4G8, B041-2_HC | GLEWIGEIDPSDSYKDYNQKFKDKATLTVDRSSNTAYMHLSSLTS | |
| | DDSAVYYCARAITTTPFDFWGQGTTLTVSSASTKGPSVFPLAPSS | |
| | KSTSGGTAALGCLVKDYFPEPVTVSWNSGALTSGVHTFPAVLQS | |
| | SGLYSLSSVVTVPSSSLGTQTYICNVNHKPSNTKVDKKVEPKSCH | |
| | HHHHH | |

Constructs for binding CD123

| | | |
|---|---|---|
| E200 + CD123 | GHNYTTRNILPGLNITSDIVLTQSPASLAVSLGQRATISCRASESVD | 95 |
| binder (nfP2X₇ | NYGNTFMHWYQQKPGQPPKLLIYRASNLESGIPARFSGSGSRTD | |
| epitope | FTLTINPVEADDVATYYCQQSNEDPPTFGAGTKLELKRTVAAPSV | |
| underlined) | FIFPPSDEQLKSGTASVVCLLNNFYPREAKVQWKVDNALQSGNS | |
| CD123, clone | QESVTEQDSKDSTYSLSSTLTLSKADYEKHKVYACEVTHQGLSSP | |
| 32716, B042- | VTKSFNRGECHHHHHH | |
| 1 LC | | |
| CD123, clone | QIQLVQSGPELKKPGETVKISCKASGYIFTNYGMNWVKQAPGKSF | 96 |
| 32716, B042- | KWMGWINTYTGESTYSADFKGRFAFSLETSASTAYLHINDLKNED | |
| 2_HC | TATYFCARSGGYDPMDYWGQGTSVTVSSASTKGPSVFPLAPSS | |
| | KSTSGGTAALGCLVKDYFPEPVTVSWNSGALTSGVHTFPAVLQS | |
| | SGLYSLSSVVTVPSSSLGTQTYICNVNHKPSNTKVDKKVEPKSCH | |
| | HHHHH | |

Constructs for binding CD105

| | | |
|---|---|---|
| E200 + CD105 | GHNYTTRNILPGLNITSQIVLSQSPAILSASPGEKVTMTCRASSSV | 97 |
| binder (nfP2X₇ | SYMHWYQQKPGSSPKPWIYATSNLASGVPVRFSGSGSGTSYSL | |
| epitope | TISRVEAEDAATYYCQQWSSNPLTFGAGTKLELKRTVAAPSVFIF | |
| underlined) | PPSDEQLKSGTASVVCLLNNFYPREAKVQWKVDNALQSGNSQE | |
| CD105, | SVTEQDSKDSTYSLSSTLTLSKADYEKHKVYACEVTHQGLSSPVT | |
| Carotuximab, | KSFNRGECHHHHHH | |
| B043-1_LC | | |
| CD105, | EVKLEESGGGLVQPGGSMKLSCAASGFTFSDAWMDWVRQSPE | 98 |
| Carotuximab, | KGLEWVAEIRSKASNHATYYAESVKGRFTISRDDSKSSVYLQMNS | |
| B043-2_HC | LRAEDTGIYYCTRWRRFFDSWGQGTTLTVSSASTKGPSVFPLAP | |
| | SSKSTSGGTAALGCLVKDYFPEPVTVSWNSGALTSGVHTFPAVL | |
| | QSSGLYSLSSVVTVPSSSLGTQTYICNVNHKPSNTKVDKKVEPKS | |
| | CHHHHHH | |

Constructs for binding ROR-1

| | | |
|---|---|---|
| E200 + ROR-1 | GHNYTTRNILPGLNITSEIVLSQSPAITAASLGQKVTITCSASSNVS | 99 |
| binder (nfP2X₇ | YIHWYQQRSGTSPRPWIYEISKLASGVPVRFSGSGSGTSYSLTIS | |
| epitope | SMEAEDAAIYYCQQWNYPLITFGSGTKLEIQRTVAAPSVFIFPPSD | |
| underlined) ROR- | EQLKSGTASVVCLLNNFYPREAKVQWKVDNALQSGNSQESVTE | |
| 1, clone D10v3, | QDSKDSTYSLSSTLTLSKADYEKHKVYACEVTHQGLSSPVTKSFN | |
| B044-1_LC | RGECHHHHHH | |
| ROR-1, clone | QVQLKESGPGLVAPSQTLSITCTVSGFSLTSYGVHWVRQPPGKG | 100 |
| D10v3, B044- | LEWLGVIWAGGFTNYNSALKSRLSISKDNSKSQVLLKMTSLQTDD | |
| 2_HC | TAMYYCARRGSSYSMDYWGQGTSVTVSSASTKGPSVFPLAPSS | |
| | KSTSGGTAALGCLVKDYFPEPVTVSWNSGALTSGVHTFPAVLQS | |
| | SGLYSLSSVVTVPSSSLGTQTYICNVNHKPSNTKVDKKVEPKSCH | |
| | HHHHH | |

Constructs for binding PD-L1

| | | |
|---|---|---|
| E200 + PD-L1 | GHNYTTRNILPGLNITSDIQMTQSPSSLSASVGDRVTITCRASQDV | 101 |
| binder (nfP2X₇ | STAVAWYQQKPGKAPKLLIYSASFLYSGVPSRFSGSGSGTDFTLT | |
| epitope | ISSLQPEDFATYYCQQYLYHPATFGQGTKVEIKRTVAAPSVFIFPP | |
| underlined) PD- | SDEQLKSGTASVVCLLNNFYPREAKVQWKVDNALQSGNSQESV | |
| L1, Atezolizumab, | TEQDSKDSTYSLSSTLTLSKADYEKHKVYACEVTHQGLSSPVTKS | |
| B045-1_LC | FNRGECHHHHHH | |
| PD-L1, | EVQLVESGGGLVQPGGSLRLSCAASGFTFSDSWIHWVRQAPGK | 102 |
| Atezolizumab, | GLEWVAWISPYGGSTYYADSVKGRFTISADTSKNTAYLQMNSLR | |
| B045-2_HC | AEDTAVYYCARRHWPGGFDYWGQGTLVTVSSASTKGPSVFPLA | |
| | PSSKSTSGGTAALGCLVKDYFPEPVTVSWNSGALTSGVHTFPAV | |
| | LQSSGLYSLSSVVTVPSSSLGTQTYICNVNHKPSNTKVDKKVEPK | |
| | SCHHHHHH | |

Constructs for binding MET-R

| | | |
|---|---|---|
| E200 + MET-R | GHNYTTRNILPGLNITSDIQMTQSPSSLSASVGDRVTITCKSSQSL | 103 |
| binder (nfP2X₇ | LYTSSQKNYLAWYQQKPGKAPKLLIYWASTRESGVPSRFSGSGS | |
| epitope | GTDFTLTISSLQPEDFATYYCQQYYAYPWTFGQGTKVEIKRTVAA | |
| underlined) MET- | PSVFIFPPSDEQLKSGTASVVCLLNNFYPREAKVQWKVDNALQS | |
| R, Onartuzumab, | GNSQESVTEQDSKDSTYSLSSTLTLSKADYEKHKVYACEVTHQG | |
| B046-1_LC | LSSPVTKSFNRGECHHHHHH | |
| MET-R, | EVQLVESGGGLVQPGGSLRLSCAASGYTFTSYWLHWVRQAPGK | 104 |
| Onartuzumab, | GLEWVGMIDPSNSDTRFNPNFKDRFTISADTSKNTAYLQMNSLR | |
| B046-2_HC | AEDTAVYYCATYRSYVTPLDYWGQGTLVTVSSASTKGPSVFPLA | |
| | PSSKSTSGGTAALGCLVKDYFPEPVTVSWNSGALTSGVHTFPAV | |
| | LQSSGLYSLSSVVTVPSSSLGTQTYICNVNHKPSNTKVDKKVEPK | |
| | SCHHHHHH | |

Constructs for binding PDGFRalpha

| | | |
|---|---|---|
| E200 + PDGFR | GHNYTTRNILPGLNITSDIQMTQSPSSLSASVGDRVSITCRPSQSF | 105 |
| binder (nfP2X₇ | SRYINWYQQKPGKAPKLLIHAASSLVGGVPSRFSGSGSGTDFTLT | |
| epitope | ISSLQPEDFATYYCQQTYSNPPITFGQGTRLEMKRTVAAPSVFIFP | |
| underlined) | PSDEQLKSGTASVVCLLNNFYPREAKVQWKVDNALQSGNSQES | |
| PDGFRalpha, | VTEQDSKDSTYSLSSTLTLSKADYEKHKVYACEVTHQGLSSPVTK | |
| Tovetumab, | SFNRGECHHHHHH | |
| B047-1_LC | | |
| PDGFRalpha, | QVQLVESGGGLVKPGGSLRLSCAASGFTFSDYYMNWIRQAPGK | 106 |
| Tovetumab, | GLEWVSYISSSGSIIYYADSVKGRFTISRDNAKNSLYLQMNSLRAE | |
| B047-2_HC | DTAVYYCAREGRIAARGMDVWGQGTTVTVSSASTKGPSVFPLAP | |
| | SSKSTSGGTAALGCLVKDYFPEPVTVSWNSGALTSGVHTFPAVL | |
| | QSSGLYSLSSVVTVPSSSLGTQTYICNVNHKPSNTKVDKKVEPKS | |
| | CHHHHHH | |
| E200 + PDGFR | GHNYTTRNILPGLNITSEIVLTQSPATLSLSPGERATLSCRASQSV | 107 |
| binder (nfP2X₇ | SSYLAWYQQKPGQAPRLLIYDASNRATGIPARFSGSGSGTDFTLT | |
| epitope | ISSLEPEDFAVYYCQQRSNWPPAFGQGTKVEIKRTVAAPSVFIFP | |
| underlined) | PSDEQLKSGTASVVCLLNNFYPREAKVQWKVDNALQSGNSQES | |
| PDGFRalpha, | VTEQDSKDSTYSLSSTLTLSKADYEKHKVYACEVTHQGLSSPVTK | |
| Olaratumab, | SFNRGECHHHHHH | |
| B048-1_LC | | |
| PDGFRalpha, | QLQLQESGPGLVKPSETLSLTCTVSGGSINSSSYYWGWLRQSPG | 108 |
| Olaratumab, | KGLEWIGSFFYTGSTYYNPSLRSRLTISVDTSKNQFSLMLSSVTAA | |
| B048-2_HC | DTAVYYCARQSTYYYGSGNYYGWFDRWDQGTLVTVSSASTKGP | |
| | SVFPLAPSSKSTSGGTAALGCLVKDYFPEPVTVSWNSGALTSGV | |
| | HTFPAVLQSSGLYSLSSVVTVPSSSLGTQTYICNVNHKPSNTKVD | |
| | KKVEPKSCHHHHHH | |

Constructs for binding Her3

| | | |
|---|---|---|
| E200 + Her3 | GHNYTTRNILPGLNITSQSALTQPASVSGSPGQSITISCTGTSSDV | 109 |
| binder (nfP2X₇ | GSYNVVSWYQQHPGKAPKLIIYEVSQRPSGVSNRFSGSKSGNTA | |
| epitope | SLTISGLQTEDEADYYCCSYAGSSIFVIFGGGTKVTVLRTVAAPSV | |
| underlined) Her3, | FIFPPSDEQLKSGTASVVCLLNNFYPREAKVQWKVDNALQSGNS | |
| Seribantumab, | QESVTEQDSKDSTYSLSSTLTLSKADYEKHKVYACEVTHQGLSSP | |
| B049-1_LC | VTKSFNRGECHHHHHH | |
| Her3, | EVQLLESGGGLVQPGGSLRLSCAASGFTFSHYVMAWVRQAPGK | 110 |
| Seribantumab, | GLEWVSSISSSGGWTLYADSVKGRFTISRDNSKNTLYLQMNSLR | |
| B049-2_HC | AEDTAVYYCTRGLKMATIFDYWGQGTLVTVSSASTKGPSVFPLAP | |
| | SSKSTSGGTAALGCLVKDYFPEPVTVSWNSGALTSGVHTFPAVL | |
| | QSSGLYSLSSVVTVPSSSLGTQTYICNVNHKPSNTKVDKKVEPKS | |
| | CHHHHHH | |

Constructs for binding FRalpha

| | | |
|---|---|---|
| E200 + FRa binder | GHNYTTRNILPGLNITSDIQLTQSPSSLSASVGDRVTITCSVSSSIS | 111 |
| (nfP2X₇ epitope | SNNLHWYQQKPGKAPKPWIYGTSNLASGVPSRFSGSGSGTDYT | |
| underlined) | FTISSLQPEDIATYYCQQWSSYPYMYTFGQGTKVEIKRTVAAPSV | |
| FRalpha, | FIFPPSDEQLKSGTASVVCLLNNFYPREAKVQWKVDNALQSGNS | |
| Farletuzumab, | QESVTEQDSKDSTYSLSSTLTLSKADYEKHKVYACEVTHQGLSSP | |
| B050-1_LC | VTKSFNRGECHHHHHH | |
| FRalpha, | EVQLVESGGGVVQPGRSLRLSCSASGFTFSGYGLSWVRQAPGK | 112 |
| Farletuzumab, | GLEWVAMISSGGSYTYYADSVKGRFAISRDNAKNTLFLQMDSLR | |
| B050-2_HC | PEDTGVYFCARHGDDPAWFAYWGQGTPVTVSSASTKGPSVFPL | |
| | APSSKSTSGGTAALGCLVKDYFPEPVTVSWNSGALTSGVHTFPA | |
| | VLQSSGLYSLSSVVTVPSSSLGTQTYICNVNHKPSNTKVDKKVEP | |
| | KSCHHHHHH | |

Constructs for binding GPC3

| | | |
|---|---|---|
| E200 + GPC3 | GHNYTTRNILPGLNITSDVVMTQSPLSLPVTPGEPASISCRSSQSL | 113 |
| binder (nfP2X₇ | VHSNRNTYLHWYLQKPGQSPQLLIYKVSNRFSGVPDRFSGSGSG | |
| epitope | TDFTLKISRVEAEDVGVYYCSQNTHVPPTFGQGTKLEIKRTVAAP | |
| underlined) | SVFIFPPSDEQLKSGTASVVCLLNNFYPREAKVQWKVDNALQSG | |
| GPC3, | NSQESVTEQDSKDSTYSLSSTLTLSKADYEKHKVYACEVTHQGL | |
| Codrituzumab, | SSPVTKSFNRGECHHHHHH | |
| B051-1_LC | | |
| GPC3, | QVQLVQSGAEVKKPGASVKVSCKASGYTFTDYEMHWVRQAPGQ | 114 |
| Codrituzumab, | GLEWMGALDPKTGDTAYSQKFKGRVTLTADKSTSTAYMELSSLT | |
| B051-2_HC | SEDTAVYYCTRFYSYTYWGQGTLVTVSSASTKGPSVFPLAPSSK | |
| | STSGGTAALGCLVKDYFPEPVTVSWNSGALTSGVHTFPAVLQSS | |
| | GLYSLSSVVTVPSSSLGTQTYICNVNHKPSNTKVDKKVEPKSCHH | |
| | HHHH | |

Constructs for binding SLAMF7

| | | |
|---|---|---|
| E200 + SLAMF7 | GHNYTTRNILPGLNITSDIQMTQSPSSLSASVGDRVTITCKASQDV | 115 |
| binder (nfP2X₇ | GIAVAWYQQKPGKVPKLLIYWASTRHTGVPDRFSGSGSGTDFTL | |
| epitope | TISSLQPEDVATYYCQQYSSYPYTFGQGTKVEIKRTVAAPSVFIFP | |
| underlined) | PSDEQLKSGTASVVCLLNNFYPREAKVQWKVDNALQSGNSQES | |
| SLAMF7, | VTEQDSKDSTYSLSSTLTLSKADYEKHKVYACEVTHQGLSSPVTK | |
| Elotuzumab, | SFNRGECHHHHHH | |
| B052-1_LC | | |
| SLAMF7, | EVQLVESGGGLVQPGGSLRLSCAASGFDFSRYWMSWVRQAPG | 116 |
| Elotuzumab, | KGLEWIGEINPDSSTINYAPSLKDKFIISRDNAKNSLYLQMNSLRAE | |
| B052-2_HC | DTAVYYCARPDGNYWYFDVWGQGTLVTVSSASTKGPSVFPLAP | |
| | SSKSTSGGTAALGCLVKDYFPEPVTVSWNSGALTSGVHTFPAVL | |
| | QSSGLYSLSSVVTVPSSSLGTQTYICNVNHKPSNTKVDKKVEPKS | |
| | CHHHHHH | |

Constructs for binding TNFRSF10B

| | | |
|---|---|---|
| E200 + | GHNYTTRNILPGLNITSDIQMTQSPSSLSASVGDRVTITCKASQDV | 117 |
| TNFRSF10B | GTAVAWYQQKPGKAPKLLIYWASTRHTGVPSRFSGSGSGTDFTL | |
| binder (nfP2X₇ | TISSLQPEDFATYYCQQYSSYRTFGQGTKVEIKRTVAAPSVFIFPP | |
| epitope | SDEQLKSGTASVVCLLNNFYPREAKVQWKVDNALQSGNSQESV | |
| underlined), | TEQDSKDSTYSLSSTLTLSKADYEKHKVYACEVTHQGLSSPVTKS | |
| Tigatuzumab, | FNRGECHHHHHH | |
| B053-1_LC | | |
| TNFRSF10B, | EVQLVESGGGLVQPGGSLRLSCAASGFTFSSYVMSWVRQAPGK | 118 |
| Tigatuzumab, | GLEWVATISSGGSYTYYPDSVKGRFTISRDNAKNTLYLQMNSLRA | |
| B053-2_HC | EDTAVYYCARRGDSMITTDYWGQGTLVTVSSASTKGPSVFPLAP | |
| | SSKSTSGGTAALGCLVKDYFPEPVTVSWNSGALTSGVHTFPAVL | |
| | QSSGLYSLSSVVTVPSSSLGTQTYICNVNHKPSNTKVDKKVEPKS | |
| | CHHHHHH | |

Constructs for binding GPNMB

| | | |
|---|---|---|
| E200 + GPNMB | GHNYTTRNILPGLNITSEIVMTQSPATLSVSPGERATLSCRASQSV | 119 |
| binder (nfP2X₇ | DNNLVWYQQKPGQAPRLLIYGASTRATGIPARFSGSGSGTEFTLT | |
| epitope | ISSLQSEDFAVYYCQQYNNWPPWTFGQGTKVEIKRTVAAPSVFIF | |
| underlined), | PPSDEQLKSGTASVVCLLNNFYPREAKVQWKVDNALQSGNSQE | |
| GPNMB, | SVTEQDSKDSTYSLSSTLTLSKADYEKHKVYACEVTHQGLSSPVT | |
| Glembatumumab, | KSFNRGECHHHHHH | |
| B054-1_LC | | |
| GPNMB, | QVQLQESGPGLVKPSQTLSLTCTVSGGSISSFNYYWSWIRHHPG | 120 |
| Glembatumumab, | KGLEWIGYIYYSGSTYSNPSLKSRVTISVDTSKNQFSLTLSSVTAA | |
| B054-2_HC | DTAVYYCARGYNWNYFDYWGQGTLVTVSSASTKGPSVFPLAPS | |
| | SKSTSGGTAALGCLVKDYFPEPVTVSWNSGALTSGVHTFPAVLQ | |
| | SSGLYSLSSVVTVPSSSLGTQTYICNVNHKPSNTKVDKKVEPKSC | |
| | HHHHHH | |

Constructs for binding VEGFR2

| | | |
|---|---|---|
| E200 + VEGFR2 | GHNYTTRNILPGLNITSDIQMTQSPSSVSASIGDRVTITCRASQGID | 121 |
| binder (nfP2X₇ | NWLGWYQQKPGKAPKLLIYDASNLDTGVPSRFSGSGSGTYFTLTI | |
| epitope | SSLQAEDFAVYFCQQAKAFPPTFGGGTKVDIKRTVAAPSVFIFPP | |
| underlined), | SDEQLKSGTASVVCLLNNFYPREAKVQWKVDNALQSGNSQESV | |
| Ramucirumab, | TEQDSKDSTYSLSSTLTLSKADYEKHKVYACEVTHQGLSSPVTKS | |
| B055-1_LC | FNRGECHHHHHH | |
| VEGFR2, | EVQLVQSGGGLVKPGGSLRLSCAASGFTFSSYSMNWVRQAPGK | 122 |
| Ramucirumab, | GLEWVSSISSSSSYIYYADSVKGRFTISRDNAKNSLYLQMNSLRA | |
| B055-2_HC | EDTAVYYCARVTDAFDIWGQGTMVTVSSASTKGPSVFPLAPSSK | |
| | STSGGTAALGCLVKDYFPEPVTVSWNSGALTSGVHTFPAVLQSS | |
| | GLYSLSSVVTVPSSSLGTQTYICNVNHKPSNTKVDKKVEPKSCHH | |
| | HHHH | |

Constructs for binding α4β7 &/or αEβ7

| | | |
|---|---|---|
| E200 + α4β7 and | GHNYTTRNILPGLNITSDIQMTQSPSSLSASVGDRVTITCRASESV | 123 |
| αEβ7binder | DDLLHWYQQKPGKAPKLLIKYASQSISGVPSRFSGSGSGTDFTLT | |
| (nfP2X₇ epitope | ISSLQPEDFATYYCQQGNSLPNTFGQGTKVEIKRTVAAPSVFIFPP | |
| underlined), | SDEQLKSGTASVVCLLNNFYPREAKVQWKVDNALQSGNSQESV | |
| Etrolizumab, | TEQDSKDSTYSLSSTLTLSKADYEKHKVYACEVTHQGLSSPVTKS | |
| B056-1_LC | FNRGECHHHHHH | |
| α4β7 & αEβ7, | EVQLVESGGGLVQPGGSLRLSCAASGFFITNNYWGWVRQAPGK | 124 |
| Etrolizumab, | GLEWVGYISYSGSTSYNPSLKSRFTISRDTSKNTFYLQMNSLRAE | |
| B056-2_HC | DTAVYYCARTGSSGYFDFWGQGTLVTVSSASTKGPSVFPLAPSS | |
| | KSTSGGTAALGCLVKDYFPEPVTVSWNSGALTSGVHTFPAVLQS | |
| | SGLYSLSSVVTVPSSSLGTQTYICNVNHKPSNTKVDKKVEPKSCH | |
| | HHHHH | |
| E200 + α4β7 | GHNYTTRNILPGLNITSDIQMTQSPSSVSASVGDRVTITCRASQGI | 125 |
| binder (nfP2X₇ | SSWLAWYQQKPGKAPKLLIYGASNLESGVPSRFSGSGSGTDFTL | |
| epitope | TISSLQPEDFANYYCQQANSFPWTFGQGTKVEIKRTVAAPSVFIF | |
| underlined), α4β7, | PPSDEQLKSGTASVVCLLNNFYPREAKVQWKVDNALQSGNSQE | |
| Abrilumab, B057- | SVTEQDSKDSTYSLSSTLTLSKADYEKHKVYACEVTHQGLSSPVT | |
| 1 LC | KSFNRGECHHHHHH | |
| α4β7, Abrilumab, | QVQLVQSGAEVKKPGASVKVSCKVSGYTLSDLSIHWVRQAPGKG | 126 |
| B057-2_HC | LEWMGGFDPQDGETIYAQKFQGRVTMTEDTSTDTAYMELSSLKS | |
| | EDTAVYYCATGSSSSWFDPWGQGTLVTVSSASTKGPSVFPLAPS | |
| | SKSTSGGTAALGCLVKDYFPEPVTVSWNSGALTSGVHTFPAVLQ | |
| | SSGLYSLSSVVTVPSSSLGTQTYICNVNHKPSNTKVDKKVEPKSC | |
| | HHHHHH | |

Constructs for binding CSPG4

| | | |
|---|---|---|
| E200 + CSPG4 | GHNYTTRNILPGLNITSRSTQSALTQPASVSGSPGQSITISCTGTS | 127 |
| binder (nfP2X₇ | SDVGGYNYVSWYQQHPGKAPKLMIYDVSNRPSGVSKRFSGSKS | |
| epitope | GNTASLTISGLQAEDEADYYCSSYTSSSTRHVFGTGTQLTVLGRT | |
| underlined), | VAAPSVFIFPPSDEQLKSGTASVVCLLNNFYPREAKVQWKVDNAL | |
| CSPG4, D2A- | QSGNSQESVTEQDSKDSTYSLSSTLTLSKADYEKHKVYACEVTH | |
| 1h10-UC12, | QGLSSPVTKSFNRGECHHHHHH | |
| B058-1_LC | | |
| CSPG4, D2A- | EVQLVESGAEVKKPGDSLKISCKGSGYSFTSYWIGWVRQMPGK | 128 |
| 1h10-UC12, | GLEWMGIIYPGDSVTTYSPAFQGDVTISVDKSISTAYLQWNSLKA | |
| B058-2_HC | SDTGIYYCARRRGNYYMDVWGNGTLVTVSSASTKGPSVFPLAPS | |
| | SKSTSGGTAALGCLVKDYFPEPVTVSWNSGALTSGVHTFPAVLQ | |
| | SSGLYSLSSVVTVPSSSLGTQTYICNVNHKPSNTKVDKKVEPKSC | |
| | HHHHHH | |

Constructs for binding CD80

| | | |
|---|---|---|
| E200 + CD80 | GHNYTTRNILPGLNITSESALTQPPSVSGAPGQKVTISCTGSTSNI | 129 |
| binder (nfP2X₇ | GGYDLHWYQQLPGTAPKLLIYDINKRPSGISDRFSGSKSGTAASL | |
| epitope | AITGLQTEDEADYYCQSYDSSLNAQVFGGGTRLTVLRTVAAPSVF | |
| underlined), | IFPPSDEQLKSGTASVVCLLNNFYPREAKVQWKVDNALQSGNSQ | |
| Galiximab, B059- | ESVTEQDSKDSTYSLSSTLTLSKADYEKHKVYACEVTHQGLSSPV | |
| 1_LC | TKSFNRGECHHHHHH | |
| CD80, Galiximab, | QVQLQESGPGLVKPSETLSLTCAVSGGSISGGYGWGWIRQPPG | 130 |
| B059-2_HC | KGLEWIGSFYSSSGNTYYNPSLKSQVTISTDTSKNQFSLKLNSMT | |
| | AADTAVYYCVRDRLFSVVGMVYNNWFDVWGPGVLVTVSSASTK | |
| | GPSVFPLAPSSKSTSGGTAALGCLVKDYFPEPVTVSWNSGALTS | |
| | GVHTFPAVLQSSGLYSLSSVVTVPSSSLGTQTYICNVNHKPSNTK | |
| | VDKKVEPKSCHHHHHH | |

Constructs for binding CCR4

| | | |
|---|---|---|
| E200 + CCR4 | GHNYTTRNILPGLNITSDVLMTQSPLSLPVTPGEPASISCRSSRNI | 131 |
| binder (nfP2X₇ | VHINGDTYLEWYLQKPGQSPQLLIYKVSNRFSGVPDRFSGSGSG | |
| epitope | TDFTLKISRVEAEDVGVYYCFQGSLLPWTFGQGTKVEIKRTVAAP | |
| underlined), | SVFIFPPSDEQLKSGTASVVCLLNNFYPREAKVQWKVDNALQSG | |
| Mogamulizumab, | NSQESVTEQDSKDSTYSLSSTLTLSKADYEKHKVYACEVTHQGL | |
| B060-1 LC | SSPVTKSFNRGECHHHHHH | |
| CCR4, | EVQLVESGGDLVQPGRSLRLSCAASGFIFSNYGMSWVRQAPGK | 132 |
| Mogamulizumab, | GLEWVATISSASTYSYYPDSVKGRFTISRDNAKNSLYLQMNSLRV | |
| B060-2_HC | EDTALYYCGRHSDGNFAFGYWGQGTLVTVSSASTKGPSVFPLAP | |
| | SSKSTSGGTAALGCLVKDYFPEPVTVSWNSGALTSGVHTFPAVL | |
| | QSSGLYSLSSVVTVPSSSLGTQTYICNVNHKPSNTKVDKKVEPKS | |
| | CHHHHHH | |

Constructs for binding CD115

| | | |
|---|---|---|
| E200 + CD115 | GHNYTTRNILPGLNITSDIQMTQSPSSLSASVGDRVTITCRASEDV | 133 |
| binder (nfP2X₇ | NTYVSWYQQKPGKAPKLLIYAASNRYTGVPSRFSGSGSGTDFTL | |
| epitope | TISSLQPEDFATYYCQQSFSYPTFGQGTKLEIKRTVAAPSVFIFPP | |
| underlined), | SDEQLKSGTASVVCLLNNFYPREAKVQWKVDNALQSGNSQESV | |
| CD115-CSF-1R, | TEQDSKDSTYSLSSTLTLSKADYEKHKVYACEVTHQGLSSPVTKS | |
| Emactuzumab, | FNRGECHHHHHH | |
| B061-1_LC | | |
| CD115-CSF-1R, | QVQLVQSGAEVKKPGASVKVSCKASGYTFTSYDISWVRQAPGQ | 134 |
| Emactuzumab, | GLEWMGVIWTDGGTNYAQKLQGRVTMTTDTSTSTAYMELRSLR | |
| B061-2_HC | SDDTAVYYCARDQRLYFDVWGQGTTVTVSSASTKGPSVFPLAPS | |
| | SKSTSGGTAALGCLVKDYFPEPVTVSWNSGALTSGVHTFPAVLQ | |
| | SSGLYSLSSVVTVPSSSLGTQTYICNVNHKPSNTKVDKKVEPKSC | |
| | HHHHHH | |

Constructs for binding ENOX-2

| | | |
|---|---|---|
| E200 + ENOX-2 | GHNYTTRNILPGLNITSENVLTQSPAIMSASPGERVTMTCSASSSI | 135 |
| binder (nfP2X₇ | RYIYWYQQKPGSSPRLLIYDTSNVAPGVPFRFSGSGSGTSYSLTI | |
| epitope | NRMEAEDAATYYCQEWSGYPYTFGGGTKLELKRTVAAPSVFIFP | |
| underlined), | PSDEQLKSGTASVVCLLNNFYPREAKVQWKVDNALQSGNSQES | |
| ENOX-2, | VTEQDSKDSTYSLSSTLTLSKADYEKHKVYACEVTHQGLSSPVTK | |
| US9459256, | SFNRGECHHHHHH | |
| B062-1_LC | | |
| ENOX-2, | EVKLQESGTEVVKPGASVKLSCKASGYIFTSYDIDWVRQTPEQGL | 136 |
| US9459256, | EWIGWIFPGEGSTEYNEKFKGRATLSVDKSSSTAYMELTRLTSED | |
| B062-2_HC | SAVYFCARGDYYRRYFDLWGQGTTVTVSSASTKGPSVFPLAPSS | |
| | KSTSGGTAALGCLVKDYFPEPVTVSWNSGALTSGVHTFPAVLQS | |
| | SGLYSLSSVVTVPSSSLGTQTYICNVNHKPSNTKVDKKVEPKSCH | |
| | HHHHH | |

Constructs for binding CD56

| | | |
|---|---|---|
| E200 + CD56 | GHNYTTRNILPGLNITSDVVMTQSPLSLPVTLGQPASISCRSSQIII | 137 |
| binder (nfP2X₇ | HSDGNTYLEWFQQRPGQSPRRLIYKVSNRFSGVPDRFSGSGSG | |
| epitope | TDFTLKISRVEAEDVGVYYCFQGSHVPHTFGQGTKVEIKRTVAAP | |
| underlined), | SVFIFPPSDEQLKSGTASVVCLLNNFYPREAKVQWKVDNALQSG | |
| CD56, | NSQESVTEQDSKDSTYSLSSTLTLSKADYEKHKVYACEVTHQGL | |
| Lorvotuzumab, | SSPVTKSFNRGECHHHHHH | |
| B063-1_LC | | |
| CD56, | QVQLVESGGGVVQPGRSLRLSCAASGFTFSSFGMHWVRQAPGK | 138 |
| Lorvotuzumab, | GLEWVAYISSGSFTIYYADSVKGRFTISRDNSKNTLYLQMNSLRA | |
| B063-2_HC | EDTAVYYCARMRKGYAMDYWGQGTLVTVSSASTKGPSVFPLAP | |
| | SSKSTSGGTAALGCLVKDYFPEPVTVSWNSGALTSGVHTFPAVL | |
| | QSSGLYSLSSVVTVPSSSLGTQTYICNVNHKPSNTKVDKKVEPKS | |
| | CHHHHHH | |

Constructs for binding huVH1-69

| | | |
|---|---|---|
| E200 + huVH1-69 | GHNYTTRNILPGLNITSDIQLTQSPSSLSASVGDRVTITCRASQGIS | 139 |
| binder (nfP2X₇ | SNIVWLQQKPGKAPKGLIYHGTNLESGVPSRFSGSGSGTDYTLTI | |
| epitope | SSLEPEDFATYYCVQYSQFPPTFGQGTKLEIKRTVAAPSVFIFPPS | |
| underlined), | DEQLKSGTASVVCLLNNFYPREAKVQWKVDNALQSGNSQESVT | |
| huVH1-69, B075- | EQDSKDSTYSLSSTLTLSKADYEKHKVYACEVTHQGLSSPVTKSF | |
| 1_LC | NRGECHHHHHH | |
| huVH1-69, B075- | QVQLVQSGAEVVKPGASVKVSCKASGYTFTSYWMHWVKQAPG | 140 |
| 2_HC | QGLEWIGAVSPGNSDTSYNEKFKGKATLTVDTSASTAYMELSSL | |
| | RSEDTAVYYCTRSRYGNNALDYWGQGTLVTVSASTKGPSVFPLA | |
| | PSSKSTSGGTAALGCLVKDYFPEPVTVSWNSGALTSGVHTFPAV | |
| | LQSSGLYSLSSVVTVPSSSLGTQTYICNVNHKPSNTKVDKKVEPK | |
| | SCHHHHHH | |

Constructs for binding CD19 (IgG1 format)

| | | |
|---|---|---|
| CD19, | EVQLVESGGGLVKPGGSLKLSCAASGYTFTSYVMHWVRQAPGK | 141 |
| Tafasitamab, | GLEWIGYINPYNDGTKYNEKFQGRVTISSDKSISTAYMELSSLRSE | |
| OR19_1, wt-IgG1, | DTAMYYCARGTYYYGTRVFDYWGQGTLVTVSSASTKGPSVFPLA | |
| HC | PSSKSTSGGTAALGCLVKDYFPEPVTVSWNSGALTSGVHTFPAV | |
| | LQSSGLYSLSSVVTVPSSSLGTQTYICNVNHKPSNTKVDKKVEPK | |
| | SCDKTHTCPPCPAPELLGGPSVFLFPPKPKDTLMISRTPEVTCVV | |
| | VDVSHEDPEVKFNWYVDGVEVHNAKTKPREEQYNSTYRVVSVLT | |
| | VLHQDWLNGKEYKCKVSNKALPAPIEKTISKAKGQPREPQVYTLP | |
| | PSRDELTKNQVSLTCLVKGFYPSDIAVEWESNGQPENNYKTTPP | |
| | VLDSDGSFFLYSKLTVDKSRWQQGNVFSCSVMHEALHNHYTQK | |
| | SLSLSPGKHHHHHH | |
| CD19, | EVQLVESGGGLVKPGGSLKLSCAASGYTFTSYVMHWVRQAPGK | 142 |
| Tafasitamab, | GLEWIGYINPYNDGTKYNEKFQGRVTISSDKSISTAYMELSSLRSE | |
| OR19_2, IgG1- | DTAMYYCARGTYYYGTRVFDYWGQGTLVTVSSASTKGPSVFPLA | |
| SDIE, HC | PSSKSTSGGTAALGCLVKDYFPEPVTVSWNSGALTSGVHTFPAV | |
| | LQSSGLYSLSSVVTVPSSSLGTQTYICNVNHKPSNTKVDKKVEPK | |
| | SCDKTHTCPPCPAPELLGGPDVFLFPPKPKDTLMISRTPEVTCVV | |
| | VDVSHEDPEVKFNWYVDGVEVHNAKTKPREEQYNSTYRVVSVLT | |
| | VLHQDWLNGKEYKCKVSNKALPAPEEKTISKAKGQPREPQVYTL | |
| | PPSRDELTKNQVSLTCLVKGFYPSDIAVEWESNGQPENNYKTTP | |
| | PVLDSDGSFFLYSKLTVDKSRWQQGNVFSCSVMHEALHNHYTQ | |
| | KSLSLSPGKHHHHHH | |

Constructs for binding CD19 (Fab format)

| | | |
|---|---|---|
| CD19, | EVQLVESGGGLVKPGGSLKLSCAASGYTFTSYVMHWVRQAPGK | 143 |
| Tafasitamab, | GLEWIGYINPYNDGTKYNEKFQGRVTISSDKSISTAYMELSSLRSE | |
| B020-2_Heavy | DTAMYYCARGTYYYGTRVFDYWGQGTLVTVSSASTKGPSVFPLA | |
| chain | PSSKSTSGGTAALGCLVKDYFPEPVTVSWNSGALTSGVHTFPAV | |
| | LQSSGLYSLSSVVTVPSSSLGTQTYICNVNHKPSNTKVDKKVEPK | |
| | SCHHHHHH | |
| CD19, | DIVMTQSPATLSLSPGERATLSCRSSKSLQNVNGNTYLYWFQQK | 144 |
| Tafasitamab, | PGQSPQLLIYRMSNLNSGVPDRFSGSGSGTEFTLTISSLEPEDFA | |
| OR19_7 | VYYCMQHLEYPITFGAGTKLEIKRTVAAPSVFIFPPSDEQLKSGTA | |
| Light chain | SVVCLLNNFYPREAKVQWKVDNALQSGNSQESVTEQDSKDSTY | |
| | SLSSTLTLSKADYEKHKVYACEVTHQGLSSPVTKSFNRGEC | |
| E200 + CD19 | GHNYTTRNILPGLNITSDIVMTQSPATLSLSPGERATLSCRSSKSL | 145 |
| binder (nfP2X₇ | QNVNGNTYLYWFQQKPGQSPQLLIYRMSNLNSGVPDRFSGSGS | |
| epitope | GTEFTLTISSLEPEDFAVYYCMQHLEYPITFGAGTKLEIKRTVAAPS | |
| underlined), | VFIFPPSDEQLKSGTASVVCLLNNFYPREAKVQWKVDNALQSGN | |
| CD19, | SQESVTEQDSKDSTYSLSSTLTLSKADYEKHKVYACEVTHQGLSS | |
| Tafasitamab, | PVTKSFNRGEC | |
| Light chain | | |
| OR19_8 | | |
| E200 + CD19 | GHNYTTRNILPGLNITSGGGGSDIVMTQSPATLSLSPGERATLSC | 146 |
| binder (nfP2X₇ | RSSKSLQNVNGNTYLYWFQQKPGQSPQLLIYRMSNLNSGVPDRF | |
| epitope | SGSGSGTEFTLTISSLEPEDFAVYYCMQHLEYPITFGAGTKLEIKR | |
| underlined), | TVAAPSVFIFPPSDEQLKSGTASVVCLLNNFYPREAKVQWKVDNA | |
| CD19, | LQSGNSQESVTEQDSKDSTYSLSSTLTLSKADYEKHKVYACEVT | |
| Tafasitamab, | HQGLSSPVTKSFNRGEC | |
| Light chain | | |
| OR19_9 | | |
| E200 + CD19 | GHNYTTRNILPGLNITSGGGGSGGGGSGGGGSDIVMTQSPATLS | 147 |
| binder (nfP2X₇ | LSPGERATLSCRSSKSLQNVNGNTYLYWFQQKPGQSPQLLIYRM | |
| epitope | SNLNSGVPDRFSGSGSGTEFTLTISSLEPEDFAVYYCMQHLEYPI | |
| underlined), | TFGAGTKLEIKRTVAAPSVFIFPPSDEQLKSGTASVVCLLNNFYPR | |
| CD19, | EAKVQWKVDNALQSGNSQESVTEQDSKDSTYSLSSTLTLSKADY | |
| Tafasitamab, | EKHKVYACEVTHQGLSSPVTKSFNRGEC | |
| Light chain | | |
| OR19_10 | | |
| E200 + CD19 | GHNYTTRNILPGLNITSTFHKTSGSGKDIVMTQSPATLSLSPGERA | 148 |
| binder (nfP2X₇ | TLSCRSSKSLQNVNGNTYLYWFQQKPGQSPQLLIYRMSNLNSGV | |
| epitope | PDRFSGSGSGTEFTLTISSLEPEDFAVYYCMQHLEYPITFGAGTKL | |
| underlined), | EIKRTVAAPSVFIFPPSDEQLKSGTASVVCLLNNFYPREAKVQWK | |
| CD19, | VDNALQSGNSQESVTEQDSKDSTYSLSSTLTLSKADYEKHKVYA | |
| Tafasitamab, | CEVTHQGLSSPVTKSFNRGEC | |
| Light chain | | |
| OR19_11 | | |
| E200 + CD19 | GHNYTTRNILPGLNITSTFHKTDIVMTQSPATLSLSPGERATLSCR | 149 |
| binder (nfP2X₇ | SSKSLQNVNGNTYLYWFQQKPGQSPQLLIYRMSNLNSGVPDRFS | |
| epitope | GSGSGTEFTLTISSLEPEDFAVYYCMQHLEYPITFGAGTKLEIKRT | |
| underlined), | VAAPSVFIFPPSDEQLKSGTASVVCLLNNFYPREAKVQWKVDNAL | |
| CD19, | QSGNSQESVTEQDSKDSTYSLSSTLTLSKADYEKHKVYACEVTH | |
| tafasitamab, Light | QGLSSPVTKSFNRGEC | |
| chain | | |
| OR19 12 | | |
| E200 + CD19 | GHNYTTRNILPGLNITSTFHKTGSDIVMTQSPATLSLSPGERATLS | 150 |
| binder (nfP2X₇ | CRSSKSLQNVNGNTYLYWFQQKPGQSPQLLIYRMSNLNSGVPD | |
| epitope | RFSGSGSGTEFTLTISSLEPEDFAVYYCMQHLEYPITFGAGTKLEI | |
| underlined), | KRTVAAPSVFIFPPSDEQLKSGTASVVCLLNNFYPREAKVQWKVD | |
| CD19, | NALQSGNSQESVTEQDSKDSTYSLSSTLTLSKADYEKHKVYACE | |
| Tafasitamab, | VTHQGLSSPVTKSFNRGEC | |
| Light chain | | |
| OR19_13 | | |
| E200 + CD19 | GHNYTTRNILPGLNITSTFHGSDIVMTQSPATLSLSPGERATLSCR | 151 |
| binder (nfP2X₇ | SSKSLQNVNGNTYLYWFQQKPGQSPQLLIYRMSNLNSGVPDRFS | |
| epitope | GSGSGTEFTLTISSLEPEDFAVYYCMQHLEYPITFGAGTKLEIKRT | |
| underlined), | VAAPSVFIFPPSDEQLKSGTASVVCLLNNFYPREAKVQWKVDNAL | |
| CD19, | QSGNSQESVTEQDSKDSTYSLSSTLTLSKADYEKHKVYACEVTH | |
| Tafasitamab, | QGLSSPVTKSFNRGEC | |
| Light chain | | |
| OR19_14 | | |
| E200 + CD19 | GHNYTTRNILPGLNITSGSDIVMTQSPATLSLSPGERATLSCRSSK | 152 |
| binder (nfP2X₇ | SLQNVNGNTYLYWFQQKPGQSPQLLIYRMSNLNSGVPDRFSGS | |
| epitope | GSGTEFTLTISSLEPEDFAVYYCMQHLEYPITFGAGTKLEIKRTVA | |
| underlined), | APSVFIFPPSDEQLKSGTASVVCLLNNFYPREAKVQWKVDNALQS | |
| CD19, | GNSQESVTEQDSKDSTYSLSSTLTLSKADYEKHKVYACEVTHQG | |
| Tafasitamab, | LSSPVTKSFNRGEC | |
| Light chain | | |
| OR19_15 | | |
| E200 + CD19 | DFPGHNYTTRNILPGLNITSGSDIVMTQSPATLSLSPGERATLSCR | 153 |
| binder (nfP2X₇ | SSKSLQNVNGNTYLYWFQQKPGQSPQLLIYRMSNLNSGVPDRFS | |
| epitope | GSGSGTEFTLTISSLEPEDFAVYYCMQHLEYPITFGAGTKLEIKRT | |
| underlined), | VAAPSVFIFPPSDEQLKSGTASVVCLLNNFYPREAKVQWKVDNAL | |
| CD19, | QSGNSQESVTEQDSKDSTYSLSSTLTLSKADYEKHKVYACEVTH | |
| Tafasitamab, | QGLSSPVTKSFNRGEC | |
| Light chain | | |
| OR19_NEW_001 | | |
| E200 + CD19 | DFPGHNYTTRNILPGLNITSGGGGSDIVMTQSPATLSLSPGERATL | 154 |
| binder (nfP2X₇ | SCRSSKSLQNVNGNTYLYWFQQKPGQSPQLLIYRMSNLNSGVP | |
| epitope | DRFSGSGSGTEFTLTISSLEPEDFAVYYCMQHLEYPITFGAGTKLE | |
| underlined), | IKRTVAAPSVFIFPPSDEQLKSGTASVVCLLNNFYPREAKVQWKV | |
| CD19, | DNALQSGNSQESVTEQDSKDSTYSLSSTLTLSKADYEKHKVYAC | |
| Tafasitamab, | EVTHQGLSSPVTKSFNRGEC | |
| Light chain | | |
| OR19_NEW_002 | | |
| E200 + CD19 | DFPGHNYTTRNILPGLNITSGGGGSGGGGSDIVMTQSPATLSLSP | 155 |
| binder (nfP2X₇ | GERATLSCRSSKSLQNVNGNTYLYWFQQKPGQSPQLLIYRMSNL | |
| epitope | NSGVPDRFSGSGSGTEFTLTISSLEPEDFAVYYCMQHLEYPITFG | |
| underlined), | AGTKLEIKRTVAAPSVFIFPPSDEQLKSGTASVVCLLNNFYPREAK | |
| CD19, | VQWKVDNALQSGNSQESVTEQDSKDSTYSLSSTLTLSKADYEKH | |
| Tafasitamab, | KVYACEVTHQGLSSPVTKSFNRGEC | |
| Light chain | | |
| OR19_NEW_003 | | |
| E200 + CD19 | DFPGHNYTTRNILPGLNITSGGGGSGGGGSGGGGSDIVMTQSPA | 156 |
| binder (nfP2X₇ | TLSLSPGERATLSCRSSKSLQNVNGNTYLYWFQQKPGQSPQLLI | |
| epitope | YRMSNLNSGVPDRFSGSGSGTEFTLTISSLEPEDFAVYYCMQHL | |
| underlined), | EYPITFGAGTKLEIKRTVAAPSVFIFPPSDEQLKSGTASVVCLLNNF | |
| CD19, | YPREAKVQWKVDNALQSGNSQESVTEQDSKDSTYSLSSTLTLSK | |
| Tafasitamab, | ADYEKHKVYACEVTHQGLSSPVTKSFNRGEC | |
| Light chain | | |
| OR19_NEW_004 | | |
| E200 + CD19 | DFPGHNYTTRNILPGLNITSTFHKTSGSGKDIVMTQSPATLSLSPG | 157 |
| binder (nfP2X₇ | ERATLSCRSSKSLQNVNGNTYLYWFQQKPGQSPQLLIYRMSNLN | |
| epitope | SGVPDRFSGSGSGTEFTLTISSLEPEDFAVYYCMQHLEYPITFGA | |
| underlined), | GTKLEIKRTVAAPSVFIFPPSDEQLKSGTASVVCLLNNFYPREAKV | |
| CD19, | QWKVDNALQSGNSQESVTEQDSKDSTYSLSSTLTLSKADYEKHK | |
| Tafasitamab, | VYACEVTHQGLSSPVTKSFNRGEC | |
| Light chain | | |
| OR19_NEW_005 | | |
| E200 + CD19 | DFPGHNYTTRNILPGLNITSTFHKTSGSGKGSDIVMTQSPATLSLS | 158 |
| binder (nfP2X₇ | PGERATLSCRSSKSLQNVNGNTYLYWFQQKPGQSPQLLIYRMSN | |
| epitope | LNSGVPDRFSGSGSGTEFTLTISSLEPEDFAVYYCMQHLEYPITF | |
| underlined), | GAGTKLEIKRTVAAPSVFIFPPSDEQLKSGTASVVCLLNNFYPREA | |
| CD19, | KVQWKVDNALQSGNSQESVTEQDSKDSTYSLSSTLTLSKADYEK | |
| Tafasitamab, | HKVYACEVTHQGLSSPVTKSFNRGEC | |
| Light | | |
| chainOR19_NEW | | |
| 006 | | |
| E200 + CD19 | DFPGHNYTTRNILPGLNITSTFHKTSGSGKGGGGSDIVMTQSPAT | 159 |
| binder (nfP2X₇ | LSLSPGERATLSCRSSKSLQNVNGNTYLYWFQQKPGQSPQLLIY | |
| epitope | RMSNLNSGVPDRFSGSGSGTEFTLTISSLEPEDFAVYYCMQHLE | |
| underlined), | YPITFGAGTKLEIKRTVAAPSVFIFPPSDEQLKSGTASVVCLLNNFY | |
| CD19, | PREAKVQWKVDNALQSGNSQESVTEQDSKDSTYSLSSTLTLSKA | |
| Tafasitamab, | DYEKHKVYACEVTHQGLSSPVTKSFNRGEC | |
| Light chain | | |
| OR19_NEW_007 | | |
| E200 + CD19 | DFPGHNYTTRNILPGLNITSTFHGGGGSDIVMTQSPATLSLSPGE | 160 |
| binder (nfP2X₇ | RATLSCRSSKSLQNVNGNTYLYWFQQKPGQSPQLLIYRMSNLNS | |
| epitope | GVPDRFSGSGSGTEFTLTISSLEPEDFAVYYCMQHLEYPITFGAG | |
| underlined), | TKLEIKRTVAAPSVFIFPPSDEQLKSGTASVVCLLNNFYPREAKVQ | |
| CD19, | WKVDNALQSGNSQESVTEQDSKDSTYSLSSTLTLSKADYEKHKV | |
| Tafasitamab, | YACEVTHQGLSSPVTKSFNRGEC | |
| Light chain | | |
| OR19_NEW_008 | | |
| E200 + CD19 | GHNYTTRNILPGLNITSTFHGGGGSDIVMTQSPATLSLSPGERATL | 161 |
| binder (nfP2X₇ | SCRSSKSLQNVNGNTYLYWFQQKPGQSPQLLIYRMSNLNSGVP | |
| epitope | DRFSGSGSGTEFTLTISSLEPEDFAVYYCMQHLEYPITFGAGTKLE | |
| underlined), | IKRTVAAPSVFIFPPSDEQLKSGTASVVCLLNNFYPREAKVQWKV | |
| CD19, | DNALQSGNSQESVTEQDSKDSTYSLSSTLTLSKADYEKHKVYAC | |
| Tafasitamab, | EVTHQGLSSPVTKSFNRGEC | |
| Light chain | | |
| OR19_NEW_009 | | |
| E200 + CD19 | DFPGHNYTTRNILPGLNITSTFHKTGGGGSDIVMTQSPATLSLSPG | 162 |
| binder (nfP2X₇ | ERATLSCRSSKSLQNVNGNTYLYWFQQKPGQSPQLLIYRMSNLN | |
| epitope | SGVPDRFSGSGSGTEFTLTISSLEPEDFAVYYCMQHLEYPITFGA | |
| underlined), | GTKLEIKRTVAAPSVFIFPPSDEQLKSGTASVVCLLNNFYPREAKV | |
| CD19, | QWKVDNALQSGNSQESVTEQDSKDSTYSLSSTLTLSKADYEKHK | |
| Tafasitamab, | VYACEVTHQGLSSPVTKSFNRGEC | |
| Light chain | | |
| OR19_NEW_010 | | |
| E200 + CD19 | GHNYTTRNILPGLNITSTFHKTGGGGSDIVMTQSPATLSLSPGER | 163 |
| binder (nfP2X₇ | ATLSCRSSKSLQNVNGNTYLYWFQQKPGQSPQLLIYRMSNLNSG | |
| epitope | VPDRFSGSGSGTEFTLTISSLEPEDFAVYYCMQHLEYPITFGAGT | |
| underlined), | KLEIKRTVAAPSVFIFPPSDEQLKSGTASVVCLLNNFYPREAKVQ | |
| CD19, | WKVDNALQSGNSQESVTEQDSKDSTYSLSSTLTLSKADYEKHKV | |
| Tafasitamab, | YACEVTHQGLSSPVTKSFNRGEC | |
| Light chain | | |
| OR19_NEW_011 | | |
| E200 + CD19 | GHNYTTRNILPGLNITSGGGGSGGGGSDIVMTQSPATLSLSPGER | 164 |
| binder (nfP2X₇ | ATLSCRSSKSLQNVNGNTYLYWFQQKPGQSPQLLIYRMSNLNSG | |
| epitope | VPDRFSGSGSGTEFTLTISSLEPEDFAVYYCMQHLEYPITFGAGT | |
| underlined), | KLEIKRTVAAPSVFIFPPSDEQLKSGTASVVCLLNNFYPREAKVQ | |
| CD19, | WKVDNALQSGNSQESVTEQDSKDSTYSLSSTLTLSKADYEKHKV | |
| Tafasitamab, | YACEVTHQGLSSPVTKSFNRGEC | |
| Light chain | | |
| OR19_NEW_012 | | |

Exemplary CAR constructs

| | | |
|---|---|---|
| CAR7 (anti- | MALPVTALLLPLALLLHAARPEVQLLESGGGLVQPGGSLRLSCAA | 165 |
| nfP2X7) | SGFTFRNHDMGWVRQAPGKGLEWVSAISGSGGSTYYANSVKGR | |
| | FTISRDNSKNTLYLQMNSLRAEDTAVYYCAEPKPMDTEFDYRSP | |
| | GTLVTVSSRAAAIEVMYPPPYLDNEKSNGTIIHVKGKHLCPSPLFP | |
| | GPSKPFWVLVVVGGVLACYSLLVTVAFIIFWVRSKRSRLLHSDYM | |
| | NMTPRRPGPTRKHYQPYAPPRDFAAYRSKRGRKKLLYIFKQPFM | |
| | RPVQTTQEEDGCSCRFPEEEEGGCELRVKFSRSADAPAYKQGQ | |
| | NQLYNELNLGRREEYDVLDKRRGRDPEMGGKPRRKNPQEGLYN | |
| | ELQKDKMAEAYSEIGMKGERRRGKGHDGLYQGLSTATKDTYDAL | |
| | HMQALPPRLEGGGEGRGSLLTCGDVEENPGPRMLLLVTSLLLCE | |
| | LPHPAFLLIPRKVCNGIGIGEFKDSLSINATNIKHFKNCTSISGDLHI | |
| | LPVAFRGDSFTHTPPLDPQELDILKTVKEITGFLLIQAWPENRTDL | |
| | HAFENLEIIRGRTKQHGQFSLAVVSLNITSLGLRSLKEISDGDVIISG | |
| | NKNLCYANTINWKKLFGTSGQKTKIISNRGENSCKATGQVCHALC | |
| | SPEGCWGPEPRDCVSCRNVSRGRECVDKCNLLEGEPREFVENS | |
| | ECIQCHPECLPQAMNITCTGRGPDNCIQCAHYIDGPHCVKTCPAG | |
| | VMGENNTLVWKYADAGHVCHLCHPNCTYGCTGPGLEGCPTNGP | |
| | KIPSIATGMVGALLLLLVVALGIGLFM | |
| CAR10 | MALPVTALLLPLALLLHAARPEVQLLESGGGLVQPGGSLRLSCAA | 166 |
| (anti-nfP2X7) | SGFTFRNHDMGWVRQAPGKGLEWVSAISGSGGSTYYANSVKGR | |
| | FTISRDNSKNTLYLQMNSLRAEDTAVYYCAEPKPMDTEFDYRSP | |
| | GTLVTVSSTTTPAPRPPTPAPTIASQPLSLRPEACRPAAGGAVHT | |
| | RGLDFACDFWVLVVVGGVLACYSLLVTVAFIIFWVRSKRSRLLHS | |
| | DYMNMTPRRPGPTRKHYQPYAPPRDFAAYRSKRGRKKLLYIFKQ | |
| | PFMRPVQTTQEEDGCSCRFPEEEEGGCELRVKFSRSADAPAYK | |
| | QGQNQLYNELNLGRREEYDVLDKRRGRDPEMGGKPRRKNPQE | |
| | GLYNELQKDKMAEAYSEIGMKGERRRGKGHDGLYQGLSTATKD | |
| | TYDALHMQALPPRLEGGGEGRGSLLTCGDVEENPGPRMLLLVTS | |
| | LLLCELPHPAFLLIPRKVCNGIGIGEFKDSLSINATNIKHFKNCTSIS | |
| | GDLHILPVAFRGDSFTHTPPLDPQELDILKTVKEITGFLLIQAWPEN | |
| | RTDLHAFENLEIIRGRTKQHGQFSLAVVSLNITSLGLRSLKEISDGD | |
| | VIISGNKNLCYANTINWKKLFGTSGQKTKIISNRGENSCKATGQVC | |
| | HALCSPEGCWGPEPRDCVSCRNVSRGRECVDKCNLLEGEPREF | |
| | VENSECIQCHPECLPQAMNITCTGRGPDNCIQCAHYIDGPHCVKT | |
| | CPAGVMGENNTLVWKYADAGHVCHLCHPNCTYGCTGPGLEGC | |
| | PTNGPKIPSIATGMVGALLLLLVVALGIGLFM | |
| CAR16 | MALPVTALLLPLALLLHAARPEVQLLESGGGLVQPGGSLRLSCAA | 167 |
| (anti-nfP2X7) | SGFTFRNHDMGWVRQAPGKGLEWVSAISGSGGSTYYANSVKGR | |
| | FTISRDNSKNTLYLQMNSLRAEDTAVYYCAEPKPMDTEFDYRSP | |
| | GTLVTVSSESKYGPPCPPCPFWVLVVVGGVLACYSLLVTVAFIIF | |
| | WVRSKRSRLLHSDYMNMTPRRPGPTRKHYQPYAPPRDFAAYRS | |
| | KRGRKKLLYIFKQPFMRPVQTTQEEDGCSCRFPEEEEGGCELRV | |
| | KFSRSADAPAYKQGQNQLYNELNLGRREEYDVLDKRRGRDPEM | |
| | GGKPRRKNPQEGLYNELQKDKMAEAYSEIGMKGERRRGKGHDG | |
| | LYQGLSTATKDTYDALHMQALPPRLEGGGEGRGSLLTCGDVEEN | |
| | PGPRMLLLVTSLLLCELPHPAFLLIPRKVCNGIGIGEFKDSLSINAT | |
| | NIKHFKNCTSISGDLHILPVAFRGDSFTHTPPLDPQELDILKTVKEIT | |
| | GFLLIQAWPENRTDLHAFENLEIIRGRTKQHGQFSLAVVSLNITSL | |
| | GLRSLKEISDGDVIISGNKNLCYANTINWKKLFGTSGQKTKIISNRG | |
| | ENSCKATGQVCHALCSPEGCWGPEPRDCVSCRNVSRGRECVD | |
| | KCNLLEGEPREFVENSECIQCHPECLPQAMNITCTGRGPDNCIQC | |
| | AHYIDGPHCVKTCPAGVMGENNTLVWKYADAGHVCHLCHPNCT | |
| | YGCTGPGLEGCPTNGPKIPSIATGMVGALLLLLVVALGIGLFM | |

Constructs for binding to CD117

| | | |
|---|---|---|
| E200 + CD117 | GHNYTTRNILPGLNITSDIQMTQSPSSLSASVGDRVTITCRASQSI | 169 |
| binder (nfP2X₇ | NSYLNWYQQKPGKAPKLLIYAASSLQSGVPSRFSGSGSGTDFTL | |
| epitope | TISSLQPEDFATYYCQQGVSDITFGGGTKVEIKRTVAAPSVFIFPP | |
| underlined), | SDEQLKSGTASVVCLLNNFYPREAKVQWKVDNALQSGNSQESV | |
| WO2019084067, | TEQDSKDSTYSLSSTLTLSKADYEKHKVYACEVTHQGLSSPVTKS | |
| mAb-55, B076- | FNRGECHHHHHH | |
| 1_LC | | |
| CD117, | QVQLVQSGAEVKKPGSSVKVSCKASGGTFRIYAISWVRQAPGQG | 170 |
| WO2019084067, | LEWMGGIIPDFGVANYAQKFQGRVTITADESTSTAYMELSSLRSE | |
| mAb-55, B076- | DTAVYYCARGGLDTDEFDLWGRGTLVTVSSASTKGPSVFPLAPS | |
| 2_HC | SKSTSGGTAALGCLVKDYFPEPVTVSWNSGALTSGVHTFPAVLQ | |
| | SSGLYSLSSVVTVPSSSLGTQTYICNVNHKPSNTKVDKKVEPKSC | |
| | HHHHHH | |

Constructs for binding to CD133

| | | |
|---|---|---|
| E200 + CD133 | GHNYTTRNILPGLNITSDIQMTQSPSSLSASVGDRVTITCRASQGS | 171 |
| binder (nfP2X₇ | SYVAWYQQKPGKAPKLLIYSASYLYSGVPSRFSGSRSGTDFTLTI | |
| epitope | SSLQPEDFATYYCQQGVWSLITFGQGTKVEIKRTVAAPSVFIFPPS | |
| underlined), | DEQLKSGTASVVCLLNNFYPREAKVQWKVDNALQSGNSQESVT | |
| CA2962157, | EQDSKDSTYSLSSTLTLSKADYEKHKVYACEVTHQGLSSPVTKSF | |
| RW03, B077- | NRGECHHHHHH | |
| 1_LC | | |
| CD133, | EVQLVESGGGLVQPGGSLRLSCAASGFNLSSSSIHWVRQAPGK | 172 |
| CA2962157, | GLEWVAYIYPYYSYTYYADSVKGRFTISADTSKNTAYLQMNSLRA | |
| RW03, B077- | EDTAVYYCAREGSVAGEDYWGQGTLVTVSSASTKGPSVFPLAPS | |
| 2_HC | SKSTSGGTAALGCLVKDYFPEPVTVSWNSGALTSGVHTFPAVLQ | |
| | SSGLYSLSSVVTVPSSSLGTQTYICNVNHKPSNTKVDKKVEPKSC | |
| | HHHHHH | |

Constructs for binding to MUC1

| | | |
|---|---|---|
| E200 + MUC1 | GHNYTTRNILPGLNITSDIVMTQSPDSLAVSLGERATINCKSSQSL | 173 |
| binder (nfP2X₇ | LNSGDQKNYLTWYQQKPGQPPKLLIYWASTRESGVPDRFSGSG | |
| epitope | SGTDFTLTISSLQAEDVAVYYCQNDYSYPLTFGQGTKVEIKRTVA | |
| underlined), | APSVFIFPPSDEQLKSGTASVVCLLNNFYPREAKVQWKVDNALQS | |
| WO2016130726A | GNSQESVTEQDSKDSTYSLSSTLTLSKADYEKHKVYACEVTHQG | |
| 1, B078-1_LC | LSSPVTKSFNRGECHHHHHH | |
| MUC1, | QVQLVQSGAEVKKTGSSVKVSCKASGYTFTDHAIHWVRQAPGQ | 174 |
| WO2016130726A | ALEWMGHFSPGNTDIKYNDKFKGRVTLTVDRSMSTAYMELSSLR | |
| 1, B078-2_HC | SEDTAMYYCKTSTFFFDYWGQGTMVTVSSASTKGPSVFPLAPSS | |
| | KSTSGGTAALGCLVKDYFPEPVTVSWNSGALTSGVHTFPAVLQS | |
| | SGLYSLSSVVTVPSSSLGTQTYICNVNHKPSNTKVDKKVEPKSCH | |
| | HHHHH | |

Constructs for binding to Mesothelin (MSLN)

| | | |
|---|---|---|
| E200 + MSLN | GHNYTTRNILPGLNITSDIQMTQSPSSLSASVGDRVTITCRASQSI | 175 |
| binder (nfP2X₇ | SSYLNWYQQKPGKAPKLLIYAASSLQSGVPSGFSGSGSGTDFTL | |
| epitope | TISSLQPEDFATYYCQQSYSTPLTFGGGTKVEIKRTVAAPSVFIFP | |
| underlined), | PSDEQLKSGTASVVCLLNNFYPREAKVQWKVDNALQSGNSQES | |
| WO2009120769, | VTEQDSKDSTYSLSSTLTLSKADYEKHKVYACEVTHQGLSSPVTK | |
| B079-1_LC | SFNRGECHHHHHH | |
| MSLN, | QVQLQESGPGLVKPSETLSLTCTVSGGSVSSGSYYWSWIRQPP | 176 |
| WO2009120769, | GKGLEWIGYIYYSGSTNYNPSLKSRVTISVDTSKNQFSLKLSSVTA | |
| B079-2_HC | ADTAVYYCAREGKNGAFDIWGQGTMVTVSSASTKGPSVFPLAPS | |
| | SKSTSGGTAALGCLVKDYFPEPVTVSWNSGALTSGVHTFPAVLQ | |
| | SSGLYSLSSVVTVPSSSLGTQTYICNVNHKPSNTKVDKKVEPKSC | |
| | HHHHHH | |

Constructs for binding to ROR2

| | | |
|---|---|---|
| E200 + ROR2 | GHNYTTRNILPGLNITSQSALTQPASVSGSPGQSITISCTGTSGDV | 177 |
| binder (nfP2X₇ | GGYNYVSWYQHHPGKAPKLIIYDVNKRPSGFSDRFSGSKSGNTA | |
| epitope | SLTISGLQAEDEADYYCSSYTSTSTVFGGGTKLTVLGKRTVAAPS | |
| underlined), | VFIFPPSDEQLKSGTASVVCLLNNFYPREAKVQWKVDNALQSGN | |
| WO2016142768A | SQESVTEQDSKDSTYSLSSTLTLSKADYEKHKVYACEVTHQGLSS | |
| 1, B080-1_LC | PVTKSFNRGECHHHHHH | |
| ROR2, | QITLKESGPELVKPTQTLTLTCTFSGFSLSTSGMSVSWIRQPPGK | 178 |
| WO2016142768A | ALEWLARIDWDDDKYYSTSLKTRLTISKDTSKNQVVLTMTNTDPV | |
| 1, B080-2_HC | DTATYYCARGFYLAYGSYDSWGQGTLVTVSSASTKGPSVFPLAP | |
| | SSKSTSGGTAALGCLVKDYFPEPVTVSWNSGALTSGVHTFPAVL | |
| | QSSGLYSLSSVVTVPSSSLGTQTYICNVNHKPSNTKVDKKVEPKS | |
| | CHHHHHH | |

Constructs for binding to IL13Ra2

| | | |
|---|---|---|
| E200 + IL13Ra2 | GHNYTTRNILPGLNITSDIQMTQSPSSLSASVGDRVTITCTASLSV | 179 |
| binder (nfP2X₇ | SSTYLHWYQQKPGSSPKLWIYSTSNLASGVPSRFSGSGSGTSFT | |
| epitope | LTISSLQPEDFATYYCHQYHRSPLTFGGGTKVEIKRTVAAPSVFIF | |
| underlined), | PPSDEQLKSGTASVVCLLNNFYPREAKVQWKVDNALQSGNSQE | |
| WO2014072888A | SVTEQDSKDSTYSLSSTLTLSKADYEKHKVYACEVTHQGLSSPVT | |
| 1, B081-1_LC | KSFNRGECHHHHHH | |
| IL13Ra2, | EVQLVESGGGLVQPGGSLRLSCAASGFSLTKYGVHWVRQAPGK | 180 |
| WO2014072888A | GLEWVGVKWAGGSTDYNSALMSRFTISKDNAKNSLYLQMNSLR | |
| 1, B081-2_HC | AEDTAVYYCARDHRDAMDYWGQGTLVTVSSASTKGPSVFPLAP | |
| | SSKSTSGGTAALGCLVKDYFPEPVTVSWNSGALTSGVHTFPAVL | |
| | QSSGLYSLSSVVTVPSSSLGTQTYICNVNHKPSNTKVDKKVEPKS | |
| | CHHHHHH | |

Constructs for binding to IL13Ra2

| | | |
|---|---|---|
| IL13Ra2, ligand, | HHHHHHGPVPPSTALRYLIEELVNITQNQKAPLCNGSMVWSINLT | 181 |
| US20180265844, | AGMYCAALESLINVSGCSAIEKTQRMLSGFCPHKVSAGQFSSLHV | |
| B082 | RDTKIEVAQFVKDLLLHLKKLFREGRFNGGGGSGHNYTTRNILPG | |
| | LNITS | |

Constructs for binding to EPHA2

| | | |
|---|---|---|
| E200 + EPHA2 | GHNYTTRNILPGLNITSDIQLTQSPSSLSASVGDRVTITCKASQDIN | 182 |
| binder (nfP2X₇ | NYLSWYQQKPGQAPRLLIYRANRLVDGVPDRFSGSGYGTDFTLTI | |
| epitope | NNIESEDAAYYFCLKYDVFPYTFGQGTKVEIKRTVAAPSVFIFPPS | |
| underlined), | DEQLKSGTASVVCLLNNFYPREAKVQWKVDNALQSGNSQESVT | |
| WO2007073499, | EQDSKDSTYSLSSTLTLSKADYEKHKVYACEVTHQGLSSPVTKSF | |
| B083-1_LC | NRGECHHHHHH | |
| EPHA2, | QVQLLESGGGLVQPGGSLRLSCAASGFTFSSYTMSWVRQAPGQ | 183 |
| WO2007073499, | ALEWMGTISSGGTYTYYPDSVKGRFTISRDNAKNSLYLQMNSLR | |
| B083-2_HC | AEDTAVYYCAREAIFTYWGRGTLVTSSASTKGPSVFPLAPSSKST | |
| | SGGTAALGCLVKDYFPEPVTVSWNSGALTSGVHTFPAVLQSSGL | |
| | YSLSSVVTVPSSSLGTQTYICNVNHKPSNTKVDKKVEPKSCHHHH | |
| | HH | |

Constructs for binding to EGFRvIII

| | | |
|---|---|---|
| E200 + EGFRvIII | GHNYTTRNILPGLNITSDIQMTQSPSSLSASVGDRVTITCRASQGI | 184 |
| binder (nfP2X₇ | RNNLAWYQQKPGKAPKRLIYAASNLQSGVPSRFTGSGSGTEFTLI | |
| epitope | VSSLQPEDFATYYCLQHHSYPLTSGGGTKVEIKRTVAAPSVFIFPP | |
| underlined), | SDEQLKSGTASVVCLLNNFYPREAKVQWKVDNALQSGNSQESV | |
| WO2013185010, | TEQDSKDSTYSLSSTLTLSKADYEKHKVYACEVTHQGLSSPVTKS | |
| B084-1_LC | FNRGECHHHHHH | |
| EGFRvIII, | EVQVLESGGGLVQPGGSLRLSCAASGFTFSSYAMSWVRQAPGK | 185 |
| WO2013185010, | GLEWVSAISGSGGSTNYADSVKGRFTISRDNSKNTLYLQMNSLR | |
| B084-2_HC | AEDTAVYYCAGSSGWSEYWGQGTLVTVSSASTKGPSVFPLAPS | |
| | SKSTSGGTAALGCLVKDYFPEPVTVSWNSGALTSGVHTFPAVLQ | |
| | SSGLYSLSSVVTVPSSSLGTQTYICNVNHKPSNTKVDKKVEPKSC | |
| | HHHHHH | |

Constructs for binding to PSMA

| | | |
|---|---|---|
| E200 + PSMA | GHNYTTRNILPGLNITSDIVMTQSPSSLSASVGDRVTITCKASQDV | 186 |
| binder (nfP2X₇ | GTAVDWYQQKPGKAPKLLIYWASTRHTGVPDRFTGSGSGTDFTL | |
| epitope | TISSLQPEDFADYFCQQYNSYPLTFGGGTKLEIKRTVAAPSVFIFP | |
| underlined), | PSDEQLKSGTASVVCLLNNFYPREAKVQWKVDNALQSGNSQES | |
| US20190300622, | VTEQDSKDSTYSLSSTLTLSKADYEKHKVYACEVTHQGLSSPVTK | |
| B085-1_LC | SFNRGECHHHHHH | |
| PSMA, | EVQLVQSGAEVKKPGASVKISCKTSGYTFTEYTIHWVKQASGKGL | 187 |
| US20190300622, | EWIGNINPNNGGTTYNQKFEDRATLTVDKSTSTAYMELSSLRSED | |
| B085-2_HC | TAVYYCAAGWNFDYWGQGTTVTVSSASTKGPSVFPLAPSSKSTS | |
| | GGTAALGCLVKDYFPEPVTVSWNSGALTSGVHTFPAVLQSSGLY | |
| | SLSSVVTVPSSSLGTQTYICNVNHKPSNTKVDKKVEPKSCHHHHH | |
| | H | |

Constructs for binding to CEA

| | | |
|---|---|---|
| E200 + CEA binder | GHNYTTRNILPGLNITSDIVLTQSPASLTVSLGLRATISCRASKSVS | 188 |
| (nfP2X₇ epitope | ASGYSYMHWYQQRPGQPPKLLIYLASNLQSGVPARFSGSGSGT | |
| underlined), | DFTLNIHPVEEEDAATYYCQHSRELPTFGGGTKLEIKRTVAAPSVF | |
| WO1999043817, | IFPPSDEQLKSGTASVVCLLNNFYPREAKVQWKVDNALQSGNSQ | |
| B086-1_LC | ESVTEQDSKDSTYSLSSTLTLSKADYEKHKVYACEVTHQGLSSPV | |
| | TKSFNRGECHHHHHH | |
| CEA, | EVQLQQSGAELVRSGASVKMSCTASGFNIKDYYMHWVKQRPEQ | 189 |
| WO1999043817, | GLEWIGWIDPENGDTEYAPKFQGKATMTTDYSSNTAYLQLSSLTS | |
| B086-2_HC | EDTAVYYCNTRGLSTMITTRWFFDVWGAGTTVAVSSASTKGPSV | |
| | FPLAPSSKSTSGGTAALGCLVKDYFPEPVTVSWNSGALTSGVHT | |
| | FPAVLQSSGLYSLSSVVTVPSSSLGTQTYICNVNHKPSNTKVDKK | |
| | VEPKSCHHHHHH | |

Constructs for binding to PSCA

| | | |
|---|---|---|
| E200 + PSCA | GHNYTTRNILPGLNITSDIQLTQSPSSLSASVGDRVTITCSASSSV | 190 |
| binder (nfP2X₇ | RFIHWYQQKPGKAPKRLIYDTSKLASGVPSRFSGSGSGTDFTLTI | |
| epitope | SSLQPEDFATYYCQQWSSSPFTFGQGTKVEIKRTVAAPSVFIFPP | |
| underlined), | SDEQLKSGTASVVCLLNNFYPREAKVQWKVDNALQSGNSQESV | |
| US20120077962, | TEQDSKDSTYSLSSTLTLSKADYEKHKVYACEVTHQGLSSPVTKS | |
| B087-1_LC | FNRGECHHHHHH | |
| PSCA, | EVQLVESGGGLVQPGGSLRLSCAASGFNIKDYYIHWVRQAPGKG | 191 |
| US20120077962, | LEWVAWIDPENGDTEFADSVKGRFTISADTSKNTAYLQMNSLRA | |
| B087-2_HC | EDTAVYYCKTGGFWGQGTLVTVSSASTKGPSVFPLAPSSKSTSG | |
| | GTAALGCLVKDYFPEPVTVSWNSGALTSGVHTFPAVLQSSGLYS | |
| | LSSVVTVPSSSLGTQTYICNVNHKPSNTKVDKKVEPKSCHHHHH | |
| | H | |

Constructs for binding to Lewis Y

| | | |
|---|---|---|
| E200 + Lewis Y | GHNYTTRNILPGLNITSDIQMTQSPSSLSASVGDRVTITCRSSQRI | 192 |
| binder (nfP2X₇ | VHSNGNTYLEWYQQTPGKAPKLLIYKVSNRFSGVPSRFSGSGSG | |
| epitope | TDFTFTISSLQPEDIATYYCFQGSHVPFTFGQGTKLQITKRTVAAP | |
| underlined), | SVFIFPPSDEQLKSGTASVVCLLNNFYPREAKVQWKVDNALQSG | |
| EP0749482, | NSQESVTEQDSKDSTYSLSSTLTLSKADYEKHKVYACEVTHQGL | |
| B088-1_LC | SSPVTKSFNRGECHHHHHH | |
| Lewis Y, | EVQLVESGGGVVQPGRSLRLSCSSSGFTFSDYYMYWVRQAPGK | 193 |
| EP0749482, | GLEWVAYMSNVGAITDYPDTVKGRFTISRDNSKNTLFLQMDSLRP | |
| B088-2_HC | EDTGVYFCARGTRDGSWFAYWGQGTPVTVSSASTKGPSVFPLA | |
| | PSSKSTSGGTAALGCLVKDYFPEPVTVSWNSGALTSGVHTFPAV | |
| | LQSSGLYSLSSVVTVPSSSLGTQTYICNVNHKPSNTKVDKKVEPK | |
| | SCHHHHHH | |

Constructs for binding to CD171 L1CAM

| | | |
|---|---|---|
| E200 + CD171_L1 | GHNYTTRNILPGLNITSDIQMTQSPSSLSASVGDRVTITCRASQDI | 194 |
| CAM binder | SNYLNWYQQKPGKAPKLLIYYTSRLHSGVPSRFSGSGSGTDYTL | |
| (nfP2X₇ epitope | TISSLQPEDFATYFCQQGNTLPWTFGGGTKLEIKRTVAAPSVFIFP | |
| underlined), | PSDEQLKSGTASVVCLLNNFYPREAKVQWKVDNALQSGNSQES | |
| WO2008151819, | VTEQDSKDSTYSLSSTLTLSKADYEKHKVYACEVTHQGLSSPVTK | |
| B089-1_LC | SFNRGECHHHHHH | |
| CD171_L1CAM, | EVQLVQSGGGLVQSGGSLRLSCRASGYTFTRYWMLWVRQRPG | 195 |
| WO2008151819, | HGLEWVGEINPRNDRTNYNEKFKTRFTISVDRSKSTAYLQMDSLR | |
| B089-2_HC | AEDTAVYFCALGGGYAMDYWGQGTLVTVSSASTKGPSVFPLAPS | |
| | SKSTSGGTAALGCLVKDYFPEPVTVSWNSGALTSGVHTFPAVLQ | |
| | SSGLYSLSSVVTVPSSSLGTQTYICNVNHKPSNTKVDKKVEPKSC | |
| | HHHHHH | |

Constructs for binding to EpCAM

| | | |
|---|---|---|
| E200 + EpCAM | GHNYTTRNILPGLNITSEIVMTQSPATLSVSPGERATLSCRASQSV | 196 |
| binder (nfP2X₇ | SSNLAWYQQKPGQAPRLIIYGASTTASGIPARFSASGSGTDFTLTI | |
| epitope | SSLQSEDFAVYYCQQYNNWPPAYTFGQGTKLEIKRTVAAPSVFIF | |
| underlined), | PPSDEQLKSGTASVVCLLNNFYPREAKVQWKVDNALQSGNSQE | |
| WO2010142990A | SVTEQDSKDSTYSLSSTLTLSKADYEKHKVYACEVTHQGLSSPVT | |
| 1, B090-1_LC | KSFNRGECHHHHHH | |
| EpCAM, | QVQLVQSGAEVKKPGSSVKVSCKASGGTFSSYAISWVRQAPGQ | 197 |
| WO2010142990A | GLEWMGGIIPIFGTANYAQKFQGRVTITADESTSTAYMELSSLRSE | |
| 1, B090-2_HC | DTAVYYCARGLLWNYWGQGTLVTVSSASTKGPSVFPLAPSSKST | |
| | SGGTAALGCLVKDYFPEPVTVSWNSGALTSGVHTFPAVLQSSGL | |
| | YSLSSVVTVPSSSLGTQTYICNVNHKPSNTKVDKKVEPKSCHHHH | |
| | HH | |

Constructs for binding to ALK

| | | |
|---|---|---|
| E200 + ALK | GHNYTTRNILPGLNITSEIVLTQSPATLSLSPGERATLSCRASESV | 198 |
| binder (nfP2X₇ | DNYGISFAWYQQKPGQAPRLLIYRASRATGIPARFSGSGSGTDFT | |
| epitope | LTISSLEPEDFAVYYCQQNNKDPPTFFGQGTKLEIKRTVAAPSVFI | |
| underlined), | FPPSDEQLKSGTASVVCLLNNFYPREAKVQWKVDNALQSGNSQ | |
| WO2015069922A | ESVTEQDSKDSTYSLSSTLTLSKADYEKHKVYACEVTHQGLSSPV | |
| 2, B091-1_LC | TKSFNRGECHHHHHH | |
| ALK, | QVQLQQSGAEVKKPGSSVKVSCKASGYAFSSYISWVRQAPGQG | 199 |
| WO2015069922A | LEWMGGQIYPGDGDTNYAQKFQGRVTITADESTSTAYMELSSLR | |
| 2, B091-2_HC | SEDTAVYYCVRYYYGSSGYFDYWWGQGTMVTVSSASTKGPSVF | |
| | PLAPSSKSTSGGTAALGCLVKDYFPEPVTVSWNSGALTSGVHTF | |
| | PAVLQSSGLYSLSSVVTVPSSSLGTQTYICNVNHKPSNTKVDKKV | |
| | EPKSCHHHHHH | |

Constructs for binding to IGF-1R CD221

| | | |
|---|---|---|
| E200 + IGF-1R | GHNYTTRNILPGLNITSDVVMTQTPLSLPVSLGDPASISCRSSQSI | 200 |
| binder (nfP2X₇ | VHSNVNTYLEWYLQKPGQSPRLLIYKVSNRFSGVPDRFSGSGAG | |
| epitope | TDFTLRISRVEAEDLGIYYCFQGSHVPPTFGGGTKLEIKRTVAAPS | |
| underlined), | VFIFPPSDEQLKSGTASVVCLLNNFYPREAKVQWKVDNALQSGN | |
| US7985842B2, | SQESVTEQDSKDSTYSLSSTLTLSKADYEKHKVYACEVTHQGLSS | |
| B092-1_LC | PVTKSFNRGECHHHHHH | |
| IGF-1R, | QVQLVQSGAEVVKPGASVKLSCKASGYTFTSYWMHWVKQRPG | 201 |
| US7985842B2, | QGLEWIGEINPSNGRTNYNQKFQGKATLTVDKSSSTAYMQLSSL | |
| B092-2_HC | TSEDSAVYYFARGRPDYYGSSKWYFDVWGQGTTVTVSSASTKG | |
| | PSVFPLAPSSKSTSGGTAALGCLVKDYFPEPVTVSWNSGALTSG | |
| | VHTFPAVLQSSGLYSLSSVVTVPSSSLGTQTYICNVNHKPSNTKV | |
| | DKKVEPKSCHHHHHH | |

Constructs for binding to Nectin 4

| | | |
|---|---|---|
| E200 + Nectin 4 | GHNYTTRNILPGLNITSSIVMTQTPKFLLVSAGDRVTITCKASQSVS | 202 |
| binder (nfP2X₇ | NDVAWYQQKPGQSPKLLIYYASNRYTGVPDRFTGSGYGTDFTFT | |
| epitope | ISAVQAEDLAVYFCQQDYSSPYTFGGGTKLEIKRTVAAPSVFIFPP | |
| underlined), | SDEQLKSGTASVVCLLNNFYPREAKVQWKVDNALQSGNSQESV | |
| US20210130459, | TEQDSKDSTYSLSSTLTLSKADYEKHKVYACEVTHQGLSSPVTKS | |
| B093-1_LC | FNRGECHHHHHH | |
| Nectin 4, | QVQLQQSGPELVKPGASVRISCKASGYTFTTYYIHWVKQRPGQG | 203 |
| US20210130459, | LEWIGWIYPGNVNTKNNEKFKVKATLTADKSSSTAYMQLSSLTSE | |
| B093-2_HC | DSAVYFCARSNPYVMDYWGQGTSVTVSSASTKGPSVFPLAPSS | |
| | KSTSGGTAALGCLVKDYFPEPVTVSWNSGALTSGVHTFPAVLQS | |
| | SGLYSLSSVVTVPSSSLGTQTYICNVNHKPSNTKVDKKVEPKSCH | |
| | HHHHH | |

Constructs for binding to FAP

| | | |
|---|---|---|
| E200 + FAP | GHNYTTRNILPGLNITSEIVLTQSPGTLSLSPGERATLSCRASQSV | 204 |
| binder (nfP2X₇ | TSSYLAWYQQKPGQAPRLLINVGSRRATGIPDRFSGSGSGTDFT | |
| epitope | LTISRLEPEDFAVYYCQQGIMLPPTFGQGTKVEIKRTVAAPSVFIFP | |
| underlined), | PSDEQLKSGTASVVCLLNNFYPREAKVQWKVDNALQSGNSQES | |
| EP2603530, | VTEQDSKDSTYSLSSTLTLSKADYEKHKVYACEVTHQGLSSPVTK | |
| B094-1_LC | SFNRGECHHHHHH | |
| FAP, EP2603530, | EVQLLESGGGLVQPGGSLRLSCAASGFTFSSYAMSWVRQAPGK | 205 |
| B094-2 HC | GLEWVSAIIGSGSITYYADSVKGRFTISRDNSKNTLYLQMNSLRAE | |
| | DTAVYYCAKGWFGGFNYWGQGTLVTVSSASTKGPSVFPLAPSS | |
| | KSTSGGTAALGCLVKDYFPEPVTVSWNSGALTSGVHTFPAVLQS | |
| | SGLYSLSSVVTVPSSSLGTQTYICNVNHKPSNTKVDKKVEPKSCH | |
| | HHHHH | |

Constructs for binding to AXL

| | | |
|---|---|---|
| E200 + AXL | GHNYTTRNILPGLNITSDVVMTQSPLSLPVTLGQPASISCRSSQNI | 206 |
| binder (nfP2X₇ | VHTNGNTYLEWYQQKPGKAPELLIYKVSNRFSGVPDRFSGSGSG | |
| epitope | TDFTLKISRVEAEDVGVYYCFQGSHLLEPFTFGQGTKLEIKRTVAA | |
| underlined), | PSVFIFPPSDEQLKSGTASVVCLLNNFYPREAKVQWKVDNALQS | |
| EP2431393, | GNSQESVTEQDSKDSTYSLSSTLTLSKADYEKHKVYACEVTHQG | |
| B095-1_LC | LSSPVTKSFNRGECHHHHHH | |
| AXL, EP2431393, | QVTLKESGPVLVKPTETLTLTCTVSGFSLSSFGVDWVRQAPGKG | 207 |
| B095-2_HC | LEWMGVIWGGGSTNYNSALKSRLTISKDNSKSQVVLTMTNMDPV | |
| | DTATYYCAGEGSKYGAWFAYWGQGTLVTVSSASTKGPSVFPLA | |
| | PSSKSTSGGTAALGCLVKDYFPEPVTVSWNSGALTSGVHTFPAV | |
| | LQSSGLYSLSSVVTVPSSSLGTQTYICNVNHKPSNTKVDKKVEPK | |
| | SCHHHHHH | |

Constructs for binding to CD138

| | | |
|---|---|---|
| E200 + CD138 | GHNYTTRNILPGLNITSDIQMTQSTSSLSASLGDRVTISCSASQGI | 208 |
| binder (nfP2X₇ | NNYLNWYQQKPDGTVELLIYYTSTLQSGVPSRFSGSGSGTDYSL | |
| epitope | TISNLEPEDIGTYYCQQYSKLPRTFGGGTKLEIKRTVAAPSVFIFPP | |
| underlined), | SDEQLKSGTASVVCLLNNFYPREAKVQWKVDNALQSGNSQESV | |
| US20090175863, | TEQDSKDSTYSLSSTLTLSKADYEKHKVYACEVTHQGLSSPVTKS | |
| B096-1_LC | FNRGECHHHHHH | |
| CD138, | QVQLQQSGSELMMPGASVKISCKATGYTFSNYWIEWVKQRPGH | 209 |
| US20090175863, | GLEWIGEILPGTGRTIYNEKFKGKATFTADISSNTVQMQLSSLTSE | |
| B096-2_HC | DSAVYYCARRDYYGNFYYAMDYWGQGTSVTVSSASTKGPSVFP | |
| | LAPSSKSTSGGTAALGCLVKDYFPEPVTVSWNSGALTSGVHTFP | |
| | AVLQSSGLYSLSSVVTVPSSSLGTQTYICNVNHKPSNTKVDKKVE | |
| | PKSCHHHHHH | |

Constructs for binding to CLDN6

| | | |
|---|---|---|
| E200 + CLDN6 | GHNYTTRNILPGLNITSDIQMTQSPSSLSASVGDRVTITCRISENIY | 210 |
| binder (nfP2X₇ | SYLAWYQQKPGKAPKLLIYNAKILVEGVPSRFSGSGSGTDFTLTIS | |
| epitope | SLQPEDFATYYCQHHYTVPWTFGQGTKLEIKRTVAAPSVFIFPPS | |
| underlined), | DEQLKSGTASVVCLLNNFYPREAKVQWKVDNALQSGNSQESVT | |
| WO2019056023, | EQDSKDSTYSLSSTLTLSKADYEKHKVYACEVTHQGLSSPVTKSF | |
| B097-1_LC | NRGECHHHHHH | |
| CLDN6, | EVQLLESGGGLVQPGGSMRLSCAASGFTFSNYWMNWVRQAPG | 211 |
| WO2019056023, | KGLEWVAQIRLKSDNYATHYADSVKGRFTISRDDSKNTVYLQMN | |
| B097-2_HC | SLRAEDTGVYYCNDGPPSGYWGQGTLLTVSSASTKGPSVFPLAP | |
| | SSKSTSGGTAALGCLVKDYFPEPVTVSWNSGALTSGVHTFPAVL | |
| | QSSGLYSLSSVVTVPSSSLGTQTYICNVNHKPSNTKVDKKVEPKS | |
| | CHHHHHH | |

Constructs for binding to Her4

| | | |
|---|---|---|
| E200 + Her4 | GHNYTTRNILPGLNITSQSVLTQPASVSGSPGQSITISCAGTSSDV | 212 |
| binder (nfP2X₇ | GGSSYVSWYQQHPGKAPKLMIYYDSYRPSGVSNRFSGSKSGNT | |
| epitope | ASLTISGLQAEDEADYYCSSNTYYSTRVFGGGTKLAVLGKRTVAA | |
| underlined), | PSVFIFPPSDEQLKSGTASVVCLLNNFYPREAKVQWKVDNALQS | |
| WO2021116119, | GNSQESVTEQDSKDSTYSLSSTLTLSKADYEKHKVYACEVTHQG | |
| B098-1_LC | LSSPVTKSFNRGECHHHHHH | |
| Her4, | EVQLVESGGSLVKPGGSLRLSCAASGFTFSNYYMNWVRQAPGK | 213 |
| WO2021116119, | GLEWISSIDGSSRYIDYADFVKGRFTISRDNATNSLYLQMNSLRAE | |
| B098-2_HC | DTAVYYCVRSSSDYFGGGMDVWGRGTLVTVSSASTKGPSVFPL | |
| | APSSKSTSGGTAALGCLVKDYFPEPVTVSWNSGALTSGVHTFPA | |
| | VLQSSGLYSLSSVVTVPSSSLGTQTYICNVNHKPSNTKVDKKVEP | |
| | KSCHHHHHH | |

Constructs for binding to Claudin 18.2

| | | |
|---|---|---|
| E200 + Claudin | GHNYTTRNILPGLNITSDIVMTQSPSSLTVTAGEKVTMSCKSSQSL | 214 |
| 18.2 binder | LNSGNQKNYLTWYQQKPGQPPKLLIYWASTRESGVPDRFTGSG | |
| (nfP2X₇ epitope | SGTDFTLTISSVQAEDLAVYYCQNDYSYPFTFGSGTKLEIKRTVAA | |
| underlined), | PSVFIFPPSDEQLKSGTASVVCLLNNFYPREAKVQWKVDNALQS | |
| SG10201609510 | GNSQESVTEQDSKDSTYSLSSTLTLSKADYEKHKVYACEVTHQG | |
| U, B099-1_LC | LSSPVTKSFNRGECHHHHHH | |
| Claudin 18.2, | QVQLQQPGAELVRPGASVKLSCKASGYTFTSYWINWVKQRPGQ | 215 |
| SG10201609510 | GLEWIGNIYPSDSYTNYNQKFKDKATLTVDKSSSTAYMQLSSPTS | |
| U, B099-2_HC | EDSAVYYCTRSWRGNSFDYWGQGTTLTVSSASTKGPSVFPLAP | |
| | SSKSTSGGTAALGCLVKDYFPEPVTVSWNSGALTSGVHTFPAVL | |
| | QSSGLYSLSSVVTVPSSSLGTQTYICNVNHKPSNTKVDKKVEPKS | |
| | CHHHHHH | |

Constructs for binding to O-acetylated GD2

| | | |
|---|---|---|
| E200 + O- | GHNYTTRNILPGLNITSDVVMTQSPLSLPVTLGQPASISCRSSQSL | 216 |
| acetylated GD2 | LKNNGNTFLHWYQQRPGQSPRLLIYKVSNRLSGVPDRFSGSGSG | |
| binder (nfP2X₇ | TDFTLKISRVEAEDVGVYFCSQSTHIPYTFGGGTKVEIKRTVAAPS | |
| epitope | VFIFPPSDEQLKSGTASVVCLLNNFYPREAKVQWKVDNALQSGN | |
| underlined), | SQESVTEQDSKDSTYSLSSTLTLSKADYEKHKVYACEVTHQGLSS | |
| EP3269739A1, | PVTKSFNRGECHHHHHH | |
| B100-1_LC | | |
| O-acetylated | EVQLVESGGGLVQPGRSLRLSCTTSEFTFTDYYMTWVRQAPGK | 217 |
| GD2, | GLEWLGFIRNRANGYTTEYNPSVKGRFTISRDNSKSILYLQMNSL | |
| EP3269739A1, | KTEDTAVYYCARVSNWAFDYWGQGTLVTVSSASTKGPSVFPLAP | |
| B100-2_HC | SSKSTSGGTAALGCLVKDYFPEPVTVSWNSGALTSGVHTFPAVL | |
| | QSSGLYSLSSVVTVPSSSLGTQTYICNVNHKPSNTKVDKKVEPKS | |
| | CHHHHHH | |

Constructs for binding to GD3

| | | |
|---|---|---|
| E200 + GD3 | GHNYTTRNILPGLNITSDIQMTQSPSSLSASVGDRVTITCSASQDI | 218 |
| binder (nfP2X₇ | SNYLNWYQQKPDKAVKLLIFYSSNLHSGVPSRFSGGGSGTDYTL | |
| epitope | TISSLQPEDIATYFCHQYSKLPWTFGQGTKVEIKRTVAAPSVFIFP | |
| underlined), | PSDEQLKSGTASVVCLLNNFYPREAKVQWKVDNALQSGNSQES | |
| US7253263, | VTEQDSKDSTYSLSSTLTLSKADYEKHKVYACEVTHQGLSSPVTK | |
| B101-1_LC | SFNRGECHHHHHH | |
| GD3, | EVQLVESGGDFVQPGGSLRVSCAASGFAFSHYAMSWVRQAPGK | 219 |
| US7253263, | GLEWVAYISSGGSGTYYSDSVKGRFTISRDNSKNTLYLQMRSLR | |
| B101-2_HC | AEDSAVYFCTRVKLGTYYFDSWGQGTLLTVSSASTKGPSVFPLA | |
| | PSSKSTSGGTAALGCLVKDYFPEPVTVSWNSGALTSGVHTFPAV | |
| | LQSSGLYSLSSVVTVPSSSLGTQTYICNVNHKPSNTKVDKKVEPK | |
| | SCHHHHHH | |

Constructs for binding to GM2

| | | |
|---|---|---|
| E200 + GM2 | GHNYTTRNILPGLNITSDIQLTQSPSSLSASPGDRVTITCSASSSVS | 220 |
| binder (nfP2X₇ | YMHWFQQKPGKAPKLWIYSTSNLASGVPSRFSGSGSGTSYSLTI | |
| epitope | SRLQPEDIATYYCQQRSSYPYTFGGGTKVEIKRTVAAPSVFIFPPS | |
| underlined), | DEQLKSGTASVVCLLNNFYPREAKVQWKVDNALQSGNSQESVT | |
| US5939532, | EQDSKDSTYSLSSTLTLSKADYEKHKVYACEVTHQGLSSPVTKSF | |
| B102-1_LC | NRGECHHHHHH | |
| GM2, | EVQLVQSGAEVKKPGASVKVSCKASGYTFTDYNMDWVRQAPGQ | 221 |
| US5939532, | GLEWMGYIYPNNGGTGYNQKFKSKVTITVDTSTSTAYMELHSLR | |
| B102-2_HC | SEDTAVYYCATYGHYYGYMFAYWGQGTLVTVSSASTKGPSVFPL | |
| | APSSKSTSGGTAALGCLVKDYFPEPVTVSWNSGALTSGVHTFPA | |
| | VLQSSGLYSLSSVVTVPSSSLGTQTYICNVNHKPSNTKVDKKVEP | |
| | KSCHHHHHH | |

Constructs for binding to TM4SF1

| | | |
|---|---|---|
| E200 + TM4SF1 | GHNYTTRNILPGLNITSAVVMTQTPLSLPVSLGDQASISCRSSQSL | 222 |
| binder (nfP2X₇ | VHSNGNTYLHWYMQKPGQSPKVLIYKVSNRFSGVPDRFSGSGS | |
| epitope | GTDFTLKISRVEADDLGIYFCSQSTHIPLAFGAGTKLELKRTVAAP | |
| underlined), | SVFIFPPSDEQLKSGTASVVCLLNNFYPREAKVQWKVDNALQSG | |
| WO2015054427A | NSQESVTEQDSKDSTYSLSSTLTLSKADYEKHKVYACEVTHQGL | |
| 1, B103-1_LC | SSPVTKSFNRGECHHHHHH | |
| TM4SF1, | EVILVESGGGLVKPGGSLKLSCAASGFTFSSFAMSWVRQTPEKR | 223 |
| WO2015054427A | LEWVATISSGSIYIYYTDGVKGRFTISRDNAKNTVHLQMSSLRSED | |
| 1, B103-2_HC | TAMYYCARRGIYYGYDGYAMDYWGQGTSVTVSSASTKGPSVFP | |
| | LAPSSKSTSGGTAALGCLVKDYFPEPVTVSWNSGALTSGVHTFP | |
| | AVLQSSGLYSLSSVVTVPSSSLGTQTYICNVNHKPSNTKVDKKVE | |
| | PKSCHHHHHH | |

Constructs for binding to CD147

| | | |
|---|---|---|
| E200 + CD147 | GHNYTTRNILPGLNITSDIQMTQSPSTLSASVGDRVTLSCKASENV | 224 |
| binder (nfP2X₇ | GTYVSWYQQKPGKAPKLLIYGASNRYTGVPSRFTGSGSGTDFTL | |
| epitope | TISSLQPEDFATYYCGQSYSYPFTFGSGTKLEIKRTVAAPSVFIFP | |
| underlined), | PSDEQLKSGTASVVCLLNNFYPREAKVQWKVDNALQSGNSQES | |
| WO2017186182, | VTEQDSKDSTYSLSSTLTLSKADYEKHKVYACEVTHQGLSSPVTK | |
| B104-1_LC | SFNRGECHHHHHH | |
| CD147, | EVQLVESGGGLVQPGGSLRLSCAASGFTFSNFWMNWVRQAPG | 225 |
| WO2017186182, | KGLEWVSEIRLKSNNYATHYAESVKGRFTISRDDSKNTLYLQMNS | |
| B104-2_HC | LKTEDTAVYYCTSYDYEYWGQGTLVTVSAASTKGPSVFPLAPSS | |
| | KSTSGGTAALGCLVKDYFPEPVTVSWNSGALTSGVHTFPAVLQS | |
| | SGLYSLSSVVTVPSSSLGTQTYICNVNHKPSNTKVDKKVEPKSCH | |
| | HHHHH | |

Constructs for binding to CEACAM5

| | | |
|---|---|---|
| E200 + CEACAM5 | GHNYTTRNILPGLNITSDIQLTQSPSSLSASVGDRVTITCKASQDV | 226 |
| binder (nfP2X₇ | GTSVAWYQQKPGKAPKLLIYWTSTRHTGVPSRFSGSGSGTDFTF | |
| epitope | TISSLQPEDIATYYCQQYSLYRSFGQGTKVEIKRTVAAPSVFIFPPS | |
| underlined), | DEQLKSGTASVVCLLNNFYPREAKVQWKVDNALQSGNSQESVT | |
| WO2015069430, | EQDSKDSTYSLSSTLTLSKADYEKHKVYACEVTHQGLSSPVTKSF | |
| B105-1_LC | NRGECHHHHHH | |
| CEACAM5, | EVQLVESGGGVVQPGRSLRLSCSASGFDFTTYWMSWVRQAPG | 227 |
| WO2015069430, | KGLEWIGEIHPDSSTINYAPSLKDRFTISRDNAKNTLFLQMDSLRP | |
| B105-2_HC | EDTGVYFCASLYFGFPWFAYWGQGTPVTVSSASTKGPSVFPLAP | |
| | SSKSTSGGTAALGCLVKDYFPEPVTVSWNSGALTSGVHTFPAVL | |
| | QSSGLYSLSSVVTVPSSSLGTQTYICNVNHKPSNTKVDKKVEPKS | |
| | CHHHHHH | |

Constructs for binding to VEGFR-1

| | | |
|---|---|---|
| E200 + VEGFR-1 | GHNYTTRNILPGLNITSDIVMTQSPDSLAVSLGERATINCSASSSV | 228 |
| binder (nfP2X₇ | SYMHWYQQKPGQPPKLLIYRTSNLASGVPDRFSGSGSGTDFTLT | |
| epitope | ISSLQAEDVAVYYCHQWSMYTFGQGTKVEIKRTVAAPSVFIFPPS | |
| underlined), | DEQLKSGTASVVCLLNNFYPREAKVQWKVDNALQSGNSQESVT | |
| US7615214, | EQDSKDSTYSLSSTLTLSKADYEKHKVYACEVTHQGLSSPVTKSF | |
| B106-1_LC | NRGECHHHHHH | |
| VEGFR-1, | QVQLVQSGAEVKKPGASVKVSCKASGYTFINYNMHWVRQAPGQ | 229 |
| US7615214, | GLEWMGAIFPGNGFTSYNQKFKGRVTITVDKSTSTAYMELSSLRS | |
| | EDTAVYYCARDGDYYFDYWGQGTLVTVSSASTKGPSVFPLAPSS | |
| B106-2_HC | KSTSGGTAALGCLVKDYFPEPVTVSWNSGALTSGVHTFPAVLQS | |
| | SGLYSLSSVVTVPSSSLGTQTYICNVNHKPSNTKVDKKVEPKSCH | |
| | HHHHH | |

Constructs for binding to Podoplanin (PDPN)

| | | |
|---|---|---|
| E200 + PDPN | GHNYTTRNILPGLNITSDVLMTQTPLSLPVSLGDQASISCRSSRNI | 230 |
| binder (nfP2X₇ | VQSTGNTYLEWYLQKPGQSPKLLIFKVSNRFSGVPDRFSGSGSG | |
| epitope | TDFTLKISRVEAEDLGVYYCFQGSHVPPWTFGGGTKLEIKRTVAA | |
| underlined), | PSVFIFPPSDEQLKSGTASVVCLLNNFYPREAKVQWKVDNALQS | |
| | GNSQESVTEQDSKDSTYSLSSTLTLSKADYEKHKVYACEVTHQG | |
| US20160347834 | LSSPVTKSFNRGECHHHHHH | |
| A1, B107-1_LC | | |
| PDPN, | DVQLVESGGGLVQPGGSRKLSCAASGFTFSGFGMHWVRQAPEK | 231 |
| US20160347834 | GLEWVAYISSVSSRIYYADTVKGRFTISRDNPKNTLFLQMTSLRSE | |
| A1, B107-2_HC | DTAMYYCAREQTGPAWFAYWGQGTLVTVSAASTKGPSVFPLAP | |
| | SSKSTSGGTAALGCLVKDYFPEPVTVSWNSGALTSGVHTFPAVL | |
| | QSSGLYSLSSVVTVPSSSLGTQTYICNVNHKPSNTKVDKKVEPKS | |
| | CHHHHHH | |

Constructs for binding to WT1

| | | |
|---|---|---|
| E200 + WT1 | GHNYTTRNILPGLNITSQTWTQPPSASGTPGQRVTISCSGSSSNI | 232 |
| binder (nfP2X₇ | GSNYVYWYQQLPGTAPKLVLLIYRSNQRPSGVPDRFSGSKSGTS | |
| epitope | ASLAISGPRSVDEADYYCAAWDDSLNGVVFGGGTKLTVLGKRTV | |
| underlined), | AAPSVFIFPPSDEQLKSGTASVVCLLNNFYPREAKVQWKVDNALQ | |
| EP2694553A2, | SGNSQESVTEQDSKDSTYSLSSTLTLSKADYEKHKVYACEVTHQ | |
| B108-1_LC | GLSSPVTKSFNRGECHHHHHH | |
| WT1, | QVQLVQSGAEVKKPGSSVKVSCKASGGTFSSYAISWVRQAPGQ | 233 |
| EP2694553A2, | GLEVHWMGGIIPIFGTANYAQKFQGRVTITADESTSTAYMELSSLR | |
| B108-2_HC | SEDTAVYYCARRIPPYYGMDVWGQGTTVTVSSASTKGPSVFPLA | |
| | PSSKSTSGGTAALGCLVKDYFPEPVTVSWNSGALTSGVHTFPAV | |
| | LQSSGLYSLSSVVTVPSSSLGTQTYICNVNHKPSNTKVDKKVEPK | |
| | SCHHHHHH | |

Constructs for binding to GPC2

| | | |
|---|---|---|
| E200 + GPC2 | GHNYTTRNILPGLNITSENVLTQSPAIMSASLGEKVTMSCRASSSV | 234 |
| binder (nfP2X₇ | NYIYWYQQKSDASPKLWIYYTSNLAPGVPARFSGSGSGNSYSLTI | |
| epitope | SSMEGEDAATYYCQQFSSSPSTFGTGTKLELKRTVAAPSVFIFPP | |
| underlined), | SDEQLKSGTASVVCLLNNFYPREAKVQWKVDNALQSGNSQESV | |
| WO2020033430, | TEQDSKDSTYSLSSTLTLSKADYEKHKVYACEVTHQGLSSPVTKS | |
| B109-1_LC | FNRGECHHHHHH | |
| GPC2, | EVQLQQSGPELVKPGASVKMSCKASRFTFTDYNIHWVKQSPGKT | 235 |
| WO2020033430, | LEWIGYINPNNGDIFYKQKFNGKATLTINKSSNTAYMELRSLTSED | |
| B109-2_HC | SAVYYCVRSSNIRYTFDRFFDVWGTGTTVTVSSASTKGPSVFPLA | |
| | PSSKSTSGGTAALGCLVKDYFPEPVTVSWNSGALTSGVHTFPAV | |
| | LQSSGLYSLSSVVTVPSSSLGTQTYICNVNHKPSNTKVDKKVEPK | |
| | SCHHHHHH | |

Constructs for binding to FGFR4

| | | |
|---|---|---|
| E200 + FGFR4 | GHNYTTRNILPGLNITSDIQMTQSPSSLSASVGDRVTITCRASESV | 236 |
| binder (nfP2X₇ | STLMHWYQQKPGKAPKLLIYGTSNLESGVPSRFSGSGSGTDFTL | |
| epitope | TISSLQPEDFATYYCQQSWNDPPTFGGGTKVEIKRTVAAPSVFIF | |
| underlined), | PPSDEQLKSGTASVVCLLNNFYPREAKVQWKVDNALQSGNSQE | |
| WO2019034427A | SVTEQDSKDSTYSLSSTLTLSKADYEKHKVYACEVTHQGLSSPVT | |
| 1, B110-1_LC | KSFNRGECHHHHHH | |
| FGFR4, | EVQLVESGGGLVQPGGSLRLSCAASGFTFSNYYMSWVRQAPGK | 237 |
| WO2019034427A | GLEWVATINPSGTRTYYPDSVKGRFTISRDNAKNSLYLQMNSLRA | |
| 1, B110-2_HC | EDTAVYYCARLYNNYAFDYWGQGTLVTVSSASTKGPSVFPLAPS | |
| | SKSTSGGTAALGCLVKDYFPEPVTVSWNSGALTSGVHTFPAVLQ | |
| | SSGLYSLSSVVTVPSSSLGTQTYICNVNHKPSNTKVDKKVEPKSC | |
| | HHHHHH | |

Constructs for binding to EphB4

| | | |
|---|---|---|
| E200 + EphB4 | GHNYTTRNILPGLNITSDIQMTQSPSSLSASVGDRVTITCRASQDV | 238 |
| binder (nfP2X₇ | STAVAWYQQKPGKAPKLLIYSASFLYSGVPSRFSGSGSGTDFTLT | |
| epitope | ISSLQPEDFATYYCQQTAQTPETFGQGTKVEIKRTVAAPSVFIFPP | |
| underlined), | SDEQLKSGTASVVCLLNNFYPREAKVQWKVDNALQSGNSQESV | |
| EP1973950, | TEQDSKDSTYSLSSTLTLSKADYEKHKVYACEVTHQGLSSPVTKS | |
| B111-1_LC | FNRGECHHHHHH | |
| EphB4, | EVQLVESGGGLVQPGGSLRLSCAASGFTISGYYIHWVRQAPGKG | 239 |
| EP1973950, | LEWVGGIYLYSGSTDYADSVKGYADSVKGRFTISADTSKNTAYLQ | |
| B111-2_HC | MNSLRAEDTAVYYCARGSGLRLGGLDYAMDYWGQGTLVTASTK | |
| | GPSVFPLAPSSKSTSGGTAALGCLVKDYFPEPVTVSWNSGALTS | |
| | GVHTFPAVLQSSGLYSLSSVVTVPSSSLGTQTYICNVNHKPSNTK | |
| | VDKKVEPKSCHHHHHH | |

Constructs for binding to STEAP-1

| | | |
|---|---|---|
| E200 + STEAP-1 | GHNYTTRNILPGLNITSDIQMTQSPSSLSASVGDRVTITCKSSQSL | 240 |
| binder (nfP2X₇ | LYRSNQKNYLAWYQQKPGKAPKLLIYWASTRESGVPSRFSGSGS | |
| epitope | GTDFTLTISSLQPEDFATYYCQQYYNYPRTFGQGTKVEIKRTVAA | |
| underlined), | PSVFIFPPSDEQLKSGTASVVCLLNNFYPREAKVQWKVDNALQS | |
| WO2015112909, | GNSQESVTEQDSKDSTYSLSSTLTLSKADYEKHKVYACEVTHQG | |
| B112-1_LC | LSSPVTKSFNRGECHHHHHH | |
| STEAP-1, | EVQLVESGGGLVQPGGSLRLSCAVSGYSITSDYAWNWVRQAPG | 241 |
| WO2015112909, | KGLEWVGYISNSGSTSYNPSLKSRFTISRDTSKNTLYLQMNSLRA | |
| B112-2_HC | EDTAVYYCARERNYDYDDYYYAMDYWGQGTLVTVSSASTKGPS | |
| | VFPLAPSSKSTSGGTAALGCLVKDYFPEPVTVSWNSGALTSGVH | |
| | TFPAVLQSSGLYSLSSVVTVPSSSLGTQTYICNVNHKPSNTKVDK | |
| | KVEPKSCHHHHHH | |

Constructs for binding to STEAP-2

| | | |
|---|---|---|
| E200 + STEAP-2 | GHNYTTRNILPGLNITSDIQMTQSPSTLSASVGDRVTITCRASQSIS | 242 |
| binder (nfP2X₇ | SWLAWYQQKPGRAPNLLISKASSLKSGVPSRFSGSGSGTEFTLT | |
| epitope | VSSLQPDDFATYYCQQYYSYSYTFGQGTKLEIKRTVAAPSVFIFP | |
| underlined), | PSDEQLKSGTASVVCLLNNFYPREAKVQWKVDNALQSGNSQES | |
| US20180104357, | VTEQDSKDSTYSLSSTLTLSKADYEKHKVYACEVTHQGLSSPVTK | |
| B113-1_LC | SFNRGECHHHHHH | |
| STEAP-2, | QVQLVESGGGVVQPGRSLRLSCVASGFTISSYGMNWVRQAPGK | 243 |
| US20180104357, | GLEWVAVISYDGGNKYSVDSVKGRFTISRDNSKNTLYLQMNSLR | |
| B113-2_HC | AEDSAVYYCARGRYFDLWGRGTLVTVSSASTKGPSVFPLAPSSK | |
| | STSGGTAALGCLVKDYFPEPVTVSWNSGALTSGVHTFPAVLQSS | |
| | GLYSLSSVVTVPSSSLGTQTYICNVNHKPSNTKVDKKVEPKSCHH | |
| | HHHH | |

Constructs for binding to IL11Ra

| | | |
|---|---|---|
| E200 + IL11Ra | GHNYTTRNILPGLNITSDIVLTQSPATLSMTPGDSVSLSCRASQSI | 244 |
| binder (nfP2X₇ | SNNLHWYQQKSHESPRLLIKYASQSISGIPSRFSGSGSGTDFTLS | |
| epitope | FNSVETEDFGVYFCQQRYSWPLTFGAGTKLEMKRTVAAPSVFIF | |
| underlined), | PPSDEQLKSGTASVVCLLNNFYPREAKVQWKVDNALQSGNSQE | |
| WO2018109170, | SVTEQDSKDSTYSLSSTLTLSKADYEKHKVYACEVTHQGLSSPVT | |
| B114-1_LC | KSFNRGECHHHHHH | |
| IL11Ra, | QVQLQQPGAELVRPGSSVKLSCKASGYTFTNYWMHWLKQRPVQ | 245 |
| WO2018109170, | GLEWIGNIGPSDSKTHYNQKFKDKATLTVDKSSSTAYMQLNSLTS | |
| B114-2_HC | EDSAVYYCARGDYVLFTYWGQGTLVTVSAASTKGPSVFPLAPSS | |
| | KSTSGGTAALGCLVKDYFPEPVTVSWNSGALTSGVHTFPAVLQS | |
| | SGLYSLSSVVTVPSSSLGTQTYICNVNHKPSNTKVDKKVEPKSCH | |
| | HHHHH | |

Constructs for binding to CD163

| | | |
|---|---|---|
| E200 + CD163 | GHNYTTRNILPGLNITSDIVMTQSPSSLSASVGDRVTITCRASQSV | 246 |
| binder (nfP2X₇ | SSDVAWFQQKPGKSPKPLIYYASNRYSGVPSRFSGSGSGTDFTL | |
| epitope | TISSLQAEDFAVYFCGQDYTSPRTFGGGTKLEIKRTVAAPSVFIFP | |
| underlined), | PSDEQLKSGTASVVCLLNNFYPREAKVQWKVDNALQSGNSQES | |
| WO2011039510A | VTEQDSKDSTYSLSSTLTLSKADYEKHKVYACEVTHQGLSSPVTK | |
| 2, B115-1_LC | SFNRGECHHHHHH | |
| CD163, | QVQLQESGPGLVKPSETLSLTCTVSGYSITSDYAWNWIRQFPGN | 247 |
| WO2011039510A | KLEWMGYITYSGSTYYNPSLKSRVTISVDTSKNQFSLKLSSVTAA | |
| 2, B115-2_HC | DTATYYCVSGTYYFDYWGQGTTLTVSSASTKGPSVFPLAPSSKS | |
| | TSGGTAALGCLVKDYFPEPVTVSWNSGALTSGVHTFPAVLQSSG | |
| | LYSLSSVVTVPSSSLGTQTYICNVNHKPSNTKVDKKVEPKSCHHH | |
| | HHH | |

Constructs for binding to Chlorotoxin

| | | |
|---|---|---|
| E200 + CLTX | GHNYTTRNILPGLNITSMCMPCFTTDHQMARKCDDCCGGKGRG | 248 |
| binder (nfP2X₇ | KCYGPQCLCRKRTVAAPSVFIFPPSDEQLKSGTASVVCLLNNFYP | |
| epitope | REAKVQWKVDNALQSGNSQESVTEQDSKDSTYSLSSTLTLSKAD | |
| underlined), | YEKHKVYACEVTHQGLSSPVTKSFNRGECHHHHHH | |
| WO2017066481A | | |
| 1, B116-1_LC | | |
| CLTX, | MCMPCFTTDHQMARKCDDCCGGKGRGKCYGPQCLCRASTKGP | 249 |
| WO2017066481A | SVFPLAPSSKSTSGGTAALGCLVKDYFPEPVTVSWNSGALTSGV | |
| 1, B116-2_HC | HTFPAVLQSSGLYSLSSVVTVPSSSLGTQTYICNVNHKPSNTKVD | |
| | KKVEPKSCHHHHHH | |

Constructs for binding to CD206 Nanobody VH

| | | |
|---|---|---|
| E200 + CD206 | GHNYTTRNILPGLNITSQVQLQESGGGLVQPGGSLRLSCAASGFS | 250 |
| binder (nfP2X₇ | LDYYAIGWFRQAPGKEREGISCISYKGGSTTYADSVKGRFTISKD | |
| epitope | NAKNTAYLQMNSLKPEDTGIYSCAAGFVCYNYDYWGQGTQVTV | |
| underlined), | SSASTKGPSVFPLAPSSKSTSGGTAALGCLVKDYFPEPVTVSWN | |
| WO2014140376A | SGALTSGVHTFPAVLQSSGLYSLSSVVTVPSSSLGTQTYICNVNH | |
| 1, B117-2_HC | KPSNTKVDKKVEPKSCHHHHHH | |

Constructs for binding to IL1RAP

| | | |
|---|---|---|
| E200 + IL1RAP | GHNYTTRNILPGLNITSDVQMTQSPSSLSASVGDRVTITCQASQSI | 251 |
| binder (nfP2X₇ | YSFLSWYQQKPGQAPKLLIYAASDLESGVPSRFSGSGSGTDFTLT | |
| epitope | ISSLQPEDFATYYCQCNYIIDYGAFGQGTKVVIKRTVAAPSVFIFPP | |
| underlined), | SDEQLKSGTASVVCLLNNFYPREAKVQWKVDNALQSGNSQESV | |
| WO2017191325A | TEQDSKDSTYSLSSTLTLSKADYEKHKVYACEVTHQGLSSPVTKS | |
| 9, B118-1_LC | FNRGECHHHHHH | |
| IL1RAP, | EVQLEESGGRLVQPGTSLRLSCAVSGFSLSSYDMSWVRQAPGK | 252 |
| WO2017191325A | GLEWVSTIYIGGTTAYASWPKGRFTISKTNSKNTLYLQMNSLRAE | |
| 9, B118-2_HC | DTAVYFCARLQGANYYNSLALWGQGTLVTVSSASTKGPSVFPLA | |
| | PSSKSTSGGTAALGCLVKDYFPEPVTVSWNSGALTSGVHTFPAV | |
| | LQSSGLYSLSSVVTVPSSSLGTQTYICNVNHKPSNTKVDKKVEPK | |
| | SCHHHHHH | |

Constructs for binding to MICA

| | | |
|---|---|---|
| E200 + MICA | GHNYTTRNILPGLNITSAIQLTQSPSSLSASVGDRVTITCRASQGIS | 253 |
| binder (nfP2X₇ | SALAWYQQKPGKVPKSLIYDASSLESGVPSRFSGSGSGTDFTLTI | |
| epitope | SSLQPEDFATYYCQQFNSYPITFGQGTRLEIKRTVAAPSVFIFPPS | |
| underlined), | DEQLKSGTASVVCLLNNFYPREAKVQWKVDNALQSGNSQESVT | |
| WO2019183551, | EQDSKDSTYSLSSTLTLSKADYEKHKVYACEVTHQGLSSPVTKSF | |
| B119-1_LC | NRGECHHHHHH | |
| MICA, | QVQLVESGGGVVQPGRSLRLSCAASGFTFSNYAMHWVRQAPGE | 254 |
| WO2019183551, | GLEWVALIWYDGSNKFYGDSVKGRFTISRDNSKNTLYLQMNSLS | |
| B119-2_HC | AEDTAVYYCAREGSGHYWGQGTLVTVSSASTKGPSVFPLAPSSK | |
| | STSGGTAALGCLVKDYFPEPVTVSWNSGALTSGVHTFPAVLQSS | |

Constructs for binding to MAGE-A1 scTCR

| | | |
|---|---|---|
| E200 + MAGE-A1 | GHNYTTRNILPGLNITSMKPTLISVLVIIFILRGTRAQRVTQPEKLLS | 255 |
| binder (nfP2X₇ | VFKGAPVELKCNYSYSGSPELFWYVQYSRQRLQLLLRHISRESIK | |
| epitope | GFTADLNKGETSFHLKKPFAQEEDSAMYYCALRSGGYQKVTFGT | |
| underlined), | GTKLQVIPGGGGSGGGGSGGGGSMGIRLLCRVAFCFLAVGLVDV | |
| AU2018234830A | KVTQSSRYLVKRTGEKVFLECVQDMDHENMFWYRQDPGLGLRLI | |
| 1, B120-1_LC | YFSYDVKMKEKGDIPEGYSVSREKKERFSLILESASTNQTSMYLC | |
| | ASNNRDSYNSPLHFGNGTRLTVTHHHHHH | |

Constructs for binding to MAGE-A1 STCR

| | | |
|---|---|---|
| E200 + MAGE-A1 | GHNYTTRNILPGLNITSMKPTLISVLVIIFILRGTRAQRVTQPEKLLS | 256 |
| binder (nfP2X₇ | VFKGAPVELKCNYSYSGSPELFWYVQYSRQRLQLLLRHISRESIK | |
| epitope | GFTADLNKGETSFHLKKPFAQEEDSAMYYCALRSGGYQKVTFGT | |
| underlined), | GTKLQVIPIQNPDPAVYQLRDSKSSDKSVCLFTDFDSQTNVSQSK | |
| AU2018234830A | DSDVYITDKCVLDMRSMDFKSNSAVAWSNKSDFACANAFNNSIIP | |
| 1, B121-1_alpha | EDTFFPSPESS | |
| chain | | |
| MAGE-A1, | MGIRLLCRVAFCFLAVGLVDVKVTQSSRYLVKRTGEKVFLECVQD | 257 |
| AU2018234830A | MDHENMFWYRQDPGLGLRLIYFSYDVKMKEKGDIPEGYSVSREK | |
| 1, B121-2_beta | KERFSLILESASTNQTSMYLCASNNRDSYNSPLHFGNGTRLTVTD | |
| chain | LKNVFPPEVAVFEPSEAEISHTQKATLVCLATGFYPDHVELSWWV | |
| | NGKEVHSGVCTDPQPLKEQPALNDSRYALSSRLRVSATFWQDP | |
| | RNHFRCQVQFYGLSENDEWTQDRAKPVTQIVSAEAWGRADHHH | |
| | HHH | |

E200 + MAGE-A1 Constructs for binding to MAGE-A1 sTCR/2

| | | |
|---|---|---|
| binder (nfP2X₇ | MKPTLISVLVIIFILRGTRAQRVTQPEKLLSVFKGAPVELKCNYSYS | 258 |
| epitope | GSPELFWYVQYSRQRLQLLLRHISRESIKGFTADLNKGETSFHLK | |
| underlined), | KPFAQEEDSAMYYCALRSGGYQKVTFGTGTKLQVIPIQNPDPAVY | |
| AU2018234830A | QLRDSKSSDKSVCLFTDFDSQTNVSQSKDSDVYITDKCVLDMRS | |
| 1, B122-1_alpha | MDFKSNSAVAWSNKSDFACANAFNNSIIPEDTFFPSPESSGGGG | |
| chain | SGHNYTTRNILPGLNITS | |
| MAGE-A1, | MGIRLLCRVAFCFLAVGLVDVKVTQSSRYLVKRTGEKVFLECVQD | 259 |
| AU2018234830A | MDHENMFWYRQDPGLGLRLIYFSYDVKMKEKGDIPEGYSVSREK | |
| 1, B122-2_beta | KERFSLILESASTNQTSMYLCASNNRDSYNSPLHFGNGTRLTVTD | |
| chain | LKNVFPPEVAVFEPSEAEISHTQKATLVCLATGFYPDHVELSWWV | |
| | NGKEVHSGVCTDPQPLKEQPALNDSRYALSSRLRVSATFWQDP | |
| | RNHFRCQVQFYGLSENDEWTQDRAKPVTQIVSAEAWGRADHHH | |
| | HHH | |

Constructs for binding TRBC1

| | | |
|---|---|---|
| E200 + TRBC1 | GHNYTTRNILPGLNITSDWMTQSPLSLPVSLGDQASISCRSSQRL | 260 |
| binder (nfP2X₇ | VHSNGNTYLHWYLQKPGQSPKLLIYRVSNRFPGVPDRFSGSGSG | |
| epitope | TDFTLKISRVEAEDLGIYFCSQSTHVPYTFGGGTKLEIKRTVAAPS | |
| underlined), Jovi- | VFIFPPSDEQLKSGTASVVCLLNNFYPREAKVQWKVDNALQSGN | |
| 1. | SQESVTEQDSKDSTYSLSSTLTLSKADYEKHKVYACEVTHQGLSS | |
| WO2015132598A | PVTKSFNRGECHHHHHH | |
| 1, B122-1_LC | | |
| TRBC1, Jovi-1, | EVRLQQSGPDLIKPGASVKMSCKASGYTFTGYVMHVWKQRPGQ | 261 |
| WO2015132598A | GLEWIGFINPYNDDIQSNERFRGKATLTSDKSSTTAYMELSSLTSE | |
| 1, B122-2_HC | DSAVYYCARGAGYNFDGAYRFFDFWGQGTTLTVSSASTKGPSV | |
| | FPLAPSSKSTSGGTAALGCLVKDYFPEPVTVSWNSGALTSGVHT | |
| | FPAVLQSSGLYSLSSVVTVPSSSLGTQTYICNVNHKPSNTKVDKK | |
| | VEPKSCHHHHHH | |

Constructs for binding TRBC2

| | | |
|---|---|---|
| E200 + TRBC2 | GHNYTTRNILPGLNITSQPQSVSESPGKTVTISCTRSSGNFASKYV | 262 |
| binder (nfP2X₇ | QWYQQRPGSSPTTVIYENYQRPSGVPDRFSGSIDSSSNSATLTIS | |
| epitope | GLKTEDEADYYCQSYDEVSWFGGGTQLTVLGQPAAKRTVAAPS | |
| underlined), F09, | VFIFPPSDEQLKSGTASVVCLLNNFYPREAKVQWKVDNALQSGN | |
| WO2015132598A | SQESVTEQDSKDSTYSLSSTLTLSKADYEKHKVYACEVTHQGLSS | |
| 1, B123-1_LC | PVTKSFNRGECHHHHHH | |
| TRBC2, F09, | QMQLVQSGAEVKKPGASVKVSCKASGYTFASYYHWVRQAPGQ | 263 |
| WO2015132598A | GLEWGIINPSGGSTSYAQKFQGRVTMTRDTSTSTVYMELSRLRS | |
| 1, B123-2_HC | DDTAVYYCASNRGGSYKSVGMDVWGQGTTVTVSSASTKGPSVF | |
| | PLAPSSKSTSGGTAALGCLVKDYFPEPVTVSWNSGALTSGVHTF | |
| | PAVLQSSGLYSLSSVVTVPSSSLGTQTYICNVNHKPSNTKVDKKV | |
| | EPKSCHHHHHH | |

Constructs for binding urokinase-type
plasminogen activator receptor (uPAR)

| | | |
|---|---|---|
| E200 + uPAR | GHNYTTRNILPGLNITSDIVLTQSPASLAVSLGQRATISCRASKSVS | 264 |
| binder (nfP2X₇ | TSGYSYMHWYQQKPGQPPKLLIYLASNLESGVPARFSGSGSGTD | |
| epitope | FTLDIHPVEEEDAATYYCQHSRELPYTFGGGTKLELKRTVAAPSV | |
| underlined), 7G1, | FIFPPSDEQLKSGTASVVCLLNNFYPREAKVQWKVDNALQSGNS | |
| WO2006094828, | QESVTEQDSKDSTYSLSSTLTLSKADYEKHKVYACEVTHQGLSSP | |
| B124-1_LC | VTKSFNRGECHHHHHH | |
| uPAR, 7G1, | VQLQESGPELKKPGETVKISCKASGYTFTDYSMHWVKQAPGKGL | 265 |
| WO2006094828, | KCMGWINTETTKSTYADDFKGRFALSLETSASTVYLQISNLKNED | |
| B124-2_HC | TATYFCAREASYGEFDYWGQGTTVTVSSASTKGPSVFPLAPSSK | |
| | STSGGTAALGCLVKDYFPEPVTVSWNSGALTSGVHTFPAVLQSS | |
| | GLYSLSSVVTVPSSSLGTQTYICNVNHKPSNTKVDKKVEPKSCHH | |
| | HHHH | |

EGFRvIII targeted CAR

| | | |
|---|---|---|
| EGFRvIII targeted | EVQVLESGGGLVQPGGSLRLSCAASGFTFSSYAMSWVRQAPGK | 266 |
| CAR scFv_(HL- | GLEWVSAISGSGGSTNYADSVKGRFTISRDNSKNTLYLQMNSLR | |
| configuration_ | AEDTAVYYCAGSSGWSEYWGQGTLVTVSSGGGGSGGGGSGG | |
| clone_139)_CD8a_C | GGSDIQMTQSPSSLSASVGDRVTITCRASQGIRNNLAWYQQKPG | |
| D8TM_CD28_CD | KAPKRLIYAASNLQSGVPSRFTGSGSGTEFTLIVSSLQPEDFATYY | |
| 137_CD3zeta | CLQHHSYPLTSGGGTKVEIKTTTPAPRPPTPAPTIASQPLSLRPEA | |
| | CRPAAGGAVHTRGLDFACDIYIWAPLAGTCGVLLLSLVITLYCRSK | |
| | RSRLLHSDYMNMTPRRPGPTRKHYQPYAPPRDFAAYRSKRGRK | |
| | KLLYIFKQPFMRPVQTTQEEDGCSCRFPEEEEGGCELRVKFSRS | |
| | ADAPAYKQGQNQLYNELNLGRREEYDVLDKRRGRDPEMGGKPR | |
| | RKNPQEGLYNELQKDKMAEAYSEIGMKGERRRGKGHDGLYQGL | |
| | STATKDTYDALHMQALPPR | |
| EGFRvIII peptide | LEEKKGNYVVTDH | 267 |

Constructs for binding CD33

| | | |
|---|---|---|
| EGFRvIII- | LEEKKGNYVVTDHDIQLTQSPSTLSASVGDRVTITCRASESLDNY | 268 |
| peptide + CD33 | GIRFLTWFQQKPGKAPKLLMYAASNQGSGVPSRFSGSGSGTEFT | |
| binder (EGFRvIII | LTISSLQPDDFATYYCQQTKEVPWSFGQGTKVEVKRTVAAPSVFI | |
| epitope | FPPSDEQLKSGTASVVCLLNNFYPREAKVQWKVDNALQSGNSQ | |
| underlined) | ESVTEQDSKDSTYSLSSTLTLSKADYEKHKVYACEVTHQGLSSPV | |
| CD33, | TKSFNRGECHHHHHH | |
| Gemtuzumab, | | |
| B030-3_LC | | |
| CD33, | EVQLVQSGAEVKKPGSSVKVSCKASGYTITDSNIHWVRQAPGQS | 269 |
| Gemtuzumab, | LEWIGYIYPYNGGTDYNQKFKNRATLTVDNPTNTAYMELSSLRSE | |
| B030-2_HC | DTAFYYCVNGNPWLAYWGQGTLVTVSSASTKGPSVFPLAPSSKS | |
| | TSGGTAALGCLVKDYFPEPVTVSWNSGALTSGVHTFPAVLQSSG | |
| | LYSLSSVVTVPSSSLGTQTYICNVNHKPSNTKVDKKVEPKSCHHH | |
| | HHH | |

Constructs for binding Her2

| | | |
|---|---|---|
| EGFRvIII- | LEEKKGNYVVTDHDIQMTQSPSSLSASVGDRVTITCRASQDVNTA | 270 |
| peptide + Her2 | VAWYQQKPGKAPKLLIYSASFLYSGVPSRFSGSRSGTDFTLTISS | |
| binder (EGFRvIII | LQPEDFATYYCQQHYTTPPTFGQGTKVEIKRTVAAPSVFIFPPSD | |
| epitope | EQLKSGTASVVCLLNNFYPREAKVQWKVDNALQSGNSQESVTE | |
| underlined) | QDSKDSTYSLSSTLTLSKADYEKHKVYACEVTHQGLSSPVTKSFN | |
| Her2, | RGECHHHHHH | |
| Gemtuzumab, | | |
| B033-3_LC | | |
| Her2, | EVQLVESGGGLVQPGGSLRLSCAASGFNIKDTYIHWVRQAPGKG | 271 |
| Trastuzumab, | LEWVARIYPTNGYTRYADSVKGRFTISADTSKNTAYLQMNSLRAE | |
| B033-2_HC | DTAVYYCSRWGGDGFYAMDYWGQGTLVTVSSASTKGPSVFPLA | |
| | PSSKSTSGGTAALGCLVKDYFPEPVTVSWNSGALTSGVHTFPAV | |
| | LQSSGLYSLSSVVTVPSSSLGTQTYICNVNHKPSNTKVDKKVEPK | |
| | SCHHHHHH | |

CLDN6 targeted CAR

| | | |
|---|---|---|
| CLDN6 targeted | EVQLLESGGGLVQPGGSMRLSCAASGFTFSNYWMNWVRQAPG | 272 |
| CAR scFv_(HL- | KGLEWVAQIRLKSDNYATHYADSVKGRFTISRDDSKNTVYLQMN | |
| configuration_ | SLRAEDTGVYYCNDGPPSGYWGQGTLLTVSSGGGGSGGGGSG | |
| clone_ | GGGSDIQMTQSPSSLSASVGDRVTITCRISENIYSYLAWYQQKPG | |
| Ab3-4)_ | KAPKLLIYNAKILVEGVPSRFSGSGSGTDFTLTISSLQPEDFATYYC | |
| CD8a_CD8TM_ | QHHYTVPWTFGQGTKLEIKTTTPAPRPPTPAPTIASQPLSLRPEA | |
| CD28_CD137_ | CRPAAGGAVHTRGLDFACDIYIWAPLAGTCGVLLLSLVITLYCRSK | |
| CD3zeta | RSRLLHSDYMNMTPRRPGPTRKHYQPYAPPRDFAAYRSKRGRK | |
| | KLLYIFKQPFMRPVQTTQEEDGCSCRFPEEEEGGCELRVKFSRS | |
| | ADAPAYKQGQNQLYNELNLGRREEYDVLDKRRGRDPEMGGKPR | |
| | RKNPQEGLYNELQKDKMAEAYSEIGMKGERRRGKGHDGLYQGL | |
| | STATKDTYDALHMQALPPR | |
| CLDN6 peptide 23- | WTAHAIIRDFYNPLVAEAQKREL | 273 |
| mer | | |
| CLDN6 peptide 13- | TAHAIIRDFYNPL | 274 |
| mer | | |
| CLDN6 peptide 10- | LVAEAQKREL | 275 |
| mer | | |

Constructs for binding CD33

| | | |
|---|---|---|
| CLDN6- | WTAHAIIRDFYNPLVAEAQKRELDIQLTQSPSTLSASVGDRVTITC | 276 |
| peptide + CD33 | RASESLDNYGIRFLTWFQQKPGKAPKLLMYAASNQGSGVPSRFS | |
| binder (CLDN6 | GSGSGTEFTLTISSLQPDDFATYYCQQTKEVPWSFGQGTKVEVK | |
| epitope | RTVAAPSVFIFPPSDEQLKSGTASVVCLLNNFYPREAKVQWKVDN | |
| underlined) | ALQSGNSQESVTEQDSKDSTYSLSSTLTLSKADYEKHKVYACEVT | |
| CD33, | HQGLSSPVTKSFNRGECHHHHHH | |
| Gemtuzumab, | | |
| B030-4 LC | | |
| CD33, | EVQLVQSGAEVKKPGSSVKVSCKASGYTITDSNIHWVRQAPGQS | 277 |
| Gemtuzumab, | LEWIGYIYPYNGGTDYNQKFKNRATLTVDNPTNTAYMELSSLRSE | |
| B030-2_HC | DTAFYYCVNGNPWLAYWGQGTLVTVSSASTKGPSVFPLAPSSKS | |
| | TSGGTAALGCLVKDYFPEPVTVSWNSGALTSGVHTFPAVLQSSG | |
| | LYSLSSVVTVPSSSLGTQTYICNVNHKPSNTKVDKKVEPKSCHHH | |
| | HHH | |

Constructs for binding Her2

| | | |
|---|---|---|
| CLDN6- | WTAHAIIRDFYNPLVAEAQKRELDIQMTQSPSSLSASVGDRVTITC | 278 |
| peptide + Her2 | RASQDVNTAVAWYQQKPGKAPKLLIYSASFLYSGVPSRFSGSRS | |
| binder (CLDN6 | GTDFTLTISSLQPEDFATYYCQQHYTTPPTFGQGTKVEIKRTVAAP | |
| epitope | SVFIFPPSDEQLKSGTASVVCLLNNFYPREAKVQWKVDNALQSG | |
| underlined) | NSQESVTEQDSKDSTYSLSSTLTLSKADYEKHKVYACEVTHQGL | |
| Her2, | SSPVTKSFNRGECHHHHHH | |
| Trastuzumab, | | |
| B033-4_LC | | |
| Her2, | EVQLVESGGGLVQPGGSLRLSCAASGFNIKDTYIHWVRQAPGKG | 279 |
| Trastuzumab, | LEWVARIYPTNGYTRYADSVKGRFTISADTSKNTAYLQMNSLRAE | |
| B033-2_HC | DTAVYYCSRWGGDGFYAMDYWGQGTLVTVSSASTKGPSVFPLA | |
| | PSSKSTSGGTAALGCLVKDYFPEPVTVSWNSGALTSGVHTFPAV | |
| | LQSSGLYSLSSVVTVPSSSLGTQTYICNVNHKPSNTKVDKKVEPK | |
| | SCHHHHHH | |

Constructs for binding B7-H7 (HHLA2)

| | | |
|---|---|---|
| E200 + B7H7 | GHNYTTRNILPGLNITSDIVMTQSPSSLAVSAGEKVTISCLSSQSLF | 280 |
| binder (nfP2X₇ | SSNTKRNYLNWYLQKPGQSPKLLIYHASTRLTGVPGRFIGSGSGT | |
| epitope | DFTLTVSTVQAEDLGDYFCQQHYETPLTFGDGTRLEIKRTVAAPS | |
| underlined), 4.5, | VFIFPPSDEQLKSGTASVVCLLNNFYPREAKVQWKVDNALQSGN | |
| WO2014100823A | SQESVTEQDSKDSTYSLSSTLTLSKADYEKHKVYACEVTHQGLSS | |
| 1, B125-1_LC | PVTKSFNRGECHHHHHH | |
| B7H7, 4.5, | QIQLQESGPGLVKPSQSLSLTCSVTGFSITTGGYYWNWIRQFPGK | 281 |
| WO2014100823A | KLEWMGYIYTSGRTSYNPSLKSRISITRDTSKNQFFLQLNSMTTE | |
| 1, B125-2_HC | DTATYYCADMADKGGWFAYWGQGTLVTVSSASTKGPSVFPLAP | |
| | SSKSTSGGTAALGCLVKDYFPEPVTVSWNSGALTSGVHTFPAVL | |
| | QSSGLYSLSSVVTVPSSSLGTQTYICNVNHKPSNTKVDKKVEPKS | |
| | CHHHHHH | |

Constructs for binding CD34

| | | |
|---|---|---|
| E200 + CD34 | GHNYTTRNILPGLNITSDVLLTQSPLSLPVTLGQPASISCRSSQTIV | 282 |
| binder (nfP2X₇ | HSNGNTYLEWFQQRPGQSPRLLIYQVSNRFSGVPDRFSGSGSG | |
| epitope | TDFTLKISRVEAEDVGVYYCFQGSHVPRTFGGGTKVEIKRTVAAP | |
| underlined), | SVFIFPPSDEQLKSGTASVVCLLNNFYPREAKVQWKVDNALQSG | |
| h4C8b, | NSQESVTEQDSKDSTYSLSSTLTLSKADYEKHKVYACEVTHQGL | |
| US20100311955 | SSPVTKSFNRGECHHHHHH | |
| A1, B126-1_LC | | |
| CD34, h4C8b, | QIQLVQSGSELKKPGASVKVSCKASGYTFTNYGMNWVRQAPGQ | 283 |
| US20100311955 | GLKWMGWINTNTGEPKYAEEFKGRFALSLDTSVSTAYLQINSLKA | |
| A1, B126-2_HC | EDTAVYFCARGYGNYARGAWLAYWGQGTLVTVSSASTKGPSVF | |
| | PLAPSSKSTSGGTAALGCLVKDYFPEPVTVSWNSGALTSGVHTF | |
| | PAVLQSSGLYSLSSVVTVPSSSLGTQTYICNVNHKPSNTKVDKKV | |
| | EPKSCHHHHHH | |

Constructs for binding CD7

| | | |
|---|---|---|
| E200 + CD7 binder | GHNYTTRNILPGLNITSDIQMTQTTSSLSASLGDRVTISCSASQGIS | 284 |
| (nfP2X₇ epitope | NYLNWYQQKPDGTVKLLIYYTSSLHSGVPSRFSGSGSGTDYSLTI | |
| underlined), seq | SNLEPEDIATYYCQQYSKLPYTFGGGTKLEIKRTVAAPSVFIFPPS | |
| 1, | DEQLKSGTASVVCLLNNFYPREAKVQWKVDNALQSGNSQESVT | |
| WO2003051926, | EQDSKDSTYSLSSTLTLSKADYEKHKVYACEVTHQGLSSPVTKSF | |
| B127-1_LC | NRGECHHHHHH | |
| CD7, seq 1, | EVQLVESGGGLVKPGGSLKLSCAASGLTFSSYAMSWVRQTPEK | 285 |
| WO2003051926, | RLEWVASISSGGFTYYPDSVKGRFTISRDNARNILYLQMSSLRSE | |
| B127-2_HC | DTAMYYCARDEVRGYLDVWGAGTTVTVSSASTKGPSVFPLAPSS | |
| | KSTSGGTAALGCLVKDYFPEPVTVSWNSGALTSGVHTFPAVLQS | |
| | SGLYSLSSVVTVPSSSLGTQTYICNVNHKPSNTKVDKKVEPKSCH | |
| | HHHHH | |

Constructs for binding CD7

| | | |
|---|---|---|
| E200 + CD7 binder | GHNYTTRNILPGLNITSMDVQLQESGGGSVQAGGSLRLSCPASG | 286 |
| (nfP2X₇ epitope | YTFSHYCMGWNRQAPGKEREEVATIDTDDTPTYADSVMGRFTIS | |
| underlined), seq | RDNANNALYLQMNDLKPEDTSMYYCAIWMKLRGSCHDRRLEVR | |
| 32, | GQGTQVTVSINASTKGPSVFPLAPSSKSTSGGTAALGCLVKDYFP | |
| US20170226204, | EPVTVSWNSGALTSGVHTFPAVLQSSGLYSLSSVVTVPSSSLGT | |
| B128-1_HC | QTYICNVNHKPSNTKVDKKVEPKSCHHHHHH | |

Constructs for binding GPRC5D

| | | |
|---|---|---|
| E200 + GPRC5D | GHNYTTRNILPGLNITSQSVLTQPPSVSAAPGQKVTIPCSGSRSN | 287 |
| binder (nfP2X₇ | VGNYYVSWYQQLPGTAPKLLIYDNNKRPSGIPDRFSGSKSGTSA | |
| epitope | TLGITGLQTGDEADYFCGTWDGSLSAHVFGTGTKVTVLGRTVAA | |
| underlined), seq | PSVFIFPPSDEQLKSGTASVVCLLNNFYPREAKVQWKVDNALQS | |
| 1. | GNSQESVTEQDSKDSTYSLSSTLTLSKADYEKHKVYACEVTHQG | |
| SG10202007326 | LSSPVTKSFNRGECHHHHHH | |
| Q, B129-1_LC | | |
| GPRC5D, seq 1, | QVQLVQSGSELKKPGASVRVSCTASGYTFTSYYMHWVRQAPGQ | 288 |
| SG10202007326 | GLEWMGVINPNAGSTRYAQKFQGRVTMSTDTSTSTAYMDLSSLR | |
| Q, B129-2_HC | SEDTAVYYCARGMYRSLLFYDPWGQGTLVTVSSASTKGPSVFPL | |
| | APSSKSTSGGTAALGCLVKDYFPEPVTVSWNSGALTSGVHTFPA | |
| | VLQSSGLYSLSSVVTVPSSSLGTQTYICNVNHKPSNTKVDKKVEP | |
| | KSCHHHHHH | |

Constructs for binding TIM-3

| | | |
|---|---|---|
| E200 + TIM-3 | GHNYTTRNILPGLNITSDIQMTQSPSSLSASVGDRVTITCLASQPIG | 289 |
| binder (nfP2X₇ | IWLAWYQQKPGKAPKLLIYAATSLADGVPSRFSGSGSGTDFTFTI | |
| epitope | SSLQPEDIATYYCQQLYSSPWTFGGGTKVEIKRTVAAPSVFIFPPS | |
| underlined), | DEQLKSGTASVVCLLNNFYPREAKVQWKVDNALQSGNSQESVT | |
| h1701-009NKG, | EQDSKDSTYSLSSTLTLSKADYEKHKVYACEVTHQGLSSPVTKSF | |
| EP3587452, | NRGECHHHHHH | |
| B130-1_LC | | |
| TIM-3, h1701- | QVQLVQSGAEVKKPGASVKVSCKASGYTFTDYYMNWVRQAPGQ | 290 |
| 009NKG, | GLEWMGDIIPNKGGSKYNQKFKDRVTMTTDTSTSTAYMELRSLR | |
| EP3587452, | SDDTAVYYCATWGYGSSYRWFDYWGQGTLVTVSSASTKGPSVF | |
| B130-2_HC | PLAPSSKSTSGGTAALGCLVKDYFPEPVTVSWNSGALTSGVHTF | |
| | PAVLQSSGLYSLSSVVTVPSSSLGTQTYICNVNHKPSNTKVDKKV | |
| | EPKSCHHHHHH | |

Constructs for binding to CD191 (CCR1)

| | | |
|---|---|---|
| E200 + CD191 | GHNYTTRNILPGLNITSDIVMTQSPLSLPVTLGEPASISCRSSQSLV | 291 |
| binder (nfP2X₇ | HRNGITFFHWYLQKPGQSPKLLIYKISNRFSGVPDRFSGSGSGTD | |
| epitope | FTLKISRVEAEDVGVYFCSQGTHVPPTFGQGTKLEIKRTVAAPSV | |
| underlined), | FIFPPSDEQLKSGTASVVCLLNNFYPREAKVQWKVDNALQSGNS | |
| hzmAb5- | QESVTEQDSKDSTYSLSSTLTLSKADYEKHKVYACEVTHQGLSSP | |
| 06_LV5HV14, | VTKSFNRGECHHHHHH | |
| EP3656791, | | |
| B131-1_LC | | |
| CD191, hzmAb5- | QVQLQQSGPGLVKPSQTLSITCTVSGFSLNNYGVHWVRQPPGK | 292 |
| 06_LV5HV14, | GLEWLGVIWSAGTTVYNAAAISRLTISKDTSKNQVSFKMSSLTAA | |
| EP3656791, | DTAVYYCAKDGSRYYTAMDYWGQGTLVTVSSASTKGPSVFPLAP | |
| B131-2_HC | SSKSTSGGTAALGCLVKDYFPEPVTVSWNSGALTSGVHTFPAVL | |
| | QSSGLYSLSSVVTVPSSSLGTQTYICNVNHKPSNTKVDKKVEPKS | |
| | CHHHHHH | |

Constructs for binding to CD66b (CEACAM8)

| | | |
|---|---|---|
| E200 + CD66b | GHNYTTRNILPGLNITSDIQMTQSPSSLSASVGDRVTITCRASSSV | 293 |
| binder (nfP2X₇ | SYMHWYQQKPGKAPKPLIYATSNLASGVPSRFSGSGSGTDFTFTI | |
| epitope | SSLQPEDIATYYCQQWSSNPLTFGQGTKVEIKRTVAAPSVFIFPPS | |
| underlined), | DEQLKSGTASVVCLLNNFYPREAKVQWKVDNALQSGNSQESVT | |
| BW250-183, | EQDSKDSTYSLSSTLTLSKADYEKHKVYACEVTHQGLSSPVTKSF | |
| EP0585570A1, | NRGECHHHHHH | |
| B132-1_LC | | |
| CD66b, BW250- | QVQLQESGPGLVRPSQTLSLTCTVSGFSDYYMNWVRQPPGRGL | 294 |
| 183, | EWIGFISNKPNGHTTEYSASVKGRVTMLRDTSKNQFSLRLSSVTA | |
| EP0585570A1, | ADTAVYYCARDKGIRWYFDVWGQGSLVTVSSASTKGPSVFPLAP | |
| B132-2_HC | SSKSTSGGTAALGCLVKDYFPEPVTVSWNSGALTSGVHTFPAVL | |
| | QSSGLYSLSSVVTVPSSSLGTQTYICNVNHKPSNTKVDKKVEPKS | |
| | CHHHHHH | |

Constructs for binding to CD11b (MAC-1)

| | | |
|---|---|---|
| E200 + CD66b | GHNYTTRNILPGLNITSDIQMTQSPSSLSASLGERVSLTCRASQEI | 295 |
| binder (nfP2X₇ | SGYLSWHQQKPDGTIKRLLYSTSTLDSGVPKRFSGSRSGSDYSL | |
| epitope | TISSLESEDFADYYCLQYAISPPTFGGGTKLEIKRTVAAPSVFIFPP | |
| underlined), | SDEQLKSGTASVVCLLNNFYPREAKVQWKVDNALQSGNSQESV | |
| seq_1, | TEQDSKDSTYSLSSTLTLSKADYEKHKVYACEVTHQGLSSPVTKS | |
| WO2017220369A | FNRGECHHHHHH | |
| 1, B133-1_LC | | |
| CD11b, seq_1, | QVTLKESGPGILQTSQTLSLTCSFSGFSLSTSGMGVSWIRQPSGK | 296 |
| WO2017220369A | GLEWLAHIYWDDDKRYNPSLKSRLTISKDTSRNQVFLKITSVDTTD | |
| 1, B133-2_HC | TATYYCALNYYNSTYNFDFWGQGTTLTVSSASTKGPSVFPLAPS | |
| | SKSTSGGTAALGCLVKDYFPEPVTVSWNSGALTSGVHTFPAVLQ | |
| | SSGLYSLSSVVTVPSSSLGTQTYICNVNHKPSNTKVDKKVEPKSC | |
| | HHHHHH | |

Constructs for binding to EMR2 (ADGRE2)

| | | |
|---|---|---|
| E200 + EMR2 | GHNYTTRNILPGLNITSDIQMTQSPSSLSASVGDRVTITCKASQNV | 297 |
| binder (nfP2X₇ | RTTVDWYQQKPGKAPKLLIYLASNRHTGVPSRFSGSGSGTDFTL | |
| epitope | TISSLQPEDFATYYCLQHRNYPLTFGGGTKVEIKRTVAAPSVFIFP | |
| underlined), | PSDEQLKSGTASVVCLLNNFYPREAKVQWKVDNALQSGNSQES | |
| hSC93.253, | VTEQDSKDSTYSLSSTLTLSKADYEKHKVYACEVTHQGLSSPVTK | |
| WO2017087800A | SFNRGECHHHHHH | |
| 1, B134-1_LC | | |
| EMR2, | EVQLVESGGGLVKPGGSLRLSCAASGFTFSSYDMSWVRQAPGK | 298 |
| hSC93.253, | GLEWVSTISSGGNYNYYPDSVKGRFTISRDNAKNSLYLQMNSLR | |
| WO2017087800A | AEDTAVYYCARHYDYPDYAMDYWGQGTTVTVSSASTKGPSVFP | |
| 1, B134-2_HC | LAPSSKSTSGGTAALGCLVKDYFPEPVTVSWNSGALTSGVHTFP | |
| | AVLQSSGLYSLSSVVTVPSSSLGTQTYICNVNHKPSNTKVDKKVE | |
| | PKSCHHHHHH | |

Constructs for binding to MUC16

| | | |
|---|---|---|
| E200 + MUC16 | GHNYTTRNILPGLNITSDIVMTQAAPSVPVTPGESVSISCRSSKSL | 299 |
| binder (nfP2X₇ | LHSNGNTYLYWFLQRPGQSPQRLIYYMSNLASGVPDRFSGRGS | |
| epitope | GTDFTLRISRVEAEDVGVYYCMQSLEYPLTFGGGTKLEIKRTVAA | |
| underlined), | PSVFIFPPSDEQLKSGTASVVCLLNNFYPREAKVQWKVDNALQS | |
| 18C6, | GNSQESVTEQDSKDSTYSLSSTLTLSKADYEKHKVYACEVTHQG | |
| WO2016149368A | LSSPVTKSFNRGECHHHHHH | |
| 1, B135-1_LC | | |
| MUC16, 18C6, | QVTLKESGPGILQPSQTLSLTCSFSGFSLSTVGMGVGWSRQPSG | 300 |
| WO2016149368A | KGLEWLAHIWWDDEDKYYNPALKSRLTISKDTSKNQVFLKIANVD | |
| 1, B135-2_HC | TADTATYYCTRIGTAQATDALDYWGQGTSVTVSSASTKGPSVFPL | |
| | APSSKSTSGGTAALGCLVKDYFPEPVTVSWNSGALTSGVHTFPA | |
| | VLQSSGLYSLSSVVTVPSSSLGTQTYICNVNHKPSNTKVDKKVEP | |
| | KSCHHHHHH | |

Constructs for binding to NYESO-1 HLA-A2 (scTCR)

| | | |
|---|---|---|
| E200 + scTCR | GHNYTTRNILPGLNITSAQSVAQPEDLVNVAEGNPLTVKCTYSVS | 301 |
| NYESO-1v1 | GNPYLFWYVQYPNRGLQFLLKYLGDSALVKGSYGFEAEFNKSQT | |
| binder (nfP2X₇ | SFHLKKPSALVSDSALYFCAVRDINSGAGSYQLTFGKGTKLSVIPG | |
| epitope | GGGSGGGGSGGGGSSAVISQKPSRDIKQRGTSLTIQCQVDKRLA | |
| underlined), | LMFWYRQQPGQSPTLIATAWTGGEATYESGFVIDKFPISRPNLTF | |
| 18C6, | STLTVSNMSPEDSSIYLCSVGGSGAADTQYFGPGTRLTVLHHHH | |
| WO2017109496A | HH | |
| 1, B136-1_v1 | | |
| E200 + scTCR | HHHHHHAQSVAQPEDLVNVAEGNPLTVKCTYSVSGNPYLFWYV | 302 |
| NYESO-1v2 | QYPNRGLQFLLKYLGDSALVKGSYGFEAEFNKSQTSFHLKKPSAL | |
| binder (nfP2X₇ | VSDSALYFCAVRDINSGAGSYQLTFGKGTKLSVIPGGGGSGGGG | |
| epitope | SGGGGSSAVISQKPSRDIKQRGTSLTIQCQVDKRLALMFWYRQQ | |
| underlined), | PGQSPTLIATAWTGGEATYESGFVIDKFPISRPNLTFSTLTVSNMS | |
| WO2017109496A | PEDSSIYLCSVGGSGAADTQYFGPGTRLTVLGGGGSGHNYTTRN | |
| 1, B136-2_v2 | ILPGLNITS | |

Constructs for binding to SURVIVIN HLA-A2 (scTCR)

| | | |
|---|---|---|
| E200 + ScTCR | GHNYTTRNILPGLNITSSQTIHQWPATLVQPVGSPLSLECTVEGTS | 303 |
| SURVIVINv1 | NPNLYWYRQAAGRGLELLFYSVGIGQISSEVPQNLFASRPQDRQ | |
| binder (nfP2X₇ | FILSSKKLLLSDSGFYLCAWSIGAEQFFGPGTRLTVLEDLKNGSAD | |
| epitope | DAKKDAAKKDGKSGGGGSGGGGSGGGGSQKEVEQNSGPLSVP | |
| underlined), | EGAIASLNCTYSDRGSQSFFWYRQYSGKSPELIMSIYSNGDKED | |
| 18C6, NZ719707, | GRFTAQLNKASQYVSLLIRDSQPSDSATYLCAVSKGYKMFGDGT | |
| B137-1_v1 | QLVVKPNIHHHHHH | |
| E200 + scTCR | HHHHHHSQTIHQWPATLVQPVGSPLSLECTVEGTSNPNLYWYR | 304 |
| NYESO-1v2 | QAAGRGLELLFYSVGIGQISSEVPQNLFASRPQDRQFILSSKKLLL | |
| binder (nfP2X₇ | SDSGFYLCAWSIGAEQFFGPGTRLTVLEDLKNGSADDAKKDAAK | |
| epitope | KDGKSGGGGSGGGGSGGGGSQKEVEQNSGPLSVPEGAIASLN | |
| underlined), | CTYSDRGSQSFFWYRQYSGKSPELIMSIYSNGDKEDGRFTAQLN | |
| NZ719707, B137- | KASQYVSLLIRDSQPSDSATYLCAVSKGYKMFGDGTQLVVKPNIG | |
| 2_v2 | GGGSGHNYTTRNILPGLNITS | |

Constructs for biding to BCMA (ligand)

| | | |
|---|---|---|
| E200 + dAPRIL | GHNYTTRNILPGLNITSSVLHLVPINATSKDDSDVTEVMWQPALR | 305 |
| binder (nfP2X₇ | RGRGLQAQGYGVRIQDAGVYLLYSQVLFQDVTFTMGQVVSREG | |
| epitope | QGRQETLFRCIRSMPSHPDRAYNSCYSAGVFHLHQGDILSVIIPR | |
| underlined), | ARAKLNLSPHGTFLGFVKLSGGGSDPHHHHHH | |
| WO2015052538A | | |
| 1, B138-1_v1 | | |
| E200 + dAPRIL | HHHHHHSVLHLVPINATSKDDSDVTEVMWQPALRRGRGLQAQG | 306 |
| binder (nfP2X₇ | YGVRIQDAGVYLLYSQVLFQDVTFTMGQVVSREGQGRQETLFRC | |
| epitope | IRSMPSHPDRAYNSCYSAGVFHLHQGDILSVIIPRARAKLNLSPHG | |
| underlined), | TFLGFVKLSGGGSDPGHNYTTRNILPGLNITS | |
| WO2015052538A | | |
| 1, B139-2_v2 | | |

Constructs for binding to CD200

| | | |
|---|---|---|
| E200 + CD200, | QVQLQQSGSELKKPGASVKISCKASGYSFTDYIILWVRQNPGKGL | 307 |
| samalizumab, | EWIGHIDPYYGSSNYNLKFKGRVTITADQSTTTAYMELSSLRSED | |
| B140-1 | TAVYYCGRSKRDYFDYWGQGTTLTVSSASTKGPSVFPLAPSSKS | |
| | TSGGTAALGCLVKDYFPEPVTVSWNSGALTSGVHTFPAVLQSSG | |
| | LYSLSSVVTVPSSSLGTQTYICNVNHKPSNTKVDKKVEPKSCHHH | |
| | HHH | |
| CD200, LC, | DIQMTQSPSSLSASIGDRVTITCKASQDINSYLSWFQQKPGKAPK | 308 |
| samalizumab, | LLIYRANRLVDGVPSRFSGSGSGTDYTLTISSLQPEDFAVYYCLQY | |
| B140-2 | DEFPYTFGGGTKLEIKRTVAAPSVFIFPPSDEQLKSGTASVVCLLN | |
| | NFYPREAKVQWKVDNALQSGNSQESVTEQDSKDSTYSLSSTLTL | |
| | SKADYEKHKVYACEVTHQGLSSPVTKSFNRGECHHHHHH | |

Further examples of tumour-specific epitope
antigen moieties (including linker and hinge regions)

| | | |
|---|---|---|
| E200 + IgG hinge | GHNYTTRNILPGLNITSEPKSSDKTHT | 365 |
| (E200 underlined) | | |
| E200 + GS | GHNYTTRNILPGLNITSGSEPKSSDKTHT | 366 |
| linker_IgG hinge | | |
| E200 + G4S | GHNYTTRNILPGLNITSGGGGSEPKSSDKTHT | 367 |
| linker + IgG hinge | | |
| Extended E200 + | GHNYTTRNILPGLNITSTFHKTSGSGKEPKSSDKTHT | 368 |
| _IgG hinge | | |
| Extended E200 + | GHNYTTRNILPGLNITSTFHKTSGSGKGSEPKSSDKTHT | 369 |
| GS linker + _IgG | | |
| hinge | | |
| Extended E200 + | GHNYTTRNILPGLNITSTFHKTSGSGKGGGGSEPKSSDKTHT | 370 |
| G4S linker + IgG | | |
| hinge | | |
| E200 + IgG | GHNYTTRNILPGLNITSEPKSSDKTHTGS | 371 |
| hinge + GS linker | | |
| E200 + GS | GHNYTTRNILPGLNITSGSEPKSSDKTHTGS | 372 |
| linker + IgG | | |
| hinge + GS linker | | |
| E200 + G4Slinker + | GHNYTTRNILPGLNITSGGGGSEPKSSDKTHTGS | 373 |
| IgG hinge + GS | | |
| linker | | |
| Extended | GHNYTTRNILPGLNITSTFHKTSGSGKEPKSSDKTHTGS | 374 |
| E200 + _IgG | | |
| hinge + GSlinker | | |
| Extended | GHNYTTRNILPGLNITSTFHKTSGSGKGSEPKSSDKTHTGS | 375 |
| E200 + GS | | |
| linker + _IgG | | |
| hinge + GSlinker | | |
| Extended | GHNYTTRNILPGLNITSTFHKTSGSGKGGGGSEPKSSDKTHTGS | 376 |
| E200 + G4S | | |
| linker + _IgG | | |
| hinge + GSlinker | | |
| E200 + IgG | GHNYTTRNILPGLNITSEPKSSDKTHTGGGGS | 377 |
| hinge + G4S linker | | |
| E200 + GS | GHNYTTRNILPGLNITSGSEPKSSDKTHTGGGGS | 378 |
| linker + IgG | | |
| hinge + G4S linker | | |
| E200 + G4S | GHNYTTRNILPGLNITSGGGGSEPKSSDKTHTGGGGS | 379 |
| linker + IgG | | |
| hinge + G4S linker | | |
| Extended | GHNYTTRNILPGLNITSTFHKTSGSGKEPKSSDKTHTGGGGS | 380 |
| E200 + IgG | | |
| hinge + G4S linker | | |
| Extended | GHNYTTRNILPGLNITSTFHKTSGSGKGSEPKSSDKTHTGGGGS | 381 |
| E200 + GS | | |
| linker + IgG | | |
| hinge + G4S linker | | |
| Extended | GHNYTTRNILPGLNITSTFHKTSGSGKGGGGSEPKSSDKTHTGG | 382 |
| E200 + G4S | GGS | |
| linker + IgG | | |
| hinge + G4S linker | | |
| N-term extended | DFPGHNYTTRNILPGLNITSEPKSSDKTHT | 383 |
| E200 + IgG hinge | | |
| N-term extended | DFPGHNYTTRNILPGLNITSGSEPKSSDKTHT | 384 |
| E200 + GS | | |
| linker + IgG hinge | | |
| N-term extended | DFPGHNYTTRNILPGLNITSGGGGSEPKSSDKTHT | 385 |
| E200 + G4S | | |
| linker + IgG hinge | | |
| N and C term | DFPGHNYTTRNILPGLNITSTFHKTSGSGKEPKSSDKTHT | 386 |
| extended | | |
| E200 + IgG hinge | | |
| N and C term | DFPGHNYTTRNILPGLNITSTFHKTSGSGKGSEPKSSDKTHT | 387 |
| extended | | |
| E200 + GS | | |
| linker + IgG hinge | | |
| N and C term | DFPGHNYTTRNILPGLNITSTFHKTSGSGKGGGGSEPKSSDKTHT | 388 |
| extended E200 + | | |
| G4S linker + IgG | | |
| hinge | | |
| N-term extended | DFPGHNYTTRNILPGLNITSEPKSSDKTHTGS | 389 |
| E200 + IgG hinge + | | |
| GS linker | | |
| N-term extended | DFPGHNYTTRNILPGLNITSGSEPKSSDKTHTGS | 390 |
| E200 + GS | | |
| linker + IgG hinge + | | |
| GS linker | | |
| N-term extended | DFPGHNYTTRNILPGLNITSGGGGSEPKSSDKTHTGS | 391 |
| E200 + G4S | | |
| linker + IgG hinge + | | |
| GS linker | | |
| N-term and C | DFPGHNYTTRNILPGLNITSTFHKTSGSGKEPKSSDKTHTGS | 392 |
| term extended | | |
| E200 + IgG | | |
| hinge + GS linker | | |
| N-term and C | DFPGHNYTTRNILPGLNITSTFHKTSGSGKGSEPKSSDKTHTGS | 393 |
| term extended | | |
| E200 + GS linker + | | |
| IgG hinge + GS | | |
| linker | | |
| N-term and C | DFPGHNYTTRNILPGLNITSTFHKTSGSGKGGGGSEPKSSDKTHT | 394 |
| term extended | GS | |
| E200 + G4S | | |
| linker + IgG | | |
| hinge + GS linker | | |
| N-term extended | DFPGHNYTTRNILPGLNITSEPKSSDKTHTGGGGS | 395 |
| E200 + IgG hinge | | |
| + G4S linker | | |
| N-term extended | DFPGHNYTTRNILPGLNITSGSEPKSSDKTHTGGGGS | 396 |
| E200 + GS | | |
| linker + IgG hinge | | |
| + G4S linker | | |
| N-term extended | DFPGHNYTTRNILPGLNITSGGGGSEPKSSDKTHTGGGGS | 397 |
| E200 + G4S | | |
| linker + IgG hinge | | |
| + G4S linker | | |
| N-term and C | DFPGHNYTTRNILPGLNITSTFHKTSGSGKEPKSSDKTHTGGGGS | 398 |
| term extended | | |
| E200 + IgG | | |
| hinge + G4S linker | | |
| N-term and C | DFPGHNYTTRNILPGLNITSTFHKTSGSGKGSEPKSSDKTHTGGG | 399 |
| term extended | GS | |
| E200 + GS linker + | | |
| IgG hinge + G4S | | |
| linker | | |
| N-term and C | DFPGHNYTTRNILPGLNITSTFHKTSGSGKGGGGSEPKSSDKTHT | 400 |
| term extended | GGGGS | |
| E200 + G4S | | |
| linker + IgG | | |
| hinge + G4S linker | | |

### DETAILED DESCRIPTION OF THE EMBODIMENTS

Reference will now be made in detail to certain embodiments of the invention. While the invention will be described in conjunction with the embodiments, it will be understood that the intention is not to limit the invention to those embodiments. On the contrary, the invention is intended to cover all alternatives, modifications, and equivalents, which may be included within the scope of the present invention as defined by the claims.

One skilled in the art will recognise many methods and materials similar or equivalent to those described herein, which could be used in the practice of the present invention. The present invention is in no way limited to the methods and materials described.

It will be understood that the invention disclosed and defined in this specification extends to all alternative combinations of two or more of the individual features mentioned or evident from the text or drawings. All of these different combinations constitute various alternative aspects of the invention.

All of the patents and publications referred to herein are incorporated by reference in their entirety.

The present invention seeks to address one or more of the deficiencies of the prior art and is based on the recognition by the inventors that it is possible to exploit the cancer-specific expression of tumour-specific antigens, such as a dysfunctional P2X₇ receptor, to:
a) expand the targets of immune cells expressing chimeric antigen receptors (CARs) which bind to a tumour-specific antigen, such as a dysfunctional P2X₇ receptor; and/or
b) provide a tunable, “switchable” approach to targeted cell killing in a variety of settings that minimise on-target, off-tumour effects; and
c) minimise aberrant immune responses to adaptor molecules.

The invention provides a new treatment modality comprising a first component and second component. The first component is the administration of an immune cell that comprises a CAR that binds to a dysfunctional P2X₇ receptor expressed on a cell surface (also referred to herein as “nfP2X-directed CAR”). The second component is the administration (or expression) of a bridging molecule that can redirect tumour-specific CARs (eg nfP2X₇-directed CARs) to other cell surface molecules, such as cancer-associated antigens, e.g. CD19, CD20, CD33, GD2, intracellularly processed proteins that are presented as peptides of various length via MHC I and II or via any other mechanism of accessible surface antigen exposure.

The targeted cell surface molecules may be associated with cancer (such as tumour associated antigens expressed on the surface of cancer cells). In further embodiments, the targeted cell surface molecules may be associated with an infection, or associated with any other disease (including autoimmune diseases). In such embodiments, the molecules may be cell surface antigens associated with the disease or may include peptide/HLA complexes presented on cells. In certain examples the molecule may comprise CD19 in B-lineage malignancy. The molecule may comprise targeted peptides related to cancer-specific proteins (genetic aberrations such as cancer testis antigens and others which are specific to the cancer patient). The molecule may be a peptide/HLA complex comprising peptides derived from an infectious agent e.g. of viral, bacterial, protozoan, virion, prion and fungal origin. The molecule may be a peptide/HLA complex comprising peptides associated with an autoimmune disease (e.g., Sm peptides associated with lupus).

Further, the present invention provides several advantages over existing “adaptor CAR” approaches. In more detail, adaptor CAR T cell technology is based on an approach to uncoupling the tumour-targeting and signalling moieties of conventional CARs, resulting in dichotomous systems consisting of an adaptor CAR and soluble, tumour-specific adaptor molecules. The basic structure of adaptor CARs corresponds to the conventional CAR design, although the extracellular domain does not interact with the tumour-associated antigen, but instead with a binding partner incorporated into the adaptor molecule. The bifunctional adaptor molecule in turn provides target cell (tumour cell) specificity and acts as a linker at the interface between the tumour and the adaptor CAR T cell. This complex can then mediate anti-tumour responses, similarly to conventional CAR T cells.

While the dual principle of the adaptor CAR systems provides an important molecular safety switch to precisely control the adaptor CAR T cell activity, several limitations have been identified with the systems developed to date.

For example, one system relies on scFv-based a-FITC CARs targeting the synthetic dye FITC that is coupled to various tumour-specific adaptor molecules. Despite the full humanisation of the a-FITC CAR T cell product, the immunogenic potential of FITC is one concern for clinical translation, as underlined by the emergence of anti-a-FITC antibodies in therapeutic mouse models.

In another example, adaptor molecules include small peptide tags to redirect standard scFv-based adaptor CARs. However, concerns again have been raised with respect to the immunogenic potential of the small peptide tags included in the adaptor molecules, particularly in the case where the tag comprises non-human sequences, or sequences derived from human nuclear proteins.

In contrast to the approaches of the prior art, the present invention takes advantage of both the specificity of CARs that bind dysfunctional P2X₇ and of the unique properties of epitopes derived from dysfunctional P2X₇ receptor.

The present invention provides an advantage over existing mono-antigen directed CAR T therapy in that it:
makes use of CAR T cells that are functional (i.e., there is no requirement for the use of a switching molecule to activate the CAR T cells);
utilises CAR T cells that do not bind to healthy cells since dysfunctional P2X₇ receptor is only exposed on the surface of cancer cells;
   this means that there is no requirement for the administration of immunosuppressing agents;
   this also means that there is a significant reduction in the side-effects as compared to other approaches that include the use of CARs that bind antigens also found on non-cancerous cells;
comprises non-immunogenic, and naturally occurring human epitopes on the bridging molecule in the form of epitopes derived from dysfunctional P2X₇ receptor.

Thus the present invention provides a new concept and approach in the use of adaptor/bridging molecules alongside CAR T therapy.

In more detail, the specificity of CARs that bind dysfunctional nfP2X₇ receptor (also referred to herein as nfP2X₇ CAR) results from the fact that dysfunctional P2X₇ is only exposed on the surface of transformed cells. Further, by including an epitope from nfP2X₇ in a bridging molecule, CAR-mediated recognition can be broadened to include any target antigen of interest via the corresponding bridging molecule. The nfP2X₇ CAR solely recognises the dysfunctional P2X₇ receptor, e.g. the E200 (or E300 or E200-300 composite) epitope, however the use of a bridging molecule facilitates unlimited targeting by means of any accessible recognition site expressed on the cell surface, e.g. an nfP2X₇ CAR can be additionally directed (or redirected) to bind to CD19 positive cells through the use of a bridging molecule that comprises a targeting moiety for binding CD19 on a cell surface, and an E200 epitope from nfP2X₇.

P2X₇ is a human receptor protein that is commonly expressed in human tissue, particularly immune and neural cells. There is no reported or registered case of autoimmune response raised against P2X₇ receptors. Exemplary targeted epitopes such as E200, E300 and E200/E300 are not genetically defined but only result from a conformational change of the tertiary structure of P2X₇. Thus, these are non-immunogenic peptide sequences that are an unaltered part of the P2X₇ sequence. Only under artificial conditions using adjuvants and conjugates can immune responses be produced against the target.

An advantage of the non-immunogenic recognition sites, e.g. the peptide sequence of E200 or E300 or the composite peptide E200-300, in the bridging molecule facilitates long-term application of bridging molecules with various specificities without induction of neutralising antibodies and/or T cell mediated rejection of cells. This represents a significant advantage over the design of bridging/adaptor molecules described in the prior art.

Moreover, nfP2X₇ CARs only target cancer tissue specifically, and therefore the approach of the present invention presents minimal risk of “on-target, off-tumour” effects and damage to healthy tissue through off-target binding of the CARs. The present invention therefore exploits the specificity of nfP2X₇ CARs in two ways: firstly by relying on the fact that nfP2X₇ CARs only target nfP2X₇ expressing cells (cancer cells only) and secondly, by relying on the fact that bridging molecules, engineered to be bound by nfP2X₇ can be used to redirect immune cells expressing nfP2X₇ CARs towards other tumour-associated and/or specific target antigens in a switchable, tuneable manner.

Thus, the use of the bridging molecules of the invention allows for the redirection of immune cells expressing nfP2X₇ CARs for the targeting of any surface expressed target antigen.

A particular advantage of the present approach is that the targeting is limited to the time period during which the bridging molecule therapeutic in vivo is persistent in circulation. This means that any toxicity arising from the “on-target, off-tumour” expression of the target antigen in healthy tissue is minimised. This is because once the bridging molecule has been cleared from the body, nfP2X₇ CAR cells are again only capable of tumour-specifically targeting nfP2X₇. In other words, as the targeting of target antigens other than nfP2X₇ is bridging molecule-dependent, the targeting can be regulated by the application of bridging molecules. This facilitates an easy to implement approach for “switching-on” and “switching-off” the targeting of cancer cells via the antigen bound by the targeting moiety of the bridging molecules such that the CAR T cells may be transiently directed to cancer cells via antigens other than nfP2X₇. Further, the length of time during which the CAR T cells are redirected to other cancer antigens can be modulated by the time for which the bridging molecules are administered to a patient in need.

It should therefore be clear that the present invention finds application in a variety of settings. For example, in the context of oncology treatment, the present invention allows for the use of a single class of CAR molecule (i.e., for binding dysfunctional P2X₇ receptor present on cancer cells), to target multiple antigens present on the cancer cells. More specifically, using a bridging molecule that comprises a targeting moiety for binding CD19, the CAR-T cells can be targeted to the cancer cells at both dysfunctional P2X₇ receptor and CD19. This maximises the likelihood of the cancer cell being killed because it is being targeted at multiple sites. Moreover, it should be evident that the use of multiple bridging molecules, or bridging molecules comprising more than one targeting moiety, facilitates the “painting” of the cancer cell surface with CAR T cells. In other words, the invention provides for the use of a variety of different bridging molecules, each comprising epitopes for being bound by a single species of CAR T cell, but comprising different targeting moieties such that the CAR T cells may be directed to bind to cancer cells via multiple cancer antigens. In this way, the cancer cells can be targeted and bound by the CAR T cell at multiple sites, increasing the efficacy of cell killing.

This approach is also particularly useful in the case of cancers that express low levels of dysfunctional P2X₇ receptor, such as Burkitt's lymphoma or subcategories of solid tumours arising from various epithelial, mesenchymal, neural or germinal origins. Another example of a low-expressing cancer cell type may be the triple negative breast cancer (e.g. MDA-MB-231 cell line). Other examples include solid tumour tissues tested in tissue arrays from PDX models, several of which show lower receptor expression than other cancers. Such examples include but are not limited to neuroblastoma, colorectal cancer, lung cancer, breast cancer or brain cancer.

In further examples, the invention finds application in the context of preventing or minimising the severity of an infection with a pathogen (preferably an intracellular pathogen). While not limited to an oncology setting, this may be particularly useful in the treatment of patients receiving cancer therapy and who are immunocompromised (and therefore susceptible to infection with opportunistic or other pathogens). Thus, a patient who has received (or is continuing to receive) a treatment with CAR T cells that bind dysfunctional P2X₇ receptor, can simultaneously be administered a bridging molecule that facilitates the redirection of the CAR T cells to cells that present peptides from an infectious agent on MHC molecules on their cell surface. In other words, the invention provides a platform for simultaneous or sequential treatment of cancer and an infectious disease.

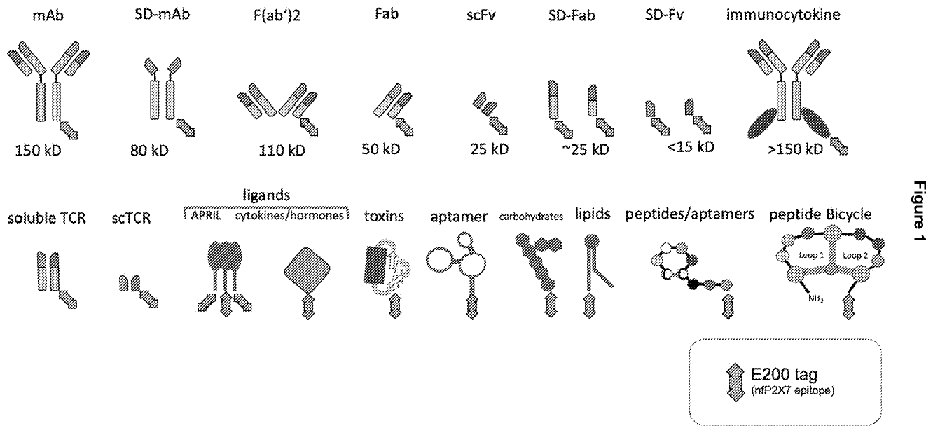

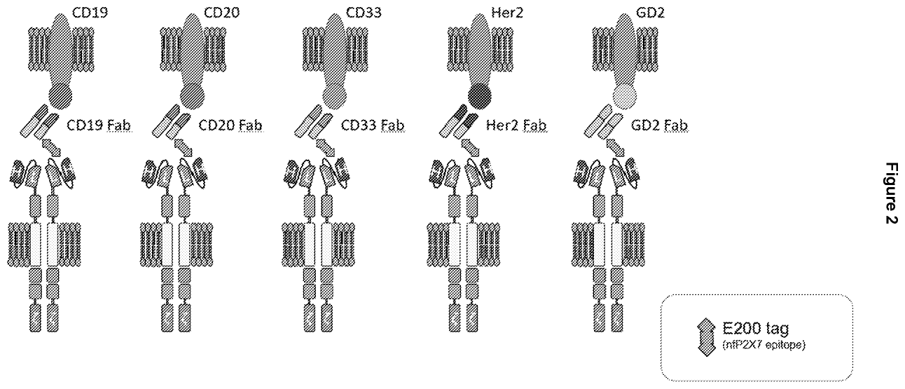

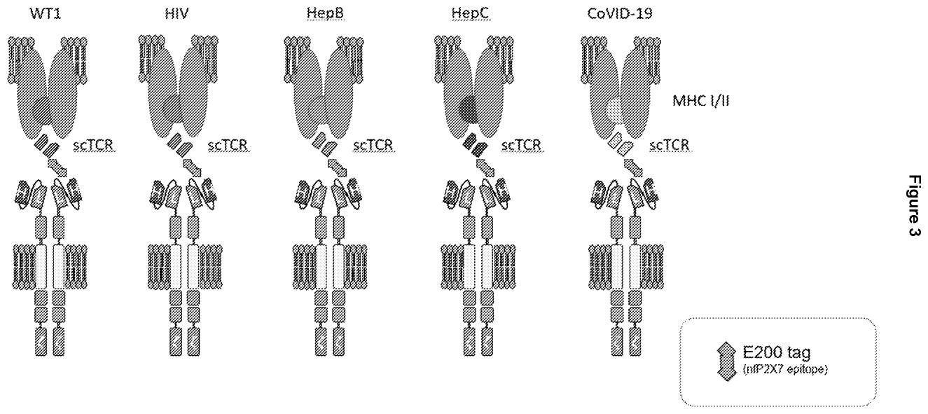

The basic principle as well as the engagement of nfPX₇ CAR expressing effector cells via nfP2X₇ E200-derived peptide tagged bridging molecules and the different formats of bridging molecules, is illustrated and outlined in FIGS. 1 to 3.

Using nfP2X₇R CAR without the presence of bridging molecules, the nfP2X₇ CAR expressing effector cells exhibit cancer-specific targeting (scenario I, see FIG. 11).

In order to broaden the applicability to nfP2X₇ functionally negative cancers (very low or negative for nfP2X₇) nfP2X₇ CAR expressing effector cells may be redirected to cancer cells via bridging molecules targeting cancer-associated antigens as illustrated for CD33 or cancer-specific antigens via TcR-like mAbs. The specificity of the bridging molecules is unlimited meaning any surface expressed target antigen or presented antigen in the context of MHC peptide presentation (class I and II) via TcR-like mAb or ligands may engage the nfP2X₇ CAR expressing effector cells in the same mode of action (scenario II. See FIG. 11).

In most cases, the dual-function of the nfP2X₇ CAR expressing effector cells is utilised (scenario 111, see FIG. 11). It is a combination of scenario 1, and II, which means that nfP2X₇ CAR expressing effector cells are engaged directly to cancer cells via nfP2X₇ expressed on the cancer cells and additionally recruited to the cancer cells via bridging molecules targeting cancer-associated antigens as illustrated for CD33 or cancer-specific antigens via TcR-like mAbs.

### Definitions

Unless defined otherwise, technical and scientific terms used herein have the same meaning as commonly understood by one of ordinary skill in the art to which this invention belongs.

For purposes of interpreting this specification, the following definitions will generally apply and whenever appropriate, terms used in the singular will also include the plural and vice versa.

“Purinergic receptor” generally refers to a receptor that uses a purine (such as ATP) as a ligand.

“P2X₇ receptor” generally refers to a purinergic receptor formed from three protein subunits or monomers, with at least one of the monomers having an amino acid sequence substantially as shown in SEQ ID NO: 1 below:

SEQ ID NO: 1
MPACCSCSDVFQYETNKVTRIQSMNYGTIKWFFHVIIFSYVCFALVSDKLYQRKEPVIS
SVHTKVKGIAEVKEEIVENGVKKLVHSVFDTADYTFPLQGNSFFVMTNFLKTEGQEQRL
CPEYPTRRTLCSSDRGCKKGWMDPQSKGIQTGRCVVYEGNQKTCEVSAWCPIEAVE
EAPRPALLNSAENFTVLIKNNIDFPGHNYTTRNILPGLNITCTFHKTQNPQCPIFRLGDIF
RETGDNFSDVAIQGGIMGIEIYWDCNLDRWFHHCRPKYSFRRLDDKTTNVSLYPGYNF
RYAKYYKENNVEKRTLIKVFGIRFDILVFGTGGKFDIIQLVVYIGSTLSYFGLAAVFIDFL
IDTYSSNCCRSHIYPWCKCCQPCVVNEYYYRKKCESIVEPKPTLKYVSFVDESHIRMVNQ
QLLGRSLQDVKGQEVPRPAMDFTDLSRLPLALHDTPPIPGQPEEIQLLRKEATPRSRD
SPVWCQCGSCLPSQLPESHRCLEELCCRKKPGACITTSELFRKLVLSRHVLQFLLLYQ
EPLLALDVDSTNSRLRHCAYRCYATWRFGSQDMADFAILPSCCRWRIRKEFPKSEGQ
YSGFKSPY

To the extent that P2X₇ receptor is formed from three monomers, it is a “trimer” or “trimeric”. “P2X₇ receptor” encompasses naturally occurring variants of P2X₇ receptor, e.g., wherein the P2X₇ monomers are splice variants, allelic variants, SNPs and isoforms including naturally-occurring truncated or secreted forms of the monomers forming the P2X₇ receptor (e.g., a form consisting of the extracellular domain sequence or truncated form of it), naturally-occurring variant forms (e.g., alternatively spliced forms) and naturally-occurring allelic variants. In certain embodiments of the invention, the native sequence P2X₇ monomeric polypeptides disclosed herein are mature or full-length native sequence polypeptides comprising the full-length amino acids sequence shown in SEQ ID NO: 1. In certain embodiments the P2X₇ receptor may have an amino acid sequence that is modified, for example various of the amino acids in the sequence shown in SEQ ID NO: 1 may be substituted, deleted, or a residue may be inserted.

“Functional P2X₇ receptor” generally refers to a form of the P2X₇ receptor having three intact binding sites or clefts for binding to ATP. When bound to ATP, the functional receptor forms a non-selective sodium/calcium channel that converts to a pore-like structure that enables the ingress of calcium ions and molecules of up to 900 Da into the cytosol, one consequence of which may be induction of programmed cell death. In normal homeostasis, expression of functional P2X₇ receptors is generally limited to cells that undergo programmed cell death such as thymocytes, dendritic cells, lymphocytes, macrophages and monocytes. There may also be some expression of functional P2X₇ receptors on erythrocytes and other cell types.

“Dysfunctional P2X₇ receptor” generally refers to a form of a P2X₇ receptor having a conformation, distinct from functional P2X₇, whereby the receptor is unable to form an apoptotic pore, but which is still able to operate as a non-selective channel through the maintenance of a single functional ATP binding site located between adjacent monomers. One example arises where one or more of the monomers has a cis isomerisation at Pro210 (according to SEQ ID NO: 1). The isomerisation may arise from any molecular event that leads to misfolding of the monomer, including for example, mutation of monomer primary sequence or abnormal post translational processing. One consequence of the isomerisation is that the receptor is unable to bind to ATP at one, or more particularly two, ATP binding sites on the trimer and as a consequence not be able to extend the opening of the channel. In the circumstances, the receptor cannot form a pore and this limits the extent to which calcium ions may enter the cytosol. Dysfunctional P2X₇ receptors are expressed on a wide range of epithelial and haematopoietic cancers. As used herein, the term “dysfunctional P2X₇ receptors” may be used interchangeably with the term “non-functional P2X₇ receptors” or “nfP2X₇ receptors”.

“Cancer associated-P2X₇ receptors” are generally P2X₇ receptors that are found on cancer cells (including, pre-neoplastic, neoplastic, malignant, benign or metastatic cells), but not on non-cancer or normal cells.

“E200 epitope” generally refers to an epitope having the sequence GHNYTTNILPGLNITC and variants thereof (e.g. SEQ ID NOs: 2-11, 15-30 and 168).

“E300 epitope” generally refers to an epitope having the sequence KYYKENNVEKRTLIK and variants thereof (SEQ ID NOs: 12 and 13).

A “composite epitope” generally refers to an epitope that is formed from the juxtaposition of the E200 and E300 epitopes or parts of these epitopes. An example of a composite epitope comprising E200 and E300 epitopes is GHNYTTRNILPGAGAKYYKENNVEK (SEQ ID NO: 14).

“Antibodies” or “immunoglobulins” or “Igs” are gamma globulin proteins that are found in blood, or other bodily fluids of vertebrates that function in the immune system to bind antigen, hence identifying and/or neutralising foreign objects.

Antibodies are generally a heterotetrameric glycoprotein composed of two identical light (L) chains and two identical heavy (H) chains. Each L chain is linked to a H chain by one covalent disulfide bond. The two H chains are linked to each other by one or more disulfide bonds depending on the H chain isotype. Each H and L chain also has regularly spaced intrachain disulfide bridges.

H and L chains define specific Ig domains. More particularly, each H chain has at the N-terminus, a variable domain (V_{H}) followed by three constant domains (CH) for each of the α and γ chains and four CH domains for p and E isotypes. Each L chain has at the N-terminus, a variable domain (V_{L}) followed by a constant domain (CL) at its other end. The V_{L} is aligned with the V_{H} and the C_{L} is aligned with the first constant domain of the heavy chain (CH1).

Antibodies can be assigned to different classes or isotypes. There are five classes of immunoglobulins: IgA, IgD, IgE, IgG, and IgM, having heavy chains designated α, δ, ε, γ, and μ, respectively. The γ and α classes are further divided into subclasses on the basis of relatively minor differences in ¾ sequence and function, e.g., humans express the following subclasses: IgG1, IgG2, IgG3, IgG4, IgA1, and IgA2. The L chain from any vertebrate species can be assigned to one of two clearly distinct types, called kappa and lambda, based on the amino acid sequences of their constant domains.

The constant domain includes the Fc portion that comprises the carboxy-terminal portions of both H chains held together by disulfides. The effector functions of antibodies such as ADCC are determined by sequences in the Fc region, which region is also the part recognised by Fc receptors (FcR) found on certain types of cells.

The pairing of a V_{H} and V_{L} together forms a “variable region” or “variable domain” including the amino-terminal domains of the heavy or light chain of the antibody. The variable domain of the heavy chain may be referred to as “V_{H}.” The variable domain of the light chain may be referred to as “V_{L}.” The V domain contains an “antigen binding site” that affects antigen binding and defines specificity of a particular antibody for its particular antigen. V regions span about 110 amino acid residues and consist of relatively invariant stretches called framework regions (FRs) (generally about 4) of 15-30 amino acids separated by shorter regions of extreme variability called “hypervariable regions” (generally about 3) that are each generally 9-12 amino acids long. The FRs largely adopt a β-sheet configuration and the hypervariable regions form loops connecting, and in some cases forming part of, the β-sheet structure.

“Hypervariable region” refers to the regions of an antibody variable domain that are hypervariable in sequence and/or form structurally defined loops. Generally, antibodies comprise six hypervariable regions; three in the V_{H} (H1, H2, H3), and three in the V_{L} (L1, L2, L3).

“Framework” or “FR” residues are those variable domain residues other than the hypervariable region residues herein defined.

An “antigen binding site” generally refers to a molecule that includes at least the hypervariable and framework regions that are required for imparting antigen binding function to a V domain. An antigen binding site may be in the form of an antibody or an antibody fragment, (such as a mAb, single domain (SD)-mAb, dAb, Fab, SD-Fab, Fd, SD-Fv, Fv, F(ab′)2 or scFv) in a method described herein.

An “intact” or “whole” antibody is one that comprises an antigen-binding site as well as a C_{L} and at least heavy chain constant domains, CH1, CH2 and CH3. The constant domains may be native sequence constant domains (e.g. human native sequence constant domains) or amino acid sequence variant thereof. [014]“Whole antibody fragments including a variable domain” include SD-mAb, Fab, Fab′, F(ab′)2, and Fv fragments; diabodies; linear antibodies, single-chain antibody molecules; and multi-specific antibodies formed from antibody fragments.

The “Fab fragment” consists of an entire L chain along with the variable region domain of the H chain (V_{H}), and the first constant domain of one heavy chain (CH1). Each Fab fragment is monovalent with respect to antigen binding, i.e., it has a single antigen-binding site.

A “Fab′ fragment” differs from Fab fragments by having additional few residues at the carboxy terminus of the CH1 domain including one or more cysteines from the antibody hinge region. Fab′-SH is the designation herein for Fab′ in which the cysteine residue(s) of the constant domains bear a free thiol group.

A “F(ab′)2 fragment” roughly corresponds to two disulphide linked Fab fragments having divalent antigen-binding activity and is still capable of cross-linking antigen.

An “Fv” is the minimum antibody fragment that contains a complete antigen-recognition and binding site. This fragment consists of a dimer of one heavy and one light chain variable region domain in tight, non-covalent association.

In a single-chain Fv (scFv) species, one heavy and one light chain variable domain can be covalently linked by a flexible peptide linker such that the light and heavy chains can associate in a “dimeric” structure analogous to that in a two-chain Fv species. From the folding of these two domains emanate six hypervariable loops (3 loops each from the H and L chain) that contribute the amino acid residues for antigen binding and confer antigen binding specificity to the antibody.

“Single-chain Fv” also abbreviated as “sFv” or “scFv” are antibody fragments that comprise the V_{H} and V_{L} antibody domains connected to form a single polypeptide chain. Preferably, the scFv polypeptide further comprises a polypeptide linker between the V_{H} and V_{L} domains that enables the scFv to form the desired structure for antigen binding.

A “single variable domain” is half of an Fv (comprising only three CDRs specific for an antigen) that has the ability to recognise and bind antigen, although generally at a lower affinity than the entire binding site.

“Diabodies” refers to antibody fragments with two antigen-binding sites, which fragments comprise a heavy-chain variable domain (V_{H}) connected to a light-chain variable domain (V_{L}) in the same polypeptide chain (V_{H}-V_{L}). The small antibody fragments are prepared by constructing sFv fragments (see preceding paragraph) with short linkers (about 5-10 residues) between the V_{H} and V_{L} domains such that interchain but not intra-chain pairing of the V domains is achieved, resulting in a bivalent fragment, i.e., a fragment having two antigen-binding sites.

Diabodies may be bivalent or bispecific. Bispecific diabodies are heterodimers of two “crossover” sFv fragments in which the V_{H} and V_{L} domains of the two antibodies are present on different polypeptide chains. Triabodies and tetrabodies are also generally known in the art.

An “isolated antibody” is one that has been identified and separated and/or recovered from a component of its pre-existing environment. Contaminant components are materials that would interfere with therapeutic uses for the antibody, and may include enzymes, hormones, and other proteinaceous or non-proteinaceous solutes.

A “human antibody” refers to an antibody that possesses an amino acid sequence that corresponds to that of an antibody produced by a human. Human antibodies can be produced using various techniques known in the art, including phage-display libraries. Human antibodies can be prepared by administering the antigen to a transgenic animal that has been modified to produce such antibodies in response to antigenic challenge, but whose endogenous loci have been disabled.

“Humanised” forms of non-human (e.g., rodent) antibodies are chimeric antibodies that contain minimal sequence derived from the non-human antibody. For the most part, humanised antibodies are human immunoglobulins (recipient antibody) in which residues from a hypervariable region of the recipient are replaced by residues from a hypervariable region of a non-human species (donor antibody) such as mouse, rat, rabbit or non-human primate having the desired antibody specificity, affinity, and capability. In some instances, framework region (FR) residues of the human immunoglobulin are replaced by corresponding non-human residues. Furthermore, humanised antibodies may comprise residues that are not found in the recipient antibody or in the donor antibody. These modifications are made to further refine antibody performance. In general, the humanised antibody will comprise substantially all of at least one, and typically two, variable domains, in which all or substantially all of the hypervariable loops correspond to those of a non-human immunoglobulin and all or substantially all of the FRs are those of a human immunoglobulin sequence. The humanised antibody optionally also will comprise at least a portion of an immunoglobulin constant region (Fc), typically that of a human immunoglobulin.

“Monoclonal antibody” refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site or determinant on the antigen. In addition to their specificity, the monoclonal antibodies are advantageous in that they may be synthesised uncontaminated by other antibodies. Monoclonal antibodies may be prepared by the hybridoma methodology. The “monoclonal antibodies” may also be isolated from phage antibody libraries using molecular engineering techniques.

The term “anti-P2X₇ receptor antibody” or “an antibody that binds to P2X₇ receptor” refers to an antibody that is capable of binding P2X₇ receptor with sufficient affinity such that the antibody is useful as a diagnostic and/or therapeutic agent in targeting P2X₇ receptor, typically non-functional P2X₇ receptor or a cancer associated P2X₇ receptor. Preferably, the extent of binding of a P2X₇ receptor antibody to an unrelated protein is less than about 10% of the binding of the antibody to P2X₇ receptor as measured, e.g., by a radioimmunoassay (RIA), Enzyme-Linked Immunosorbent Assay (ELISA), Biacore or Flow Cytometry. In certain embodiments, an antibody that binds to P2X₇ receptor has a dissociation constant (Kd) of <1 μM, <100 nM, <10 nM, <1 nM, or <0.1 nM. An anti nfP2X₇ receptor antibody is generally one having some or all of these serological characteristics and that binds to dysfunctional P2X₇ receptors but not to functional P2X₇ receptors.

An “affinity matured” antibody is one with one or more alterations in one or more hypervariable regions thereof that result in an improvement in the affinity of the antibody for the antigen, compared to a parent antibody that does not possess those alteration(s). Preferred affinity matured antibodies will have nanomolar or even picomolar affinities for the target antigen. Affinity matured antibodies are produced by procedures known in the art.

A “blocking” “antibody” or an “antagonist” antibody is one that inhibits or reduces biological activity of the antigen it binds. Preferred blocking antibodies or antagonist antibodies substantially or completely inhibit the biological activity of the antigen.

An “agonist antibody”, as used herein, is an antibody, which mimics at least one of the functional activities of a polypeptide of interest.

“Binding affinity” generally refers to the strength of the sum total of non-covalent interactions between a single binding site of a molecule (e.g., an antibody) and its binding partner (e.g., an antigen). Unless indicated otherwise, as used herein, “binding affinity” refers to intrinsic binding affinity, which reflects a 1:1 interaction between members of a binding pair (e.g., antibody and antigen). The affinity of a molecule X for its partner Y can generally be represented by the dissociation constant (Kd). Affinity can be measured by common methods known in the art, including those described herein. Low-affinity antibodies generally bind antigen slowly and tend to dissociate readily, whereas high-affinity antibodies generally bind antigen faster and tend to remain bound longer. A variety of methods of measuring binding affinity are known in the art, any of which can be used for purposes of the present invention.

As used herein, the term “antigen” is intended to include substances that bind to or evoke the production of one or more antibodies and may comprise, but is not limited to, proteins, peptides, polypeptides, oligopeptides, lipids, carbohydrates, and combinations thereof, for example a glycosylated protein or a glycolipid. The term “antigen” as used herein refers to a molecular entity that may be expressed on a target cell and that can be recognised by means of the adaptive immune system including but not restricted to antibodies or TCRs, or engineered molecules including but not restricted to transgenic TCRs, CARs, scFvs or multimers thereof, Fab-fragments or multimers thereof, antibodies or multimers thereof, single chain antibodies or multimers thereof, or any other molecule that can execute binding to a structure with high affinity.

“Epitope” generally refers to that part of an antigen that is bound by the antigen binding site of an antibody. An epitope may be “linear” in the sense that the hypervariable loops of the antibody CDRs that form the antigen binding site bind to a sequence of amino acids as in a primary protein structure. In certain embodiments, the epitope is a “conformational epitope” i.e. one in which the hypervariable loops of the CDRs bind to residues as they are presented in the tertiary or quaternary protein structure.

The term “target cell” as used herein refers to a cell that expresses a dysfunctional P2X₇ receptor (e.g. nfP2X₇ receptor) or a cell surface molecule to which the targeting moiety of the bridging molecule binds. The target cell may be a cancer cell or any other diseased cell.

The term “disorder” or “condition” means a functional abnormality or disturbance in a subject such as a cancer, an autoimmune disorder, or an infection by virus, bacteria, parasite, or others.

For example, a nucleic acid or a peptide naturally present in a living animal is not “isolated”, but the same nucleic acid or peptide partially or completely separated from the coexisting materials of its natural state is “isolated”. An isolated nucleic acid or protein can also exist in a non-native environment such as, for example, in a host cell.

As used herein, the term “subject” refers to a mammal such as mouse, rat, cow, pig, goat, chicken, dog, monkey or human. Preferentially, the subject is a human.

The subject may be a subject suffering from a disorder such as cancer (a patient), but the subject also may be a healthy subject.

The term “autologous” as used herein refers to any material derived from the same subject to whom it is later re-introduced.

The term “allogeneic” as used herein refers to any material derived from a different subject of the same species as the subject to whom the material is re-introduced.

The terms “therapeutically effective amount” or “therapeutically effective population” mean an amount of, for example, a cell population that provides a therapeutic benefit in a subject.

The terms “binds to”, “specifically binds to” or “specific for” with respect to a targeting moiety, as used e.g. in the bridging molecule as disclosed herein, or of a CAR referring to an antigen-binding domain that recognises and binds to a specific antigen, does not substantially recognise or bind to other molecules in a sample. An antigen-binding domain or targeting moiety that binds specifically to an antigen from one species also may bind to that antigen from another species. This cross-species reactivity is typical of many antibodies and therefore not contrary to the definition that the antigen-binding domain is specific. An antigen-binding domain that specifically binds to an antigen may bind also to different allelic forms of the antigen (allelic variants, splice variants, isoforms etc.) or homologous variants of this antigen from the same gene family. This cross reactivity is typical of many antibodies and therefore not contrary to the definition that the antigen-binding domain is specific.

The terms “engineered cell” and “genetically modified cell” as used herein can be used interchangeably. The terms mean containing and/or expressing a foreign gene or nucleic acid sequence that in turn modifies the genotype or phenotype of the cell or its progeny. Especially, the terms refer to the fact that cells, preferentially immune cells, can be manipulated by recombinant methods well known in the art to express stably or transiently peptides or proteins that are not expressed in these cells in the natural state. For example, immune cells are engineered to express an artificial construct such as a chimeric antigen receptor on their cell surface. For example, the CAR sequences may be delivered into cells using an adenoviral, adeno-associated viral (AAV)-based, retroviral or lentiviral vector or any other pseudotyped variations thereof or any other gene delivery mechanism such as electroporation or lipofection with CRISPR/Cas9, transposons (e.g. sleeping-beauty) or variations thereof. The gene delivery may be in the form of mRNA (transient) or DNA (transient or permanent).

The terms “immune cell” or “immune effector cell” refer to a cell that may be part of the immune system and executes a particular effector function such as alpha-beta T cells, NK cells, NKT cells, B cells, Breg cells, Treg cells, innate lymphoid cells (ILC), cytokine induced killer (CIK) cells, lymphokine activated killer (LAK) cells, gamma-delta T cells, mesenchymal stem cells or mesenchymal stromal cells (MSC), monocytes or macrophages or any hematopoietic progenitor cells such as pluripotent stem cells and early progenitor subsets that may mature or differentiate into somatic cells. The cells may be naturally occurring or generated by cytokine exposure, artificial/genetically modified cells (such as iPSCs and other artificial cell types). The immune cell may be an artificial cell subset including induced pluripotent stem cells and cells maturated therefrom. Preferred immune cells are cells with cytotoxic effector function such as alpha-beta T cells, NK cells, NKT cells, ILC, CIK cells, LAK cells or gamma-delta T cells. “Effector function” means a specialised function of a cell, e.g. in a T cell an effector function may be cytolytic activity or helper cell activity including the secretion of cytokines.

The term “treat” (treatment of) a disorder as used herein means to reduce the frequency or severity of at least one sign or symptom of a disease or disorder experienced by a subject.

The term “expression” as used herein is defined as the transcription and/or translation of a particular nucleotide sequence driven by its promoter in a cell.

### Chimeric Antigen Receptor (CAR)

The term cancer-specific CAR (T cell) targeting refers to the use of a CAR T cell for binding to a target antigen that is presented on the cell surface of tumour cells, but is not typically found on the surface of a healthy cell. In other words, normal cells under normal circumstances may be characterised by the absence of the target antigen on the extracellular membrane (and therefore the presence of the antigen on the cell surface cannot be detected). However, such cells may express mRNA encoding the antigen at an intracellular level. As CAR T cells only recognize surface-expressed antigens, the intracellular expression of the targeted proteins will not lead to CAR engagement.

The targeted epitopes E200 and E300 of the P2X₇ receptor are not exposed on the form of the receptor found in healthy tissue and thus these epitopes can be regarded as cancer specific. In other words, the E200 and E300 epitopes are only exposed, and available for binding when the P2X₇ receptor has an altered non-functional conformation, such as occurs in the context of cancer (in which case the receptor is referred to as nfP2X₇ receptor). Another example of a cancer-specific targeted epitope may be derived from the splice variant EGFRvIII. Still another example is the antigen CLDN6 which is mostly restricted to embryonic and foetal life and has very limited expression in healthy cells after the early phase in life and may be regarded as highly restricted and relatively overexpressed in cancer. The present invention contemplates the binding any such tumour-specific antigen, including nfP2X₇, EGFRvIII and CLDN6 for cancer-specific targeting and engaging CAR T cells via the bridging molecules described herein to cancer-associated antigens.

In general, a CAR may comprise an extracellular domain (extracellular part) comprising the antigen binding domain, a transmembrane domain and an intracellular signaling domain. The extracellular domain may be linked to the transmembrane domain by a linker. The extracellular domain may also comprise a signal peptide. The extracellular part of the CAR of the present invention comprises a tumour-specific antigen binding domain. For example, the tumour-specific antigen may be any one described herein, including nfP2X₇, EGFRvIII or CLDN6.

The tumour-specific antigen binding domain may be a nfP2X₇ binding domain that recognises the E200 (or E300 or E200-300 composite) epitope as disclosed herein.

Specifically, the CAR as disclosed herein has an extracellular nfP2X₇ E200 (or E300 or E200-300 composite) binding domain as an antigen binding domain. Alternatively, the tumour-specific antigen binding domain may be an EGFRvIII binding domain that recognises an epitope resulting out of the fusion of the amino acid sequence starting at position 25-29 LEEKK, followed by the insertion of G and the subsequent amino acid sequence 298-304 NYVVTDH, the total epitope is a 13-mer comprised of the sequence LEEKKGNYVVTDH (SEQ ID NO: 267). Alternatively, the tumour-specific antigen binding domain may be a CLDN6 binding domain that recognises an epitope in the second extracellular domain of CLDN6 [UniProtKB—P56747 (CLDN6_HUMAN)] via the amino acid sequence of SEQ ID NO: 273, 274 or 275.

Typically, the antigen-recognition domain includes a binding polypeptide that includes amino acid sequence homology to one or more complementarity determining regions (CDRs) of an antibody that binds to a tumour-specific antigen (such as a dysfunctional P2X₇ receptor, EGFRvIII or CLDN6). In any embodiment, the binding polypeptide includes amino acid sequence homology to the CDR1, 2 and 3 domains of the V_{H} and/or V_{L} chain of an antibody that binds to a tumour-specific antigen (such as a dysfunctional P2X₇ receptor, EGFRvIII or CLDN6).

In particularly preferred embodiments of the invention, the antigen-recognition domain of the CAR binds to an epitope of the tumour-specific antigen nfP2X₇.

In such embodiments, the binding polypeptide comprises the amino acid sequence of the CDRs of the V_{H} and/or V_{L} chain of an antibody described in any one of: PCT/AU2002/000061 or PCT/AU20021001204 (or in any one of the corresponding U.S. Pat. Nos. 7,326,415, 7,888,473, 7,531,171, 8,080,635, 8,399,617, 8,709,425, 9,663,584, or U.S. Pat. No. 10,450,380), PCT/AU2007/001540 (or in corresponding U.S. Pat. No. 8,067,550), PCT/AU2007/001541 (or in corresponding US publication US 2010-0036101), PCT/AU2008/001364 (or in any one of the corresponding U.S. Pat. Nos. 8,440,186, 9,181,320, 9,944,701 or U.S. Pat. No. 10,597,451), PCT/AU2008/001365 (or in any one of the corresponding U.S. Pat. No. 8,293,491 or U.S. Pat. No. 8,658,385), PCT/AU2009/000869 (or in any one of the corresponding U.S. Pat. No. 8,597,643, 9,328,155 or 10,238,716), PCT/AU2010/001070 (or in any one of the corresponding publications WO/2011/020155, U.S. Pat. Nos. 9,127,059, 9,688,771, or U.S. Pat. No. 10,053,508), and PCT/AU2010/001741 (or in any one of the corresponding publications WO 2011/075789 or U.S. Pat. No. 8,835,609) the entire contents of which are hereby incorporated by reference. Preferably the binding polypeptide comprises the amino acid sequence of the CDRs of the V_{H} and/or V_{L} chain of antibody 2-2-1 described in PCT/AU2010/001070 (or in any one of the corresponding U.S. Pat. Nos. 9,127,059, 9,688,771, or U.S. Pat. No. 10,053,508) or BPM09 described in PCT/AU2007/001541 (or in corresponding US publication US 2010-0036101) and produced by the hybridoma AB253 deposited with the European Collection of Cultures (ECACC) under Accession no. 06080101, WO2013185010A1 or WO2019056023. Alternatively, the binding polypeptide of the CAR may comprise the amino acid sequences of the CDRs of the antibody sdAbs 2-2-3, 2-472-2, or 2-2-12 described in WO 2017/041143 (also published as US 2019/0365805), and WO 2019/222796 (corresponding to U.S. application Ser. No. 17/057,060), incorporated herein by reference.

The binding polypeptide of the CAR may comprise the amino acid sequence of the V_{H} and/or V_{L} chains of an antibody described in any one of: PCT/AU2002/000061 or PCT/AU2002/001204 (or in any one of the corresponding U.S. Pat. Nos. 7,326,415, 7,888,473, 7,531,171, 8,080,635, 8,399,617, 8,709,425, 9,663,584, or U.S. Pat. No. 10,450,380), PCT/AU2007/001540 (or in corresponding U.S. Pat. No. 8,067,550), PCT/AU2007/001541 (or in corresponding US publication US 2010-0036101), PCT/AU2008/001364 (or in any one of the corresponding U.S. Pat. Nos. 8,440,186, 9,181,320, 9,944,701 or U.S. Pat. No. 10,597,451), PCT/AU2008/001365 (or in any one of the corresponding U.S. Pat. No. 8,293,491 or U.S. Pat. No. 8,658,385), PCT/AU2009/000869 (or in any one of the corresponding U.S. Pat. No. 8,597,643, 9,328,155 or 10,238,716), PCT/AU2010/001070 (or in any one of the corresponding publications WO/2011/020155, U.S. Pat. Nos. 9,127,059, 9,688,771, or U.S. Pat. No. 10,053,508), and PCT/AU2010/001741 (or in any one of the corresponding publications WO 2011/075789 or U.S. Pat. No. 8,835,609) the entire contents of which are hereby incorporated by reference. Preferably the binding polypeptide comprises the amino acid sequence of the V_{H} and/or V_{L} chains of the antibody 2-2-1 described in PCT/AU2010/001070 (or in any one of the corresponding U.S. Pat. Nos. 9,127,059, 9,688,771, or U.S. Pat. No. 10,053,508) or BPM09 described in PCT/AU2007/001541 (or in corresponding US publication US 2010-0036101) and produced by the hybridoma AB253 deposited with the European Collection of Cultures (ECACC) under Accession no. 06080101, WO2013185010A1 or WO2019056023. Alternatively, the binding polypeptide of the CAR may comprise the amino acid sequences of the V_{H} and/or V_{L} chains of the antibody sdAbs 2-2-3, 2-472-2, or 2-2-12 described in WO 2017/041143 (also published as US 2019/0365805), and WO 2019/222796 (corresponding to U.S. application Ser. No. 17/057,060), incorporated herein by reference.

The binding polypeptide of the CAR may comprise the amino acid sequence of an antibody or fragment thereof described in any one of: PCT/AU2002/000061 or PCT/AU2002/001204 (or in any one of the corresponding U.S. Pat. Nos. 7,326,415, 7,888,473, 7,531,171, 8,080,635, 8,399,617, 8,709,425, 9,663,584, or U.S. Pat. No. 10,450,380), PCT/AU2007/001540 (or in corresponding U.S. Pat. No. 8,067,550), PCT/AU2007/001541 (or in corresponding US publication US 2010-0036101), PCT/AU2008/001364 (or in any one of the corresponding U.S. Pat. Nos. 8,440,186, 9,181,320, 9,944,701 or U.S. Pat. No. 10,597,451), PCT/AU2008/001365 (or in any one of the corresponding U.S. Pat. No. 8,293,491 or U.S. Pat. No. 8,658,385), PCT/AU2009/000869 (or in any one of the corresponding U.S. Pat. No. 8,597,643, 9,328,155 or 10,238,716), PCT/AU2010/001070 (or in any one of the corresponding publications WO/2011/020155, U.S. Pat. Nos. 9,127,059, 9,688,771, or U.S. Pat. No. 10,053,508), and PCT/AU2010/001741 (or in any one of the corresponding publications WO 2011/075789 or U.S. Pat. No. 8,835,609) the entire contents of which are hereby incorporated by reference. Preferably the binding polypeptide comprises the amino acid sequence of sdAb 2-2-1 described in PCT/AU2010/001070 (or in any one of the corresponding U.S. Pat. Nos. 9,127,059, 9,688,771, or U.S. Pat. No. 10,053,508) or antibody BPM09 described in PCT/AU2007/001541 (or in corresponding US publication US 2010-0036101) and produced by the hybridoma AB253 deposited with the European Collection of Cultures (ECACC) under Accession no. 06080101, WO2013185010A1 or WO2019056023. Alternatively, the binding polypeptide may comprise the amino acid sequences of sdAbs 2-2-3, 2-472-2, or 2-2-12 described in WO 2017/041143 (also published as US 2019/0365805), and WO 2019/222796 (corresponding to U.S. application Ser. No. 17/057,060), incorporated herein by reference.

A “signal peptide” refers to a peptide sequence that directs the transport and localisation of the protein within a cell, e.g. to a certain cell organelle (such as the endoplasmic reticulum) and/or the cell surface.

Generally, an “antigen binding domain” refers to the region of the CAR that specifically binds to an antigen (and thereby is able to target a cell containing the antigen). The CARs of the invention may comprise one or more antigen binding domains. Generally, the targeting regions on the CAR are extracellular. The antigen binding domain may comprise an antibody or an antibody binding fragment thereof. The antigen binding domain may comprise, for example, full length heavy chain, Fab fragments, single chain Fv (scFv) fragments, divalent single chain antibodies or diabodies. Any molecule that binds specifically to a given antigen such as affibodies or ligand binding domains from naturally occurring receptors may be used as an antigen binding domain. Often the antigen binding domain is a scFv. Normally, in a scFv the variable regions of an immunoglobulin heavy chain and light chain are fused by a flexible linker to form a scFv. Such a linker may be for example the “(G₄/S₁)₃-linker” and variations thereof but the skilled person will appreciate that various linker sequences and formats may be used.

In some instances, it is beneficial for the antigen binding domain to be derived from the same species in which the CAR will be used in. For example, when it is planned to use it therapeutically in humans, it may be beneficial for the antigen binding domain of the CAR to comprise a human or humanised antibody or antigen binding fragment thereof. Human or humanised antibodies or antigen binding fragments thereof can be made by a variety of methods well known in the art. The CAR as disclosed herein has an extracellular linker/label epitope binding domain as an antigen binding domain allowing it to bind indirectly via a target cell binding molecule as disclosed herein to an antigen expressed on a target cell.

“Spacer” or “hinge” as used herein refers to the hydrophilic region that is between the antigen binding domain and the transmembrane domain. The CARs of the invention may comprise an extracellular spacer domain but it is also possible to leave out such a spacer. The spacer may include e.g. Fc fragments of antibodies or fragments thereof, hinge regions of antibodies or fragments thereof, CH2 or CH3 regions of antibodies, accessory proteins, artificial spacer sequences or combinations thereof. A prominent example of a spacer is the CD8alpha hinge.

The transmembrane domain of the CAR may be derived from any desired natural or synthetic source for such a domain. When the source is natural, the domain may be derived from any membrane-bound or transmembrane protein. The transmembrane domain may be derived for example from CD8alpha or CD28. When the key signalling and antigen recognition modules (domains) are on two (or even more) polypeptides, then the CAR may have two (or more) transmembrane domains. The splitting of key signalling and antigen recognition modules enables small molecule-dependent, titratable and reversible control over CAR cell expression (Wu et al, 2015, Science 350: 293-303) due to small molecule-dependent heterodimerising domains in each polypeptide of the CAR.

The cytoplasmic domain (or the intracellular signaling domain) of the CAR is responsible for activation of at least one of the normal effector functions of the immune cell in which the CAR is expressed. “Effector function” means a specialised function of a cell, e.g. in a T cell an effector function may be cytolytic activity or helper cell activity including the secretion of cytokines. The intracellular signalling domain refers to the part of a protein that transduces the effector function signal and directs the cell expressing the CAR to perform a specialised function. The intracellular signalling domain may include any complete, mutated or truncated part of the intracellular signalling domain of a given protein sufficient to transduce a signal that initiates or blocks immune cell effector functions.

The function of the intracellular domains may be pro- or anti-inflammatory and/or immunomodulatory, or a combination of such.

Prominent examples of intracellular signalling domains for use in the CARs include the cytoplasmic signaling sequences of the T cell receptor (TCR) and co-receptors that initiate signal transduction following antigen receptor engagement.

Generally, T cell activation can be mediated by two distinct classes of cytoplasmic signalling sequences, firstly those that initiate antigen-dependent primary activation through the TCR (primary cytoplasmic signalling sequences) and secondly those that act in an antigen-independent manner to provide a secondary or co-stimulatory signal (secondary cytoplasmic signalling sequences, co-stimulatory signalling domain). Therefore, an intracellular signalling domain of a CAR may comprise one or more primary cytoplasmic signalling domains and/or one or more secondary cytoplasmic signalling domains.

Primary cytoplasmic signalling sequences that act in a stimulatory manner may contain ITAMs (immunoreceptor tyrosine-based activation motifs) signalling motifs.

Examples of ITAM containing primary cytoplasmic signalling sequences often used in CARs are those derived from TCR zeta (CD3 zeta), FcR gamma, FcR beta, CD3 gamma, CD3 delta, CD3 epsilon, CD5, CD22, CD79a, CD79b and CD66d. Most prominent is the sequence derived from CD3 zeta.

The cytoplasmic domain of the CAR may be designed to comprise the CD3-zeta signaling domain by itself or combined with any other desired cytoplasmic domain(s). The cytoplasmic domain of the CAR can comprise a CD3 zeta chain portion and a co-stimulatory signalling region. The co-stimulatory signalling region refers to a part of the CAR comprising the intracellular domain of a co-stimulatory molecule. A co-stimulatory molecule is a cell surface molecule other than an antigen receptor or their ligands that is required for an efficient response of lymphocytes to an antigen. Examples for a co-stimulatory molecule are CD27, CD28, 4-1 BB (CD137), OX40, CD30, CD40, PD-1, ICOS, lymphocyte function-associated antigen-1 (LFA-1), CD2, CD7, LIGHT, NKG2C and B7-H3.

The cytoplasmic signalling sequences within the cytoplasmic signalling part of the CAR may be linked to each other with or without a linker in a random or specified order. A short oligo- or polypeptide linker, which is preferably between 2 and 10 amino acids in length, may form the linkage. A prominent linker is the glycine-serine doublet.

As an example, the cytoplasmic domain may comprise the signalling domain of CD3-zeta and the signalling domain of CD28. In another example the cytoplasmic domain may comprise the signalling domain of CD3-zeta and the signalling domain of CD27. In a further example, the cytoplasmic domain may comprise the signalling domain of CD3-zeta, the signalling domain of CD28, and the signalling domain of CD27.

As aforementioned, either the extracellular part or the transmembrane domain or the cytoplasmic domain of a CAR may also comprise a heterodimerising domain for the aim of splitting key signalling and antigen recognition modules of the CAR.

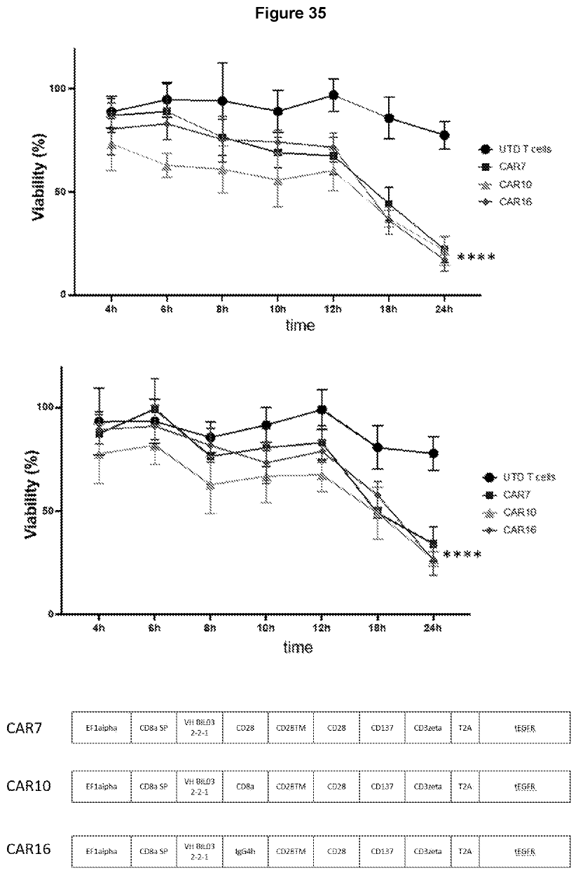

Non-limiting examples of CARs that may be used in accordance with the present invention are set forth in SEQ ID NOs: 165-167, 266 or 272. An example of the architecture of various CAR molecules is also provided herein in FIG. 35. Further examples of CARs that may be used in accordance with the invention, are provided in WO 2017/041143 (also published as US 2019/0365805), and WO 2019/222796 (corresponding to U.S. application Ser. No. 17/057,060), incorporated herein by reference.

A CAR for use in accordance with the present invention, i.e. a CAR comprising an nfP2X₇ E200 binding domain, may be designed to comprise any portion or part of the above-mentioned domains as described herein in any order and/or combination resulting in a functional CAR.

The CARs as disclosed herein, or polypeptide(s) derived therefrom, nucleic acid molecule(s) or recombinant expression vectors cells encoding said CARs, or populations of cells expressing said CARs, may be isolated and/or purified. The term “isolated” means altered or removed from the natural state. For example, an isolated population of cells means an enrichment of such cells and separation from other cells that are normally associated in their naturally occurring state with said isolated cells. An isolated population of cells means a population of substantially purified cells that are a more homogenous population of cells than found in nature. Preferably, the enriched cell population comprises at least about 90% of the selected cell type. In particular aspects, the cell population comprises at least about 90%, 91%, 92%, 93%, 94%, 95%, 96%, 97%, 98%, 99%, or even 100% of the selected cell type.

The affinity at which the dysfunctional P2X₇ receptor binding domain of the CAR binds to the nfP2X₇ recognition site E200 of the bridging molecule can vary, but generally the binding affinity may be in the range of 100 μM, 1 nM, 10 nM, or 100 nM, preferably at least about 1 pM or 10 pM, even more preferably at least about 100 pM.

CAR T cells targeted to EGFRvIII may be used to treat solid cancers. EGFRvIII is a frequent splice variant of EGFR skipping exons 2-7. EGFRvIII is tumour specific and does not occur in healthy cells as EGFR is tightly regulated in normal cells. EGFRvIII is commonly expressed in glioblastoma but also in breast cancer and head and neck cancer. The EGFRvIII-CAR T in this context may have the sequence (SEQ ID NO: 266) and is targeted to the epitope resulting out of the fusion of the amino acid sequence starting at position 25-29 LEEKK, followed by the insertion of G and the subsequent amino acid sequence 298-304 NYVVTDH, the total epitope comprises or consists of the sequence LEEKKGNYVVTDH (SEQ ID NO: 267). The complete EGFR sequence is found at UniProtKB—P00533 (EGFR_HUMAN) and the complete protein counts 1210 amino acids in isoform 1.

EGFRvIII targeted CAR T cells may be used to treat glioblastoma in a conventional way to target EGFRvIII on cancer cells, but may also be redirected to other cancer-associated target antigens via the bridging molecules described herein if the EGFRvIII epitope moiety is integrated into the sequence of bridging molecules. The EGFRvIII CAR T cells then can be used in the same manner as outlined for the nfP2X₇ CAR targeted approach described herein. The peptide tag may be the 13-mer peptide LEEKKGNYVVTDH or a shortened or extended natural or artificial variant thereof, of SEQ ID NO: 267.

The amino acid sequence of EGFRvIII CAR compatible bridging molecules targeted to CD33 and Her2 are described in Table 1 as SEQ ID NO: 268 and 269, and SEQ ID NO: 270 and 271, respectively.

CLDN6 targeted CAR T cells may be used to treat solid cancers e.g. ovarian cancer. The CLDN6-CAR T in this context may have the sequence (SEQ ID NO: 272) and is targeted to the second extracellular domain of CLDN6 [UniProtKB-P56747 (CLD6_HUMAN] cells directly via the amino acid sequence [ECD2, >sp|P56747|138-160 WTAHAIIRDFYNPLVAEAQKREL (SEQ ID NO: 273)] but may also be redirected to other cancer-associated target antigens, e.g. CD33 or Her2 via the bridging molecules described herein, if the CLDN6 epitope moiety is integrated into the sequence of bridging molecules. The CLDN6 CAR T cells then can be used in the same manner as outlined for the nfP2X₇ CAR targeted approach described herein. The peptide tag may be the 23-mer peptide WTAHAIIRDFYNPLVAEAQKREL or a shortened or extended natural or artificial variant thereof, such as SEQ ID NO: 274 or 275.

### Bridging Molecule

It will be appreciated that the bridging molecule may be in any form, provided that it comprises a) a targeting moiety for binding a target cell and b) a tumour-specific antigen epitope moiety. Preferably, the tumour-specific antigen epitope moiety is, or comprises, a dysfunctional P2X₇ receptor epitope moiety, a EGFRvIII epitope moiety or a CLDN6 epitope moiety, preferably such that the epitope moiety is recognised by the tumour-specific CAR which is being redirected, in accordance with the methods of the invention.

Typically, the targeting moiety is in the form of a fusion protein in which the targeting moiety is linked to the tumour-specific antigen epitope moiety, preferably dysfunctional P2X₇ receptor epitope moiety, directly or via a linker.

It will be appreciated that the bridging molecule may have any of a different number of architectures. For example, and as described further herein, the targeting moiety for binding a target cell may be in the form of any suitable binding domain, such as derived from an antibody, or fragments thereof. The targeting moiety may be linked to the tumour-specific antigen epitope moiety in any configuration. For example, the tumour-specific antigen epitope moiety may be linked to the N- or C-terminus of the targeting moiety. Examples of suitable architectures are provided in the Figures herein. In particularly preferred embodiments, the tumour-specific antigen epitope moiety is linked to the targeting moiety via its C terminal region, such that the N-terminal region of tumour-specific antigen epitope moiety is freely accessible for binding by the tumour-specific CAR.

Any suitable linker may be used for linking targeting moiety to the tumour-specific antigen epitope moiety. The linker may comprise a polypeptide, a peptide or a chemical group.

A linker may be a peptide having a length of up to 20 amino acids. The term “linked to” or “fused to” refers to a covalent bond, e.g., a peptide bond, formed between two moieties. Accordingly, in the context of the present invention the linker may have a length of 1, 2, 3, 4, 5, 6, 7, 8, 9, 10, 11, 12, 13, 14, 15, 16, 17, 18, 19, 20, 21 or 22 amino acids. For example, the herein provided bridging molecule may comprise a linker between the targeting moiety and tumour-specific antigen epitope moiety, preferably the dysfunctional P2X₇ receptor epitope moiety. Such linkers have the advantage that they can make it more likely that the different polypeptides of the fusion protein fold independently and behave as expected.

The skilled person will be familiar with the design and use of various peptide linkers comprised of various amino acids, and of various lengths, which would be suitable for use as linkers in accordance with the present invention. The linker may comprise various combinations of repeated amino acid sequences. The linker may be a flexible linker (such as those comprising repeats of glycine and serine residues), a rigid linker (such as those comprising glutamic acid and lysine residues, flanking alanine repeats) and/or a cleavable linker (such as sequences that are susceptible by protease cleavage).

The peptide linker may be any one or more repeats of Gly-Ser (GS), Gly-Gly-Ser (GGS), Gly-Gly-Gly-Ser (GGGS) or Gly-Gly-Gly-Gly-Ser (GGGGS) or variations thereof. In any embodiment, the linker may comprise or consist of the sequence GGGGSGGGGSGGGGS, i.e. (G4S)₃.

In any embodiment, the peptide linker can include the amino acid sequence GGGGGS (a linker of 6 amino acids in length) or even longer. The linker may be a series of repeating glycine and serine residues (GS) of different lengths, i.e., (GS)n where n is any number from 1 to 15 or more. For example, the linker may be (GS)₃ (i.e., GSGSGS) or longer (GS)₁₁ or longer. It will be appreciated that n can be any number including 1, 2, 3, 4, 5, 6, 7, 8, 9, 10, 11 or more.

In further embodiments, the linker may comprise inclusion of an amino acid that provides rigidity, such as lysine. For example, in certain embodiments, the linker region may also comprise the sequence GSGK.

The peptide linker may consist of a series of repeats of Thr-Pro (TP) comprising one or more additional amino acids N and C terminal to the repeat sequence. For example, the linker may comprise or consist of the sequence GTPTPTPTPTGEF (also known as the TP5 linker). In further aspects, the linker may be a short and/or alpha-helical rigid linker (e.g. A(EAAAK)3A, PAPAP or a dipeptide such as LE or CC).

In further embodiments, as an alternative or in addition to a glycine-serine-based linker region as described above, the targeting moiety may be in the form of a fusion protein in which the targeting moiety is linked to the tumour-specific antigen epitope moiety, preferably dysfunctional P2X₇ receptor epitope moiety, via a hinge region. The linking between the targeting moiety and tumour-specific antigen epitope moiety may comprise a combination of hinge region and linker regions.

Examples of suitable hinge regions include hinge regions derived from immunoglobulins. The hinge region may be derived from an IgG1, IgG2, IgG3 or IgG4, and may comprise one or more amino acid substitutions, (for example to prevent or reduce the likelihood of disulphide bridge formation). Alternative hinge sequences may be derived from alternative immunoglobulin domains, CD8A, CD8B, CD4 or CD28, TRAC, TRBC, TRGC, TRDC.

Table 2 below provides non-limiting examples of suitable hinge regions for use in joining the targeting moiety and tumour-specific antigen epitope moiety, in the bridging molecules of the invention.

It will be appreciated that the targeting moiety may be joined to the tumour-specific antigen epitope moiety by more than one linker and/or more than one hinge region. For example, the fusion protein may comprise (N to C terminus), the tumour-specific antigen epitope moiety, conjugated directly to the targeting moiety. Alternatively, the fusion protein may comprise the tumour-specific antigen epitope moiety, followed by a linker region, then the targeting moiety. Further still, the fusion protein may comprise the tumour-specific antigen epitope moiety, followed by a linker region, then a hinge region, and then the targeting moiety. In a further embodiment still, the fusion protein may comprise the tumour-specific antigen epitope moiety, followed by a linker region, then a hinge region, a further linker region, then the targeting moiety. Of course, the skilled person will appreciate that the alternative configuration is possible (ie wherein the tumour-specific antigen epitope moiety is joined to the C terminus of the targeting moiety, via one or more linker and/or hinge regions.

TABLE 2
exemplary hinge regions

| Amino acid sequence | SEQ ID NO: | Description |
|---|---|---|
| EPKSSDKTHTSPPSP | 310 | Sequences derived from IgG1 (ie |
| EPKSSDKTHTSPP | 311 | modified IgG1 hinge regions) |
| EPKSSDKTHTSPPP | 312 | |
| EPKSSDKTHT | 313 | |
| DKTHTSPPSP | 314 | |
| DKTHTSPP | 315 | |
| EPKSDKTHTPPP | 316 | |
| EPKSDKTHTSPPSP | 317 | |
| EPKSGDKTHTGPPGP | 318 | |
| EPKSGDKTHTGPP | 319 | |
| EPKSGDKTHTGPPP | 320 | |
| EPKSGDKTHT | 321 | |
| DKTHTGPPGP | 322 | |
| DKTHTGPP | 323 | |
| EPKSDKTHTGPPGP | 324 | |
| EPKSGDKTHTSPPSP | 325 | |
| EPKSGDKTHTGPPSP | 326 | |
| EPKSSDKTHTGPPGP | 327 | |
| EPKSSDKTHTGPPSP | 328 | |
| EPKSSDKTHTGPP | 329 | |
| EPKSGDKTHTSPP | 330 | |
| EPKSTDKTHTTPPIP | 331 | |
| EPKSTDKTHTTPP | 332 | |
| EPKSTDKTHTTPPP | 333 | |
| EPKSTDKTHT | 334 | |
| DKTHTTPPTP | 335 | |
| DKTHTTPP | 336 | |
| EPKSDKTHTTPPTP | 337 | |
| EPKSSDKTHTTPPTP | 338 | |
| EPKSSDKTHTSPPTP | 339 | |
| EPKSADKTHTLPPMP | 340 | |
| EPKSVDKTHTLPPTP | 341 | |
| EPKSLDKTHTAPPAP | 342 | |
| EPKSYDKTHTAPP | 343 | |
| EPKSIDKTHTLPP | 344 | |
| DKTHTAPPLP | 345 | |
| DKTHTVPPLP | 346 | |
| EPKSSDKTHTSP | 406 | |
| EPKSCDKTHTCPPQP | 347 | |
| ERKCCVECPPCP | 348 | |
| ERKXXVEXPPXP | 349 | |
| ERKXXVEXPP | 350 | Mutated IgG2 |
| VEXPPXP | 351 | Mutated IgG2 |
| ELKTPLGDTTHTCPRCP | 352 | Wild-type IgG3 |
| ELKTPLGQTTHTXPRXP | 353 | Mutated IgG3 |
| ELKTPLGDTTHTXPR | 354 | Mutated IgG3 |
| ELKTPLGDTTHT | 355 | Mutated IgG3 |
| EPKSCDTPPPCPRQP | 356 | Wild-type IgG3 |
| EPKSXDTPPPXPRXP | 357 | Mutated IgG3 |
| EPKSXDTPPPXPR | 358 | Mutated IgG3 |
| EPKSXDTPPP | 359 | Mutated IgG3 |
| DTPPPXPRXP | 360 | Mutated IgG3 |
| ESKYGPPCPSCP | 361 | Wild-type IgG4 |
| EXKYGPPCPXCP | 362 | Mutated IgG4 |
| EXKYGPPCP | 363 | Mutated IgG4 |
| KYGPPCPXCP | 364 | Mutated IgG4 |
| EPKSCDKTHTCP | 401 | Wild type IgG 1 (v2) |
| EPKSCDTPPPCP | 402 | Wild type IgG3 (v2) |
| ESKYGPPSPSSP | 403 | Mutated IgG4 |
| ERKSSVESPPSP | 404 | Mutated IgG2 |
| EPKSSDTPPPSP | 405 | Mutated IgG3 |

### Targeting Moiety of the Bridging Molecule

The targeting moiety of the bridging molecule may bind to a cell surface molecule on a target cell. The cell surface molecule may comprise an antigen. The cell surface molecule may be selected from a protein, a lipid moiety, a glycoprotein, a glycolipid, a carbohydrate, a polysaccharide, a nucleic acid, an MHC-bound peptide, or a combination thereof. The cell surface molecule may comprise parts (e.g., coats, capsules, cell walls, flagella, fimbriae, and toxins) of bacteria, viruses, and other microorganisms. The cell surface molecule may be expressed by the target cell. The cell surface molecule may not be expressed by the target cell. By way of non-limiting example, the cell surface molecule may be a ligand expressed by a cell that is not the target cell and that is bound to the target cell or a cell surface molecule of the target cell. Also, by non-limiting example, the cell surface molecule may be a toxin, exogenous molecule or viral protein that is bound to a cell surface or cell surface receptor of the target cell.

The bridging molecules may interact with a plurality of target cells. The target cell may be an infected cell. The target cell may be a pathogenically infected cell. The target cell may be a diseased cell. The target cell may be a genetically modified cell. The target cell may not be a host cell. The target cell may come from an invading organism (e.g. yeast, worm, bacteria, fungus). Further disclosed herein are bridging molecules that interact with a molecule on a non-cell target. The non-cell target may be a virus or a portion thereof. The non-cell target may be a fragment of a cell. The non-cell target may be an extracellular matrix component or protein.

The target cell may be derived from a tissue. The tissue may be selected from brain, oesophagus, breast, gut, intestine, colon, lung, glia, ovary, uterus, testes, prostate, gastrointestinal tract, bladder, liver, spleen, thymus, bone, fat and skin. The target cell may be derived from one or more endocrine glands. Alternatively, or additionally, the target cell may be derived from one or more endocrine glands. The endocrine gland may be a lymph gland, pituitary gland, thyroid gland, parathyroid gland, pancreas, gonad or pineal gland.

The target cell may be selected from a stem cell, a pluripotent cell, a hematopoietic stem cell or a progenitor cell. The target cell may be a circulating cell.

The target cell may be an immune cell.

The target cell may be a cancer stem cell. The target cell may be a cancer cell. The cancer cell may be derived from a tissue. The tissue may be selected from, by way of non-limiting example, a brain, an oesophagus, a breast, a colon, a lung, a glia, an ovary, a uterus, a testicle, a prostate, a gastrointestinal tract, a bladder, a liver, a thyroid and skin. The cancer cell may be derived from bone. The cancer cell may be derived from blood. The cancer cell may be derived from a B cell, a T cell, a monocyte, a thrombocyte, a leukocyte, a neutrophil, an eosinophil, a basophil, a lymphocyte, a hematopoietic stem cell or an endothelial cell progenitor. The cancer cell may be derived from a CD19-positive B lymphocyte. The cancer cell may be derived from a stem cell. The cancer cell may be derived from a pluripotent cell. The cancer cell may be derived from one or more endocrine glands. The endocrine gland may be a lymph gland, pituitary gland, thyroid gland, parathyroid gland, pancreas, gonad or pineal gland.

The cell surface molecule of the target cell may be a receptor. The receptor may be an extracellular receptor. The receptor may be a cell surface receptor. By way of non-limiting example, the receptor may bind a hormone, a neurotransmitter, a cytokine, a growth factor or a cell recognition molecule. The receptor may be a transmembrane receptor. The receptor may be an enzyme-linked receptor. The receptor may be a G-protein couple receptor (GPCR). The receptor may be a growth factor receptor. By way of non-limiting example, the growth factor receptor may be selected from an epidermal growth factor receptor, a fibroblast growth factor receptor, a platelet derived growth factor receptor, a nerve growth factor receptor, a transforming growth factor receptor, a bone morphogenic protein growth factor receptor, a hepatocyte growth factor receptor, a vascular endothelial growth factor receptor, a stem cell factor receptor, an insulin growth factor receptor, a somatomedin receptor, an erythropoietin receptor and homologs and fragments thereof. The receptor may be a hormone receptor. The receptor may be an insulin receptor. By way of non-limiting example, the receptor may be selected from an eicosanoid receptor, a prostaglandin receptor, an oestrogen receptor, a follicle stimulating hormone receptor, a progesterone receptor, a growth hormone receptor, a gonadotropin-releasing hormone receptor, homologs thereof and fragments thereof. The receptor may be an adrenergic receptor. The receptor may be an integrin. The receptor may be an Eph receptor. The receptor may be a luteinising hormone receptor. The cell surface molecule may be at least about 50% homologous to a luteinising hormone receptor. The receptor may be an immune receptor. By way of non-limiting example, the immune receptor may be selected from a pattern recognition receptor, a toll-like receptor, a NOD-like receptor, a killer-activated receptor, a killer inhibitor receptor, an Fc receptor, a B cell receptor, a complement receptor, a chemokine receptor and a cytokine receptor. By way of non-limiting example, the cytokine receptor may be selected from an interleukin receptor, an interferon receptor, a transforming growth factor receptor, a tumour necrosis factor receptor, a colony stimulating factor receptor, homologs thereof and fragments thereof. The receptor may be a receptor kinase. The receptor kinase may be a tyrosine kinase receptor. The receptor kinase may be a serine kinase receptor. The receptor kinase may be a threonine kinase receptor. By way of non-limiting example, the receptor kinase may activate a signalling protein selected from a Ras, a Raf, a PI3K, a protein kinase A, a protein kinase B, a protein kinase C, an AKT, an AMPK, a phospholipase, homo logs thereof and fragments thereof. The receptor kinase may activate a MAPK/ERK signalling pathway. The receptor kinase may activate Jak, Stat or Smad.

The cell surface molecule may be a non-receptor cell surface protein. The cell surface molecule may be a cluster of differentiation proteins. By way of non-limiting example, the cell surface molecule may be selected from CD3, CD4, CD8, CD11a, CD11b, CD13, CD14, CD15, CD16, CD22, CD24, CD25, CD30, CD31, CD33, CD34, CD38, CD45, CD56, CD61, CD91, CD114, CD117, CD182, CD200, fragments thereof, and homologs thereof.

The cell surface molecule of the target cell may be a molecule that does not comprise a peptide. The cell surface molecule may comprise a lipid. The cell surface molecule may comprise a lipid moiety or a lipid group. The lipid moiety may comprise a sterol. The lipid moiety may comprise a fatty acid. The antigen may comprise a glycolipid. The cell surface molecule may comprise a carbohydrate.

The cell surface molecule of the target cell may be an antigen. The antigen may be at least a portion of a surface antigen or a cell surface marker on a cell. The antigen may be a receptor or a co-receptor on a cell. The antigen may refer to a molecule or molecular fragment that may be bound by a major histocompatibility complex (MHC) and presented to a T-cell receptor. The term “antigen” may also refer to an immunogen. The immunogen may provoke an adaptive immune response if injected on its own into a subject. The immunogen may induce an immune response by itself. The antigen may be a superantigen, T-dependent antigen or a T-independent antigen. The antigen may be an exogenous antigen. Exogenous antigens are typically antigens that have entered the body from the outside, for example by inhalation, ingestion, or injection. Some antigens may start out as exogenous antigens, and later become endogenous (for example, intracellular viruses). The antigen may be an endogenous antigen. The endogenous antigen may be an antigen that has been generated within cells as a result of normal cell metabolism, or because of pathogenic infections (e.g., viral, bacterial, fungal, parasitic). The antigen may be an autoantigen. The autoantigen may be a normal protein or complex of proteins (and sometimes DNA or RNA) that is recognised by the immune system of patients suffering from a specific autoimmune disease. These antigens should, under normal conditions, not be the target of the immune system, but, due to genetic and/or environmental factors, the normal immunological tolerance for such an antigen is not present in these patients. The antigen may be present or over-expressed due to a condition or disease. The condition or disease may be a cancer or a leukaemia. The condition may be an inflammatory disease or condition. The condition or disease may be a metabolic disease. The condition may be a genetic disorder.

The present invention also may find application for the treatment of specific B- or T-lineage associated autoimmune diseases, for example using anti-idiotypic antibodies or fragments thereof or ligands thereof for targeting the B cell receptor and/or the T cell receptor. Such diseases include myasthenia gravis, systemic lupus erythematosus (SLE), rheumatoid arthritis (RA), multiple sclerosis (MS), solid organ transplant hyperacute, acute, chronic or mix-type rejection, bone marrow or stem cell transplant rejection, and graft versus host disease.

The cell surface molecule of the target cell may be an antigen that has been designated as a tumour antigen. Tumour antigens or neo-antigens may be antigens that are presented by MHC I or MHC II molecules on the surface of tumour cells. These antigens may sometimes be presented by tumour cells and never by the normal ones. In this case, they are called tumour-specific antigens (TSAs) and, in general, result from a tumour-specific mutation. More common are antigens that are presented by tumour cells and normal cells, and they are called tumour-associated antigens (TAAs). A TAA associated antigen is not unique to a tumour cell and instead is also expressed on a normal cell under conditions that fail to induce a state of immunologic tolerance to the antigen. The expression of the antigen on the tumour may occur under conditions that enable the immune system to respond to the antigen. TAAs may be antigens that are expressed on normal cells during foetal development when the immune system is immature and unable to respond or they may be antigens that are normally present at extremely low levels on normal cells but which are expressed at much higher levels on tumour cells. Cytotoxic T lymphocytes that recognise these antigens may be able to destroy the tumour cells before they proliferate or metastasise. Tumour antigens may also be on the surface of the tumour in the form of, for example, a mutated receptor, in which case they may be recognised by B cells.

Non-limiting examples of TSA or TAA antigens include the following: Differentiation antigens such as MART-1/MelanA (MART-1), gp 100 (Pmel 17), tyrosinase, TRP-1, TRP-2 and tumour-specific multilineage antigens such as MAGE-1, MAGE-3, BAGE, GAGE-1, GAGE-2, p15; overexpressed embryonic antigens such as CEA; overexpressed oncogenes and mutated tumour-suppressor genes such as p53, Ras, HER-2/neu; unique tumour antigens resulting from chromosomal translocations such as BCR-ABL, E2A-PRL, H4-RET, IGH-IGK, MYL-RAR; and viral antigens, such as the Epstein Barr virus antigens EBVA and the human papillomavirus (HPV) antigens E6 and E7. Other large, protein-based antigens include TSP-180, MAGE-4, MAGE-5, MAGE-6, RAGE, NY-ESO, p185erbB2, p180erbB-3, c-met, nm-23HI, PSA, TAG-72, CA 19-9, CA 72-4, CAM 17.1, NuMa, K-ras, beta-Catenin, CDK4, Mum-1, p 15, p 16, 43-9F, 5T4, 791Tgp72, alpha-fetoprotein, beta-HCG, BCA225, BTAA, CA 125, CA 15-3\CA 27.29\BCAA, CA 195, CA 242, CA-50, CAM43, 20 CD68\PI, CO-029, FGF-5, G250, Ga733\EpCAM, HTgp-175, M344, MA-50, MG7-Ag, MOV18, NB/70K, NY-CO-1, RCASI, SDCCAG16, TA-90Mac-2 binding protein\cyclophilin C-associated protein, TAAL6, TAG72, TLP, and TPS.

The cell surface molecule of the target cell may be an antigen selected from the group consisting of any surface expressed antigens. Exemplary target antigens may comprise but are not limited to: CD33 (Siglec-3), CD123 (IL3RA), CD135 (FLT-3), CD44 (HCAM), CD44V6, CD47, CD184 (CXCR4), CLEC12A (CLL1), LeY, FRβ, MICA/B, CD305 (LAIR-1), CD366 (TIM-3), CD96 (TACTILE), CD133, CD56, CD29 (ITGB1), CD44 (HCAM), CD47 (IAP), CD66 (CEA), CD112 (Nectin2), CD117 (c-Kit), CD133, CD146 (MCAM), CD155 (PVR), CD171 (L1CAM), CD200 (OX-2), CD221 (IGF1), CD227 (MUC1), CD243 (MRD1), CD246 (ALK), CD271 (LNGFR), CD19, CD20, GD2, and EGFR. Other target antigens include TCR, sugars, lipids, carbohydrates or any other molecule expressed on the surface of the target cell. The antigen may be any antigen referred to in Table 1 in the context of a bridging molecule or binding construct.

In preferred embodiments, the target cell is a cancer cell and the cell surface molecule of the cancer cell is an antigen that is associated with the cancer. The antigen may be a tumour-specific antigen or a tumour-associated antigen. The antigen may be one which is associated with a particular type of cancer. For example, overexpression of the antigen may be associated with a specific cancer or specific class of cancer. For example, where the cancer is breast cancer, the antigen may be associated with breast cancer but not with another form of cancer. Alternatively, the antigen may be associated with a class of cancers such as solid tumours, but not associated with haematological (ie “liquid”) tumours, or vice versa. The antigen may be associated with cancers of a particular lineage but not with others. For example the antigen may be associated with sarcomas but not lymphomas or carcinoma. As used herein the term “associated with” in relation to cancer, will be understood to mean that the antigen's expression (whether increased or decreased) is considered a marker of the cancer. It will be appreciated that there may be low levels of expression of an antigen but this does not equate to the antigen being “associated” with a given cancer.

Suitable cancer antigens which may be bound by the targeting moiety of the bridging molecule include, but are not limited to, mesothelin (MSLN), prostate specific membrane antigen (PSMA), prostate stem cell antigen (PCSA), carbonic anhydrase IX (CAIX), carcinoembryonic antigen (CEA), CD5, CD7, CD10, CD19, CD20, CD22, CD30, CD33, CD34, CD38, CD41, CD44, CD49f, CD56, CD74, CD123, CD133, CD138, epithelial glycoprotein (EGP 2), epithelial glycoprotein-40 (EGP-40), epithelial cell adhesion molecule (EpCAM), folate-binding protein (FBP), foetal acetylcholine receptor (AChR), folate receptor-α and β (FRα and β), Ganglioside G2 (GD2), Ganglioside G3 (GD3), human Epidermal Growth Factor Receptor 2 (HER-2/ERB2), HER3, Epidermal Growth Factor Receptor vIII (EGFRvIII), ERB3, ERB4, human telomerase reverse transcriptase (hTERT), Interleukin-13 receptor subunit alpha-2 (IL-13Rα2), κ-light chain, kinase insert domain receptor (KDR), Lewis A (CA19.9), Lewis Y (LeY), L1 cell adhesion molecule (L1CAM), melanoma-associated antigen 1 (melanoma antigen family A1, MAGE-A1), Mucin 16 (Muc-16), Mucin 1 (Muc-1), NKG2D ligands, cancer-testis antigen NY-ESO-1, oncofoetal antigen (h5T4), tumour-associated glycoprotein 72 (TAG-72), vascular endothelial growth factor R2 (VEGF-R2), Wilms' tumour protein (WT-1), type 1 tyrosine-protein kinase transmembrane receptor (ROR1), B7-H3 (CD276), B7-H6 (Nkp30), Chondroitin sulfate proteoglycan-4 (CSPG4), DNAX Accessory Molecule (DNAM-1), Ephrin type A Receptor 2 (EpHA2), Fibroblast Associated Protein (FAP), Gp100/HLA-A2, Glypican 3 (GPC3), HA-1H, HERK-V, IL-1 1Ra, Latent Membrane Protein 1 (LMP1), Neural cell-adhesion molecule (N-CAM/CD56), and Trail Receptor (TRAIL R). It is understood that these or other cancer antigens can be utilised for targeting by a bridging molecule in the present invention.

The targeting moiety of the bridging molecule may be any binding molecule, for example, a full-size antibody, or fragment thereof, or any antibody or fragment thereof described herein, an immunocytokine (antibody linked to a cytokine, or fragments thereof), a ligand (protein related, peptides, sugar molecules, processed molecules, lipids, cytokines, hormones), a soluble T cell receptor (TcR), a single chain (sc) TcR, single chain T cell receptor binding motifs and a T cell receptor like mAb, an aptamer (such as DNA or RNA), a peptide (e.g. aptamers or bicyclic peptides), a toxin, a lipid or a carbohydrate.

The targeting moiety of the bridging molecule may be a polypeptide and may be a targeting antibody or antibody fragment. The targeting antibody or antibody fragment may be an immunoglobulin (Ig). The immunoglobulin may be selected from an IgG, an IgA, an IgD, an IgE, an IgM, a fragment thereof or a modification thereof. The immunoglobulin may be IgG. The IgG may be IgG1. The IgG may be IgG2. The IgG may be IgG3. The IgG may be IgG4. The IgG may have one or more Fc mutations for modulating endogenous T cell FcR binding to the bridging molecule. The IgG may have one or more Fc mutations for removing the Fc binding capacity to the FcR of FcR-positive cells. The one or more Fc mutations may remove a glycosylation site. The one or more Fc mutations may be selected from E233P, L234V, L235A, delG236, A327G, A330S, P331S, N297Q and any combination thereof. The one or more Fc mutations may be in IgG1. The one or more Fc mutations in the IgG1 may be L234A, L235A, or both. Alternatively, or additionally, the one or more Fc mutations in the IgG1 may be L234A, L235E, or both. Alternatively, or additionally, the one or more Fc mutations in the IgG1 may be N297A. Alternatively, or additionally, the one or more mutations may be in IgG2. The one or more Fc mutations in the IgG2 may be V234A, V237A, or both.

The targeting antibody or antibody fragment may be an Fc null immunoglobulin or a fragment thereof.

As used herein, the term “antibody fragment” refers to any form of an antibody other than the full-length form. Antibody fragments herein include antibodies that are smaller components that exist within full-length antibodies, and antibodies that have been engineered. Antibody fragments include, but are not limited to, Fv, Fc, Fab, and (Fab′)2, single chain Fv (scFv), diabodies, triabodies, tetrabodies, bifunctional hybrid antibodies, CDR1, CDR2, CDR3, combinations of CDRs, variable regions, framework regions, constant regions, heavy chains, light chains, alternative scaffold non-antibody molecules, and bispecific antibodies. Unless specifically noted otherwise, statements and claims that use the term “antibody” or “antibodies” may specifically include “antibody fragment” and “antibody fragments.”

The targeting antibody fragment may be human, fully human, humanised, human engineered, non-human, and/or chimeric antibody. The non-human antibody may be humanised to reduce immunogenicity to humans, while retaining the specificity and affinity of the parental non-human antibody. Chimeric antibodies may refer to antibodies created through the joining of two or more antibody genes that originally encoded for separate antibodies. A chimeric antibody may comprise at least one amino acid from a first antibody and at least one amino acid from a second antibody, wherein the first and second antibodies are different. At least a portion of the antibody or antibody fragment may be from a bovine species, a human species, or a murine species. At least a portion of the antibody or antibody fragment may be from a rat, a goat, a guinea pig or a rabbit. At least a portion of the antibody or antibody fragment may be from a human. At least a portion of the antibody or antibody fragment antibody may be from cynomolgus monkey.

The targeting antibody or antibody fragment may be based on or derived from an antibody or antibody fragment from a mammal, bird, fish, amphibian or reptile.

Mammals include, but are not limited to, carnivores, rodents, elephants, marsupials, rabbits, bats, primates, seals, anteaters, cetaceans, odd-toed ungulates and even-toed ungulates. The mammal may be a human, non-human primate, mouse, sheep, cat, dog, cow, horse, goat, or pig.

The targeting antibody or an antibody fragment may recognise or bind an antigen selected from, by non-limiting example, CD19, Her2, CLL-1, CD33, EGFRvIII, CD20, CD22, BCMA or a fragment thereof. The antigen may comprise a wild-type antigen. The antigen may comprise one or more mutations.

The targeting antibody or antibody fragment may be an anti-CD19 antibody or a fragment thereof. The targeting polypeptide may be an anti-CD22 antibody. The targeting polypeptide may be an anti-BCMA antibody or a fragment thereof. The targeting polypeptide may be an anti-EGFRvIII antibody or a fragment thereof. The targeting polypeptide may be an anti-Her2 antibody or a fragment thereof. The targeting polypeptide may comprise an anti-CD20 antibody or antibody fragment. The targeting polypeptide may comprise rituximab. The targeting polypeptide may comprise an anti-EGFR antibody or antibody fragment. The targeting polypeptide may comprise an anti-CEA antibody or antibody fragment. The targeting polypeptide may comprise an anti-CLL-1 antibody or antibody fragment. The targeting polypeptide may comprise an anti-CD33 antibody or antibody fragment. The targeting polypeptide may comprise an anti-EpCAM antibody or fragment thereof. The targeting polypeptide may comprise an anti-CD30 antibody or fragment thereof. The targeting polypeptide may comprise an anti-CD79B antibody or fragment thereof. The targeting polypeptide may comprise an anti-CD37 antibody or fragment thereof. The targeting polypeptide may comprise an anti-CD38 antibody or fragment thereof. The targeting polypeptide may comprise an anti-CD70 antibody or fragment thereof. The targeting polypeptide may comprise an anti-CD276 antibody or fragment thereof. The targeting polypeptide may comprise an anti-GD2 antibody or fragment thereof. The targeting polypeptide may comprise an anti-CD371 antibody or fragment thereof. The targeting polypeptide may comprise an anti-CD135 antibody or fragment thereof. The targeting polypeptide may comprise an anti-CD105 antibody or fragment thereof. The targeting polypeptide may comprise an anti-CD123 antibody or fragment thereof. The targeting polypeptide may comprise an anti-ROR-1 antibody or fragment thereof. The targeting polypeptide may comprise an anti-PD-L1 antibody or fragment thereof. The targeting polypeptide may comprise an anti-MET-R antibody or fragment thereof. The targeting polypeptide may comprise an anti-PDGFRalpha antibody or fragment thereof. The targeting polypeptide may comprise an anti-Her3 antibody or fragment thereof. The targeting polypeptide may comprise an anti-FRalpha antibody or fragment thereof. The targeting polypeptide may comprise an anti-GPC3 antibody or fragment thereof. The targeting polypeptide may comprise an anti-SLAMf7 antibody or fragment thereof. The targeting polypeptide may comprise an anti-TNFRSF10B antibody or fragment thereof. The targeting polypeptide may comprise an anti-GPNMB antibody or fragment thereof. The targeting polypeptide may comprise an anti-VEGFR2 antibody or fragment thereof. The targeting polypeptide may comprise an anti-alpha4beta7/alphaEbeta7 antibody or fragment thereof. The targeting polypeptide may comprise an anti-CSPG4 antibody or fragment thereof. The targeting polypeptide may comprise an anti-CD80 antibody or fragment thereof. The targeting polypeptide may comprise an anti-CCR4 antibody or fragment thereof. The targeting polypeptide may comprise an anti-CD115 antibody or fragment thereof. The targeting polypeptide may comprise an anti-ENOX-2 antibody or fragment thereof. The targeting polypeptide may comprise an anti-CD56 antibody or fragment thereof. The targeting polypeptide may comprise an anti-huVH1-69 antibody or fragment thereof. The targeting polypeptide may comprise an anti-CD117 antibody or fragment thereof. The targeting polypeptide may comprise an anti-CD133 antibody or fragment thereof. The targeting polypeptide may comprise an anti-MUC1 antibody or fragment thereof. The targeting polypeptide may comprise an anti-MSLN antibody or fragment thereof. The targeting polypeptide may comprise an anti-ROR-2 antibody or fragment thereof. The targeting polypeptide may comprise an anti-IL13Ra2 antibody or fragment thereof. The targeting polypeptide may comprise an anti-EPHA2 antibody or fragment thereof. The targeting polypeptide may comprise an anti-EGFRvIII antibody or fragment thereof. The targeting polypeptide may comprise an anti-PSMA antibody or fragment thereof. The targeting polypeptide may comprise an anti-CEA antibody or fragment thereof. The targeting polypeptide may comprise an anti-Lewis Y antibody or fragment thereof. The targeting polypeptide may comprise an anti-PSCA antibody or fragment thereof. The targeting polypeptide may comprise an anti-MUC1 antibody or fragment thereof. The targeting polypeptide may comprise an anti-CD181L1CAM antibody or fragment thereof. The targeting polypeptide may comprise an anti-EpCAM antibody or fragment thereof. The targeting polypeptide may comprise an anti-ALK antibody or fragment thereof. The targeting polypeptide may comprise an anti-IGF-1R CD221 antibody or fragment thereof. The targeting polypeptide may comprise an anti-Nectin 4 antibody or fragment thereof. The targeting polypeptide may comprise an anti-FAP antibody or fragment thereof. The targeting polypeptide may comprise an anti-AXL antibody or fragment thereof. The targeting polypeptide may comprise an anti-CD138 antibody or fragment thereof. The targeting polypeptide may comprise an anti-CLDN6 antibody or fragment thereof. The targeting polypeptide may comprise an anti-Her4 antibody or fragment thereof. The targeting polypeptide may comprise an anti-Claudin 18.2 antibody or fragment thereof. The targeting polypeptide may comprise an anti-O-acetylated GD2 antibody or fragment thereof. The targeting polypeptide may comprise an anti-GD3 antibody or fragment thereof. The targeting polypeptide may comprise an anti-GM2 antibody or fragment thereof. The targeting polypeptide may comprise an anti-TM4SF1 antibody or fragment thereof. The targeting polypeptide may comprise an anti-CD147 antibody or fragment thereof. The targeting polypeptide may comprise an anti-CEACAM5 antibody or fragment thereof. The targeting polypeptide may comprise an anti-VEGFR-1 antibody or fragment thereof. The targeting polypeptide may comprise an anti-PDPN antibody or fragment thereof. The targeting polypeptide may comprise an anti-WT1 antibody or fragment thereof. The targeting polypeptide may comprise an anti-GPC2 antibody or fragment thereof. The targeting polypeptide may comprise an anti-FGFR4 antibody or fragment thereof. The targeting polypeptide may comprise an anti-EphB4 antibody or fragment thereof. The targeting polypeptide may comprise an anti-STEAP-1 antibody or fragment thereof. The targeting polypeptide may comprise an anti-STEAP-2 antibody or fragment thereof. The targeting polypeptide may comprise an anti-MUC1 antibody or fragment thereof. The targeting polypeptide may comprise an anti-chlorotoxin antibody or fragment thereof. The targeting polypeptide may comprise an anti-206 antibody or fragment thereof. The targeting polypeptide may comprise an anti-ILRAP1 antibody or fragment thereof. The targeting polypeptide may comprise an anti-MICA antibody or fragment thereof. The targeting polypeptide may comprise an anti-MAGE-A1 scTcR antibody or fragment thereof. The targeting polypeptide may comprise an anti-MAGE-A1 sTCR antibody or fragment thereof. The targeting polypeptide. The targeting polypeptide may comprise an anti-MICA antibody or fragment thereof. The targeting polypeptide may comprise an anti-TRBC1/2 antibody or fragment thereof. The targeting polypeptide may comprise an anti-B7-H7 antibody or fragment thereof. The targeting polypeptide may comprise an anti-CD34 antibody or fragment thereof. The targeting polypeptide may comprise an anti-CD7 antibody or fragment thereof. The targeting polypeptide may comprise an anti-TIM3 antibody or fragment thereof. The targeting polypeptide may comprise an anti-CD191 antibody or fragment thereof. The targeting polypeptide may comprise an anti-CD66b antibody or fragment thereof. The targeting polypeptide may comprise an anti-CD11b antibody or fragment thereof. The targeting polypeptide may comprise an anti-EMR2 antibody or fragment thereof. The targeting polypeptide may comprise an anti-MUC16 antibody or fragment thereof. The targeting polypeptide may comprise an anti-NYESO-1 HLA-A2 antibody or fragment thereof or a soluble TcR or variant thereof. The targeting polypeptide may comprise an anti-SURVIVIN HLA-A2 antibody or fragment thereof or a soluble TcR or variant thereof. The targeting polypeptide may comprise an anti-CD200 antibody or fragment thereof.

The targeting antibody or antibody fragment may be selected from any commercially available antibody. The targeting antibody or antibody fragment may be selected from trastuzumab (for binding to Her2), alemtuzumab (for binding CD52), bevacizumab (for binding VEGF-A), brentuximab (for binding CD30, gemtuzumab (for binding CD33), ipilimumab (for binding VTLA-4), ibritumomab (for binding CD20), panitumumab (for binding EGFR), cetuximab (for binding EGFR), rituximab (for binding CD20), and fragments thereof.

The targeting antibody or antibody fragment may be any referred to in Table 1, or an antigen binding fragment at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical thereto.

The targeting moiety of the bridging molecule may target peptide MHC complexes and in such embodiments, the target moiety may be a soluble TcR molecule or single chain TcR molecule.

Non-limiting examples of the sequences of various targeting antibodies, or antigen binding fragments thereof, are provided herein in Table 1.

It will be well within the purview of the skilled person to be able to determine the appropriate design of a bridging molecule as described herein, including selection of the targeting moiety of the bridging molecule.

More specifically, the skilled person, having knowledge of the CAR requiring “redirection” as described herein would be able to select an alternative targeting moiety to which the CAR should be directed, to facilitate treatment of the cancer or disease requiring treatment.

As described herein, the targeting moiety may be for binding to any cell surface molecule on a target cell (such as a tumour-associated antigen expressed on a cancer cell). The skilled person can select an appropriate cell surface molecule depending on the disease (eg cancer) requiring treatment. For example, if the disease is a Her2+ cancer, then the skilled person may select a targeting moiety that is capable of specifically binding to Her2 on the cancer cell. Similarly, if the disease is a CD19+ cancer cell, then the skilled person may select a targeting moiety that is capable of specifically binding to CD19 on the cancer. The skilled person will be familiar with methods for determining the antigen expression profile of the cancer or condition to be treated in order to identify suitable surface molecules expressed on the target cell.

Having identified a suitable cell surface molecule (target antigen) on the target cell (eg: CD19, Her2, CLL-1, CD33, EGFRvIII, CD20, CD22, BCMA or any other tumour-associated antigen described herein or that may be selected for targeting), the skilled person can readily determine the sequence of a binding domain that can bind to said target antigen, including by reference to commercially available antibodies or to published literature describing antigen binding domains of known antibodies that bind to the antigen. The skilled person can then formulate the structure of a bridging molecule (fusion protein) as described herein, comprising a targeting moiety that binds to the cell surface molecule on a target cell. The composition of the remaining components of the bridging molecule (eg: the tumour-specific antigen epitope moiety) are described further herein.

Finally, the skilled person, having identified a suitable cell surface molecule (target antigen) on the target cell, and tumour-specific antigen epitope moiety in order to arrive at a bridging molecule of the invention, can readily determine, using routine techniques, whether the bridging molecule: a) binds to the target cell, b) binds to the CAR T cell and c) can successfully redirect the CAR T cell to the target cells to induce cell killing. Methods of determining binding to target antigens, and cytotoxicity are well known in the art. Non-limiting methods and various experimental protocols for determining binding to target cell, CAR T cell and induction of cell killing are described in detail herein in the Examples.

### Dysfunctional P2X₇ Receptor Epitope Moiety

The present invention contemplates the use of a tumour-specific antigen epitope moiety in the form of an epitope from dysfunctional P2X₇ receptor (ie wherein the tumour-specific antigen is dysfunctional P2X₇ receptor).

A dysfunctional P2X₇ receptor epitope moiety may be provided in the form of a dysfunctional P2X₇ receptor, or a fragment of a dysfunctional P2X₇ receptor, that has at least one of the three ATP binding sites that are formed at the interface between adjacent correctly packed monomers that are unable to bind ATP. Such receptors are unable to extend the opening of the non-selective calcium channels to apoptotic pores.

A range of peptide fragments of a dysfunctional P2X₇ receptor are known and discussed in PCT/AU2002/000061 (and in corresponding publications WO 2002/057306 and U.S. Pat. Nos. 7,326,415, 7,888,473, 7,531,171, 8,080,635, 8,399,617, 8,709,425, 9,663,584, or U.S. Pat. No. 10,450,380), PCT/AU2008/001364 (and in corresponding publications WO 2009/033233 and U.S. Pat. No. 8,440,186, 9,181,320, 9,944,701 or 10,597,45) and PCT/AU2009/000869 (and in corresponding publications WO 2010/000041 and U.S. Pat. No. 8,597,643, 9,328,155 or 10,238,716) the contents of all of which are incorporated in entirety. Exemplary peptides within these specifications that include epitopes contemplated for use in this invention are described below.

| PCT publication | Peptide sequence |
|---|---|
| WO 2002/057306 | GHNYTTRNILPGLNITC |
| | (SEQ ID NO: 2) (also referred to |
| | herein as the “E200” epitope) |
| WO 2009/033233 | KYYKENNVEKRTLIKVF |
| | (SEQ ID NO: 12) (also referred |
| | toherein as the “E300” epitope) |
| WO 2010/000041 | GHNYTTRNILPGAGAKYYKENNVEK |
| | (SEQ ID NO: 14) (also referred |
| | to herein as the “E200/E300” |
| | or “composite” epitope) |

In any embodiment, the amino acid sequence of the dysfunctional P2X₇ receptor epitope moiety of any bridging molecule described herein, comprises or consists of a sequence as set forth in any of SEQ ID Nos: 2 to 30, 168, and SEQ ID NOs: 365-400, or sequences at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto. Preferably, the dysfunctional P2X₇ receptor epitope moiety comprises at least the sequence of SEQ ID NO: 11.

The dysfunctional P2X₇ receptor epitope moiety may have any functional chemical group such as a carboxyl group, an active ester, an acetamide or maleimide capable of coupling to a targeting moiety as disclosed herein, for example an antibody or fragment thereof using NH₂ or SH groups for coupling thereto.

Various examples of dysfunctional P2X₇ receptor epitope moieties are described herein, particularly in Table 1. As is clear from Table 1, the minimum E200 sequence comprises at least the sequence of SEQ ID NO: 11. In preferred embodiments, the minimum E200 sequence comprises at least the sequence of SEQ ID NO: 2, or a variant thereof, such as the sequence of SEQ ID NO: 4.

In certain examples, the E200 epitope for inclusion in the bridging molecules of the invention, may comprise extensions in the N-terminal and/or C-terminal region. The extensions may be derived from the naturally occurring nfP2X7 receptor sequence, and may comprise extensions of at least 1 amino acid residue, at least 2 amino acid residues, at least 3, at least 4, at least 5 or more amino acid residues. For example, the E200 epitope defined in SEQ ID NO: 4 may comprise an N-terminal extension of the sequence DFP, which corresponds to the three amino acid residues immediately N terminal to the E200 sequence in SEQ ID NO: 1. Further, the E200 epitope defined in SEQ ID NO: 4 may comprise a C terminal extension, as depicted, for example in SEQ ID NO: 6 and 7. It will be appreciated that such N and C terminal extensions may serve to improve the efficacy of binding of the CAR-T cell to the epitope on the bridging molecule.

In any embodiment, the dysfunctional P2X₇ receptor epitope moiety is conjugated to the targeting moiety so as to enable sufficient accessibility for binding by an nfP2X₇ receptor CAR. Thus, while the minimum dysfunctional P2X₇ receptor epitope moiety sequence comprises the minimum E200 sequence at least the sequence of SEQ ID NO: 11, in preferred embodiments, the minimum E200 sequence comprises at least the sequence of SEQ ID NO: 2, or a variant thereof, such as the sequence of SEQ ID NO: 4, in addition to one or more linker or hinge regions for enabling binding by an nfP2X₇ receptor CAR. In certain embodiments, the total length of the dysfunctional P2X₇ receptor epitope moiety (including linker and hinge region) is at least 17 amino acids in length, or at least, 18, 19, 20, 21, 22, 23, 24, 25, 26, 27, 28, 29, 30, 31, 32, 33,34, 35, 36, 37, 38, 39, 40, 41, 42, 43, 44, 45, 45, 47, 48, 49, or 50 amino acids in length. Preferably the dysfunctional P2X₇ receptor epitope moiety is no more than about 100 amino acids in length, more preferably, no more than about 99, 98, 97, 96, 95, 94, 93, 92, 91, 90, 89, 88, 87, 86, 85, 84, 83, 82, 81, 80, 79, 78, 77, 76, 75, 74, 73, 72, 71, 70, 69, 68, 67, 66, 65, 64, 63, 62, 61, 60, 59, 58, 57, 56, 55, 54, 53, 52, 51 or 50 amino acids in length.

In the context of a bridging molecule for targeting CD19, preferably the dysfunctional P2X₇ receptor epitope moiety is no more than about 35 amino acids in length and at least 20 amino acids in length, preferably at least 21, 22, 23, 24, 25, or 26 amino acids in length.

In the context of a bridging molecule for targeting CD33, preferably the dysfunctional P2X₇ receptor epitope moiety is no more than about 45 amino acids in length and at least 20 amino acids in length, preferably at least 21, 22, 23, 24, 25, or 26 amino acids in length.

### EGFRvIII Epitope Moiety

In any embodiment, the amino acid sequence of the EGFRvIII epitope moiety of any bridging molecule described herein, comprises or consists of a sequence as set forth in any of SEQ ID Nos: 267, or sequences at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto. Preferably, the EGFRvIII epitope moiety comprises at least the sequence of SEQ ID NO: 267.

### CLDN6 Epitope Moiety

In any embodiment, the amino acid sequence of the CLDN6 epitope moiety of any bridging molecule described herein, comprises or consists of a sequence as set forth in any of SEQ ID Nos: 273, 274 or 275, or sequences at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto. Preferably, the CLDN6 epitope moiety comprises at least the sequence of SEQ ID NO: 273, 274 or 275.

### Exemplary Bridging Molecules

The present specification provides various non-limiting examples of tumour-specific antigen epitope moiety (e.g. dysfunctional P2X₇ receptor epitope moiety)/targeting moiety pairs.

Exemplary bridging molecules of the invention are described in Table 1. For those bridging molecules that are described in Table 1 that include a nfP2X₇ epitope moiety, the specification includes those bridges but with the nfP2X₇ epitope moiety substituted for a EGFRvIII or CLDN6 epitope moiety.

In examples where the bridging molecules comprise a targeting moiety for binding to CD19, the targeting moiety may comprise or consist of a heavy and paired light variable chain combination as set forth in SEQ ID NOs: 31 and 32; or 143 and 144 (heavy and light chain, respectively; or sequences at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In the above examples, the bridging molecules may comprise the tumour-specific antigen epitope moiety (e.g. dysfunctional P2X₇ receptor epitope moiety) conjugated to the heavy chain, or the tumour-specific antigen epitope moiety (e.g. dysfunctional P2X₇ receptor epitope moiety) conjugated to the light chain. Preferably, the tumour-specific antigen epitope moiety (e.g. dysfunctional P2X₇ receptor epitope moiety) is conjugated to the light chain of the target binding moiety.

In any embodiment wherein the bridging molecule comprises CD19-binding heavy/light chain pairs where the heavy chain comprises or consists of the dysfunctional P2X₇ receptor epitope moiety, the sequences of the variable sequences of the heavy and light chain pairs are preferably selected from: SEQ ID NOs: 33 and 32; 34 and 32, 37 and 32; 37 and 38; (heavy and light chain sequences recited, respectively) or sequences at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment wherein the bridging molecule comprises CD19-binding heavy/light chain pairs where the light chain comprises or consists of the dysfunctional P2X₇ receptor epitope moiety, the sequences of the variable sequences of the heavy and light chain pairs are preferably selected from: SEQ ID NOs: 31 and 35; 31 and 36; 39 and 31; 52 and 51; 143 and 145; 143 and 146; 143 and 147; 143 and 148; 143 and 149; 143 and 150; 143 and 151; 143 and 152; 143 and 153; 143 and 154; 143 and 155; 143 and 156; 143 and 157; 143 and 158; 143 and 159; 143 and 160; 143 and 161; 143 and 162; 143 and 163; or 143 and 164 (heavy and light chain sequences recited, respectively) or sequences at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto. In another embodiment wherein the bridging molecule comprises CD19-binding heavy/light chain pairs where the light chain is any one of the light chains above and a heavy chain selected from SEQ ID NO: 141 or 142.

The targeting moiety may be in the form of an scFv comprising a heavy and a light chain.

In any embodiment, a CD19-binding scFv for use in the bridging molecules of the invention may be one having a sequence as set forth in SEQ ID NOs: 40 or 41 or sequences at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto. It will be appreciated that in the context of an scFv, the dysfunctional P2X₇ receptor epitope moiety may be conjugated to the light chain of the scFv, such as in any of SEQ ID NOs: 42, 43, 46, 48, or to the heavy chain of the scFv, such as in any of SEQ ID NOs: 44, 45, 47, 49, 50 or sequences at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In preferred embodiments, the bridging molecule for binding to CD19 comprises a targeting moiety comprising an antigen binding domain for specifically binding to CD19, preferably as described herein (more preferably, comprising the amino acid sequence as set forth in SEQ ID NOs: 52/143 or 143/144 (heavy and light chains respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto) and comprises a tumour-specific antigen epitope moiety comprising or consisting of the amino acid sequence set forth in any of SEQ ID NOs: 365, 371, 377, 383, 389, and 395.

In any embodiment, a bridging molecule for binding to CD20 may comprise or consist of the sequences set forth in SEQ ID NOs: 53 and 54, or in 55 and 56 (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to CD22 may comprise or consist of the sequences set forth in SEQ ID NOs: 57 and 58; or in 59 and 60 (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to CD79B may comprise or consist of the sequences set forth in SEQ ID NOs: 61 and 62, (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to CD37 may comprise or consist of the sequences set forth in SEQ ID NOs: 63 and 64, (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to CD38 may comprise or consist of the sequences set forth in SEQ ID NOs: 65 and 66, (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to CD70 may comprise or consist of the sequences set forth in SEQ ID NOs: 67 and 68, (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to CD30 may comprise or consist of the sequences set forth in SEQ ID NOs: 39 and 70, (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to CD33 may comprise or consist of the sequences set forth in SEQ ID NOs: 71 and 72 or 73 and 74, (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In preferred embodiments, the bridging molecule for binding to CD33 comprises a targeting moiety comprising an antigen binding domain for specifically binding to CD33, preferably as described herein (more preferably, comprising the amino acid sequence as set forth in SEQ ID NOs: 73 and 72 or 74, (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto), and comprises a tumour-specific antigen epitope moiety comprising or consisting of the amino acid sequence set forth in any of SEQ ID NOs: 365, 373, 377, 388, 390, and 395.

In any embodiment, a bridging molecule for binding to Her2 may comprise or consist of the sequences set forth in SEQ ID NOs: 75 and 75; or 77 and 78, (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to EGFR may comprise or consist of the sequences set forth in SEQ ID NOs: 79 and 80 or 81 and 82 or 83 and 84, (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to CD276 may comprise or consist of the sequences set forth in SEQ ID NOs: 85 and 86, (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to GD2 may comprise or consist of the sequences set forth in SEQ ID NOs: 87 and 88, (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to BCMA may comprise or consist of the sequences set forth in SEQ ID NOs: 89 and 90, (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to CD371 may comprise or consist of the sequences set forth in SEQ ID NOs: 91 and 92, (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to CD135 may comprise or consist of the sequences set forth in SEQ ID NOs: 93 and 94, (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to CD123 may comprise or consist of the sequences set forth in SEQ ID NOs: 95 and 95, (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to CD105 may comprise or consist of the sequences set forth in SEQ ID NOs: 97 and 98, (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to ROR-1 may comprise or consist of the sequences set forth in SEQ ID NOs: 99 and 100, (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to PD-L1 may comprise or consist of the sequences set forth in SEQ ID NOs: 101 and 102, (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to MET-R may comprise or consist of the sequences set forth in SEQ ID NOs: 103 ad 104, (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to PDGFRalpha may comprise or consist of the sequences set forth in SEQ ID NOs: 105 and 106 or 107 and 108 (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to Her3 may comprise or consist of the sequences set forth in SEQ ID NOs: 109 and 110, (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to FRalpha may comprise or consist of the sequences set forth in SEQ ID NOs: 111 and 112, (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to CGPC3 may comprise or consist of the sequences set forth in SEQ ID NOs: 113 and 114, (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to SLAMF7 may comprise or consist of the sequences set forth in SEQ ID NOs: 115 and 116, (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to TNFRSF10B may comprise or consist of the sequences set forth in SEQ ID NOs: 117 and 118, (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to GPNMB may comprise or consist of the sequences set forth in SEQ ID NOs: 119 and 120, (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to VEGFR2 may comprise or consist of the sequences set forth in SEQ ID NOs: 121 and 122, (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to α4β7 and/or Eβ7 may comprise or consist of the sequences set forth in SEQ ID NOs: 123 and 124; or 125 and 126, (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to CSPG4 may comprise or consist of the sequences set forth in SEQ ID NOs: 127 and 128, (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to CD80 may comprise or consist of the sequences set forth in SEQ ID NOs: 129 and 130, (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to CCR4 may comprise or consist of the sequences set forth in SEQ ID NOs: 131 and 132, (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to CD115 may comprise or consist of the sequences set forth in SEQ ID NOs: 133 and 134, (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to ENOX-2 may comprise or consist of the sequences set forth in SEQ ID NOs: 135 and 136, (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to CD56 may comprise or consist of the sequences set forth in SEQ ID NOs: 137 and 138, (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to huVH1-69 may comprise or consist of the sequences set forth in SEQ ID NOs: 139 and 140, (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to CD117 may comprise or consist of the sequences set forth in SEQ ID NOs: 169 and 170, (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to CD133 may comprise or consist of the sequences set forth in SEQ ID NOs: 171 and 172, (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to MUC1 may comprise or consist of the sequences set forth in SEQ ID NOs: 173 and 174, (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to mesothelin may comprise or consist of the sequences set forth in SEQ ID NOs: 175 and 176, (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to ROR2 may comprise or consist of the sequences set forth in SEQ ID NOs: 177 and 178, (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to IL13Ra2 may comprise or consist of the sequences set forth in SEQ ID NOs: 179 and 180, (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to IL13Ra2 may comprise or consist of the sequences set forth in SEQ ID NOs: 181, or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to EPHA2 may comprise or consist of the sequences set forth in SEQ ID NOs: 182 and 183, (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to EGFRvIII may comprise or consist of the sequences set forth in SEQ ID NOs: 184 and 185, (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to PSMA may comprise or consist of the sequences set forth in SEQ ID NOs: 186 and 187, (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to CEA may comprise or consist of the sequences set forth in SEQ ID NOs: 188 and 189, (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to PSCA may comprise or consist of the sequences set forth in SEQ ID NOs: 190 and 191, (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to Lewis Y may comprise or consist of the sequences set forth in SEQ ID NOs: 192 and 193, (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to CD171 L1CAM may comprise or consist of the sequences set forth in SEQ ID NOs: 194 and 195, (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to EpCAM may comprise or consist of the sequences set forth in SEQ ID NOs: 196 and 197, (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to ALK may comprise or consist of the sequences set forth in SEQ ID NOs: 198 and 199, (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to IGF-1R CD221 may comprise or consist of the sequences set forth in SEQ ID NOs: 200 and 201, (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to Nectin 4 may comprise or consist of the sequences set forth in SEQ ID NOs: 202 and 203, (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to FAP may comprise or consist of the sequences set forth in SEQ ID NOs: 204 and 205, (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to AXL may comprise or consist of the sequences set forth in SEQ ID NOs: 206 and 207, (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to CD138 may comprise or consist of the sequences set forth in SEQ ID NOs: 208 and 209, (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to CLDN6 may comprise or consist of the sequences set forth in SEQ ID NOs: 210 and 211, (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to Her4 may comprise or consist of the sequences set forth in SEQ ID NOs: 212 and 213, (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to Claudin 18.2 may comprise or consist of the sequences set forth in SEQ ID NOs: 214 and 215, (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to 0-acetylated GD2 may comprise or consist of the sequences set forth in SEQ ID NOs: 216 and 217, (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to GD3 may comprise or consist of the sequences set forth in SEQ ID NOs: 218 and 219, (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to GM2 may comprise or consist of the sequences set forth in SEQ ID NOs: 220 and 221, (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to TM4SF1 may comprise or consist of the sequences set forth in SEQ ID NOs: 222 and 223, (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to CD147 may comprise or consist of the sequences set forth in SEQ ID NOs: 224 and 225, (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to CEACAM5 may comprise or consist of the sequences set forth in SEQ ID NOs: 226 and 227, (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to VEGFR-1 may comprise or consist of the sequences set forth in SEQ ID NOs: 228 and 229, (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to Podoplanin (PDPN) may comprise or consist of the sequences set forth in SEQ ID NOs: 230 and 231, (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to WT1 may comprise or consist of the sequences set forth in SEQ ID NOs: 232 and 233, (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to GPC2 may comprise or consist of the sequences set forth in SEQ ID NOs: 234 and 235, (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to FGFR4 may comprise or consist of the sequences set forth in SEQ ID NOs: 236 and 237, (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to EphB4 may comprise or consist of the sequences set forth in SEQ ID NOs: 238 and 239, (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to STEAP-1 may comprise or consist of the sequences set forth in SEQ ID NOs: 240 and 241, (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to STEAP-2 may comprise or consist of the sequences set forth in SEQ ID NOs: 242 and 243, (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to IL11Ra may comprise or consist of the sequences set forth in SEQ ID NOs: 244 and 245, (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to CD163 may comprise or consist of the sequences set forth in SEQ ID NOs: 246 and 247, (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to Chlorotoxin may comprise or consist of the sequences set forth in SEQ ID NOs: 248 and 249, (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to CD206 may comprise or consist of the sequences set forth in SEQ ID NOs: 250, (heavy chain sequence recited) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to IL1RAP may comprise or consist of the sequences set forth in SEQ ID NOs: 251 and 252, (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to MICA may comprise or consist of the sequences set forth in SEQ ID NOs: 253 and 254, (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to MAGE-A1 may comprise or consist of the sequences set forth in SEQ ID NOs: 255, or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to MAGE-A1 may comprise or consist of the sequences set forth in SEQ ID NOs: 256 and 257, or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to MAGE-A1 may comprise or consist of the sequences set forth in SEQ ID NOs: 258 and 259, or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to TRBC1 may comprise or consist of the sequences set forth in SEQ ID NOs: 260 and 261, (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to TRBC2 may comprise or consist of the sequences set forth in SEQ ID NOs: 262 and 263, (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to urokinase-type plasminogen activator receptor (uPAR) may comprise or consist of the sequences set forth in SEQ ID NOs: 264 and 265, (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to CD33 may comprise or consist of the sequences set forth in SEQ ID NOs: 268 and 269, (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to Her2 may comprise or consist of the sequences set forth in SEQ ID NOs: 276 and 277, (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to CD33 may comprise or consist of the sequences set forth in SEQ ID NOs: 278 and 279, (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to Her2 may comprise or consist of the sequences set forth in SEQ ID NOs: 270 and 271, (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to B7-H7 (HHLA2) may comprise or consist of the sequences set forth in SEQ ID NOs: 280 and 281, (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to CD34 may comprise or consist of the sequences set forth in SEQ ID NOs: 282 and 283, (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to CD7 may comprise or consist of the sequences set forth in SEQ ID NOs: 284 and 285, (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to CD7 may comprise or consist of the sequences set forth in SEQ ID NOs: 286, (heavy chain sequence) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to GPRC5D may comprise or consist of the sequences set forth in SEQ ID NOs: 287 and 288, (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to TIM-3 may comprise or consist of the sequences set forth in SEQ ID NOs: 289 and 290, (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to CD191 (CCR1) may comprise or consist of the sequences set forth in SEQ ID NOs: 291 and 292, (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to CD66b (CEACAM8) may comprise or consist of the sequences set forth in SEQ ID NOs: 293 and 294, (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to CD11b (MAC-1) may comprise or consist of the sequences set forth in SEQ ID NOs: 295 and 296, (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to EMR2 (ADGRE2) may comprise or consist of the sequences set forth in SEQ ID NOs: 297 and 298, (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to MUC16 may comprise or consist of the sequences set forth in SEQ ID NOs: 299 and 300, (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to NYESO-1 HLA-A2 may comprise or consist of the sequences set forth in SEQ ID NOs: 301 and 302, or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to Survivin HLA-A2 may comprise or consist of the sequences set forth in SEQ ID NOs: 303 and 304, or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to BCMA may comprise or consist of the sequences set forth in SEQ ID NOs: 305, or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to BCMA may comprise or consist of the sequences set forth in SEQ ID NOs: 306, or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to C200 may comprise or consist of the sequences set forth in SEQ ID NOs: 308 and 307, (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any aspect, the bridging molecule described herein does not have a HIS tag.

Also contemplated, is a bridging molecule that comprises an amino acid sequence specified in the Sequence information table above, but without a HIS tag specified in the sequence, or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto. Further, in one embodiment, the bridging molecule may comprise a tag other than a HIS tag, or may comprise an amino acid sequence specified in the Sequence information table above but with a different tag in the position of the HIS tag specified in the sequence.

### Nucleic Acids

In a second aspect, the present invention provides a nucleic acid molecule encoding a bridging molecule of the invention, or part thereof.

The nucleic acid molecule may comprise any polyribonucleotide or polydeoxyribonucleotide, which may be unmodified, or modified, RNA or DNA. For example, the nucleic acid molecule may include single- and/or double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, the nucleic acid molecule may comprise triple-stranded regions comprising RNA or DNA or both RNA and DNA. The nucleic acid molecule may also comprise one or more modified bases or DNA or RNA backbones modified for stability or for other reasons. A variety of modifications can be made to DNA and RNA; thus the term “nucleic acid molecule” embraces chemically, enzymatically, or metabolically modified forms.

In some embodiments of the second aspect of the invention, the nucleic acid molecule comprises a nucleic acid sequence encoding the amino acid sequence of any one of SEQ ID NOs: 2 to 308. Preferably, the nucleic acid comprises a nucleotide sequence encoding the heavy chain and light chain pairs described above.

Further, the present invention provides a nucleic acid construct including a nucleic acid molecule encoding a bridging molecule of the invention, or part thereof. The nucleic acid construct may further comprise one or more of: an origin of replication for one or more hosts; a selectable marker gene that is active in one or more hosts; and/or one or more transcriptional control sequences.

As used herein, the term “selectable marker gene” includes any gene that confers a phenotype on a cell in which it is expressed, to facilitate the identification and/or selection of cells that are transfected or transformed with the construct.

“Selectable marker genes” include any nucleotide sequences which, when expressed by a cell transformed with the construct, confer a phenotype on the cell that facilitates the identification and/or selection of these transformed cells. A range of nucleotide sequences encoding suitable selectable markers are known in the art (for example Mortesen, R M. and Kingston R E. Curr Protoc Mol Biol, 2009; Unit 9.5). Exemplary nucleotide sequences that encode selectable markers include: Adenosine deaminase (ADA) gene; Cytosine deaminase (CDA) gene; Dihydrofolate reductase (DHFR) gene; Histidinol dehydrogenase (hisD) gene; Puromycin-N-acetyl transferase (PAC) gene; Thymidine kinase (TK) gene; Xanthine-guanine phosphoribosyltransferase (XGPRT) gene or antibiotic resistance genes such as ampicillin-resistance genes, puromycin-resistance genes, Bleomycin-resistance genes, hygromycin-resistance genes, kanamycin-resistance genes and ampicillin-resistance genes; fluorescent reporter genes such as the green, red, yellow or blue fluorescent protein-encoding genes; and luminescence-based reporter genes such as the luciferase gene, amongst others which permit optical selection of cells using techniques such as Fluorescence-Activated Cell Sorting (FACS).

Furthermore, it should be noted that the selectable marker gene may be a distinct open reading frame in the construct or may be expressed as a fusion protein with another polypeptide (e.g. the CAR).

As set out above, the nucleic acid construct may also comprise one or more transcriptional control sequences. The term “transcriptional control sequence” should be understood to include any nucleic acid sequence that effects the transcription of an operably connected nucleic acid. A transcriptional control sequence may include, for example, a leader, polyadenylation sequence, promoter, enhancer or upstream activating sequence, and transcription terminator. Typically, a transcriptional control sequence at least includes a promoter. The term “promoter” as used herein, describes any nucleic acid that confers, activates or enhances expression of a nucleic acid in a cell.

In some embodiments, at least one transcriptional control sequence is operably connected to the nucleic acid molecule of the second aspect of the invention.

For the purposes of the present specification, a transcriptional control sequence is regarded as “operably connected” to a given nucleic acid molecule when the transcriptional control sequence is able to promote, inhibit or otherwise modulate the transcription of the nucleic acid molecule. Therefore, in some embodiments, the nucleic acid molecule is under the control of a transcription control sequence, such as a constitutive promoter or an inducible promoter.

The “nucleic acid construct” may be in any suitable form, such as in the form of a plasmid, phage, transposon, cosmid, chromosome, vector, etc., which is capable of replication when associated with the proper control elements and which can transfer gene sequences, contained within the construct, between cells. Thus, the term includes cloning and expression vehicles, as well as viral vectors. In some embodiments, the nucleic acid construct is a vector. In some embodiments the vector is a viral vector.

A promoter may regulate the expression of an operably connected nucleic acid molecule constitutively, or differentially, with respect to the cell, tissue, or organ at which expression occurs. As such, the promoter may include, for example, a constitutive promoter, or an inducible promoter. A “constitutive promoter” is a promoter that is active under most environmental and physiological conditions. An “inducible promoter” is a promoter that is active under specific environmental or physiological conditions. The present invention contemplates the use of any promoter that is active in a cell of interest. As such, a wide array of promoters would be readily ascertained by one of ordinary skill in the art.

Mammalian constitutive promoters may include, but are not limited to, Simian virus 40 (SV40), cytomegalovirus (CMV), P-actin, Ubiquitin C (UBC), elongation factor-1 alpha (EF1A), phosphoglycerate kinase (PGK) and CMV early enhancer/chicken β actin (CAGG).

Inducible promoters may include, but are not limited to, chemically inducible promoters and physically inducible promoters. Chemically inducible promoters include promoters that have activity that is regulated by chemical compounds such as alcohols, antibiotics, steroids, metal ions or other compounds. Examples of chemically inducible promoters include: tetracycline regulated promoters (e.g. see U.S. Pat. Nos. 5,851,796 and 5,464,758); steroid responsive promoters such as glucocorticoid receptor promoters (e.g. see U.S. Pat. No. 5,512,483), ecdysone receptor promoters (e.g. see U.S. Pat. No. 6,379,945) and the like; and metal-responsive promoters such as metallothionein promoters (e.g. see U.S. Pat. Nos. 4,940,661, 4,579,821 and 4,601,978) amongst others.

In the context of the present invention, it will be appreciated that it may be desirable in certain circumstances for the expression of the bridging molecule to be under the control of an inducible promoter. This enables a switching on and switching off of the expression of the nucleic acid encoding the bridging molecule.

In certain embodiment, and in the case of an inducible expression construct, an immune cell expressing a CAR can be genetically modified with a) a nucleic acid encoding an antigen binding receptor and b) an inducible expression construct encoding the bridging molecule. Upon binding of dysfunctional P2X₇ receptor, the immune cell induces expression of the gene encoding the bridging molecule. In certain embodiments, expression of such gene facilitates and/or improves treatment of cancer.

As mentioned above, the control sequences may also include a terminator. The term “terminator” refers to a DNA sequence at the end of a transcriptional unit that signals termination of transcription. Terminators are 3′-non-translated DNA sequences generally containing a polyadenylation signal, which facilitate the addition of polyadenylate sequences to the 3′-end of a primary transcript. As with promoter sequences, the terminator may be any terminator sequence that is operable in the cells, tissues or organs in which it is intended to be used. Suitable terminators would be known to a person skilled in the art.

As will be understood, the nucleic acid constructs of the invention can further include additional sequences, for example sequences that permit enhanced expression, cytoplasmic or membrane transportation, and location signals. Specific non-limiting examples include an Internal Ribosome Entry Site (IRES) or cleavage site (e.g. P2A, T2A).

The present invention extends to all genetic constructs essentially as described herein. These constructs may further include nucleotide sequences intended for the maintenance and/or replication of the genetic construct in eukaryotes and/or the integration of the genetic construct or a part thereof into the genome of a eukaryotic cell.

Methods are known in the art for the deliberate introduction (transfection/transduction) of exogenous genetic material, such as the nucleic acid construct of the third aspect of the present invention, into eukaryotic cells. As will be understood, the method best suited for introducing the nucleic acid construct into the desired host cell is dependent on many factors, such as the size of the nucleic acid construct, the type of host cell, the desired rate of efficiency of the transfection/transduction and the final desired, or required, viability of the transfected/transduced cells. Non-limiting examples of such methods include; chemical transfection with chemicals such as cationic polymers, calcium phosphate, or structures such as liposomes and dendrimers; non-chemical methods such as electroporation, sonoporation, heat-shock or optical transfection; particle-based methods such as ‘gene gun’ delivery, magnetofection, or impalefection or viral transduction.

The nucleic acid construct will be selected depending on the desired method of transfection/transduction. In some embodiments of the third aspect of the invention, the nucleic acid construct is a viral vector, and the method for introducing the nucleic acid construct into a host cell is viral transduction. Methods are known in the art for utilising viral transduction to elicit expression of a CAR in a PBMC (Parker, L L. et al. Hum Gene Ther. 2000; 11: 2377-87) and more generally utilising retroviral systems for transduction of mammalian cells (Cepko, C. and Pear, W. Curr Protoc Mol Biol. 2001, unit 9.9). In other embodiments, the nucleic acid construct is a plasmid, a cosmid, an artificial chromosome or the like, and can be transfected into the cell by any suitable method known in the art.

### Modified Cells

As described herein, the invention includes the use of a cell expressing a chimeric antigen receptor comprising an antigen-recognition domain, wherein the antigen-recognition domain recognises a tumour-specific antigen (such as dysfunctional P2X₇ receptor) expressed on a cell surface. The cell may be an “engineered cell”, “genetically modified cell”, “immune cell” or “immune effector cell” as described herein. Further, the cell may be capable of differentiating into an immune cell. A cell that is capable of differentiating into an immune cell (e.g. T cell that will express the dysfunctional P2X₇ CAR) may be a stem cell, multi-lineage progenitor cell or induced pluripotent stem.

In any embodiment, the cell may be a T cell, wherein optionally said T cell does not express TcRαβ, PD1, CD3 or CD96 (e.g. by way of knocking down or knocking out one of these genes on a genetic level or functional level).

In any embodiment, the cell may be an immune cell, wherein optionally said cell does not express accessory molecules that can be checkpoint, exhaustion or apoptosis-associated signalling receptors as well as ligands such as PD-1, LAG-3, TIGIT, CTLA-4, FAS-L and FAS-R, (e.g. by way of knocking out one of these genes on a genetic level or functional level).

In some embodiments, the genetically modified cell includes two or more different CARs.

In some embodiments of the invention, the genetically modified cell includes a nucleic acid molecule, or a nucleic acid construct, that encodes for two or more different CARs. In some embodiments of the invention, the genetically modified cell includes two or more nucleic acid molecules, or two or more nucleic acid constructs, each of which encodes for a different CAR.

As referred to herein, a “genetically modified cell” includes any cell comprising a non-naturally occurring and/or introduced nucleic acid molecule or nucleic acid construct encompassed by the present invention. The introduced nucleic acid molecule or nucleic acid construct may be maintained in the cell as a discreet DNA molecule, or it may be integrated into the genomic DNA of the cell.

Genomic DNA of a cell should be understood in its broadest context to include any and all endogenous DNA that makes up the genetic complement of a cell. As such, the genomic DNA of a cell should be understood to include chromosomes, mitochondrial DNA and the like. As such, the term “genomically integrated” contemplates chromosomal integration, mitochondrial DNA integration, and the like. The “genomically integrated form” of the construct may be all or part of the construct. However, in some embodiments the genomically integrated form of the construct at least includes the nucleic acid molecule of the second aspect of the invention.

As used herein, the term “different CARs” or “different chimeric antigen receptors” refers to any two or more CARs that have either non-identical antigen-recognition and/or non-identical signalling domains. In one example, “different CARs” includes two CARs with the same antigen-recognition domains (e.g. both CARs may recognise a dysfunctional P2X₇ receptor), but have different signalling domains, such as one CAR having a signalling domain with a portion of an activation receptor and the other CAR having a signalling domain with a portion of an co-stimulatory receptor. As will be understood, at least one of the two or more CARs within this embodiment will have an antigen-recognition domain that recognises the dysfunctional P2X₇ receptor and the other CAR(s) may take any suitable form and may be directed against any suitable antigen.

Accordingly, in some embodiments of the invention the two or more different CARs have different signalling domains, and may have identical, or different, antigen-recognition domains. Specifically, the genetically modified cell of the invention may include a first chimeric antigen receptor with a signalling domain that includes a portion derived from an activation receptor and a second chimeric antigen receptor with a signalling domain including a portion derived from a co-stimulatory receptor.

In some embodiments, the activation receptor (from which a portion of signalling domain is derived) is the CD3 co-receptor complex or is an Fc receptor.

In some embodiments, the co-stimulatory receptor (from which a portion of signalling domain is derived) is selected from the group consisting of CD27, CD28, CD-30, CD40, DAP10, OX40, 4-1BB (CD137) and ICOS.

In some embodiments, the co-stimulatory receptor (from which a portion of signalling domain is derived) is selected from the group consisting of CD28, OX40 or 4-1BB.

In some embodiments, the genetically modified cell is further modified to constitutively express co-stimulatory receptors.

As described above, a cellular immune response is typically only induced when an activation signal (typically in response to an antigen) and a co-stimulation signal are simultaneously experienced. Therefore, by having a genetically modified cell in accordance with some of the above embodiments, which includes two or more CARs that in combination provide both an intracellular activation signal and an intracellular co-stimulation signal, ensures that a sufficient immune response can be induce in response to the recognition by the CAR(s) of their cognate antigen. Alternatively, the genetically modified cell may include only one CAR, which has an antigen-recognition domain that recognises a dysfunctional P2X₇ receptor, and may constitutively express co-stimulatory receptors, thereby increasing the likelihood of co-stimulation being provided simultaneously when the CAR is activated. Alternatively, the genetically modified cell may be further modified to constitutively express both co-stimulatory receptor(s) and its/their ligand(s). In this way the cell is continuously experiencing co-stimulation and only needs the activation of a CAR, with a signalling domain including a portion from an activation receptor, for immune activation of the cell.

Therefore in some embodiments, the genetically modified cell expressing the CAR is further modified so as to constitutively express co-stimulatory receptors. In further embodiments, the genetically modified cell is further modified so as to express ligands for the co-stimulatory receptors, thereby facilitating auto-stimulation of the cell. Examples of CAR-expressing T cells that also express both co-stimulatory receptors and their cognate ligands (so as to induce auto-stimulation) are known in the art and include, inter alia, those disclosed in Stephen MT. et al. Nat Med, 2007; 13: 1440-9.

The potency of a genetically modified cell including a CAR can be enhanced by further modifying the cell so as to secrete cytokines, preferably pro-inflammatory or pro-proliferative cytokines. This secretion of cytokines provide both autocrine support for the cell expressing the CAR, and alters the local environment surrounding the CAR-expressing cell such that other cells of the immune system are recruited and activated. Consequently, in some embodiments of the fourth or fifth aspects of the invention the genetically modified cell is further modified to secret cytokines. This secretion may be constitutive, or may be inducible upon recognition of a CAR of its cognate antigen of ligand.

Whilst any one or more cytokines can be selected depending on the desired immune response, preferable cytokines and/or chemokines include IL-2, IL-7, IL-12, IL-15, IL-17, IL-18 and IL-21, CCL19, CCL21 or a combination thereof.

The immune cell of the invention can be any suitable immune cell, or progenitor cell thereof, or can be a homogeneous or a heterogeneous cell population. In some embodiments, the cell is a leukocyte, a Peripheral Blood Mononuclear Cell (PBMC), a lymphocyte, a T cell, a CD4+ T cell, a CD8+ T cell, a natural killer cell, a natural killer T cell, or a γδ T cell.

The immune cell may be a T cell, wherein optionally said T cell does not express TcRαβ, PD1, CD3 or CD96 (e.g. by way of knocking down or knocking out one of these genes on a genetic level or functional level).

The immune cell may not express accessory molecules that can be checkpoint, exhaustion or apoptosis-associated signalling receptors as well as ligands such as PD-1, LAG-3, TIGIT, CTLA-4, FAS-L and FAS-R, (e.g. by way of knocking out, or knocking down, one of these genes on a genetic level or functional level).

### Methods of Treatment and Administration

As discussed further in this document, the present invention finds application in the treatment of a variety of conditions, although preferably in the treatment of cancers.

The present invention also contemplates various scenarios for the use of the two components of the therapeutics described herein.

In one scenario, the individual requiring treatment is administered a single composition comprising both the CAR T cells and the bridging molecule.

In further scenarios, the individual requiring treatment is administered a population of CAR T cells, which cells comprise an expression vector encoding the bridging molecule. The expression vector may facilitate constitutive or inducible expression of the nucleic acid sequence encoding the bridging molecule.

Further still, the individual requiring treatment may be administered the CAR T cells, and at a later date, be administered a composition comprising the bridging molecule (e.g., via infusion), or a nucleic acid sequence encoding the bridging molecule.

Such a scenario may be appropriate in circumstances where the individual is first treated with the CAR T cells for targeted treatment of cancers that are positive for dysfunctional P2X₇ receptor and wherein the subsequent administration of the bridging molecule is for the purposes of redirecting the CARs to alternative cancer antigens, or to peptides derived from an infectious agent and which are presented on MHC I or II molecules of cells.

Thus the bridging molecule may be administered prior to, at the same time as, or after the subject receives treatment with the CAR T cell.

Where the bridging molecule and CAR T cells are administered to the subject at the same time, they can be administered via the same route of administration (including in a single composition), or alternatively via different routes of administration. For example, the CAR T cells may be administered by injection into the blood stream of the subject, while the bridging molecule may be administered via another route of administration such as intramuscularly, intradermally, subcutaneously or intraperitoneally.

A bridging molecule may be produced or expressed inside the body by genetically engineered cells secreting bridging molecules spontaneously or upon stimulation via a stimulating agent e.g. a small molecule. Alternatively, cells may continuously secrete bridging molecules and will stop secreting them upon application of a stimulating agent, e.g. a small molecule.

It will be clearly understood that, although this specification refers specifically to applications in humans, the invention is also useful for veterinary purposes. Thus in all aspects the invention is useful for domestic animals such as cattle, sheep, horses and poultry; for companion animals such as cats and dogs; and for zoo animals. Therefore, the general term “subject” or “subject to be/being treated” is understood to include all animals (such as humans, apes, dogs, cats, horses, and cows).

The term “administered” means administration of a therapeutically effective dose of the aforementioned composition including the respective cells to an individual. By “therapeutically effective amount” is meant a dose that produces the effects for which it is administered. The exact dose will depend on the purpose of the treatment, and will be ascertainable by one skilled in the art using known techniques. As is known in the art and described above, adjustments for systemic versus localised delivery, age, body weight, general health, sex, diet, time of administration, drug interaction and the severity of the condition may be necessary, and will be ascertainable with routine experimentation by those skilled in the art.

Subjects requiring treatment include those already having a benign, pre-cancerous, or non-metastatic tumour as well as those in which the occurrence or recurrence of cancer is to be prevented. Subjects may have metastatic cells, including metastatic cells present in the ascites fluid and/or lymph node.

The objective or outcome of treatment may be to reduce the number of cancer cells; reduce the primary tumour size; inhibit (i.e., slow to some extent and preferably stop) cancer cell infiltration into peripheral organs; inhibit (i.e., slow to some extent and preferably stop) tumour metastasis; inhibit, to some extent, tumour growth; and/or relieve to some extent one or more of the symptoms associated with the disorder.

Efficacy of treatment can be measured by assessing the duration of survival, time to disease progression, the response rates (RR), duration of response, and/or quality of life.

The method is particularly useful for extending time to disease progression.

The method is particularly useful for extending survival of the human, including overall survival as well as progression free survival.

The method is particularly useful for providing a complete response to therapy whereby all signs of cancer in response to treatment have disappeared. This does not always mean the cancer has been cured.

The method is particularly useful for providing a partial response to therapy whereby there has been a decrease in the size of one or more tumours or lesions, or in the extent of cancer in the body, in response to treatment.

The objective or outcome of treatment may be any one or more of the following:
to reduce the number of cancer cells;
reduce the primary tumour size;
inhibit (i.e., slow to some extent and preferably stop) cancer cell infiltration into peripheral organs;
inhibit (i.e., slow to some extent and preferably stop) tumour metastasis;
inhibit, to some extent, tumour growth;
relieve to some extent one or more of the symptoms associated with the disorder.

In one embodiment, subjects requiring treatment include those having a benign, pre-cancerous, non-metastatic tumour.

In one embodiment, the cancer is pre-cancerous or pre-neoplastic.

In one embodiment, the cancer is a secondary cancer or metastasis. The secondary cancer may be located in any organ or tissue, and particularly those organs or tissues having relatively higher haemodynamic pressures, such as lung, liver, kidney, pancreas, bowel and brain. The secondary cancer may be detected in the ascites fluid and/or lymph nodes.

In one embodiment, the cancer may be substantially undetectable.

“Pre-cancerous” or “preneoplasia” generally refers to a condition or a growth that typically precedes or develops into a cancer. A “pre-cancerous” growth may have cells that are characterised by abnormal cell cycle regulation, proliferation, or differentiation, which can be determined by markers of cell cycle.

The cancer may be a solid or a “liquid” tumour. In other words, the cancer may be growth in a tissue (carcinoma, sarcoma, adenomas etc) or it may be a cancer present in bodily fluid such as in blood or bone marrow (e.g., lymphomas and leukaemias).

In certain preferred embodiments, the cancer requiring treatment may be a cancer characterised by low levels of expression of dysfunctional P2X₇ receptor.

Examples of such cancers include Burkitt's lymphoma. However, immunohistochemical analyses of surface expression of the dysfunctional P2X₇ (nfP2X₇) receptor on patient tumour biopsies reveals a range from 1+ to 3+ in IHC score. Samples with low expression may therefore be found in a wide range of tumour types. Examples are found in solid tumours of various types, including but not limited to neuroblastoma, colorectal cancers, lung cancers, kidney cancers, skin cancers, breast cancers, brain cancers and prostate cancer. Such differences in expression level in different tissues may be due to the formation of tumours from cells that are at an earlier state of transformation (the tissues with the highest receptor expression may be those undergoing the highest rate of proliferation).

Other examples of cancers that can be treated in accordance with the methods of the present invention include blastoma (including medulloblastoma and retinoblastoma), sarcoma (including liposarcoma and synovial cell sarcoma), neuroendocrine tumours (including carcinoid tumours, gastrinoma, and islet cell cancer), mesothelioma, schwannoma (including acoustic neuroma), meningioma, adenocarcinoma, melanoma, leukaemia or lymphoid malignancies, lung cancer including small-cell lung cancer (SCKC), non-small cell lung cancer (NSCLC), adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer (including metastatic breast cancer), colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, testicular cancer, oesophageal cancer, tumours of the biliary tract, as well as head and neck cancer.

In further examples, the methods of treatment contemplated within the scope of the present invention, include methods for treating or preventing an infectious disease. Thus, the bridging molecules of the invention can be utilised to redirect the CAR T cells towards an additional surface accessible antigen, for example wherein the antigen is a non-cancer associated pathogenic antigen presented on an MHC I or MHC II molecule as further described herein.

The subject requiring treatment for an infectious disease may be at risk or have been diagnosed with the disease. Subjects at risk include those who are immunocompromised. Thus, the methods of the present invention also allow for the prevention of onset of infectious disease in individuals receiving therapy (such as for treating cancer) that renders them immunocompromised and therefore susceptible to infection.

Examples of intracellular pathogens from which peptides are presented on MHC I or MHC II molecules include: viral infections, intracellular bacterial infections, protozoan infections, and intracellular fungal infections.

Examples of viral infections that may be treated using the methods of the present invention include: HIV, hepatitis (e.g., Hepatitis A, B or C), a coronavirus (e.g. SARS-CoV-2), an influenza virus, varicella zoster virus, mumps virus.

Examples of intracellular bacterial infections which may be treated using the methods of the present invention include: mycobacterial infections (e.g., Mycobacterium tuberculosis), Bartonella henselae, Francisella tularensis, Listeria monocytogenes, Salmonella Typhi, Brucella, Legionella, Nocardia, Neisseria, Rhodococcus, Yersinia, Staphylococcus aureus, Chlamydia, Rickettsia, Coxiella, and Chlamydophila pneumoniae.

Examples of intracellular infections caused by fungal pathogens: Histoplasma capsulatum, Cryptococcus neoformans, and Pneumocystitis jirovecii.

Examples of obligate intracellular protozoan pathogens include: Apicomplexans (Plasmodium spp., Toxoplasma gondii and Cryptosporidium parvum), and Trypanosomatids (Leishmania spp. and Trypanosoma cruzi).

Immune cells that may be targeted to modulate the immune system in the context of cancer and/or autoimmune disease may be B cells (CD19, CD20, CD22), plasma cells (BCMA, CD38, CD138), T cell subsets via (TRBC1 or TRBC2, α4β7 & αEβ7, CD7), macrophages and TAMs (CD163 and CD206). In the context of allogeneic stem cell transplantation, immune-based conditioning may be undertaken by targeting (CD34, CD117, CD133, CD33 and CD38) especially in case of non-malignant diseases e.g. thalassaemia major or sickle cell anaemia and/or in case of DNA-repair defects like Fanconi anaemia.

Targeting senescent tumour cells via the marker (uPAR) will help to eliminate tumour cells in a resting state and which are likely to expand at later time points and promote even faster proliferation of cancer cells in the latter by secreting tumour promoting cytokines and shaping a tumour-suppressive environment protecting new cancerous subclones.

CAR T cells may be constructed in a way that they are able to immunosuppress other immune cells, e.g. TREG CAR T cells or by secreting immunosuppressive cytokines (TGFbeta, IL10) and chemokines by introducing the corresponding inducible expression cassette [NFAT-dependent cytokine secretion] and the signalling in the construct.

The bridging molecules of the invention may be formulated for administration to a subject using techniques known to the skilled artisan. Formulations of the bridging molecules may include pharmaceutically acceptable excipient(s) (carriers or diluents). Examples of generally used excipients include, without limitation: saline, buffered saline, dextrose, water-for-injection, glycerol, ethanol, and combinations thereof, stabilising agents, solubilising agents and surfactants, buffers and preservatives, tonicity agents, bulking agents, and lubricating agents.

A formulation of bridging molecules may include one type of bridging molecule, or more than one type of bridging molecule (i.e., wherein the bridging molecules may have the same or different targeting and/or dysfunctional P2X₇ receptor epitope moieties).

The bridging molecules may be administered to a subject using modes and techniques known to the skilled artisan. Exemplary modes include, but are not limited to, intravenous, intraperitoneal, and intratumoural injection. Other modes include, without limitation, intradermal, subcutaneous (s.c, s.q., sub-Q, Hypo), intramuscular (i.m.), intra-arterial, intramedullary, intracardiac, intra-articular (joint), intrasynovial (joint fluid area), intracranial, intraspinal, and intrathecal (spinal fluids).

Formulations comprising the bridging molecule are administered to a subject in an amount that is effective for treating the specific indication or disorder. In general, formulations comprising at least about 0.01 μg/kg to about 100 mg/kg body weight of the bridging molecule may be administered to a subject in need of treatment. In most cases, the dosage may be from about 100 μg/kg to about 10 mg/kg body weight of the bridging molecules daily, taking into account the routes of administration, symptoms, etc. However, the amount of bridging molecules in formulations administered to a subject may vary between wide limits, depending upon the location, source, identity, extent and severity of the disorder, the age and condition of the individual to be treated, etc. A physician may ultimately determine appropriate dosages to be used. The bridging molecules may be administered as a continuous infusion or a bolus application.

The timing between the administration of the CAR T cell and the bridging molecule formulation may range widely depending on factors that include the type of (immune) cells being used, the binding specificity of the CAR, the identity of the targeting moiety and the identity of the target cell, e.g. cancer cell to be treated, the location of the target cell in the subject, the means used to administer the formulations to the subject, and the health, age and weight of the subject being treated. Indeed, the TCBM formulation may be administered prior to, simultaneous with, or after the genetically engineered (immune) cell formulation.

It will be understood that the invention disclosed and defined in this specification extends to all alternative combinations of two or more of the individual features mentioned or evident from the text or drawings. All of these different combinations constitute various alternative aspects of the invention.

### EXAMPLES

### Example 1—Materials and Methods Including Generation of Bridging Molecules

Cultivation, transfection and protein production were performed as per ExpiCHO Expression System User Guide (Thermo—ExpiCHO™ Expression System USER GUIDE. For transfection of ExpiCHO-S™ Cells in a defined, serum-free medium Catalogue Number A29133, Publication Number MAN0014337). In summary, ExpiCHO were routinely passaged and maintained at less than 4-6×10⁶ cell/mL in ExpiCHO medium. Cells in the mid-log growth phase were transfected when cell number was in the range of 5-7×10⁶ cell/mL. For transfection, liposome complex was prepared with 1 μg DNA for each mL of culture. For co-transfection of vectors coded with heavy and light chains separately, the vector ratio was set at 1:1 unless specified otherwise. “High Titer” or “Max Titer” expression protocols were followed after transfections, and cultures were harvested when cell viabilities dropped below 70%. Harvest was done by centrifugation at 300×g for 5 min at 20° C. Cells were discarded and the supernatant was centrifuged again at 4000× g for 30 mins at 4° C. The harvested supernatants were clarified by 0.2 μm filtration using PES membrane before freezing for storage.

Harvested samples could be enriched and buffer-exchanged by spin-columns or TFF cassette with nominal molecular size cut off of 5, 10 or 30 kDa.

For HIS-tagged column purification, the harvested supernatants were dialysed via SnakeSkin dialysis tube with nominal molecular size cut off 5, 10 or 30 kDa depending on the protein of interest. For large scale production, the supernatants were washed through a TFF cassette with a certain molecular size cut off membrane. Buffer-exchange to the desired column loading buffer also was achieved through the above-mentioned procedures to prepare the sample for the His-tagged column purification. The purification was performed on either a HisTrap excel column (Cytiva) or PureCube 100 Compact Cartridge Ni-INDOGO affinity Column (Cat #75302, Cube Biotech) or other equivalent column. Purification was performed on a AKTA Pure system (Cytiva) equipped with UV detector at 280 nm wavelength, conductivity detector and pH probe. Loading and washing buffer consisted of 50 mM Sodium Phosphate Monobasic and 0.3 M Sodium Chloride, pH 8.0. The elution buffer contained 500 mM Imidazole. The eluted protein was buffer exchanged to PBS using Vivaspin (Sartorius) and stored under 4° C.

Protein was quantified via Nanodrop at 280 nm wavelength and standard Bicinchoninic acid (BCA) protein assay. The protein purity was confirmed by SDS PAGE gel electrophoresis.

The detailed experimental data generated by the inventor(s) and described herein includes the generation of a wide variety of bridging molecules that include:
1. Targeting moieties generated in multiple antibody formats, e.g. Fab and scFv;
2. Various positioning of the dysfunctional P2X₇ receptor epitope moiety on the targeting moiety including, for example, on the V_{L} and V_{H};
3. Inclusion of linkers between the targeting moiety and the dysfunctional P2X₇ receptor epitope moiety;
4. Targeting moieties that bind to a wide range of cell surface antigens that are present on tumour cells from different tissue origins.

### Antibody/Fab Conjugation

Conjugation of BILO3s 2-2-1-Fc (an anti-nfP2X₇ receptor antibody) with fluorochrome Alexa Fluor® 647 (AF647) was performed according to manufacturer's instruction (Cat #A20186, ThermoFisher). The AF647 labelled BILO3s 2-2-1-Fc antibody was reconstituted in PBS, pH 7.2, with 2 mM sodium azide.

### Testing Binding of Bridging Molecules to Cells by Flow Cytometry

### Reagents

Abs and Fabs:
BILO3 pure antibody: prediluted to 100 ug/mL by PBS.
BILO3-AF647 antibody: prediluted to 100 ug/mL by PBS
Anti-His antibody-FITM (1 mgdmL) (Abcam Cat #abi206, Cat #GR3361939-1)
Rabbit IgG-FITC isotype control (Abcam Cat #ab3706, Cat #GR3356160-1)
Bridging molecules, generated in-house and harvested from supernatant see below

Cell lines used for binding assays: JeKo-1 (CD19, CD20, CD79B, CD37, CD22, ROR1, Her2), MOLM-13 (CD33, CD38, CD37, CD135, CD123), PC3 (Her2), MDA-MB-231 (EGFR, PD-L1), Raji (CD22, CD70, CD79B), Karpas299 (CD30), U937 (CD105), HL60, RPM18226 (BCMA, CD38, CD33).

Cells were resuspended at a density of in 5×10⁶ cells/ml and 100 μL aliquots used per well for staining (0.5×10⁶/sample for testing).

| | | | Growth | | Culture |
|---|---|---|---|---|---|
| Cell Line | Disease Type | Source | Properties | Subculture Ratio | Medium |
| MOLM-13 | Acute Myeloid | ATCC | Suspension | 4 × 10⁵-2 × 10⁶ | RPMI |
| | Leukaemia, AML | | | cells/mL | 1640 + |
| | | | | | 10% FBS |
| JeKo-1 | Mantle Cell | ATCC | Suspension | 2 × 10⁵-2 × 10⁶ | RPMI |
| | Lymphoma, MCL | | | cells/mL | 1640 + |
| | | | | | 20% FBS |
| MDA-MB | Mammary | NCI-60 | Adherent | 1:4 | Leibovitz's |
| 231 | | panel | | | L-15 |
| | | | | | medium + |
| | | | | | 10% FBS; |
| | | | | | RPMI1640 + |
| | | | | | 10% FBS |
| PC-3 | Prostate | CellBank | Adherent | 1:6 | F12K or |
| | | Australia | | | RPMI |
| | | | | | 1640 + |
| | | | | | 10% FBS |
| U937 | Histiocytic | CellBank | Suspension | 2 × 10⁵-9 × 10⁵ | RPMI |
| | Lymphoma | Australia | | cells/mL | 1640 + |
| | | | | | 2 mM |
| | | | | | Glutamine + |
| | | | | | 10% FBS |
| Raji | Burkitt's lymphoma | CellBank | Suspension | 3 × 10⁵-9 × 10⁵ | RPMI |
| | | Australia | | cells/mL | 1640 + |
| | | | | | 2 mM |
| | | | | | Glutamine + |
| | | | | | 10% FBS |
| HL-60 | Acute | NCI-60 | Suspension | 1 × 10⁵-1 × 10⁶ | IMDM + |
| | promyelocytic | panel | | cells/mL | 10% FBS |
| | leukaemia | | | | |
| RPMI- | Multiple Myeloma | NCI-60 | Semi adhesion | 5 × 10⁵-2 × 10⁶ | RPMI |
| 8226 | | panel | | cells/mL | 1640 + |
| | | | | | 10% FBS |
| Karpas299 | anaplastic large | CellBank | Suspension | 0.5-2 × 10⁶ | RPMI |
| | cell lymphoma | Australia | | cells/mL | 1640 + |
| | | | | | 2 mM |
| | | | | | Glutamine + |
| | | | | | 10-20% |
| | | | | | FBS |

### Procedure

### 1. Staining Condition List:

Bridging molecule binding to BILO3s Antibody Staining and His Tag Antibody detection

### Control Conditions:

Control bridging molecule binding to BILO3s Antibody Staining and His Tag Antibody detection
No bridging molecule binding to BILO3s Antibody Staining and His Tag Antibody detection
Unstained cells

### 2. Staining Procedure

Block the cells with human FcR blocker (20% v/v, Miltenyi) on ice for 10 min and wash off the unbounded FCR blocker.
Incubate the cells in 50 μL of crude prep supernatant and stain on ice for 15 min.
Wash the cell suspension by FACS buffer x2
Stain the cells with 1 μg/mL of BILO3s-AF647 and 1 μL of His Tag antibody per 100 μL cell suspension.
Wash the cells by FACS buffer x2.
Cells ready to be analysed on MacsQuant16.

### Lentiviral Vector Production Using Adherent Lenti-X HEK293T (Takara) Cells and PEI

### Lenti-X 293T Culture Media

For cell culture pre transduction:

90% Dulbecco's Modified Eagle's Medium (DMEM) with high glucose (4.5 g/L), 4 mM L-glutamine, and sodium bicarbonate (Sigma-Aldrich, D5796); 10% Foetal Bovine Serum (FBS); 1 mM sodium pyruvate (Sigma-Aldrich, S8636).

### For Cell Culture Post Transduction:

90% Dulbecco's Modified Eagle's Medium (DMEM) with high glucose (4.5 g/L), 4 mM L-glutamine, and sodium bicarbonate (Sigma-Aldrich, D5796); 10% Foetal Bovine Serum (FBS); 1 mM sodium pyruvate (Sigma-Aldrich, S8636), and 10 mM sodium butyrate.

| Plasmid DNA ID# | Construct Name |
|---|---|
| Transfer | Various |
| A | pRSV/REV (expresses HIV-1 REV) |
| B | pMDL/RRE (expresses HIV GAG/POL) |
| C | pMD2.G (expresses VSV glycoprotein) |

### Protocol, Part I Lenti-X 293T Cells Lentivirus Transfection

### Day 0:

1. Seed 1.7×10⁶ cells per 15 cm dish so that they will be ˜80% confluent on the day of transfection—the following Monday (˜16×10⁶ cells).

### Day 1:

1. Check cells under microscope. The cells should be about 75-90% confluent.
2. Gently aspirate media, add 20 mL fresh DMEM supplemented with 10% FCS to each 15 cm dish, and incubate for at least two hours before transfection.
3. Perform the transfection procedure in the afternoon (˜2.30-4.30 pm). Warm an aliquot of serum-free DMEM to 37° C.
4. Prepare the Mixture A (Plasmid DNA solution) and Mixture B (PEIpro solution).
5. Determine the required volumes of DMEM and plasmid DNA in Mixture A according to the table below.

| | | | | |
|---|---|---|---|---|
| | T175 or 15 cm | T75 | 10 cm dish | 6 well |
| Mixture A (DNA) | dish (175 cm²) | (75 cm²) | (60 cm²) | (9.6 cm²) |

| | |
|---|---|
| Plasmid DNA* | VSV-G 0.06 μg/cm² |
| | Rev 0.06 μg/cm² |
| | Gag/pol 0.12 μg/cm² |
| | Transfer plasmid 0.08 μg/cm² |
| DMEM (w/o | 5% final culture volume |
| additives) | |

6. Determine the volumes required for each plasmid DNA component for the number of plates required. 15 cm dish has an area of ˜175 cm².

| | μg/plate | | μg/plate | |
|---|---|---|---|---|
| Mixture A | CCT | #_{——}_{——}_{——}plates | VGEF | #_{——}_{——}_{——}plates |
| Transfer plasmid | 14 | | 2 | |
| pMDL/RRE | 21 | | 7.5 | |
| pRSV/REV | 10.5 | | 7.5 | |
| pMD2.G | 10.5 | | 5 | |
| DMEM high | 0.75 mL | | 0.75 mL | |
| glucose (w/o | | | | |
| additives) | | | | |

7. Determine the volumes required for each component of Mixture B (PEIpro solution).
For each 15 cm dish,

| Mixture B | | μL/plate | | | μL/plate | | |
|---|---|---|---|---|---|---|---|
| (PEIpro) | | CCT | | #_{——}_{——}_{——}plates | VGEF | | #_{——}_{——}_{——}plates |
| PEIpro | PEIpro:DNA = | 56 | uL | | 22 | uL | |
| 1 mg/mL | 1:1 ratio | | | | | | |
| DMEM high | | 0.75 | mL | | 0.75 | mL | |
| glucose | | | | | | | |
| (w/o | | | | | | | |
| additives) | | | | | | | |

8. Vortex PEIpro for 5 seconds and then spin down if needed to collect liquid in the bottom of the tube.
9. Prepare Mixture B (PEIpro in media) in a 15 mL tube by adding PEIpro into the DMEM high glucose without any additives. Add PEI into DMEM Invert up and down a few times and spin down quickly.
10. Prepare Mixture A (plasmid DNA dilution in media) in a 50 mL conical tube by adding DNA into the media. Mix gently by inverting up and down and spin down quickly.
11. Mix and prepare transfection mixture (PEIpro/DNA solution) by adding Mixture B (PEIpro solution) to Mixture A (plasmid DNA dilution). Using a p1000 micropipette, add the PEIpro solution dropwise to DNA solution and immediately invert 3-4 times to mix. Do not vortex.
12. Incubate at room temperature for 15 min, no longer than 30 min. Do not agitate the tube during this time.
13. After the 15-min incubation, add the transfection mixture to the flasks/dishes containing cells and fresh media (dropwise if possible). Mix by gently rocking the flask/dish horizontally with back and forth and left and right motions.
14. Incubate the flasks/dishes at 37′C with 5% CO2 overnight.

### Day 2:

15. 16-18 hours after transfection, replace media. Working with 2 plates at a time, aspirate old media. Using a 25 mL pipette, carefully add 15 mL fresh DMEM supplemented with 10% FCS, and 10 mM sodium butyrate.

Day 3 first harvest 24 hour post media change:
16. Collect the supernatants from the culture flasks/dishes in 50 mL Falcon tubes. Carefully replace each plate with 15 mL fresh DMEM supplemented with 10% FCS and 10mMm sodium butyrate and return plates to incubator.
17. Centrifuge the supernatant at 500×g for 10 min at 4 C (for concentration by Lenti-X concentrator). For ultracentrifugation, centrifuge at 3800 rpm for 30 min at RT.
18. Draw up the virus-containing supernatant with a 20 mL syringe and filter through a 0.45 μm PES filter (Millipore) into a new 50 mL Falcon tube. This can be used as (a) Crude preparation for transduction, or proceed to (b) concentration by ultracentrifugation, or (c) concentration by Lenti-X concentrator. Alternatively, store crude virus 4′C overnight and pool with 48 hour harvest. Store concentrated virus in aliquots at −80° C. for extended periods.
19. Discard plates, tubes and filters in a biohazard bag in the tissue culture hood. Seal the biohazard bag before taking it out of the hood for disposal.

Day 4 second harvest 48 hour post media change:
20. Collect the supernatants from the culture flasks/dishes in 50 mL Falcon tubes.
21. Centrifuge the supernatant at 2000×g for 30 min at room temp.
22. Draw up the virus-containing supernatant with a 20 mL syringe and filter through a 0.45 μm filter (Millipore) into a new 50 mL Falcon tube. This can be pooled with the 24 hr harvest or process separately and proceed to (b) concentration by ultracentrifugation, or (c) concentration by Lenti-X concentrator.
23. Discard plates, tubes and filters in a biohazard bag in the tissue culture hood. Seal the biohazard bag before taking it out of the hood for disposal.

### Part II Concentrating Lentiviruses by Lenti-X Concentrator

24. Harvest the lentivirus-containing supernatants. Caution: supernatants contain live lentivirus. Pool similar stocks together, if desired. Centrifuge briefly at 500×g for 10 min or filter through a 0.45 μm filter.
25. Transfer clarified supernatants to a sterile container and combine 1 volume of Lenti-X Concentrator with 3 volumes of clarified supernatant. Mix by gentle inversion. Larger volumes may be accommodated through the use of larger (i.e., 250 mL or 500 mL) centrifuge tubes.
26. The incubation with the Lenti-X concentrator is done once. Either at harvest (after 1 day) or after 2 days (pooled harvest). Incubation at least for 30 min or overnight and then centrifugation of the pre-incubated fluid.
27. Centrifuge samples at 1,500×g for 45 minutes at 4° C. After centrifugation, an off-white pellet will be visible.
28. Carefully remove supernatant, taking care not to disturb the pellet. Residual supernatant can be removed with either a pipette tip or by brief centrifugation at 1,500×g.
29. Gently resuspend the pellet in 1/10 to 1/100th of the original volume using complete DMEM, PBS, or TNE. The pellet can be somewhat sticky at first, but will go into suspension quickly.
30. Immediately titrate sample or store at −80° C. in single-use aliquots.

### Car T Cell Generation Protocol

nfP2X₇ BRIDGE CAR T cells were generated by lentiviral transduction of CD4/CD8 positive selected T cells (1:1 ratio) via magnetic activated cell sorting (MACS) stimulated with TransAct (all according to manufacturer's instructions) cultivated in IL7/IL15 supplemented TecsMACS media (both 10 ng/mL). The donor source was a buffy coat.

CAR T cells were treated in the very same way but underwent lentiviral transduction to express the nfP2X₇ BRIDGE CAR. Activated untransduced T cells (aUT) do not express any receptor that can either engage with the EGFR nor the CD33 bridging molecules.

### Hypothesis:

nfP2X₇ BRIDGE CAR T cells have a superior effector function over aUT as they are redirected towards cancer cells directly via nfP2X₇ recognition on the cell surface of MOLM-13 leukaemic cells.

nfP2X₇ BRIDGE CAR T cells have a superior effector function over aUT as they are redirected towards cancer cells indirectly via nfP2X₇ E200 derived epitope on the CD33 Fab-bridging molecules on the surface of MOLM-13 leukaemic cells.

### Reagent and Equipment Preparation

CAR T culture medium: TexMACS with human IL-7 and IL-15. IL-7 stock concentration was 100 μg/mL, each vial has 55 μL. IL-15 stock concentration is 50 μg/mL, each vial had 55 μL.

For preparation of TexMACS with final concentration of 10 ng/mL of IL-7, 5 ng/mL of IL-15 and 3% FBS, add 50 ul of IL-7, 50 ul of IL-15 stock, and 15 mL FBS into each bottle (500 mL) of TexMACS medium. Label the date of adding of cytokines on the medium bottle.

Freezing medium preparation on the day of harvest: 10% of DMSO, 90% of FBS. Note: Add the reagent into 50 mL falcon tube according to the following order: DMSO to FBS.

### Part I: T Cell Activation and T Cell Transduction

Day 1: T cell activation
1. CD4+ and CD8+ CAR T cells separation from whole blood or buffy coat. Refer to protocol of PBMC separation and CD4 and CD8 cell separation.
2. Wash the CD4+ and CD8+ cells twice with pre-warmed TexMACS Medium (without supplement cytokines) by filling the falcon tube to the maximum volume and centrifuge at 300×g for 5 minutes. Aspirate supernatant completely.
3. Resuspend the CD4+ and CD8+ cells in pre-warmed TexMACS medium supplemented with 10 ng/mL IL-7, 5 ng/mL IL-15 and 3% FBS to a final concentration of 10\hat{ }6 cells/mL.
4. Plate the CD4+ cells and the CD8+ cells with a ratio of 1:1 by adding 0.5 ml of CD4+ cells and 0.5 mL of CD8+ cells into each well in a 24 well plate.
5. Add 10 μL of T cell TransAct to a final dilution of 1:100 in the cell culture and carefully resuspend.
6. Incubate for −36 hours at 37° C. with 5% CO₂ before transduction.

### Day 3: T Cell Transduction

7. Use fresh viral vectors for transduction if possible. Otherwise, thaw slowly frozen viral vectors on ice.
8. Remove 800 μL of the media slowly using a P1000 pipette from the side of the wells, taking care not to disrupt the bottom cell layer.
9. Add in dropwise 150 μL (one plate's worth of viral vectors) or 300 μL (2 plates' worth of viral vectors) to the T cells. Top up the wells with fresh supplemented TexMACS media up to 600 μL and add polybrene to each well at a final concentration of 4 μg/mL. From this time onwards, the T cells should be kept in an incubator for lentiviral work only.
10. After transduction, appropriate clean up procedures should be performed. Decontaminate the biosafety cabinet and the aspirator line by cleaning surfaces and running the line with 2% Virkon solution following by 70% ethanol. Dispose of contaminated waste, such as tips and serological pipettes etc. in a biohazard bag inside of the biosafety cabinet, seal the bag before taking the waste outside for disposal.

### Day 4 Onwards—T Cell Maintenance

11. Transduced T cells were maintained at in IL-7, IL-15 and 3% FBS containing TexMACS media.
12. Observe the T cell growth in the 24 well plate, transfer the cells to T75 flasks on day 2 post transduction.
13. Monitor the lactate levels in the media using CCS Analyzer daily. Replenish fresh media if the lactate level is >10 mmol/L; Ideally, keep the lactate level <4 mmol/L.

Day 9−Day 5 post T cell transduction—Expression analysis

Transduced T cells are counted on day 5 using a flow cytometer. A sample is taken for flow cytometry analysis to determine expression efficiency based on a standard flow cytometry protocol.

### Luciferase Expressing Cancer Cell Line Generation

Firefly luciferase lentiviral transfer plasmids used for viral vector production:
pRRLsin18.cPPT.EF1a_firefly_luciferase_T2A_EGFP.WPRE
pRRLsin18.cPPT.hPGK_firefly_luciferase_T2A_EGFP.WPRE

1. Plate cells at a density of 300,000 cells/well in a 24-well plate in a total volume of 850 μL.

2. Viral vectors containing the firefly luciferase gene are produced using a 4-plasmid transfection protocol as described in the lentiviral production section. Use fresh concentrated viral vectors for transduction if possible. Otherwise, thaw slowly frozen viral vectors on ice.

3. Add in dropwise 150 μL (one plate's worth of viral vectors) to the cells. From this time onwards, the cell should be kept in an incubator for lentiviral work only.

4. Incubator cells at 37° C. with 5% CO₂.

5. After transduction, appropriate clean up procedures should be performed. Decontaminate the biosafety cabinet and the aspirator line by cleaning surfaces and running the line with 2% Virkon solution following by 70% ethanol. Dispose of contaminated waste, such as tips and serological pipettes etc. in a biohazard bag inside of the biosafety cabinet, seal the bag before taking the waste outside for disposal.

6. On day 2 post transduction, transfer cells to T25 flasks.

7. On day 5 post transduction, count cells and take a sample for expression analysis by flow cytometry.

8. On day 7 post transduction, transduced cells are bulk sorted based on eGFP expression using a live cell sorter.

9. After expansion, bulk sorted cells are further sorted by single cell clones. Single cell clones are grown, expanded and frozen down to make stocks.

### Functional Assays

Target cells constitutively expressing firefly luciferase and eGFP (sorted and grown as single cell clones defined as high performance cell lines) were used in the functional assays to measure viability via bioluminescence and/or fluorescence. The amount of light emitted correlates to the total number of cells in bioluminescence and the fluorescent target cells identified via flow cytometry correlate with the total number of cells alive.

Effector and target cells were seeded according to indicated effector to target ratio (ET). The indicated ET ratio, e.g. 10:1 is always referred to the total number of T cells and the total number of target cells. As the CAR expressing fraction is different from the total number of T cells the ET ratio referred to the CAR expressing cells is indicated separately. Target cells were seeded with 25,000 or 50,000 cells per 96 well plate.

### Luciferase Killing Assay

Effector and target cells were seeded according to indicated effector to target ratios (ET). The BRIDGE molecules were added in the indicated format (Fab, IgG1) at the indicated concentrations. D-luciferin was added and bioluminescence was measured at the indicated time points after incubation was started under standard conditions in incubators at 37° C. and 5% CO₂ on a SpectraMaxi3.

Viability of cells was calculated according to a serial dilution derived bioluminescence activity curve of cells (100%, 75%, 50%, 25%, 10% and 0% target cells) and depicted in percent viable cells. In general, the lysis was calculated by (bioluminescence of testing condition−0% bioluminescence)/(100% bioluminescence−0% bioluminescence).

### Flow Based Kill Assay

Effector and target cells were seeded according to indicated effector to target ratios (ET), the BRIDGE molecules were added in the indicated format (Fab, IgG1) at the indicated concentrations. Cell number was measured at the indicated time points (24h or 48h) after incubation was started under standard conditions in incubators at 37° C. and 5% CO₂ on a MACSQuant16 flow cytometer according to standard protocols. The staining of cells included a viability dye to exclude all dead cells from the analysis. T cells were clearly differentiated from eGFP positive cancer cells via CD3. Further T cells were characterised by CD25 and CD69 as a measure for specific T cell activation according to standard protocols after 24h or 48 h. The final data analysis was performed by FlowJo10.

Antibody cocktail

| Channel | Antibody | Source | Cat# |
|---|---|---|---|
| R1 | CD3 APC | Miltenyi | 130-113-135 |
| R3 | CD25 APCVio770 | Miltenyi | 130-123-469 |
| V2 | CD69 VioGreen | Miltenyi | 130-112-611 |
| B1 | eGFP | Constitutive stable | Target cell marker |
| | | expression | |
| V1 | Viobility405/452 | Miltenyi | 130-109-816 |

### Flow Based Acquisition of Cytokine Secretion

Effector and target cells were seeded according to indicated effector to target ratios (ET), the BRIDGE molecules were added in the indicated format (Fab, IgG1) at the indicated concentrations. Supernatant was collected after 24h or 48h and measured at the indicated time points after incubation was started under standard conditions in incubators at 37° C. and 5% CO₂ on a MACSQuant16 flow cytometer according to standard protocols using the Miltenyi cytokine beads. The final data analysis was performed by FlowJo10.

### Example 2—Characterisation of Various Bridging Molecules

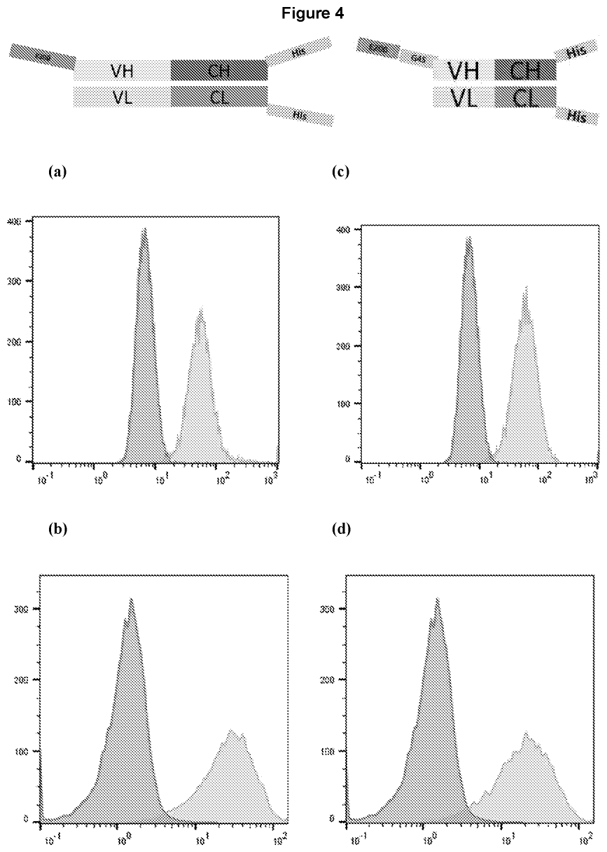

The results in FIGS. 4 to 7 show that bridging molecules comprising a targeting moiety in the form of a Fab or scFv (FMC63 clone; amino acid sequences described herein) that can bind to CD19, binds to CD19 on the surface of live cells and can present the dysfunctional P2X₇ receptor epitope moiety (e.g. E200 moiety) such that it is accessible by an anti-P2X₇ receptor antibody (BILO3s 2-2-1-Fc). The location of the dysfunctional P2X₇ receptor epitope moiety can vary and the targeting moiety can still bind to its target cell surface antigen and the dysfunctional P2X₇ receptor epitope moiety is still available for binding to an antibody. Note that BILO3s 2-2-1-Fc-AF647/HIS-FITC did not bind to control bridging molecules that did not contain the dysfunctional P2X₇ receptor epitope moiety, nor did the anti-HIS antibody bind to control bridging molecules that did contain a HIS tag (data not shown).

### Fab Format+/−Linker—Epitope on V_{H}

FIG. 4 shows that bridging molecules in Fab format with a single E200 epitope either directly linked to the V_{H} or via a linker binds to CD19 on JeKo-1 (mantle cell lymphoma) cell line and the E200 epitope is available for binding to an antibody.

### Scfv Format+/−Linker—Epitope on V_{H}

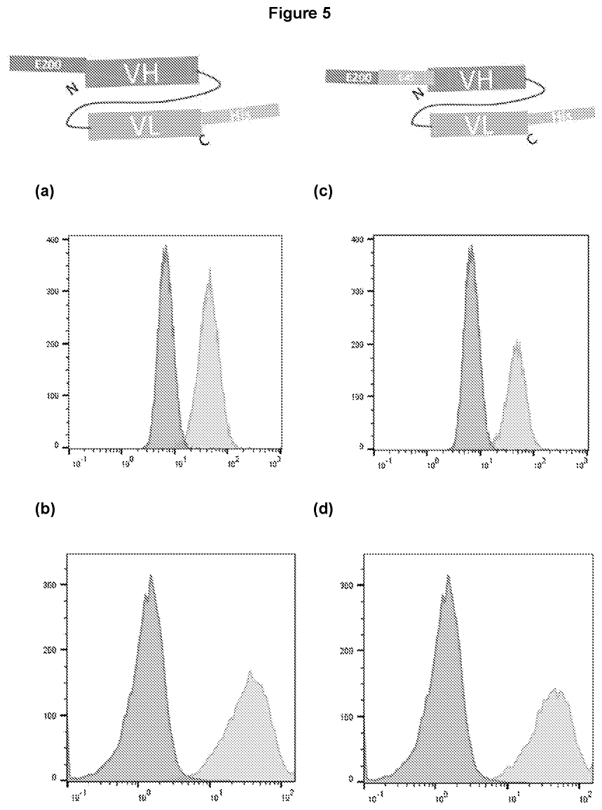

FIG. 5 shows that bridging molecules in scFv format with a single E200 epitope either directly linked to the V_{H} or via a linker binds to CD19 on JeKo-1 (mantle cell lymphoma) cell line and the E200 epitope is available for binding to an antibody.

### Fab Format+/−Linker—Epitope on V_{L}

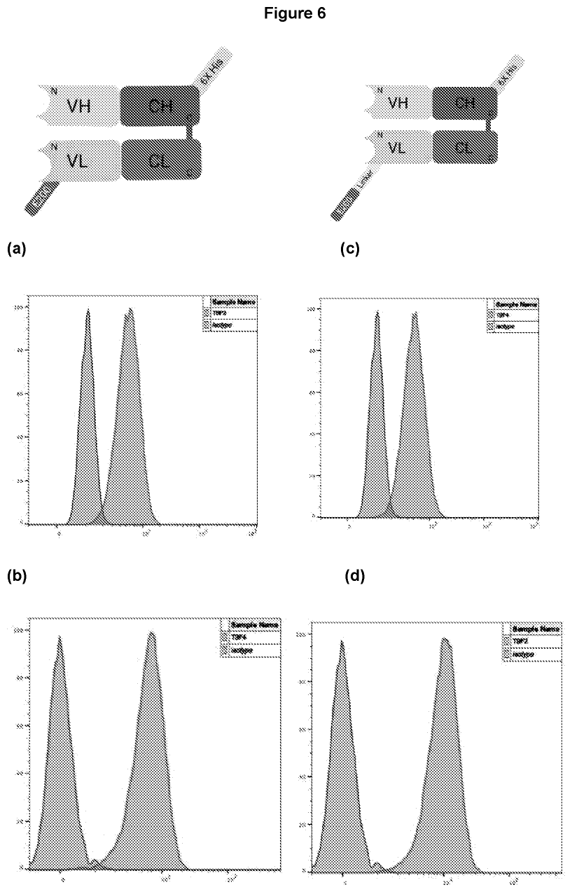

FIG. 6 shows that bridging molecules in Fab format with a single E200 epitope either directly linked to the V_{L} or via a linker binds to CD19 on JeKo-1 (mantle cell lymphoma) cell line and the E200 epitope is available for binding to an antibody.

### Scfv Format+/−Linker—Epitope on V_{L}

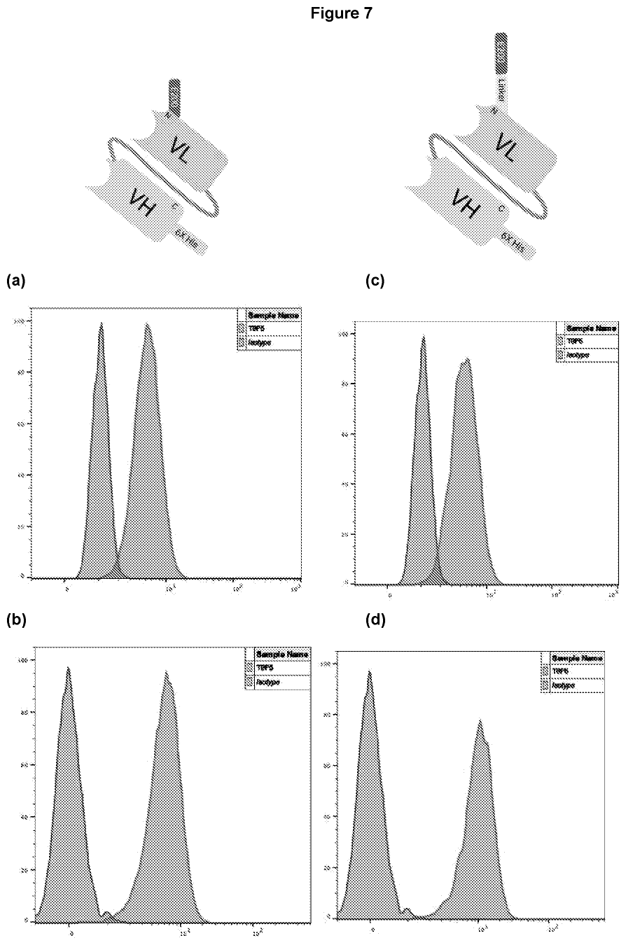

FIG. 7 shows that bridging molecules in scFv format with a single E200 epitope either directly linked to the V_{L} or via a linker binds to CD19 on JeKo-1 (mantle cell lymphoma) cell line and the E200 epitope is available for binding to an antibody.

### Bridging Molecules Targeting Various Antigens

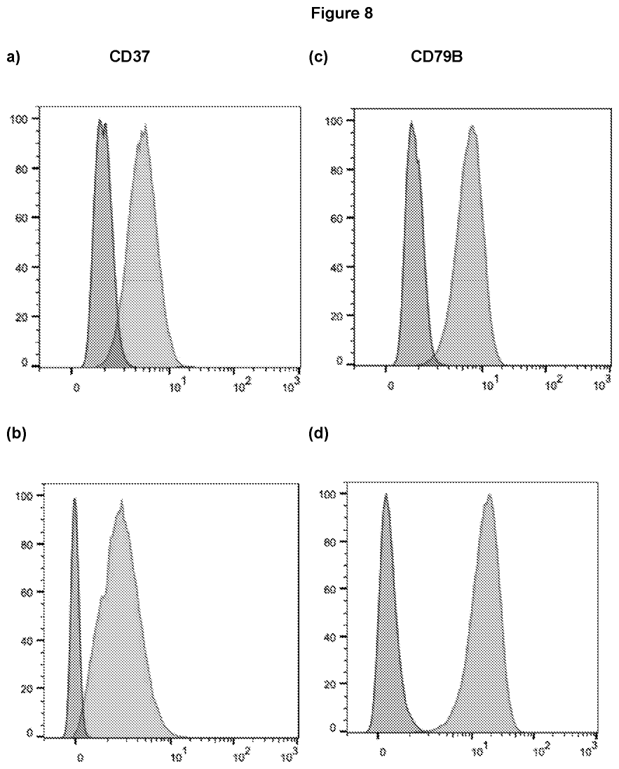

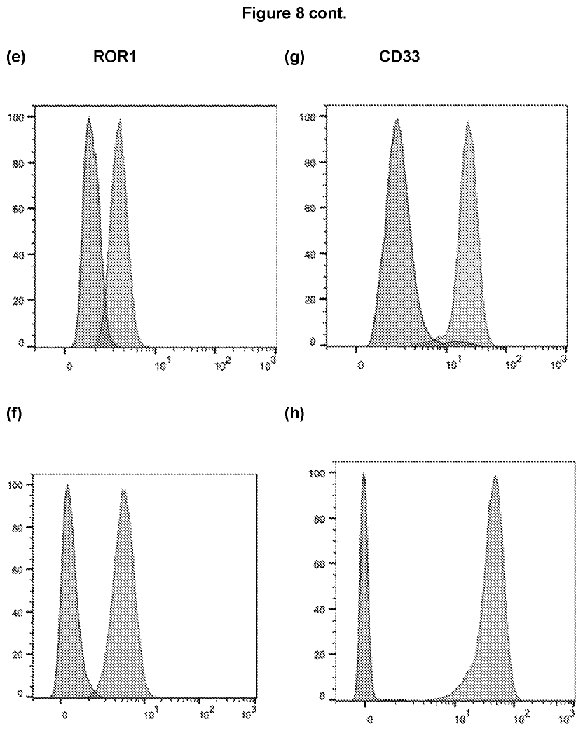

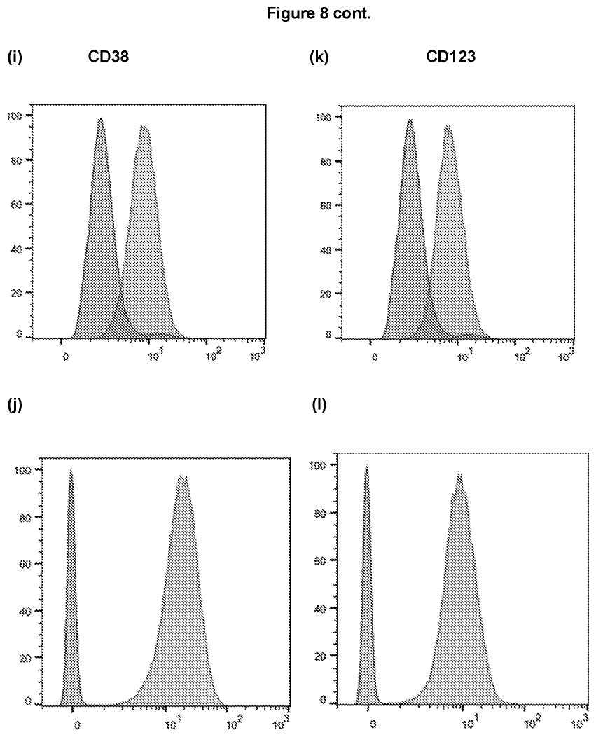

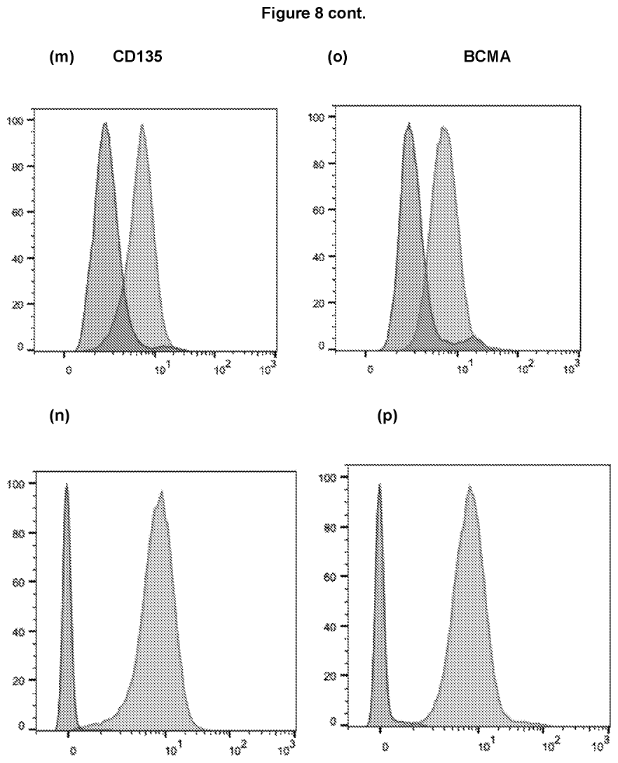

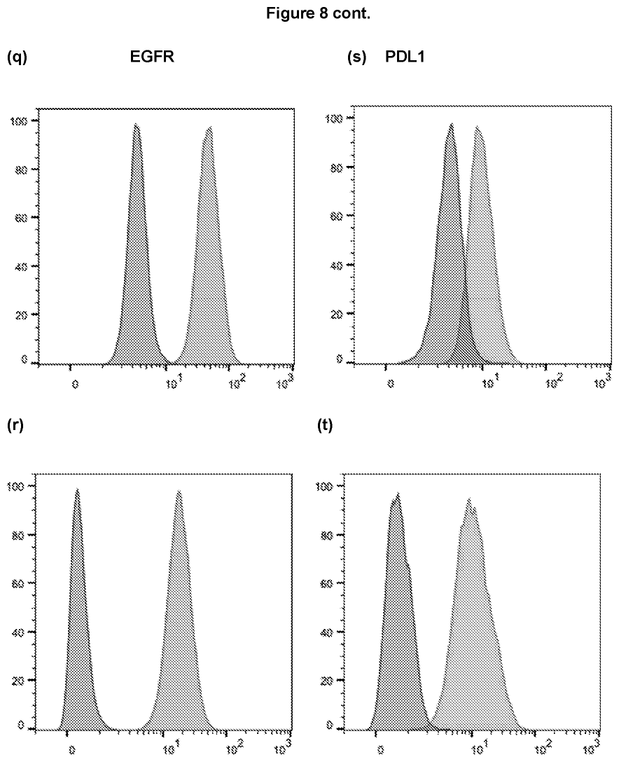

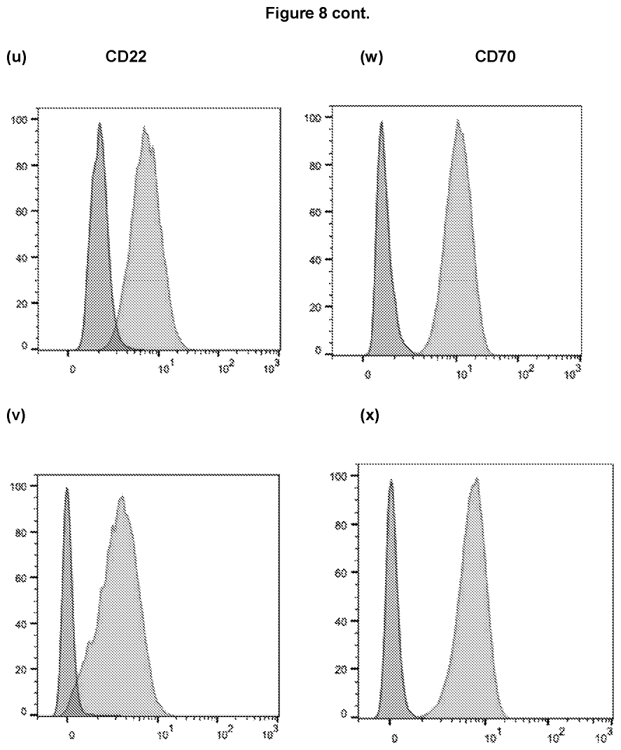

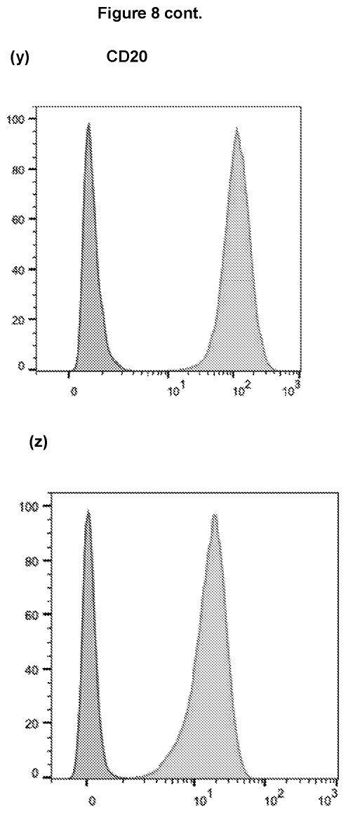

FIG. 8: Binding of bridging molecules to various antigens CD37, CD79B, ROR1, CD33, CD38, CD123, CD135, BCMA, EGFR, PDL1, CD22, CD70 and CD20. (a), (c), (e), (g), (i), (k), (m), (o), (q), (s), (u), (w) and (y) show anti-HIS antibody binding, (b), (d), (f), (h), (j), (1), (n), (p), (r), (t), (v), (x) and (z) show binding of antibody to dysfunctional P2X₇ receptor epitope.

CD37 targeting moieties derived from otlertuzumab, CD79B targeting moieties derived from polatuzumab, ROR1 targeting moieties derived from ROR1 APC (WO2016016344A1_D10v3), CD33 targeting moieties derived from lintuzumab, CD38 targeting moieties derived from daratumumab, CD123 targeting moieties derived from clone 32716, CD135 targeting moieties derived from 4G8, BCMA targeting moieties derived from clone CA8 J9M0, EGFR targeting moieties derived from necitumumab or matuzumab, PDL1 targeting moieties derived from atezolizumab, CD22 targeting moieties derived from m971-L7 (or inotuzumab (data not shown)), CD70 targeting moieties derived from cusatuzumab, CD19 targeting moieties derived from tafasitamab, CD20 targeting moieties derived from ofatumumab (or ocrelizumab (data not shown)). Exemplary amino acid sequences of bridging molecules targeting these antigens and derived from the antibodies mentioned immediately above are described in the sequence information table herein.

FIG. 8 shows binding of various bridging molecules to JeKo-1 (MCL) wild type cell line (CD37, CD79B, ROR1) Raji (Burkitt's lymphoma) wild type cell line (CD22, CD70, CD19, CD20, CD22), MOLM-13 (AML) wild type cell line (CD33, CD38, CD123 and CD135), RPMI 8226 (multiple myeloma) wild type cell line (CD33, BCMA and CD38), MDA-MB 231 (breast cancer) wildtype cell line (EGFR and PDL1) and PC-3 (prostate cancer) wild type cell line (EGFR).

### Example 3—Dose Dependent Increasing in Cell Binding by CD19 Targeting Bridging Molecule

JeKo-1 (MCL) CRL-3006™ wild type cell line purchased from ATCC as part of the NCI60 panel. The cells were cultured according to general recommendations and standards for this particular cell line.

FIG. 9 shows “painting” of JeKo-1 with CD19 targeted Fab bridging molecules in the illustrated format.

Cells were incubated at indicated concentrations with Fab bridging molecules.

CD33-targeted Fab-bridging molecules served as negative control in JeKo-1 at 10 ng/mL and 1000 ng/mL. CD19 targeted Fab-bridging molecule were used at 1 ng/mL, 10 ng/mL, 100 ng/mL and 1000 ng/mL.

The flow cytometric staining was undertaken in two steps according to standards in flow cytometric staining using the Fc block reagent (Miltenyi). First the target cells were incubated with Fab-bridging molecules at indicated concentrations for 15 min, washed three times and then the secondary antibodies anti-HIS FITC and the single domain antibody BIL03 2-2-1 AF647 was used at saturating concentrations (1 ug/mL) to indirectly stain the target cells via the 6×HIS and the nfP2X₇ E200 derived epitope on the bound Fab-bridging molecules. After 15 min of incubation the sample was washed and then analysed on a MACSQuant16 (Miltenyi). The flow data was analysed via FlowJo v10.7 (BD).

There is no expression of CD33 in JeKo-1 cells. CD19 staining showed increasing expression with increasing concentrations of CD19-targeted bridging molecules.

### Example 4—Dose Dependent Increase in Cell Binding by CD33 Targeting Bridging Molecule

MOLM-13 (AML) wild type cell line purchased from ATCC as part of the NCI60 panel. The cells were cultured according to general recommendations and standards for this particular cell line.

FIG. 10 “painting” of MOLM-13 with CD33 targeted Fab bridging molecules in the illustrated format.

Cells were incubated at indicated concentrations with Fab bridging molecules. CD19 targeted Fab bridging molecule served as negative control in MOLM-13 at 10 ng/mL and 1000 ng/mL, while CD33 targeted Fab-bridging molecule was used at 1 ng/mL, 10 ng/mL, 100 ng/mL and 1000 ng/mL.

The flow cytometric staining was undertaken in two steps according to standards in flow cytometric staining using the Fc block reagent (Miltenyi). First the target cells were incubated with Fab-bridging molecules at indicated concentrations for 15 min, washed three times and then the secondary antibodies anti-HIS FITC and the single domain antibody BIL03 2-2-1 AF647 was used at saturating concentrations (1 ug/mL) to indirectly stain the target cells via the 6×HIS and the nfP2X₇ E200 derived epitope on the bound Fab-bridging molecules. After 15 min of incubation the sample was washed and then analysed on a MACSQuand16 (Miltenyi). The flow data was analysed via FlowJo v10.7 (BD).

There is no expression of CD19 in MOLM-13 cells. CD33 staining showed increasing expression with increasing concentrations of CD33-targeted bridging molecules.

### Example 5—Detection of Marker Genes and Direct CAR Detection Via nfP2X₇ E200 Derived Peptide and Bridging Molecule

nfP2X₇ targeted bridging CAR effector cells (such as T cells or NK cells but not limited to) recognise cancer cells specifically. The epitope targeted in nfP2X₇ arises from a conformational change of the P2X₇ protein trimer and is exposed solely on cancer cells and not exposed on healthy cells.

The basic principle as well as the engagement of nfPX₇ CAR expressing effector cells via nfP2X₇ E200 derived peptide tagged bridging molecules and the different formats of bridging molecules is illustrated and outlined in FIGS. 1 to 3.

Thus using nfP2X₇ CAR in the absence of bridging molecules, the nfP2X₇ CAR expressing effector cells exhibit cancer-specific targeting.

In order to broaden the applicability to nfP2X₇ functionally negative cancers (very low or possibly negative for nfP2X₇) nfP2X₇ CAR expressing effector cells may be redirected to cancer cells via bridging molecules targeting cancer-associated antigens as illustrated for CD33 or cancer-specific antigens via TcR-like mAbs. The specificity of the bridging molecules is unlimited, which means any surface expressed target antigen or presented antigen in the context of MHC peptide presentation (class I and II) via TcR-like mAb or ligands may engage the nfP2X₇ bridging CAR expressing effector cells in the same mode of action.

In most cases, the dual-function of the nfP2X₇ bridging CAR expressing effector cells is utilised. It is a combination of scenario I and II which means that nfP2X₇ bridging CAR expressing effector cells are engaged directly to cancer cells via nfP2X₇ expressed on the cancer cells and additionally recruited to the cancer cells via bridging molecules targeting cancer-associated antigens as illustrated for CD33 or cancer-specific antigens via TcR-like mAbs.

### Example 6—“Painting” of MOLM-13 (AML) Wildtype Cell Line with CD33 Targeted Fab-Bridging Molecules

FIG. 11 illustrates the “painting” of MOLM-13 (AML) cells via CD33 targeted Fab-bridging molecules. The flow data shows isotype control (black), staining with BILO3s 2-2-1-Fc sd-mAb only at 1 ug/mL (blue) and the increase of staining via the combination of CD33 targeted Fab-bridging molecules and BILO3s 2-2-1-Fc (green). The increase of target molecules that can be recognised by the nfP2X₇ bridging CAR expressing effector cells translates into CAR-mediated effector function. The bridging technology therefore enhances the CAR function by increasing the targeting epitopes on the cancer cells.

### Example 7—Titration of CD33 Targeted Bridging Molecule Derived from Lintuzumab

MOLM-13 cells constitutively expressing Luciferase were co-incubated with increasing concentrations of CD33 targeted Fab-bridging molecules at the indicated concentrations. The Fab-bridging molecules bound to CD33 on MOLM-13 but do not exert any direct toxicity arising from complement-dependent cytotoxicity (CDC) or recruitment of effector cells (ADCC). No cells other than MOLM-13 were used in the assay. The CD33 targeted Fab-bridging molecules do not have any functional sites for CDC or ADCC like full-size antibodies.

There was no significant impact on viability at 4 hours and no consistent or dose-dependent toxicity after 24 hour incubation.

### Example 8—Activation of nfP2X₇ CAR-T Cells by CD33-Targeting Fab-Bridging Molecules

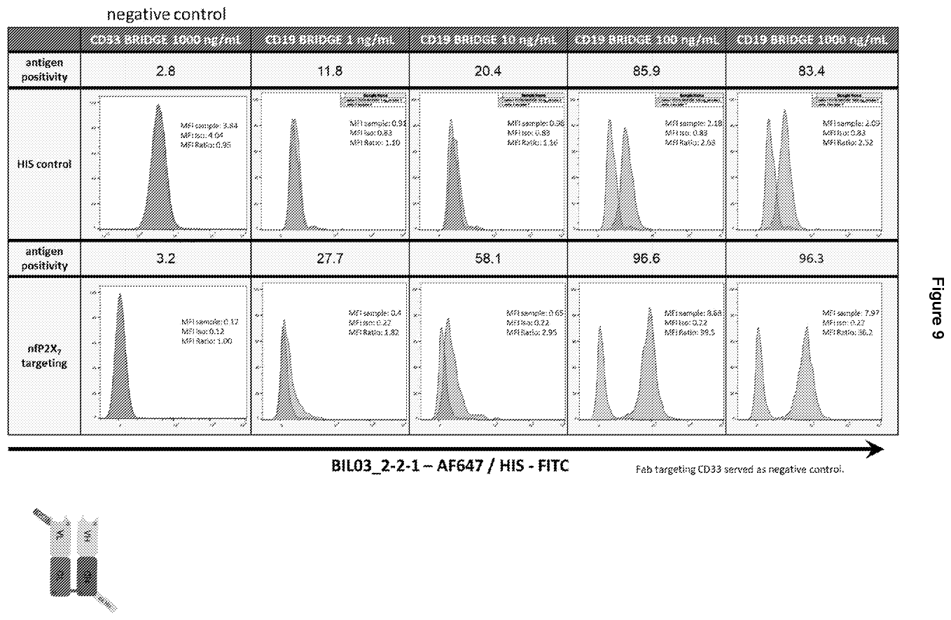

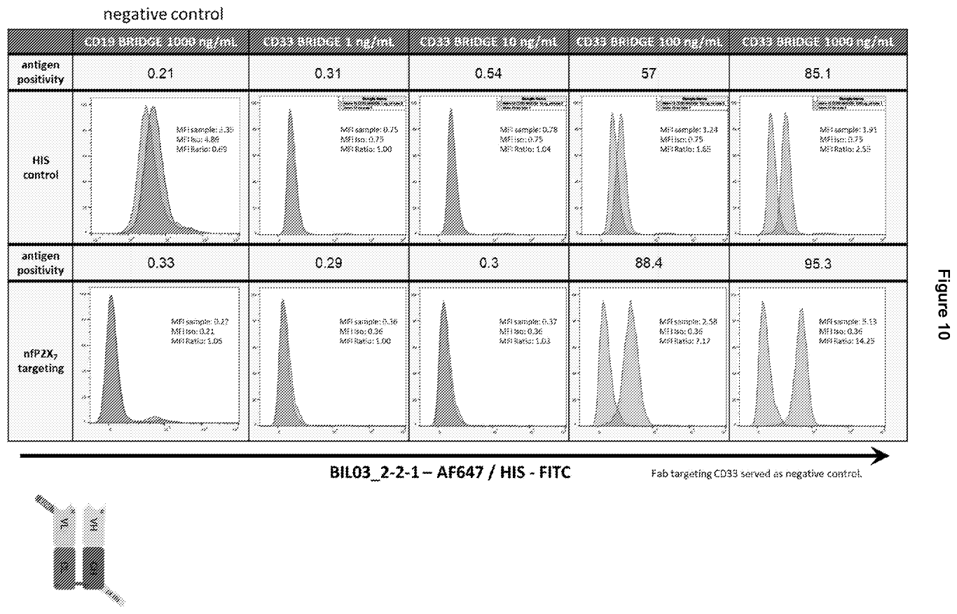

FIG. 12 shows a representative flow cytometric plot with direct comparison of untransduced T cells (left panel), CARCD07_hPGK (middle panel; CARCD07 is also referred to herein as CAR7) and CARCD07_EF1a (right panel; CARCD07 is also referred to herein as CAR7) expressing T cells. Both CAR T cells generated increased expression of the activation markers CD25 and CD69 in incubation with MOLM-13 at 20:1 ET ratio and CD33 targeted Fab-bridging molecules at 1000 ng/mL after 48 hours.

The CD33 Fab bridging molecule used in the experiments in Examples 8 and 9 was derived from Lintuzumab and the EGFR targeted bridging molecule derived from Necitumumab. The dysfunctional P2X₇ receptor epitope was linked directly to the V_{L} with a free N-terminus.

The CARCD07_hPGK CAR has the following domain structure from N-terminus to C-terminus: hPGK-CD8a SP-V_{H} BIL03 2-2-1-CD28-CD28T-CD28-CD137-CD3zeta-T2A-tEGFR.

CARCD07_EF1a CAR has the following domain structure from N-terminus to C-terminus: EF1a-CD8a SP-V_{H} BIL03 2-2-1-CD28-CD28T-CD28-CD137-CD3zeta-T2A-tEGFR.

### Example 9—Clearance of Leukaemic Cells by nfP2X₇ CAR-T Cells in the Presence of CD33 Bridging Molecules

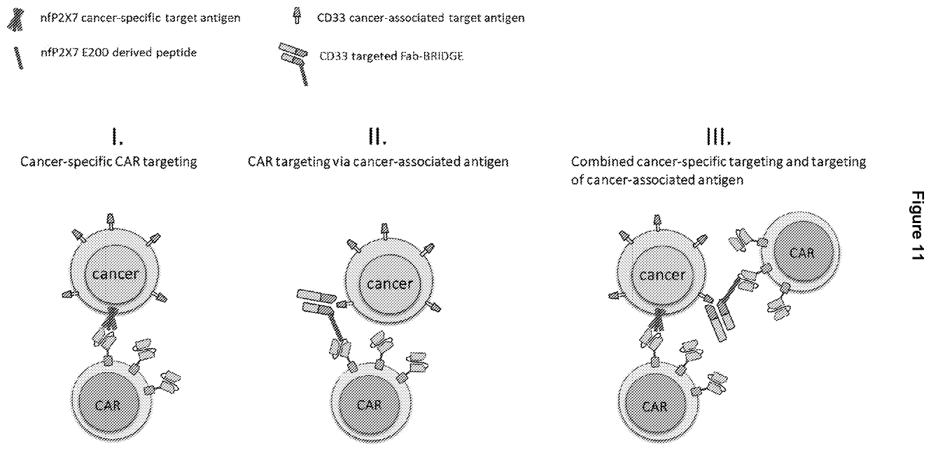

The flow cytometric plots of T cells and MOLM-13 at 20:1 ET ratio and CD33 targeted Fab-bridging molecules at 1000 ng/mL after 48 hours (corresponding to FIG. 12) showed a complete clearance of leukaemic cells in the presence of CARCD07_hPGK and CARCD07_EF1a whereas there was no impact on leukaemic cell number in the untransduced T cells (FIG. 13).

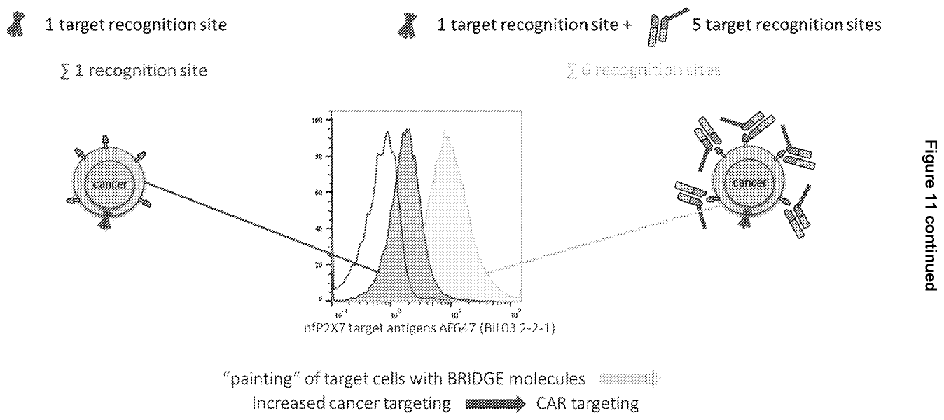

FIG. 14 shows flow cytometric plots that illustrate the dose-dependent clearance of leukaemic cells at indicated concentrations of CD33 targeted Fab bridging molecule concentrations of CARCD07_EF1a containing T cells and MOLM-13 at 20:1 ET ratio after 48 hours. Concentrations as low as 40 ng/mL showed almost complete elimination of leukaemic cells and complete elimination of leukaemic cells at 200 and 1000 ng/mL.

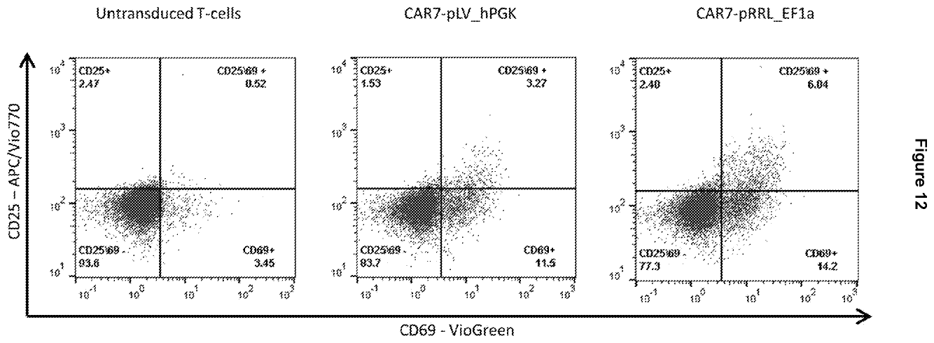

Specific lysis of MOLM-13 leukaemic cells by CARCD07_hPGK T cells at an ET ratio of 20:1 after 48 hour incubation with and without EGFR and CD33 targeted bridging molecules at indicated concentrations is illustrated in FIG. 15(a). Significant lysis in a dose dependent manner was found for increasing concentrations (40, 200 and 1000 ng/mL) of CD33 targeted Fab bridging molecules.

Specific lysis of MOLM-13 leukaemic cells by CARCD07_hEF1a T cells at an ET ratio of 20:1 after 48 hour incubation with and without EGFR and CD33 targeted bridging molecules at indicated concentrations is illustrated in FIG. 15(b). Significant lysis in a dose-dependent manner was found for increasing concentrations (40, 200 and 1000 ng/mL) of CD33 targeted Fab bridging molecules.

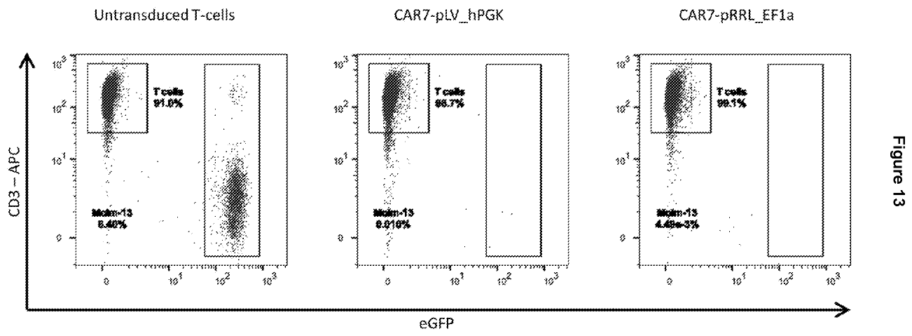

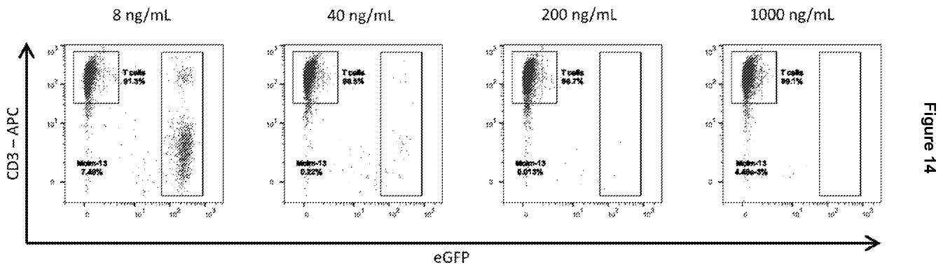

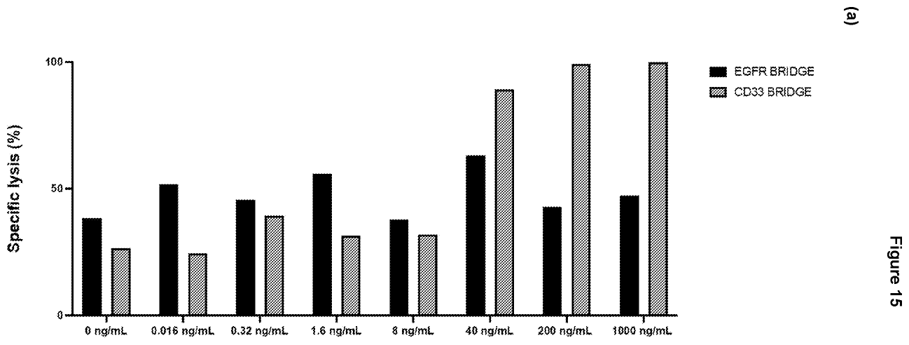

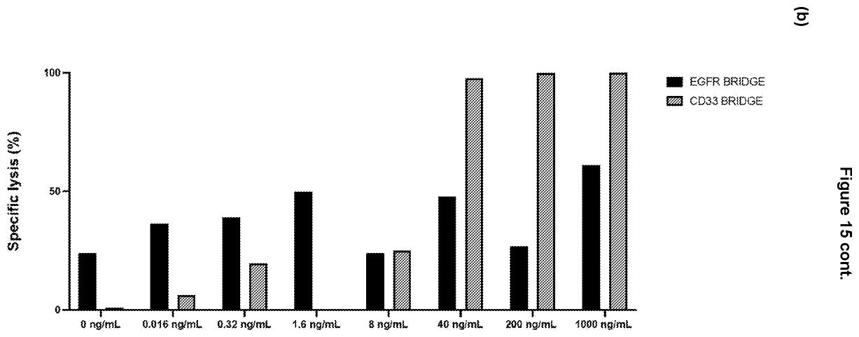

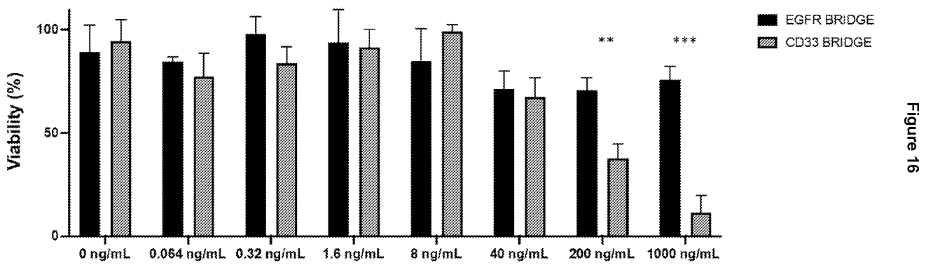

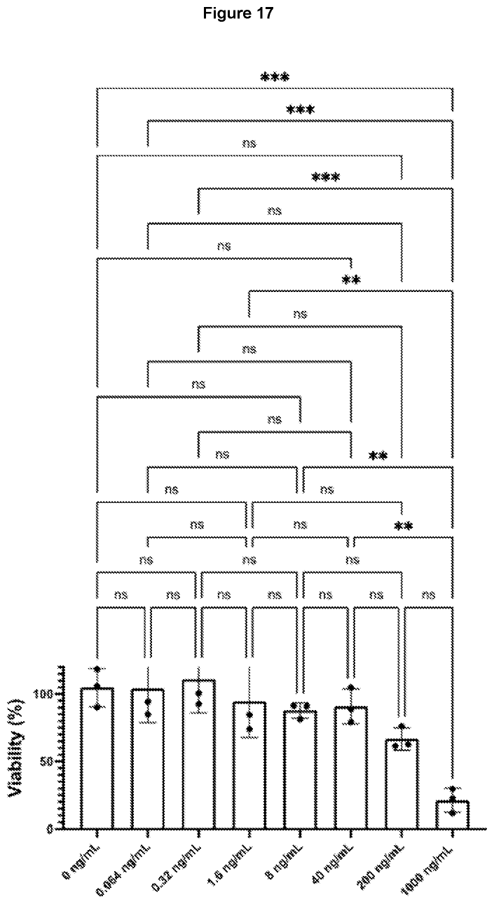

In a titration experiment EGFR and CD33 targeted Fab-bridging molecules were used to test the impact on killing of MOLM-13 by CARCD07_hPGK (FIG. 16). There was a significant difference in the viability of MOLM-13 after 24 hour incubation at 10:1 ET ratio between EGFR targeted bridging molecules and CD33 targeted bridging molecules at 1000 ng/mL. Statistical analysis was done by t-test. Alternative representation of the data from FIG. 16, shown in FIG. 17. There was no significant difference in the titration of the EGFR bridging molecules (data not shown). There were significant differences in the titration of the CD33 bridging molecules. Statistical analyses were performed by One-Way ANOVA and post-hoc test Tukey.

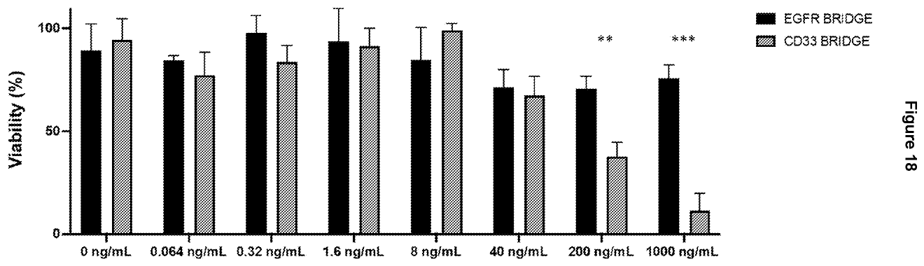

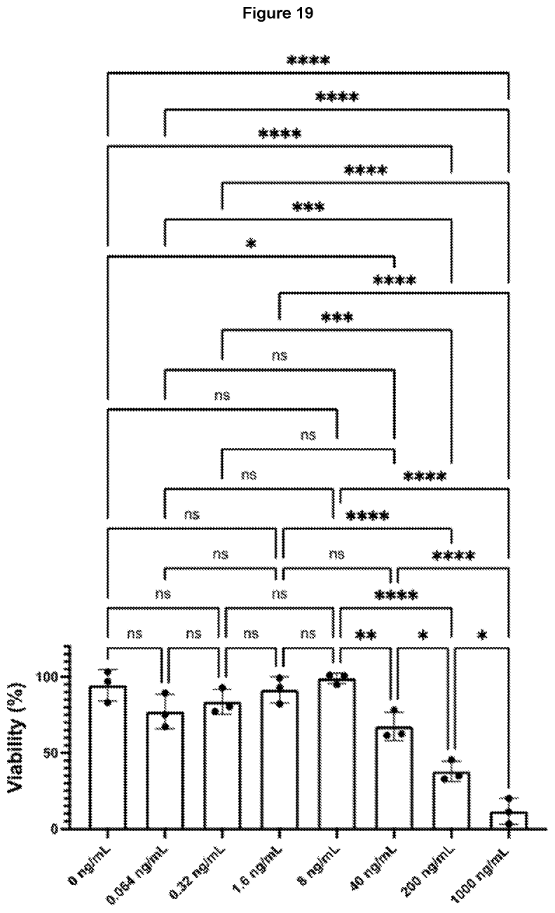

In a titration experiment, EGFR and CD33 targeted Fab-bridging molecules were used to test the effect of CARCD07_hPGK on killing of MOLM-13 (FIG. 18). There was a significant difference in the viability of MOLM-13 after 24 hour incubation at 20:1 ET ratio between the condition with EGFR targeted bridging molecules and CD33 targeted bridging molecules at 200 ng/mL and 1000 ng/mL. Statistical analysis was done by t-test. Alternative representation of the data from FIG. 18, shown in FIG. 19. There was no significant difference in the titration of the EGFR bridging molecules (data not shown). There were significant differences in the titration of the CD33 bridging molecules. Statistical analyses were performed by One-Way ANOVA and post-hoc test Tukey.

In a titration experiment, EGFR and CD33 targeted Fab-bridging molecules were used to test the impact on killing of MOLM-13 by CARCD07_hPGK (FIG. 20). There was a significant difference in the viability of MOLM-13 after 48 hour incubation at 10:1 ET ratio between EGFR targeted bridging molecules and CD33 targeted bridging molecules at 200 ng/mL and 1000 ng/mL. Statistical analysis was performed by t-test.

In a titration experiment, EGFR and CD33 targeted Fab-bridging molecules were used to test the difference in killing of MOLM-13 by CARCD07_hPGK (FIG. 21). There was a significant impact on the viability of MOLM-13 after 48 hour incubation at 20:1 ET ratio between EGFR targeted bridging molecules and CD33 targeted bridging molecules at 200 ng/mL and 1000 ng/mL. Statistical analysis was performed by t-test.

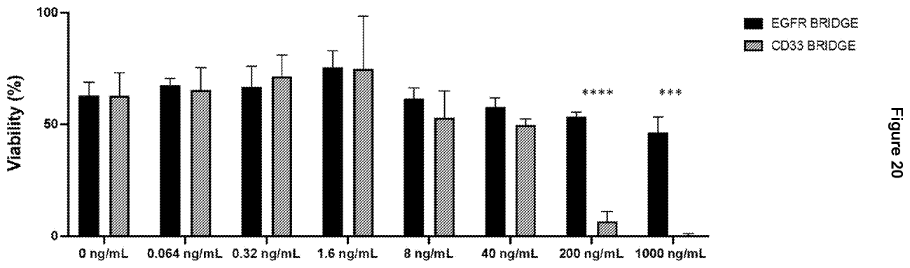

In a titration experiment, EGFR and CD33 targeted Fab-bridging molecules were used to test the effect of CARCD07_EF1a on killing of MOLM-13 by (FIG. 22).

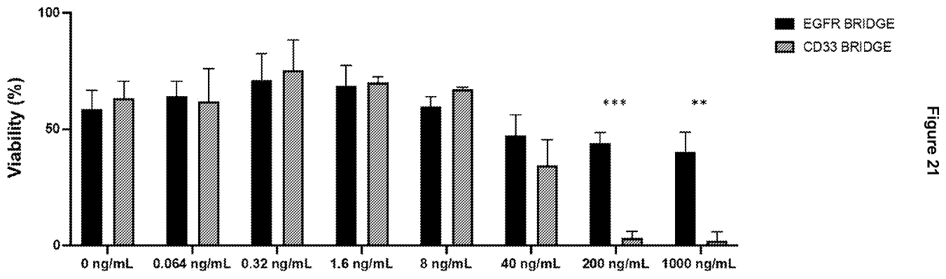

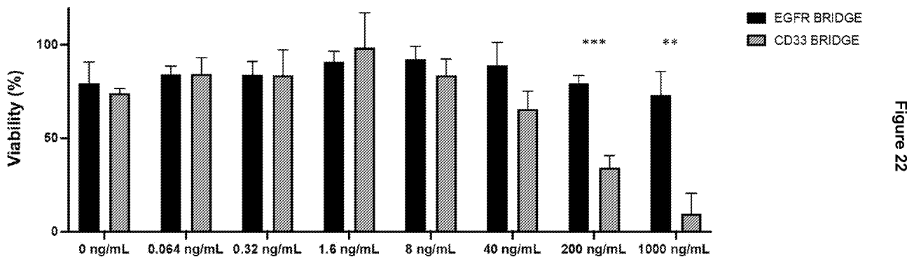

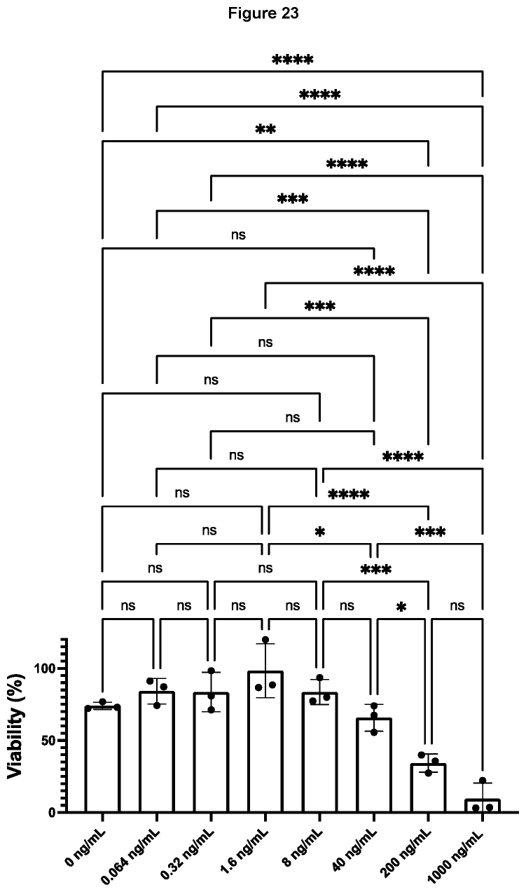

There was a significant difference in the viability of MOLM-13 after 24 hour incubation at 10:1 ET ratio between EGFR targeted bridging molecules and CD33 targeted bridging molecules at 200 ng/mL and 1000 ng/mL. Statistical analysis was done by t-test. Alternative representation of the data from FIG. 22, shown in FIG. 23. There was no significant difference in the titration of the EGFR bridging molecules (data not shown). There were significant differences in the titration of the CD33 bridging molecules. Statistical analyses were performed by One-Way ANOVA and post-hoc test Tukey.

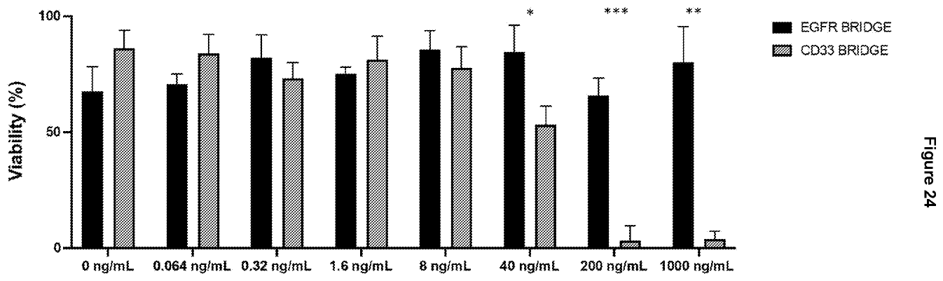

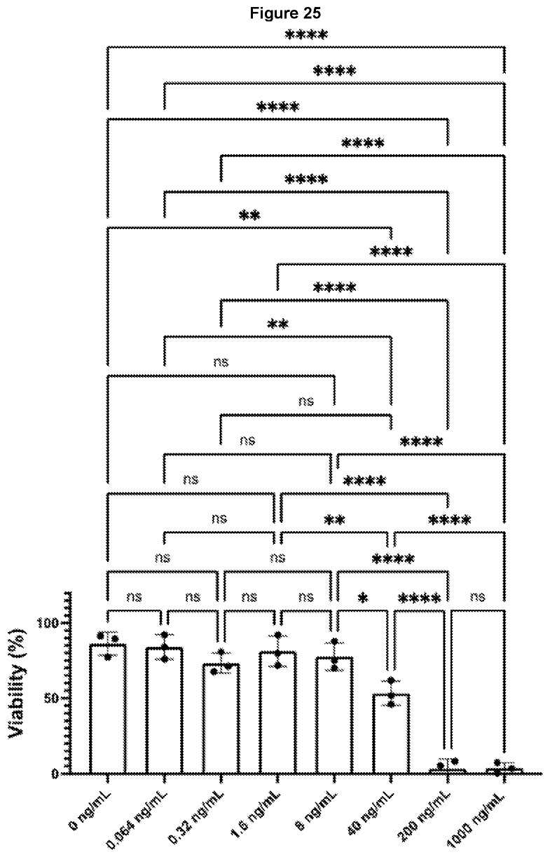

In a titration experiment, EGFR and CD33 targeted Fab-bridging molecules were used to test the effect of CARCD07_EF1a on killing of MOLM-13 by (FIG. 24). There was a significant difference in the viability of MOLM-13 after 24 hour incubation at 20:1 ET ratio between the EGFR targeted bridging molecules and CD33 targeted bridging molecules at 40 ng/mL, 200 ng/mL and 1000 ng/mL. Statistical analysis was done by t-test. An alternative representation of the data from FIG. 24 is shown in FIG. 25. There was no significant difference in the titration of the EGFR bridging molecules (data not shown). There were significant differences in the titration of the CD33 bridging molecules. Statistical analyses were performed by One-Way ANOVA and post-hoc test Tukey.

In a titration experiment EGFR and CD33 targeted Fab-bridging molecules were used to test the impact on killing of MOLM-13 by CARCD07_EF1a (FIG. 26). There was a significant impact on the viability of MOLM-13 after 48 hour incubation at 10:1 ET ratio between the condition with EGFR targeted bridging molecules and CD33 targeted bridging molecules at 40 ng/mL, 200 ng/mL and 1000 ng/mL. Statistical analysis was performed by t-test.

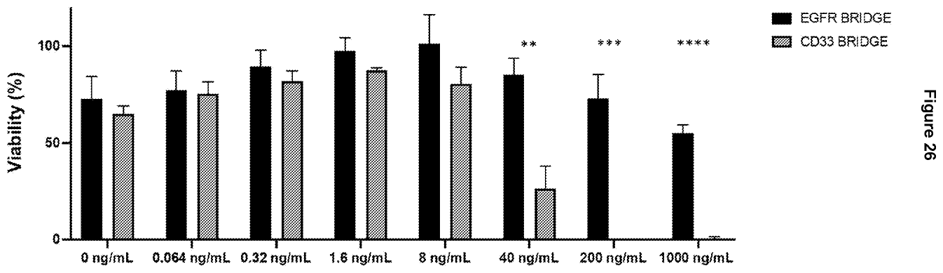

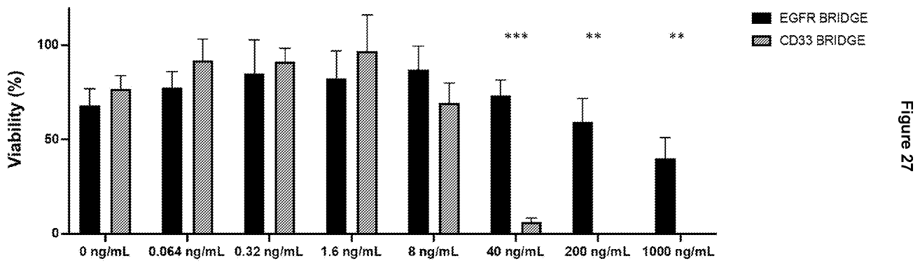

In a titration experiment, EGFR and CD33 targeted Fab-bridging molecules were used to test the effect of CARCD07_EF1a on killing of MOLM-13 by (FIG. 27). There was a significant difference in the viability of MOLM-13 after 48 hour incubation at 20:1 ET ratio between the EGFR targeted bridging molecules and CD33 targeted bridging molecules at 40 ng/mL, 200 ng/mL and 1000 ng/mL. Statistical analysis was performed by t-test.

In a titration experiment, EGFR targeted Fab-bridging molecules were used to test the effect of untransduced T cells, CARCD07_hPGK and CARCD07_EF1a effector cells on the killing of MOLM-13 at an ET ratio 10:1 after 24 hour incubation. There was no significant difference in the titration of the EGFR bridging molecules between the three effector cell populations (data not shown).

### Example 10—Activation of Various nfP2X₇ CAR-T Cells by CD33-Targeting Fab-Bridging Molecules-Titration

In a flow based assay, activation was assessed by measuring the up-regulation of CD25 and CD69 expression on MOLM-13 induced by untransduced T cells, and CARCD07_hPGK and CARCD07_EF1a expressing T cells at a 20:1 ET ratio after 48 hour incubation both with and without EGFR or CD33 targeted Fab-bridging molecules.

Both the CARCD07_hPGK as well as CARCD07_EF1a expressing T cells showed a significantly increased expression of the activation markers CD25 and CD69 in the presence of EGFR and the CD33 bridging molecules compared to untransduced T cells (data not shown).

### Example 11—Cell Killing by Various nfP2X₇ CAR-T Cells in the Presence of CD33-Targeting Fab-Bridging Molecules—Titration

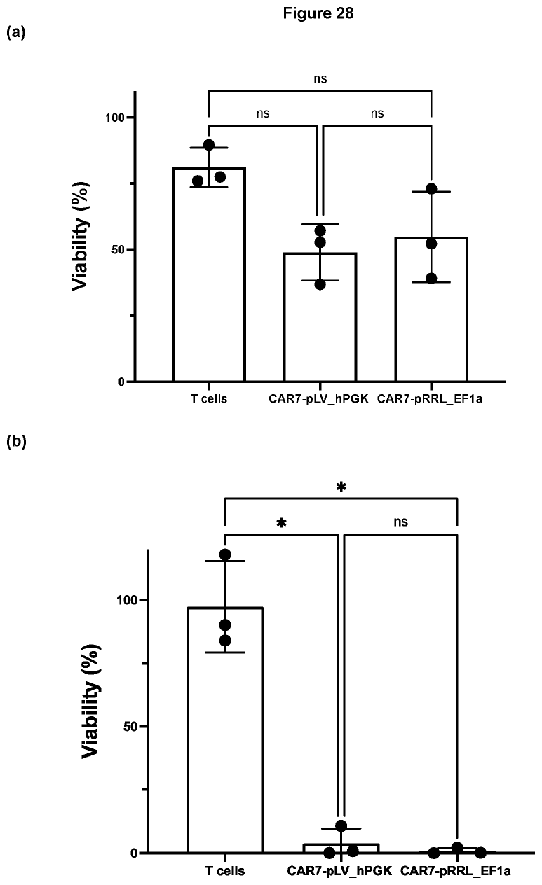

In a cell killing assay, cytotoxicity was measured by quantification of residual leukaemic cells compared with a control. Elimination of leukaemic cells at concentrations of 40, 200 and 1000 ng/mL of the EGFR bridging molecules showed there was no significant difference between untransduced T cells compared with CAR T cells (FIG. 28(a)). However, the elimination of leukaemic cells in the presence of CD33 bridging molecules at the same concentrations of 40, 200 and 1000 ng/mL showed a significant difference between untransduced T cells compared with both CARCD07 transduced T cells (hPGK and EF1a) (FIG. 28(b)).

### Example 12—Cell Killing by Various nfP2X₇ CAR-T Cells in the Presence of CD33-Targeting Fab-Bridging Molecules'Titration

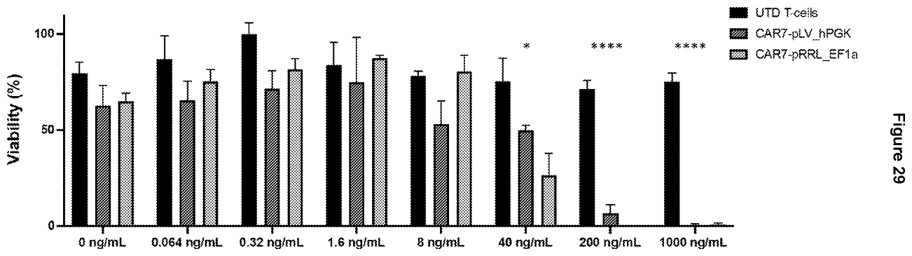

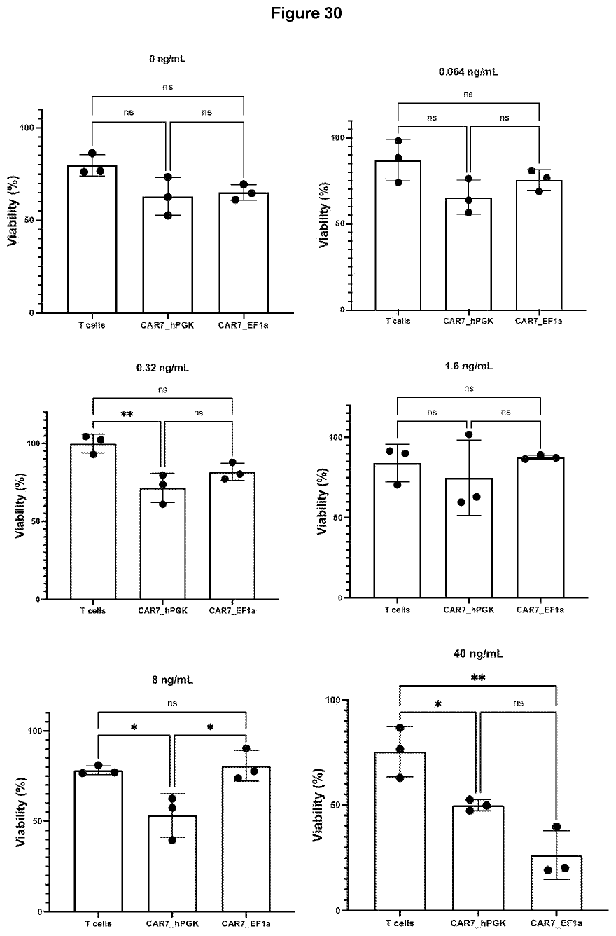

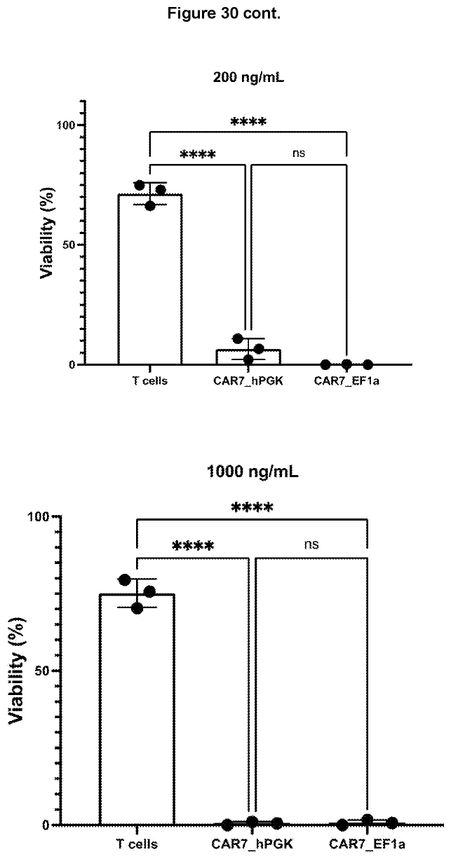

In a titration experiment, CD33 targeted Fab-bridging molecules were used to test the effect of untransduced T cells, CARCD07_hPGK and CARCD07_EF1a effector cells on the killing of MOLM-13 by at an ET ratio of 10:1 after 48 hour incubation (FIG. 29). There was a consistent significant difference observed between the three effector cell populations resulting from titrating the CD33 bridging molecules over the concentration range 40 ng/mL, 200 ng/mL and 1000 ng/mL (FIG. 30). Statistical analysis was done by One-Way ANOVA and post-hoc test Tukey to compare the effects of the named three effector cell types compared with the concentration of EGFR bridging molecules.

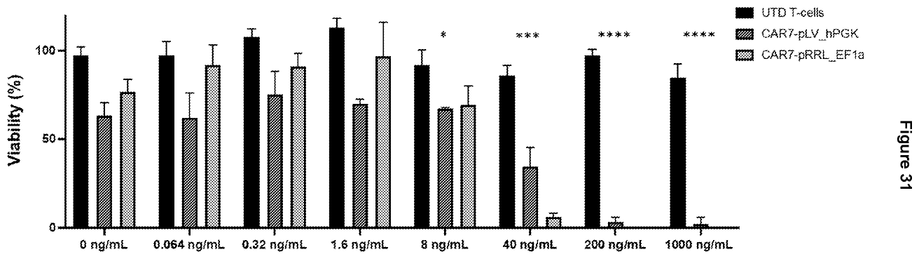

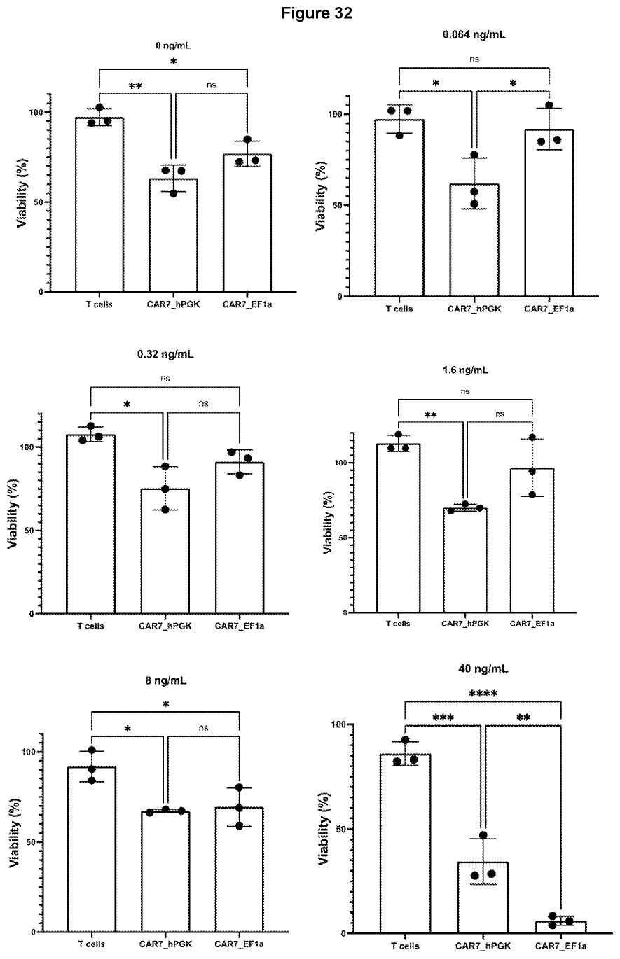

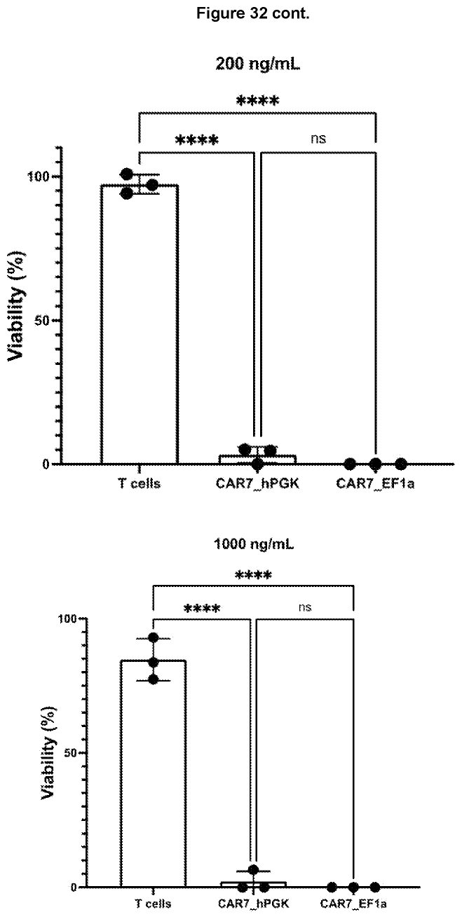

In a titration experiment, CD33 targeted Fab-bridging molecules were used to test the effect of untransduced T cells, CARCD07_hPGK and CARCD07_EF1a effector cells on the killing of MOLM-13 at an ET ratio of 20:1 after 48 hour incubation (FIG. 31). There was a consistent significant difference observed between the three effector cell populations resulting from titrating the CD33 bridging molecules over the concentration range 40 ng/mL, 200 ng/mL and 1000 ng/mL (FIG. 32). Statistical analyses were performed by One-Way ANOVA and post-hoc test Tukey to compare the effects of the named three effector cell types compared with the concentration of EGFR bridging molecules.

### Example 13—Cell Killing by nfP2X₇ CAR-T Cells in the Presence of CD19-Targeting Bridging Molecules in Fab and IgG1 Format and with Different Dysfunctional P2X₇ Receptor Epitope Moieties

Bridging molecules were generated with CD19 binding targeting moieties in different formats'Fab and IgG1. Moreover, those bridging molecules in those different formats were generated with different dysfunctional P2X₇ receptor epitope moieties.

In these experiments the nfP2X₇ CAR was as per CARCD07_EF1a CAR but with a CD8alpha spacer between the V_{H} BIL03 2-2-1 and the CD28TM (described herein as CAR10).

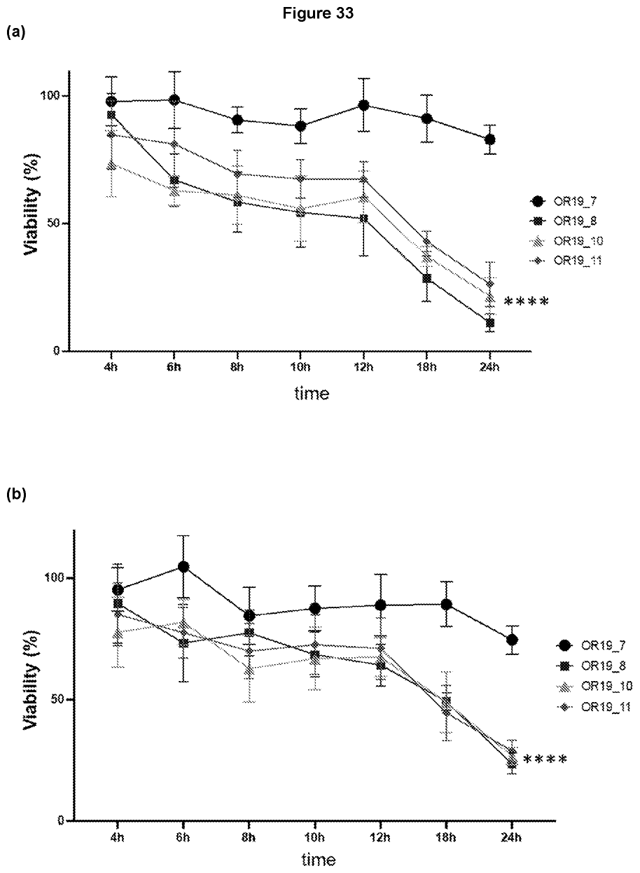

These CD19 targeted Fab or IgG1-bridging molecules were used to test the impact on killing of JeKo-1 by CAR10 (FIG. 33; Fab formats shown in (a), IgG1 formats shown in (b)). There was a significant difference in the viability of JeKo1 after, at least, a 24 hour incubation at 10:1 ET ratio between all CD19 targeted bridging molecules at 100 ng/mL that contain a dysfunctional P2X₇ receptor epitope moiety (ie OR19_8, 10 and 11) compared with the control bridging molecule that does not have a dysfunctional P2X₇ receptor epitope moiety (OR19_7). Statistical analysis was performed by One-Way ANOVA and post-hoc test Tukey. There was no significant difference in the titration of the CD19 bridging molecules containing a dysfunctional P2X₇ receptor epitope moiety. Similar results were achieved with other nfP2X₇ CARs (data not shown).

These data show that the cell killing of CAR-T cells can be potentiated significantly by bridging molecules irrespective of targeting moiety format and dysfunctional P2X₇ receptor epitope moiety.

### Example 14—Cell Killing by nfP2X₇ CAR-T Cells in the Presence of CD19-Targeting Bridging Molecules in Fab Format and with Different Dysfunctional P2X₇ Receptor Epitope Moieties

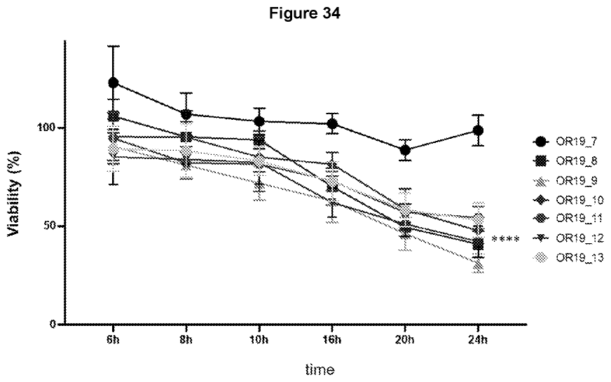

Similar experiments were performed as described above in Example 13, however CD19 bridging molecules with a larger array of dysfunctional P2X₇ receptor epitope moieties were tested. As shown in FIG. 34, CD19 bridging molecules comprising a dysfunctional P2X₇ receptor epitope moiety significantly reduced the cell viability of the target JeKo-1 cells whereas the control bridging molecule without a dysfunctional P2X₇ receptor epitope moiety (OR19_7), did not.

A summary of the various Fab bridging molecules with different dysfunctional P2X₇ receptor epitope moieties (or the absence of) is shown below (all Fab light chains are paired with the Fab heavy chain described herein—CD19, tafasitamab, B020-2_HC, SEQ ID NO: 52/143)

TABLE 3

| | Fab Light | dysfunctional | |
|---|---|---|---|
| | chain | P2X₇ receptor | Moiety |
| Bridging | SEQ ID | epitope | SEQ ID |
| molecule | NO | moiety | NO |
| OR19_7 | 144 | None | — |
| OR19_8 | 144 | E200 (C to S) | 4 |
| OR19_9 | 144 | E200_G4S | 15 |
| OR19_10 | 144 | E200_3*G4S | 168 |
| OR19_11 | 144 | E200_extended | 10 |
| | | peptide 17 | |
| | | (27 aa) | |
| OR19_12 | 144 | E200_extended | 6 |
| | | peptide 17v2 | |
| | | (22 aa) | |
| OR19_13 | 144 | E200_extended | 17 |
| | | peptide 17v3 | |
| | | (24 aa) | |
| OR19_14 | 144 | E200_extended | 18 |
| | | peptide 17v4 | |
| | | (22 aa) | |
| OR19_15 | 144 | E200_extended | 19 |
| | | peptide 17v5 | |
| | | (19 aa) | |
| OR19_NEW_001 | 144 | E200_extended | 20 |
| | | peptide 17v6 | |
| | | (22 aa) | |
| OR19_NEW_002 | 144 | E200_extended | 21 |
| | | peptide 17v7 | |
| | | (25 aa) | |
| OR19_NEW_003 | 144 | E200_extended | 22 |
| | | peptide 17v8 | |
| | | (30 aa) | |
| OR19_NEW_004 | 144 | E200_extended | 23 |
| | | peptide 17v9 | |
| | | (35 aa) | |
| OR19_NEW_005 | 144 | E200_extended | 24 |
| | | peptide 17v10 | |
| | | (30 aa) | |
| OR19_NEW_006 | 144 | E200_extended | 25 |
| | | peptide 17v11 | |
| | | (32 aa) | |
| OR19_NEW_007 | 144 | E200_extended | 26 |
| | | peptide 17v12 | |
| | | (35 aa) | |
| OR19_NEW_008 | 144 | E200_extended | 27 |
| | | peptide 17v13 | |
| | | (25 aa) | |
| OR19_NEW_009 | 144 | E200_extended | 28 |
| | | peptide 17v14 | |
| | | (22 aa) | |
| OR19_NEW_010 | 144 | E200_extended | 29 |
| | | peptide 17v15 | |
| | | (30 aa) | |
| OR19_NEW_011 | 144 | E200_extended | 30 |
| | | peptide 17v16 | |
| | | (27 aa) | |
| OR19_NEW_012 | 144 | E200_2*G4S | 16 |

### Example 15—Cell Killing by Different nfP2X₇ CAR-T Cells in the Presence of CD19-Targeting Bridging Molecules in Fab or IgG Format

Similar experiments were performed as described above in Example 13, using CAR-T cells having one of three different CAR architectures. As shown in FIG. 35, three different CARs (CAR7, CAR10 and CAR16), when expressed on T cells, and in combination with CD19 bridging molecules comprising a dysfunctional P2X₇ receptor epitope moiety, significantly reduced the cell viability of the target JeKo-1 cells. In all experiments, the bridging molecule OR19_10 was used.

These results demonstrate that the bridging molecule system is effective at reducing cell viability when using CAR-T cells having differing CAR architectures.

### Example 16—Binding and Cell Killing Using Alternative Bridging Molecule Constructs

The inventors developed a further generation of bridging molecules, this time comprising IgG1 hinge regions in the region linking the E200 epitope to the targeting moiety. The sequences of the tumour-specific antigen epitope moiety of these bridging molecules is provided in SEQ ID NOs: 365 to 400 (correlating to epitope binding moiety of the bridges referred to as B1 to B36 in Table 4 below). The tumour-specific antigen epitope moieties were linked to anti-CD19 or anti-CD33 Fabs, as described herein in Table 1.

The bridging molecules were then assessed for their ability to bind both anti-nfP2X₇ CARs and target cells (ie cells expressing the antigen to which the targeting moiety is designed). As summarised in the table below, all anti-CD19 bridging molecules were determined to be able to specifically bind to Jeko-1 cells (expressing CD19) and to T cells expressing nfP2X₇ CAR (data shown for anti-CD19 bridging molecule only).

### Table 4: Binding Capacity of New CD19 BRIDGE Variants to the CD19 Positive Cell Line JeKo-1.

JeKo-1 cells were incubated with different CD19 BRiDGE variants at saturating concentrations. After thorough washing, either a secondary anti-HIS-tag antibody or a single-domain antibody BIL03s (conjugated with AF647) staining at saturating concentrations was used to semi-quantify the binding capacity to CD19 on JeKo-1. The MFI intensity correlates with the number of secondary bound antibodies bound to the CD19 BRiDGE variants that are only detected if they are bound to the JeKo-1 cells. MFI: median fluorescence intensity. BLitz: Bio-Layer interferometry (BLI) technology using HIS-tag identification tips. MFIR: median fluorescence intensity ratio corresponds to a relative index that is calculated by the testing condition MFI divded by the control MFI (HIS-tag or BIL03s) This corresponds to the MFI measured without the CD19 BRIDGE primary staining (=isotype control) and the testing condition with the CD19 BRIDGE and subsequent staining with the secondary antibody. The higher the MFIR, the stronger the binding. Targeting CD19 via a tafasitamab-based CD19 BRIDGE shows the highest binding capacity to CD19 with the original E200 BRIDGE.

| | MFI HisTag | MFI Bil03 | MFIR | MFIR BIL03 | | MFIR BIL03 to |
|---|---|---|---|---|---|---|
| | FITC | AF647 | HisTag FITC | AF647 | BLItz | HisTag |
| B1 | 3.21 | 3.19 | 4.168831169 | 6.934782609 | 0.1865 | 1.663483679 |
| B2 | 3.47 | 4.17 | 4.506493506 | 9.065217391 | 0.1861 | 2.011590026 |
| B3 | 3.63 | 5.43 | 4.714285714 | 11.80434783 | 0.2941 | 2.503952569 |
| B4 | 3.89 | 9.02 | 5.051948052 | 19.60869565 | 0.1344 | 3.881412764 |
| B5 | 3.4 | 5.5 | 4.415584416 | 11.95652174 | 0.2225 | 2.707800512 |
| B6 | 3.33 | 5.29 | 4.324675325 | 11.5 | 0.1863 | 2.659159159 |
| B7 | 3.31 | 7.57 | 4.298701299 | 16.45652174 | 0.2533 | 3.828254302 |
| B8 | 3.58 | 10.6 | 4.649350649 | 23.04347826 | 0.1661 | 4.956278844 |
| B9 | 3 | 4.82 | 3.896103896 | 10.47826087 | 0.2446 | 2.68942029 |
| B10 | 3.27 | 7.71 | 4.246753247 | 16.76086957 | 0.2415 | 3.946749103 |
| B11 | 3.08 | 6.43 | 4 | 13.97826087 | 0.2553 | 3.494565217 |
| B12 | 3.36 | 9.08 | 4.363636364 | 19.73913043 | 0.2533 | 4.523550725 |
| B13 | 2.99 | 5.28 | 3.883116883 | 11.47826087 | 0.2805 | 2.95594009 |
| B14 | 3.12 | 7.27 | 4.051948052 | 15.80434783 | 0.2564 | 3.900431996 |
| B15 | 2.91 | 8.58 | 3.779220779 | 18.65217391 | 0.1317 | 4.935454953 |
| B16 | 2.86 | 8.08 | 3.714285714 | 17.56521739 | 0.2583 | 4.72909699 |
| B17 | 2.98 | 9.3 | 3.87012987 | 20.2173913 | 0.1844 | 5.223956814 |
| B18 | 3.03 | 10.9 | 3.935064935 | 23.69565217 | 0.3574 | 6.021667384 |
| B19 | 2.7 | 4.9 | 3.506493506 | 10.65217391 | 0.2721 | 3.03784219 |
| B20 | 2.61 | 4.17 | 3.38961039 | 9.065217391 | 0.1823 | 2.674412794 |
| B21 | 2.56 | 4.06 | 3.324675325 | 8.826086957 | 0.2726 | 2.654721467 |
| B22 | 2.54 | 4.85 | 3.298701299 | 10.54347826 | 0.23 | 3.196251284 |
| B23 | 2.39 | 6.82 | 3.103896104 | 14.82608696 | 0.2123 | 4.776605421 |
| B24 | 2.47 | 8.45 | 3.207792208 | 18.36956522 | 0.216 | 5.726544622 |
| B25 | 2.32 | 5 | 3.012987013 | 10.86956522 | 0.2368 | 3.607571214 |
| B26 | 2.36 | 5.53 | 3.064935065 | 12.02173913 | 0.232 | 3.922347089 |
| B27 | 2.5 | 9.6 | 3.246753247 | 20.86956522 | 0.1628 | 6.427826087 |
| B28 | 2.38 | 8.78 | 3.090909091 | 19.08695652 | 0.2021 | 6.175191816 |
| B29 | 2.33 | 7.44 | 3.025974026 | 16.17391304 | 0.184 | 5.345027057 |
| B30 | 2.35 | 7.48 | 3.051948052 | 16.26086957 | 0.2042 | 5.328029602 |
| B31 | 2.13 | 5.83 | 2.766233766 | 12.67391304 | 0.25 | 4.581649316 |
| B32 | 2.3 | 8.99 | 2.987012987 | 19.54347826 | 0.1712 | 6.542816635 |
| B33 | 2.25 | 7.12 | 2.922077922 | 15.47826087 | 0.1655 | 5.297004831 |
| B34 | 2.19 | 7.04 | 2.844155844 | 15.30434783 | 0.2197 | 5.380980743 |
| B35 | 2.31 | 9.49 | 3 | 20.63043478 | 0.2333 | 6.876811594 |
| B36 | 2.29 | 9.15 | 2.974025974 | 19.89130435 | 0.2135 | 6.68834251 |
| CD19 | 2.67 | 11.6 | 3.467532468 | 25.2173913 | | 7.2724312 |
| PURIFIED | | | | | | |
| BRIDGE | | | | | | |
| NO BRIDGE | 0.77 | 0.46 | 1 | 1 | | |

Table 5: Binding Capacity of the New CD19 BRIDGE Variants to nfP2X₇ CAR 1.

nfP2X₇ CAR T cells were incubated with different BRiDGE variants at saturating concentrations. After thorough washing, secondary anti-HIS-tag antibody staining at saturating concentrations was used to semi-quantify the binding capacity of nfP2X₇ CAR T cells to the E200 tag variant on the CD19 BRiDGE. The MFI intensity correlates with the number of secondary bound antibodies bound to the BRiDGEs that are only detected if they are bound to the CAR T cells. EGFR was used as a marker gene to detect the CAR expression. Thus, the EGFR+ T cell population was defined as the CAR expressing cells and the EGFR− T cell population was defined as the CAR negative subset. MFI: median fluorescence intensity. EGFR: epidermal growth factor receptor. BLItz: Bio-Layer interferometry (BLI) technology using HIS-tag identification tips. HIS-tag MFI Ratio: relative index that is calculated by the testing condition MFI divided by the control MFI, which corresponds to the MFI measured on EGFR+ T cells (defined as CAR positive cells) divided by the MFI measured on the EGFR− T cells (defined as CAR negative cells). Only CAR positive cells should be able to specifically bind the BRiDGE molecules via the E200 tag (nfP2X₇-derived CAR targeted epitope). The higher the MFIR, the stronger the binding. Some of the new BRiDGE variants lead to a significantly improved binding to the nfP2X₇ targeted CAR.

TABLE 6
Binding capacity of new the CD33 BRiDGE variants to the CD33 positive cell line MOLM-13.

| | | | | HisTag MFI | |
|---|---|---|---|---|---|
| | MFI HisTag FITC | MFI HisTag | HisTag MFI | Ratio | |
| | in EGFR+ | FITC in EGFR− | Ratio of | (background | |
| | Population | Population | EGFR+/EGFR− | 0.44) | BLItz |
| B1 | 2.1 | 0.52 | 4.0 | 4.8 | 0.1865 |
| B2 | 2.07 | 0.51 | 4.1 | 4.7 | 0.1861 |
| B3 | 2.05 | 0.5 | 4.1 | 4.7 | 0.2941 |
| B4 | 2.05 | 0.5 | 4.1 | 4.7 | 0.1344 |
| B5 | 2.08 | 0.5 | 4.2 | 4.7 | 0.2225 |
| B6 | 1.99 | 0.49 | 4.1 | 4.5 | 0.1863 |
| B7 | 2.22 | 0.55 | 4.0 | 5.0 | 0.2533 |
| B8 | 2.53 | 0.58 | 4.4 | 5.8 | 0.1661 |
| B9 | 2.32 | 0.56 | 4.1 | 5.3 | 0.2446 |
| B10 | 2.26 | 0.53 | 4.3 | 5.1 | 0.2415 |
| B11 | 2 | 0.5 | 4.0 | 4.5 | 0.2553 |
| B12 | 2.12 | 0.51 | 4.2 | 4.8 | 0.2533 |
| B13 | 2.13 | 0.52 | 4.1 | 4.8 | 0.2805 |
| B14 | 2.41 | 0.54 | 4.5 | 5.5 | 0.2564 |
| B15 | 2.61 | 0.54 | 4.8 | 5.9 | 0.1317 |
| B16 | 2.34 | 0.51 | 4.6 | 5.3 | 0.2583 |
| B17 | 2.25 | 0.53 | 4.2 | 5.1 | 0.1844 |
| B18 | 2.25 | 0.53 | 4.2 | 5.1 | 0.3574 |
| B19 | 2.16 | 0.53 | 4.1 | 4.9 | 0.2721 |
| B20 | 2.11 | 0.51 | 4.1 | 4.8 | 0.1823 |
| B21 | 2.19 | 0.54 | 4.1 | 5.0 | 0.2726 |
| B22 | 2.21 | 0.55 | 4.0 | 5.0 | 0.23 |
| B23 | 2.34 | 0.53 | 4.4 | 5.3 | 0.2123 |
| B24 | 2.13 | 0.54 | 3.9 | 4.8 | 0.216 |
| B25 | 2.28 | 0.57 | 4.0 | 5.2 | 0.2368 |
| B26 | 2.17 | 0.52 | 4.2 | 4.9 | 0.232 |
| B27 | 2.35 | 0.57 | 4.1 | 5.3 | 0.1628 |
| B28 | 2.24 | 0.53 | 4.2 | 5.1 | 0.2021 |
| B29 | 2.25 | 0.54 | 4.2 | 5.1 | 0.184 |
| B30 | 2.12 | 0.49 | 4.3 | 4.8 | 0.2042 |
| B31 | 2.67 | 0.68 | 3.9 | 6.1 | 0.25 |
| B32 | 2.61 | 0.66 | 4.0 | 5.9 | 0.1712 |
| B33 | 2.39 | 0.61 | 3.9 | 5.4 | 0.1655 |
| B34 | 2.41 | 0.56 | 4.3 | 5.5 | 0.2197 |
| B35 | 2.51 | 0.61 | 4.1 | 5.7 | 0.2333 |
| B36 | 2.41 | 0.58 | 4.2 | 5.5 | 0.2135 |
| CD19 PURIFIED | 1.2 | 0.44 | 2.7 | 2.7 | |
| BRIDGE | | | | | |
| NO BRIDGE | 0.62 | 0.35 | 1.8 | | |

MOLM-13 cells were incubated with different CD33 BRIDGE variants at saturating concentrations. After thorough washing, secondary anti-HIS-tag antibody or single-domain antibody BIL03s (conjugated with AF647) staining at saturating concentrations was used to semi-quantify the binding capacity to CD33 on MOLM-13. The MFI intensity correlates with the number of secondary antibodies bound to the CD33 BRIDGE variants that are only detected if they are bound to the MOLM-13 cells. MFI: median fluorescence intensity. BLItz: Bio-Layer interferometry (BLI) technology using HIS-tag identification tips. MFIR: median fluorescence intensity ratio corresponds to a relative index that is calculated by the testing condition MF divided by the control MB (HIS-tag or BIL03s), which corresponds to the MFI measured without the CD33 BRIDGE primary staining (=isotype control) and the testing condition with CD33 BRIDGE and subsequent staining with the secondary antibody. The higher the MFIR, the stronger the binding. There is a significant increase in the BIL03s MFIR with the new BRIDGE variants compared to the old variants for the targeting of CD33 with lintuzumab-based BRIDGEs.

TABLE 7
Binding capacity of new CD33 BRIDGE variants to nfP2X₇ CAR 1.

| | MFI HisTag | MFI Bil03 | MFIR HisTag | MFIR BIL03 | | MFIR BIL03 |
|---|---|---|---|---|---|---|
| | FITC | AF647 | FITC | AF647 | BLItz | to HisTag |
| B1 | 6.11 | 25.6 | 9.119402985 | 40 | 0.1865 | 4.386252046 |
| B2 | 5.59 | 23.8 | 8.343283582 | 37.1875 | 0.1861 | 4.457177996 |
| B3 | 6.76 | 27.6 | 10.08955224 | 43.125 | 0.2941 | 4.274223373 |
| B4 | 5.53 | 24.3 | 8.253731343 | 37.96875 | 0.1344 | 4.600192134 |
| B5 | 5.86 | 24.8 | 8.746268657 | 38.75 | 0.2225 | 4.430460751 |
| B6 | 6.25 | 25.8 | 9.328358209 | 40.3125 | 0.1863 | 4.3215 |
| B7 | 5.52 | 24.7 | 8.23880597 | 38.59375 | 0.2533 | 4.684386322 |
| B8 | 6.53 | 29.7 | 9.746268657 | 46.40625 | 0.1661 | 4.761437596 |
| B9 | 6.33 | 28.7 | 9.447761194 | 44.84375 | 0.2446 | 4.746494866 |
| B10 | 6.86 | 31.4 | 10.23880597 | 49.0625 | 0.2415 | 4.791818513 |
| B11 | 7.39 | 34.2 | 11.02985075 | 53.4375 | 0.2553 | 4.844807172 |
| B12 | 6.08 | 26.9 | 9.074626866 | 42.03125 | 0.2533 | 4.631733141 |
| B13 | 6.78 | 28.5 | 10.11940299 | 44.53125 | 0.2805 | 4.400580752 |
| B14 | 6.68 | 28 | 9.970149254 | 43.75 | 0.2564 | 4.388098802 |
| B15 | 7.64 | 31.7 | 11.40298507 | 49.53125 | 0.1317 | 4.343709097 |
| B16 | 6.27 | 28.7 | 9.358208955 | 44.84375 | 0.2583 | 4.791915869 |
| B17 | 7.36 | 32.6 | 10.98507463 | 50.9375 | 0.1844 | 4.636973505 |
| B18 | 5.79 | 24.2 | 8.641791045 | 37.8125 | 0.3574 | 4.375539724 |
| B19 | 5.35 | 21.5 | 7.985074627 | 33.59375 | 0.2721 | 4.207067757 |
| B20 | 5.67 | 21.5 | 8.462686567 | 33.59375 | 0.1823 | 3.969631834 |
| B21 | 6.04 | 25.8 | 9.014925373 | 40.3125 | 0.2726 | 4.471750828 |
| B22 | 4.61 | 19.8 | 6.880597015 | 30.9375 | 0.23 | 4.496339479 |
| B23 | 6.82 | 28.3 | 10.17910448 | 44.21875 | 0.2123 | 4.344070748 |
| B24 | 6.25 | 26.7 | 9.328358209 | 41.71875 | 0.216 | 4.47225 |
| B25 | 7.29 | 28.5 | 10.88059701 | 44.53125 | 0.2368 | 4.092721193 |
| B26 | 5.75 | 25.9 | 8.582089552 | 40.46875 | 0.232 | 4.71548913 |
| B27 | 5.55 | 25.1 | 8.28358209 | 39.21875 | 0.1628 | 4.734515766 |
| B28 | 6.33 | 27.8 | 9.447761194 | 43.4375 | 0.2021 | 4.597650079 |
| B29 | 5.38 | 23.4 | 8.029850746 | 36.5625 | 0.184 | 4.553322491 |
| B30 | 6.29 | 27.9 | 9.388059701 | 43.59375 | 0.2042 | 4.643531399 |
| B31 | 7.09 | 30.3 | 10.58208955 | 47.34375 | 0.25 | 4.473950987 |
| B32 | 6.69 | 27.7 | 9.985074627 | 43.28125 | 0.1712 | 4.334594544 |
| B33 | 6.96 | 29.8 | 10.3880597 | 46.5625 | 0.1655 | 4.482309626 |
| B34 | 6.98 | 29.8 | 10.41791045 | 46.5625 | 0.2197 | 4.469466332 |
| B35 | 6.62 | 27.4 | 9.880597015 | 42.8125 | 0.2333 | 4.33298716 |
| B36 | 6.72 | 29 | 10.02985075 | 45.3125 | 0.2135 | 4.517764137 |
| CD33 | 7.12 | 11.6 | 10.62686567 | 18.125 | | 1.705582865 |
| PURIFIED | | | | | | |
| BRIDGE | | | | | | |
| NO BRIDGE | 0.67 | 0.64 | 1 | 1 | | |

nfP2X₇ CAR T cells were incubated with different BRiDGE variants at saturating concentrations. After thorough washing, secondary anti-HIS-tag antibody staining at saturating concentrations was used to semi-quantify the binding capacity of nfP2X₇ CAR T cells to the E200 tag variant on the CD33 BRiDGE. The MFI intensity correlates with the number of secondary bound antibodies bound to the BRiDGEs that are only detected if they are bound to the CAR T cells. EGFR was used as a marker gene to detect the CAR expression. Thus, the EGFR+ T cell population was defined as the CAR expressing cells and the EGFR− T cell population was defined as the CAR negative subset. MFI: median fluorescence intensity. EGFR: epidermal growth factor receptor. BLItz: Bio-Layer interferometry (BLI) technology using HIS-tag identification tips. HIS-tag MFI Ratio: relative index that is calculated by the testing condition MFI divided by the control MFI, which corresponds to the MFI measured on EGFR+ T cells (defined as CAR positive cells) divided by the MFI measured on the EGFR− T cells (defined as CAR negative cells). Only CAR positive cells should be able to specifically bind the BRiDGE molecules via the E200 tag (nfP2X₇-derived CAR targeted epitope). The higher the MFIR, the stronger the binding. Some of the new BRiDGE variants lead to a significantly improved binding to the nfP2X₇ targeted CAR.

| | | | | HisTag MFI | |
|---|---|---|---|---|---|
| | MFI HisTag | MFI HisTag | HisTag MFI | Ratio | |
| | FITC in EGFR+ | FITC in EGFR− | Ratio of | (background | |
| | Population | Population | EGFR+/EGFR− | 0.4) | BLItz |
| B1 | 1.08 | 0.44 | 2.5 | 2.7 | 0.1865 |
| B2 | 1.11 | 0.45 | 2.5 | 2.8 | 0.1861 |
| B3 | 1.04 | 0.44 | 2.4 | 2.6 | 0.2941 |
| B4 | 1.68 | 0.54 | 3.1 | 4.2 | 0.1344 |
| B5 | 1.4 | 0.5 | 2.8 | 3.5 | 0.2225 |
| B6 | 1.96 | 0.57 | 3.4 | 4.9 | 0.1863 |
| B7 | 1.45 | 0.49 | 3.0 | 3.6 | 0.2533 |
| B8 | 1.34 | 0.47 | 2.9 | 3.4 | 0.1661 |
| B9 | 1.16 | 0.42 | 2.8 | 2.9 | 0.2446 |
| B10 | 1.99 | 0.57 | 3.5 | 5.0 | 0.2415 |
| B11 | 1.2 | 0.45 | 2.7 | 3.0 | 0.2553 |
| B12 | 1.55 | 0.5 | 3.1 | 3.9 | 0.2533 |
| B13 | 1.35 | 0.47 | 2.9 | 3.4 | 0.2805 |
| B14 | 1.44 | 0.49 | 2.9 | 3.6 | 0.2564 |
| B15 | 1.5 | 0.49 | 3.1 | 3.8 | 0.1317 |
| B16 | 1.83 | 0.54 | 3.4 | 4.6 | 0.2583 |
| B17 | 1.58 | 0.5 | 3.2 | 4.0 | 0.1844 |
| B18 | 1.79 | 0.52 | 3.4 | 4.5 | 0.3574 |
| B19 | 1.29 | 0.45 | 2.9 | 3.2 | 0.2721 |
| B20 | 1.29 | 0.45 | 2.9 | 3.2 | 0.1823 |
| B21 | 1.14 | 0.41 | 2.8 | 2.9 | 0.2726 |
| B22 | 1.44 | 0.47 | 3.1 | 3.6 | 0.23 |
| B23 | 1.71 | 0.51 | 3.4 | 4.3 | 0.2123 |
| B24 | 1.82 | 0.52 | 3.5 | 4.6 | 0.216 |
| B25 | 1.82 | 0.54 | 3.4 | 4.6 | 0.2368 |
| B26 | 1.22 | 0.43 | 2.8 | 3.1 | 0.232 |
| B27 | 1.2 | 0.43 | 2.8 | 3.0 | 0.1628 |
| B28 | 1.58 | 0.49 | 3.2 | 4.0 | 0.2021 |
| B29 | 1.73 | 0.51 | 3.4 | 4.3 | 0.184 |
| B30 | 1.63 | 0.49 | 3.3 | 4.1 | 0.2042 |
| B31 | 1.34 | 0.45 | 3.0 | 3.4 | 0.25 |
| B32 | 1.59 | 0.48 | 3.3 | 4.0 | 0.1712 |
| B33 | 1.69 | 0.5 | 3.4 | 4.2 | 0.1655 |
| B34 | 1.31 | 0.43 | 3.0 | 3.3 | 0.2197 |
| B35 | 2.15 | 0.56 | 3.8 | 5.4 | 0.2333 |
| B36 | 1.65 | 0.5 | 3.3 | 4.1 | 0.2135 |
| CD33 PURIFIED | 1.58 | 0.4 | 4.0 | 4.0 | |
| BRIDGE | | | | | |
| NO BRIDGE | 0.23 | 0.4 | 0.6 | | |

Subsequently, the bridging molecules were assessed functionally, for their ability to induce cell death (cytotoxicity). Experiments conducted were similar to those described in Examples 11-15.

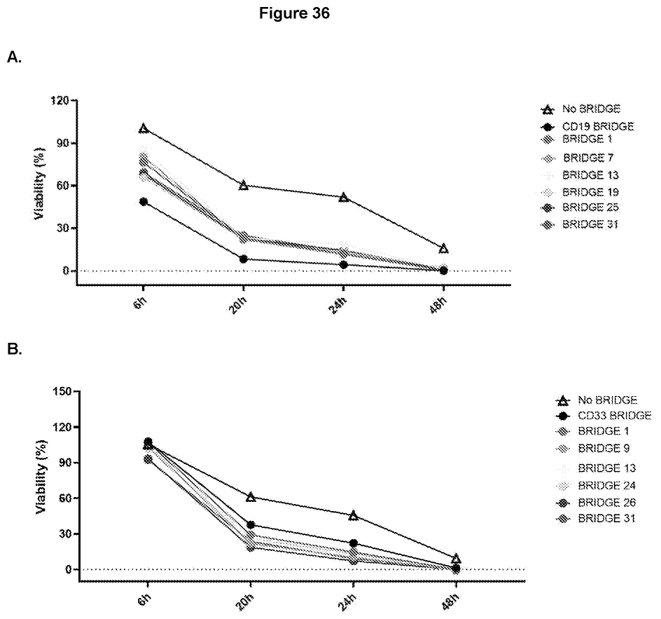

All bridging molecules tested demonstrated an ability to induce cell killing. Thus, the data indicate that the bridging molecule successfully re-directed anti-nfP2X₇ CAR T cells to either CD19 or CD33 antigen expressed by JeKo-1 cells or MOLM-13 cells, respectively. Representative data are shown in FIG. 36 (A: showing cell killing of JeKo-1 cells by anti-CD19 bridging molecule+anti-nfP2X₇ CAR T cells; B: showing cell killing of MOLM-13 cells by anti-CD33 bridging molecule+anti-nfP2X₇ CAR T cells).

### Example 17—In Vivo Efficacy of Two Component Therapeutic System of the Invention

6-8-week-old NSG mice were inoculated with the single cell sorted reporter cell line JeKo-1_LUC_eGFP at 1×10⁶ cells per mouse via tail vein injection on day 0. On day 7, mice underwent bioluminescence imaging (BLI) to determine JeKo-1 engraftment. Further, BLI was used to quantify the leukaemic load and treatment was initiated on day 7.

Mice were divided into the following treatment groups:
1. Tumour only (PBS)
2. Activated untransduced T cells
3. Bridging molecule (anti-CD19 Fab based on tafasitamab with E200 epitope)
4. Activated untransduced T cells+bridging molecule
5. T cells expressing 3rd generation anti-CD19-CAR (antigen binding domain
derived from FMC63 and having general CAR architecture of: CD8-CD28-41 BB-CD3ζ)
6. T cells expressing anti-nfP2X₇ receptor CAR 1 (antigen binding domain 2-2-1)
7. T cells expressing CAR1+bridging molecule
8. T cells expressing anti-nfP2X₇ receptor CAR2 (anti-nfP2X₇ receptor CAR antigen
binding domain 2-2-1)
9. T cells expressing CAR2+bridging molecule
10. PBS control (duplicate)

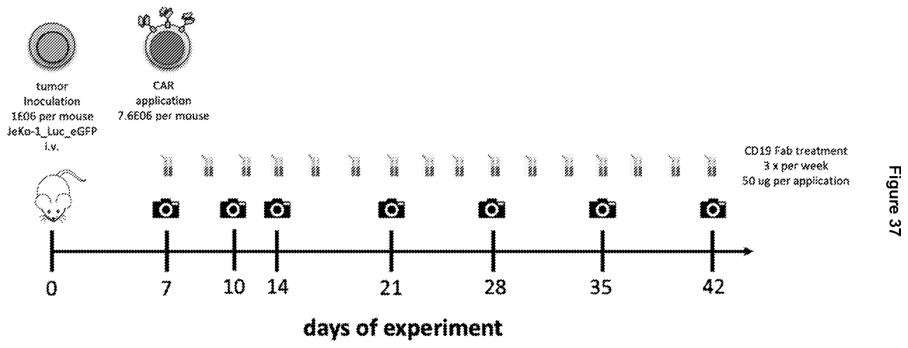

On day 7, intraperitoneal administration of bridging molecule was commenced at a dose regimen of 3× per week at 50 μg per mouse in the indicated groups. Leukaemic burden was evaluated via BLI at the indicated time points and mouse blood was analysed on day 42 to detect cancer cells and immune cells in the circulating peripheral blood. The experimental design is illustrated in FIG. 37.

The bridging molecule used comprised similar architecture to the molecules described in Example 14 (ie, having a targeting moiety comprised of a Fab derived from tafasitamab), and a tumour-specific antigen epitope moiety comprising an E200 peptide based on the SEQ ID NO: 143 and 145.

The positive control (group 5) comprised T cells expressing a 3^{rd} generation anti-CD19 CAR. The CAR comprised an antigen binding domain derived from FMC63 as described elsewhere herein (eg SEQ ID NO: 31 without the 6×HIStag and 32 in light-heavy orientation) and having the domains CD8-CD8TM-CD28-41BB-CD3zeta, a commonly used conventional CAR domain structure.

The ability of the bridging molecule to redirect T cells expressing two different nfP2X₇ receptor CARs was assessed. Briefly nfP2X₇ receptor CAR1 and nfP2X₇ receptor CAR2 comprised the general structure of a CAR, as described above in example 8 (ie having an antigen binding domain comprising the 2-2-1 sdAb, capable of binding an E200 epitope).

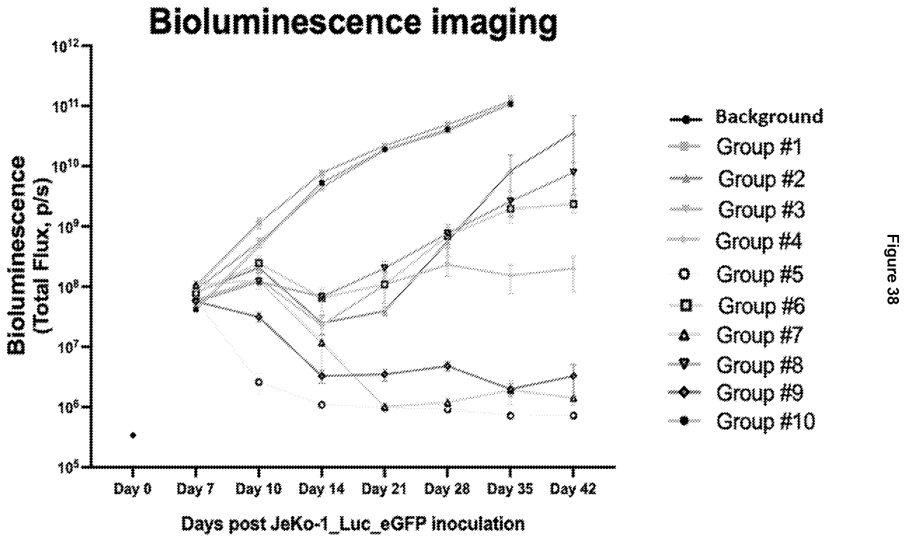

FIG. 38 shows bioluminescence (as a marker of tumour burden) for each of the treatment groups. The results indicate that mice administered T cells expressing anti-nfP2X₇ receptor CAR+anti-CD19 bridging molecule, had similarly low levels of tumour burden as compared to mice that received T cells expressing an anti-CD19 CAR.

These results demonstrate that in an in vivo setting, the bridging molecules of the present invention can be used to redirect a CAR T cell to bind to an alternative target. In this case, CAR T cells directed to nfP2X₇ receptor were successfully redirected to the CD19 antigen on cancer cells and achieved similar levels of therapeutic efficacy as CAR T cells with an antigen binding domain for binding CD19.

1. A two component therapeutic comprising:
(a) an immune cell or progenitor thereof, expressing a receptor comprising an antigen-recognition domain and a signalling domain, wherein the antigen-recognition domain binds to a tumour-specific antigen expressed on a cell surface; and
(b) a bridging molecule comprising:
(i) a targeting moiety that binds to a cell surface molecule on a target cell; and
(ii) a tumour-specific antigen epitope moiety that is bound by the antigen recognition domain.

2. A two component therapeutic of claim 1, wherein the tumour-specific antigen is an antigen expressed on a solid tumour.

3. A two component therapeutic of claim 1, wherein the tumour-specific antigen is any one of nfP2X₇, EGFRvIII or CLDN6.

4. A two component therapeutic of claim 1, wherein the tumour-specific antigen epitope moiety is a dysfunctional P2X₇ receptor epitope moiety.

5.-6. (canceled)

7. The therapeutic according to claim 1, wherein the bridging molecule is a polypeptide, or antibody or antibody fragment.

8. The therapeutic according to claim 7, wherein the polypeptide is expressed by the immune cell or progenitor thereof.

9. (canceled)

10. An immune cell, or progenitor thereof comprising:
(i) a chimeric antigen receptor comprising an antigen-recognition domain that binds a first tumour antigen, preferably wherein the first tumour antigen is a dysfunctional P2X₇ receptor, and
(ii) an inducible expression construct encoding a bridging molecule in the form of a fusion protein comprising (a) an antibody, or antigen binding fragment thereof, that binds a second tumour antigen, and (b) a peptide or a fragment of the first tumour antigen, preferably the dysfunctional P2X₇ receptor.

11. A bridging molecule comprising:
(i) a targeting moiety that binds to a cell surface molecule on a target cell; and
(ii) a tumour-specific antigen epitope moiety, preferably a dysfunctional P2X₇ receptor epitope moiety, that is bound by an antigen recognition domain.

12. (canceled)

13. A nucleic acid comprising a nucleotide sequence encoding a bridging molecule as described in claim 1.

14. The nucleic acid of claim 13, wherein the nucleic acid comprises a first nucleotide sequence encoding the targeting moiety and a second nucleotide sequence encoding the tumour-specific antigen epitope moiety, preferably a dysfunctional P2X₇ receptor epitope moiety.

15. A vector or expression construct comprising a nucleic acid of claim 14.

16. (canceled)

17. A method of treating a disorder in a subject, the method comprising administering to the subject:
(a) a cell expressing a receptor comprising an antigen-recognition domain and a signalling domain, wherein the antigen-recognition domain binds to a tumour-specific antigen, preferably a dysfunctional P2X₇ receptor, expressed on a cell surface; and
(b) a bridging molecule comprising:
(i) a targeting moiety that binds to a cell surface molecule on a target cell; and
(ii) a tumour-specific antigen epitope moiety, preferably a dysfunctional P2X₇ receptor epitope moiety, that is bound by the antigen recognition domain,
thereby treating the disorder in the subject.

18. A method of treating a disorder of claim 17, wherein the disorder is cancer.

19. A method of killing a target cell, the method including exposing the target cell to:
(a) an immune cell expressing a receptor comprising an antigen-recognition domain and a signalling domain, wherein the antigen-recognition domain binds to a tumour-specific antigen, preferably a dysfunctional P2X₇ receptor, expressed on a cell surface; and
(b) a bridging molecule comprising:
(i) a targeting moiety that binds to a cell surface molecule on the target cell; and
(ii) a tumour-specific antigen epitope moiety, preferably a dysfunctional P2X₇ receptor epitope moiety, that is bound by the antigen recognition domain,
thereby killing the target cell.

20. The method of claim 19, wherein the target cell is a cancer cell, or a cell on which an MHC I or II molecule presents a peptide from an infectious agent.

21. The method of claim 19, wherein the target cell does not express dysfunctional P2X₇ receptor.

22. The method of claim 17, wherein 2 or more bridging molecules are administered to a subject, each bridging molecule comprising a targeting moiety that binds to a different cell surface molecule on a target cell.

23. (canceled)

24. The method of claim 17, wherein the bridging molecule is a polypeptide encoded by a nucleic acid sequence that is expressed by the immune cell expressing the chimeric antigen receptor.

25. (canceled)

26. The therapeutic of claim 1, wherein the bridging molecule comprises targeting moieties for more than one class of cell surface molecule on a target cell.

27. The therapeutic of claim 1, wherein the bridging molecule comprises targeting moieties for more than one epitope of the same cell surface molecule on a target cell.

28. The therapeutic of claim 1, wherein the antigen-recognition domain of the CAR binds to an epitope associated with an adenosine triphosphate (ATP)-binding site of the dysfunctional P2X₇ receptor.

29. The therapeutic of claim 28, wherein the dysfunctional P2X₇ receptor has a reduced capacity to bind ATP at the ATP-binding site compared to an ATP-binding capacity of a functional P2X₇ receptor.

30.-42. (canceled)

43. The therapeutic of claim 11, wherein the targeting moiety of the bridging molecule comprises a ligand or binding partner for a protein or receptor present on the target cell surface.

44.-49. (canceled)

50. The therapeutic of claim 1, wherein the cell surface molecule is a tumour-associated antigen.

51. The therapeutic of claim 50, wherein the tumour associated antigen is selected from the group consisting of: CD33 (Siglec-3), CD123 (IL3RA), CD135 (FLT-3), CD44 (HCAM), CD44V6, CD47, CD184 (CXCR4), CLEC12A (CLL1), LeY, FRp, MICA/B, CD305 (LAIR-1), CD366 (TIM-3), CD96 (TACTILE), CD133, CD56, CD29 (ITGB1), CD44 (HCAM), CD47 (IAP), CD66 (CEA), CD112 (Nectin2), CD117 (c-Kit), CD133, CD146 (MCAM), CD155 (PVR), CD171 (LI CAM), CD200 (OX-2), CD221 (IGF1), CD227 (MUC1), CD243 (MRD1), CD246 (ALK), CD271 (LNGFR), CD19, CD20, GD2, CD276, PSMA and EGFR.

52.-54. (canceled)

55. The therapeutic of claim 1, wherein the dysfunctional P2X₇ receptor epitope moiety is bound by an antibody that binds to dysfunctional P2X₇ receptors, but is not bound by antibodies which bind to functional P2X₇ receptors.

56. The therapeutic of claim 4, wherein the bridging molecule comprises 2 or more dysfunctional P2X₇ receptor epitope moieties.

57.-60. (canceled)
